(19) United States
(12) Patent Application Publication
(10) Pub. No.: US 2026/0234590 A1
(43) Pub. Date: Aug. 13, 2026

(54) ENGINEERED GLUCOCEREBROSIDASE VARIANTS

(71) Applicant: Embold Therapeutics, Inc., Redwood City, CA (US)

(72) Inventors: Jacob Hansen Artz, Black Hawk, CO (US); Rachel Cathleen Botham, Burlingame, CA (US); Xingxiu Chen, Redwood City, CA (US); Victorino Miguel Francisco, San Jose, CA (US); William Casey Hallows, Redwood City, CA (US); Jeremiah Dallas Heredia, Sunnyvale, CA (US); Londen Christine Johnson, Santa Cruz, CA (US); Douglas Chamberlain Millar, Berkeley, CA (US); Ouma Onguka, Redwood City, CA (US); Christopher Edward Schmitt, San Francisco, CA (US); Adam P. Silverman, San Carlos, CA (US)

(21) Appl. No.: 19/153,026

(22) PCT Filed: Feb. 1, 2024
(86) PCT No.: PCT/US2024/014039

§ 371 Date: Aug. 1, 2025

Related U.S. Application Data
(60) Provisional application No. 63/482,947, filed on Feb. 2, 2023.
(60) Provisional application No. 63/502,058, filed on May 12, 2023.

(51) Int. Cl. C12N 9/42; A61K 38/47; A61K 48/00; A61P 3/00

(52) U.S. Cl. CPC C12N 9/2434; A61K 38/47; A61K 48/005; A61P 3/00; C12Y 302/01045

(57) ABSTRACT

The present disclosure provides engineered glucocerebrosidases, polynucleotides encoding the engineered glucocerebrosidases, and methods of treating a deficiency in glucocerebroside activity using the engineered glucocerebrosidases or the recombinant polynucleotides encoding the engineered glucocerebrosidases.

### CROSS-REFERENCE TO RELATED APPLICATIONS

This application claims the benefit of U.S. Provisional Application 63/482,947, filed Feb. 2, 2023, and U.S. Provisional Application 63/502,058, filed May 12, 2023, the contents of all of which are incorporated by reference herein.

### REFERENCE TO SEQUENCE LISTING, TABLE OR COMPUTER PROGRAM

The Sequence Listing concurrently submitted herewith as file name CX7-244WO3_ST26, created on Jan. 13, 2024, with a file size of 3,846,697 bytes, is part of the specification and is incorporated by reference herein.

### TECHNICAL FIELD

The present disclosure relates to engineered β-glucocerebrosidase (GCase) polypeptides, polynucleotides encoding the engineered β-glucocerebrosidase polypeptides, and uses of the engineered polypeptides or recombinant polynucleotides, or compositions thereof, in therapeutic applications.

### BACKGROUND

Gaucher disease (GD) is a lipid storage disease characterized by the accumulation of glucocerebroside in cells of the patient, including the macrophage-monocyte system. GD is caused by mutations leading to deficiency in the activity of the enzyme β-Glucocerebrosidase (GCase), an enzyme with glucosylceramidase activity (EC 3 2 1.45) that hydrolyses the beta-glycosidic linkage of glucocerebroside (glucosylceramide or GL 1) to glucose and ceramide. The monocyte-macrophage system is particularly afflicted in the disease due to the role of macrophages in the removal of dead and dying red and white blood cells whose membranes are rich in GL 1. This leads to accumulation of GL1 in these macrophages, also referred to as Gaucher cells. These Gaucher cells infiltrate the bone marrow, spleen, liver and other organs, leading to organomegaly in addition to a heightened inflammatory state of the macrophage-monocyte system. The deficiency in GCase is generally attributed to mutations in the GBA1 gene that impair activity and/or production of the enzyme. GD is a recessive genetic disorder.

GD manifests generally in three primary forms. GD type 1 (GD1), also referred to as non-neuropathic GD, does not involve the central nervous system and is the most common form. Individuals with GD type 1 show easy bruising, chronic fatigue, enlarged liver or spleen, and bone pain or degeneration. Some of the symptoms are attributable to low levels of platelets (thrombocytopenia) and low levels of circulating blood cells (anemia).

GD type 2 (GD2), also referred to as acute neuronopathic GD, manifests in newborns and infants, and is characterized by neurological complications due to abnormal accumulation of glucocerebroside and the deacylated product of glucocerebroside, glucosphingosine, in the central nervous system. Affected infants may display loss of motor skills, low muscle tone, and involuntary muscle spasms, resulting in slow and stiff movement of limbs and crossed eyes. Patients with GD type 2 may also have anemia and thrombocytopenia. GD type 2 often progresses into life threatening complications, including respiratory distress. Severely affected newborns show skin abnormalities, and may die within the first few weeks of life. Children with GD type 2 have reduced life span of about 1-3 years.

A perinatal-lethal form or fetal/neonatal GD is a rare form of GD type 2, occurring in less than 5% of GD patients. This GD type is severe and associated with death before 3 months of age or in the womb. The fetus/newborn may present with widespread swelling of the skin (edema or anasarca) leading to fluid buildup in the heart, skin, or lungs (hydrops fetalis). Other symptoms include bleeding within the skull (intracranial hemorrhage), scaling of the skin (non-bullous ichthyosiform erythroderma) with a reddish appearance, and contraction of the joints in fixed, bent position (arthrogryposis multiplex congenita).

GD type 3 (GD3), also referred to as chronic neuronopathic GD, occurs during the first decade of life. In addition to the blood and bone abnormalities, affected individuals develop neurological complications that develop and progress slower than in GD type 2. Neurological complications include mental deterioration, involuntary movements (ataxia); and muscle spasms of limbs or entire body (myoclonic seizures). A significant number of patients also develop pulmonary disease, including interstitial lung disease. There can be wide variability in presentation and clinical course among patients with type 3 Gaucher disease. Some affected patients may live into their teens and early 20s, while others have lived for much longer (30s and 40s). With increasing difficulties, affected individuals may require assistance to fulfill the task of daily living (for example, with eating, bathing, and ambulation).

Gaucher disease type 1 (GD1) is treatable with enzyme replacement therapy (ERT) or substrate reduction therapy (SRT). ERT uses recombinant forms of human β-glucocerebrosidase, which are administered by intravenous infusion typically every two weeks. ERTs approved for treating GD include Cerezyme® (imiglucerase), VPRIV® (velaglucerase alfa), and Elelyso® (taliglucerase alfa). The amino acid composition of imiglucerase and taliglucerase alfa differ from human GCase, while velaglucerase has the same amino acid sequence found in humans. Taliglucerase alfa also differs from velaglucerase alfa and imiglucerase in its glycosylation due to its production in plants. While ERT results in clinically significant reduction of GD symptoms, some patients can develop antibodies against the recombinant enzyme, thereby reducing its effectiveness.

SRT reduces buildup of toxic GL1 by targeting for inhibition the enzyme UDP-glucose ceramide glucosyltransferase, the first enzyme in the pathway for glycosylating sphingolipids. This enzyme is responsible for synthesis of GL 1 and other glycosphingolipids. Approved SRTs include miglustat (Zavesca®) and eliglustat (Cerdelga®). Miglustat is used primarily as a second-line treatment for patients who are unable to receive ERTs or are intolerant of them.

ERTs are effective in treating mild forms of GD and improving the quality of life by alleviating the visceral and hematologic aspects of the disease. However, immune response against the administered enzyme can reduce its effectiveness, and disease progression involving the central nervous system are not treated by ERT.

Accordingly, there is a need for improved and effective therapies for the treatment of GD, one that increases the availability of functioning GCase to affected target organs, which would avoid the need for frequent intravenous injections of GCase formulations approved for treating GD.

### SUMMARY

The present disclosure provides engineered glucocerebrosidases, recombinant polynucleotides encoding the engineered glucocerebrosidases, and pharmaceutical compositions comprising the engineered glucocerebrosidases or recombinant polynucleotides. The present disclosure further provides methods or uses of the engineered glucocerebrosidases or recombinant polynucleotides for treating a condition or disease associated with a deficiency in glucocerebrosidase activity, such as Gaucher disease.

In one aspect, an engineered glucocerebrosidase, or a functional fragment thereof, comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 40-536 of an even-numbered SEQ ID NO. of SEQ ID NOs: 2-1648, or to a reference sequence corresponding to an even-numbered SEQ ID NO. of SEQ ID NOs: 2-1648, wherein the amino acid sequence comprises one or more substitutions relative to a reference sequence corresponding to residues 40-536 of SEQ ID NO: 2, 54, 220, 984, or 1150, or relative to a reference sequence corresponding to SEQ ID NO: 2, 54, 220, 984, or 1150.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 40-536 of SEQ ID NO: 2, 54, 220, 984, or 1150, or to a reference sequence corresponding to SEQ ID NO: 2, 54, 220, 984, or 1150, wherein the amino acid sequence comprises one or more substitutions relative to the reference sequence corresponding to residues 40-536 of SEQ ID NO: 2, 54, 220, 984, or 1150, or relative to the reference sequence corresponding to SEQ ID NO: 2, 54, 220, 984, or 1150.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 2, or to the reference sequence corresponding to SEQ ID NO: 2, wherein the amino acid sequence comprises one or more substitutions relative to the reference sequence corresponding to residues 40-536 of SEQ ID NO: 2, or relative to the reference sequence corresponding to SEQ ID NO: 2.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, 220, 984, or 1150, to the reference sequence corresponding to SEQ ID NO: 54, 220, 984, or 1150, wherein the amino acid sequence comprises one or more substitutions relative to the reference sequence corresponding to residues 40-536 of SEQ ID NO: 2, or relative to the reference sequence corresponding to SEQ ID NO: 2.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 40-536 of an even-numbered SEQ ID NO. of SEQ ID NOs: 4-1648, or to a reference sequence corresponding to an even-numbered SEQ ID NO. of SEQ ID NOs: 4-1648, wherein the amino acid sequence comprises one or more substitutions relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution at amino acid position 23, 29, 35, 40, 41, 42, 44, 46, 50, 51, 53, 59, 63, 65, 68, 69, 70, 71, 75, 77, 78, 79, 82, 84, 86, 88, 89, 90, 91, 92, 93, 94, 95, 96, 97, 98, 99, 100, 101, 102, 103, 104, 105, 106, 107, 108, 109, 110, 111, 113, 114, 115, 116, 117, 133, 134, 137, 138, 141, 142, 144, 150, 151, 154, 169, 171, 172, 175, 180, 182, 184, 186, 189, 190, 191, 194, 195, 201, 202, 204, 206, 207, 211, 212, 214, 220, 222, 224, 230, 233, 235, 236, 239, 243, 246, 258, 260, 261, 262, 263, 264, 265, 271, 276, 298, 299, 301, 314, 315, 316, 319, 325, 329, 332, 333, 335, 336, 337, 338, 339, 341, 342, 359, 360, 361, 368, 370, 372, 373, 374, 382, 385, 386, 390, 400, 404, 408, 412, 413, 415, 434, 445, 446, 449, 459, 467, 468, 469, 471, 473, 474, 476, 477, 478, 479, 480, 481, 484, 485, 487, 489, 490, 493, 494, 495, 497, 502, 504, 505, 506, 507, 508, 509, 510, 512, 514, 515, 516, 518, 519, 520, 522, 524, 525, 529, 532, 533, 534, or 536, or combinations thereof, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution at amino acid position 65, 68, 70, 99, 154, 195, 207, 214, 230, 263, 319, 337, 361, 374, 408, 471, or 489, or combinations thereof, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set at amino acid position(s) 265/372/489, 243, 23, 109, 265, 109/195/243, 23/70/207/214/265, 23/70, 23/214/243/265, 65/70/109/214/319/372/481, 23/265/319, 23/65/70/141/319/481, 23/265, 372, 214, 23/65/70/109/141/214, 319, 65/372, 109/195, 23/65, 207/265/319, 109/207/214/372, 23/319/372/489, 65/109/214/243, 23/141/195, 70/207/214/489, 65/70/265/319/372, 214/265, 265/319, 65/243, 65/70/195, 489, 23/65/141, 23/372, 70/141, 23/195, 214/319, 23/207/214/243/265/481, 65/214/243/265/319, 214/243, 243/265, 265/489, 214/243/265, 207/243/372, 195/243, 65/481, 207/214/243/319, 70/141/195/265, 23/265/372, 46/99/134/230/373/449, 68/211/230, 373/449, 46/68/336, 68/230/314/373, 68, 68/336/373, 99/314, 46, 46/230/261, 373, 230/314, 68/211, 68/336/449, 46/336, 336/449, 46/68/336/373, 46/99, 99/261, 99, 68/134, 46/68/134/230/336/373, 46/68, 68/99/211/230, 99/134, 68/99/336, 449, 68/99, 46/230/336, 68/154/449, 336/373/449, 134/336, 68/99/230, 46/68/134/154, 68/99/230/373/449, 134, 46/68/154/211/230/261/336, 68/99/154/261/336, 230/449, 46/68/314, 154, 68/449, 99/449, 68/154/230/314/336/449, 46/68/154/314/336/373, 46/230, 336, or 46/99/230/373, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set at amino acid position(s) the amino acid sequence of the engineered glucocerebrosidase comprises at least a substitution or substitution set at amino acid position(s) 65/68/70/99/154/195/207/214/230/263/319/337/361/374/408/471/489, 65/68/70/99/154/195/207/214/230/263/319/337/361/374/408, 65/68/154/195/214/230/319/337/374/408, 65/68/70/99/154/195/207/214/230/263/374/408, 65/68/70/154/195/214/230/319/374/408/489, 65/68/70/99/154/195/207/214/230/319/337/361/374/408/471/489, 65/68/70/99/154/195/207/214/230/263/319/337/374/408, 65/68/154/195/207/214/230/263/319/337/361/374/408/471/489, 65/68/70/99/154/195/207/214/230/263/319/337/361/374/408/489, 65/68/70/99/154/195/207/214/230/374/408/471, 68/70/99/154/195/207/214/230/263/319/408, 65/68/70/207/230/374/408, 65/68/70/214/230/263/337/374/408, 214/230/263/374/408, 65/68/70/99/154/195/207/230/408, 65/68/70/99/154/207/230/319/408, 65/68/70/214/230/408, 65/68/70/195/214/230/263/361/471, 195/207/230/319/374/408/489, 65/68/70/214/230/408/489, 65/68/70/195/207/230/408, 207/230/319/374/408, 65/68/70/99/195/207/230/408, 195/214/230/263/408, 195/207/230/374/408, 65/68/70/99/154/195/214/408, 207/214/230/319/374/408, 207/214/230/374/408, 154/207/230/374/408/489, 207/230/263/408, 207/230/408/471, 207/230/374/408, 214/230/319/408, 214/230/374/408/489, 207/230/408, 65/68/70/154/207/408, 207/230/263/337/408, 195/214/230/408, 207/230/408/489, 214/230/408, 230/361/408, 207/214/408, 214/263/408, 230/374/408, 207/408, 195/214/263, 374/408, or 408, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least one substitution set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set of an engineered glucocerebrosidase variant set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, or relative to the reference sequence corresponding to SEQ ID NO: 2.

In some embodiments, the engineered glucocerebrosidase comprises an amino acid sequence having 95%, 96%, 97%, 98%, 99%, or more sequence identity to a reference sequence comprising a substitution or substitution set of an engineered glucocerebrosidase variant set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, or relative to the reference sequence corresponding to SEQ ID NO: 2.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, 220, 984, or 1150, or to the reference sequence corresponding to SEQ ID NO: 54, 220, 984, or 1150.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 4-1648, or to the reference sequence corresponding to an even-numbered SEQ ID NO. of SEQ ID NOs: 4-1648.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, 220, 984, or 1150, or to the reference sequence corresponding to SEQ ID NO: 54, 220, 984, or 1150, wherein the amino acid sequence comprises one or more substitutions relative to the reference sequence corresponding to residues 40-536 of SEQ ID NO: 54, 220, or 1150, or relative to the reference sequence corresponding to SEQ ID NO: 54, 220, 984, or 1150.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 4-1648, or to the reference sequence corresponding to an even-numbered SEQ ID NO. of SEQ ID NOs: 4-1648, wherein the amino acid sequence comprises one or more substitutions relative to the reference sequence corresponding to residues 40-536 of SEQ ID NO: 54, 220, 984, or 1150, or relative to the reference sequence corresponding to SEQ ID NO: 54, 220, 984 or 1150.

In some embodiments, the amino acid sequence of the engineered glucocerebrosidase comprises at least a substitution at amino acid position 23, 29, 35, 40, 41, 42, 44, 46, 50, 51, 53, 59, 63, 65, 68, 69, 70, 71, 75, 77, 78, 79, 82, 84, 86, 88, 89, 90, 91, 92, 93, 94, 95, 96, 97, 98, 99, 100, 101, 102, 103, 104, 105, 106, 107, 108, 109, 110, 111, 113, 114, 115, 116, 117, 133, 134, 137, 138, 141, 142, 144, 150, 151, 154, 169, 171, 172, 175, 180, 182, 184, 186, 189, 190, 191, 194, 195, 201, 202, 204, 206, 207, 211, 212, 214, 220, 222, 224, 230, 233, 235, 236, 239, 243, 246, 258, 260, 261, 262, 263, 264, 265, 271, 276, 298, 299, 301, 314, 315, 316, 319, 325, 329, 332, 333, 335, 336, 337, 338, 339, 341, 342, 359, 360, 361, 368, 370, 372, 373, 374, 382, 385, 386, 390, 400, 404, 408, 412, 413, 415, 434, 445, 446, 449, 459, 467, 468, 469, 471, 473, 474, 476, 477, 478, 479, 480, 481, 484, 485, 487, 489, 490, 493, 494, 495, 497, 502, 504, 505, 506, 507, 508, 509, 510, 512, 514, 515, 516, 518, 519, 520, 522, 524, 525, 529, 532, 533, 534, or 536, or combinations thereof, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

In some embodiments, the amino acid sequence of the engineered glucocerebrosidase comprises at least a substitution at amino acid position 65, 68, 70, 99, 154, 195, 207, 214, 230, 263, 319, 337, 361, 374, 408, 471, or 489, or combinations thereof, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, or to the reference sequence corresponding to SEQ ID NO: 54, wherein the amino acid sequence comprises one or more substitutions relative to the reference sequence corresponding to residues 40-536 of SEQ ID NO: 54, or relative to the reference sequence corresponding to SEQ ID NO: 54.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 200-922, or to the reference sequence corresponding to SEQ ID NO: 200-922, wherein the amino acid sequence comprises one or more substitutions relative to the reference sequence corresponding to residues 40-536 of SEQ ID NO: 54, or relative to the reference sequence corresponding to SEQ ID NO: 54.

In some embodiments, the amino acid sequence of the engineered glucocerebrosidase comprises at least a substitution or substitution set at amino acid position(s) 68/336, 65/99/319, 65/68/319, 68/319, 68, 65/319, 195, 65/154/319, 65/68/230, 65, 65/68/99/154/230/319, 68/230, 195/230, 65/230, 141/319, 154/336, 68/154, 68/195/230/319/336, 68/99/195/230/319, 68/195, 65/68, 68/141/230/319, 65/68/99/230/319, 68/99/141/319, 68/154/319, 141/154, 68/99, 99/230/319, 99, 230, 68/230/319, 154/319, 65/68/154/319, 141, 336, 141/230, 68/154/336, 65/68/99/319, 154, 319, 230/319, 68/154, 99/141/319, 509, 108, 108/117/509, 519, 108/519, 88, 117/413/509, 509/519, 413, 108/117/315, 117/271, 117/413/509/529, 413/509, 469, 524, 507, 525, 489, 104, 95, 359, 374,107, 89, 260, 110, 186, 370,191, 333, 189, 468, 473, 115, 53, 449, 518, 236, 481, 59/220, 258, 467, 51, 415, 103, 493, 78, 335, 520, 101, 341, 471, 211, 206, 93, 490, 71, 79, 180, 172, 207, 63, 142, 529, 77, 212, 474, 106, 386/507, 117, 373, 337, 107/246, 516, 233, 91, 194, 84, 361, 171, 536, 50, 116, 239, 44, 40, 113, 243, 169, 504, 505, 109, 479, 477, 97, 182, 111,508, 314, 102, 338, 96, 138, 42, 263, 222, 522, 265, 514, 515, 246, 46, 133, 332, 506, 512, 137, 190, 105, 301, 329, 339, or 151, wherein the amino acid positions are relative to the reference sequence corresponding to residues 40-536 of SEQ ID NO: 54, or relative to the reference sequence corresponding to SEQ ID NO: 54.

In some embodiments, the engineered glucocerebrosidase comprises an amino acid sequence having 95%, 96%, 97%, 98%, 99%, or more sequence identity to the reference sequence corresponding to residues 40-536 of SEQ ID NO: 220, or to the reference sequence corresponding to SEQ ID NO: 220, wherein the amino acid sequence comprises one or more substitutions relative to the reference sequence corresponding to residues 40-536 of SEQ ID NO: 220, or relative to the reference sequence corresponding to SEQ ID NO: 220.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 924-1020, or to the reference sequence corresponding to SEQ ID NO: 924-1020, wherein the amino acid sequence comprises one or more substitutions relative to the reference sequence corresponding to residues 40-536 of SEQ ID NO: 220, or relative to the reference sequence corresponding to SEQ ID NO: 220.

In some embodiments, the amino acid sequence of the engineered glucocerebrosidase comprises at least a substitution or substitution set at amino acid position(s) 65/195/361, 514, 79/195/263/361, 65/374, 79/195, 65/230, 65/233/263/337/359/361/374, 79/536, 79/230/233/239/359/361/471, 65/471, 374, 65/195/230/233/337/374/514, 65/195/233/359/536, 230, 65/195/263/276/359/361/374/471, 79, 195/230/233/337/374/536, 195/230, 195/230/337/359/361, 230/263, 65/230/233, 263, 195/230/337/361/374/471, 65/195, 374/471, 374/536, 239, 65, 230/233/536, 230/263/374, 65/195/230/263/337/361/374/471, 79/374, 65/361/374/471/536, 195, 233, 79/230/359/374, 536, 230/359/361/374/514, 65/79/230, 195/239/263/361/374/514, 195/263/374, 195/374, 65/230/233/361/536, 230/233/374/471, 65/233/359/361/471, 65/359/471, 65/79, or 65/359, wherein the amino acid positions are relative to the reference sequence corresponding to residues 40-536 of SEQ ID NO: 220, or relative to the reference sequence corresponding to SEQ ID NO: 220.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 984, or to the reference sequence corresponding to SEQ ID NO: 984, wherein the amino acid sequence comprises one or more substitutions relative to the reference sequence corresponding to residues 40-536 of SEQ ID NO: 984, or relative to the reference sequence corresponding to SEQ ID NO: 984.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 1022-1216, or to the reference sequence corresponding to SEQ ID NO: 1022-1216, wherein the amino acid sequence comprises one or more substitutions relative to the reference sequence corresponding to residues 40-536 of SEQ ID NO: 984, or relative to the reference sequence corresponding to SEQ ID NO: 984.

In some embodiments, the amino acid sequence of the engineered glucocerebrosidase comprises at least a substitution or substitution set at amino acid position(s) 359, 79/359, 79/97/182, 97/359, 97, 79/182, 201, 404, 484, 446, 494/495, 495, 412, 385, 100, 184, 82, 75, 71, 41, 99, 94, 92, 98, 114, 95, 102, 96, 105, 113, 325, 336, 262, 386, 360, 400, 342, 390, 264, 298, 382, 408, 449, 459, 434, 476,502, 29/534, 497, 485, 533, 536, 510, 532, 489, 534, 494, 182, 137, 204, 194, 202, 144, 142,175, 220, 150, or 206, wherein the amino acid positions are relative to the reference sequence corresponding to residues 40-536 of SEQ ID NO: 984, or relative to the reference sequence corresponding to SEQ ID NO: 984.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 1150, or to the reference sequence corresponding to SEQ ID NO: 1150, wherein the amino acid sequence comprises one or more substitutions relative to the reference sequence corresponding to residues 40-536 of SEQ ID NO: 1150, or relative to the reference sequence corresponding to SEQ ID NO: 1150.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 1218-1554, or to the reference sequence corresponding to SEQ ID NO: 1218-1554, wherein the amino acid sequence comprises one or more substitutions relative to the reference sequence corresponding to residues 40-536 of SEQ ID NO: 1150, or relative to the reference sequence corresponding to SEQ ID NO: 1150.

In some embodiments, the amino acid sequence of the engineered glucocerebrosidase comprises at least a substitution or substitution set at amino acid position(s) 96/194/382/484, 359/382/412/494/495, 92/96/175/182/382, 96/359/382/484/494/495, 92/175/194/359/382, 359/382/400/412, 92/96/182/382/400/484, 92/96/382/494/495, 92/96/175/382/494/495, 96/182/359/382/484, 92/96/382, 92/382/494/495/534, 92/96/382/400/484, 92/96/175/382, 92/175/382/400/484, 92/96/175/182/382/412/484, 92/96/201/382/484, 92/182/194/382, 92/96/175/194/359/382/400, 92/359/382, 96/382, 92/175/382/484/494/495, 96/201/359/382, 92, 194, 201, 359, 96, 92/96/382/400/484/495, 382/495, 382, 92/382, 71/175/325/385, 325/385, 71/325/502, 71/175/325/385/502, 202/325/385, 97/175/385/510, 325/385/510, 100/175/325, 71/175/325/360/385, 97/175/510, 71/97/100/175/325/385, 71/175/510, 71/97/325/502, 71/100/325/385, 202/385/489, 325/385/502, 71/100/325/385/510, 71/100/175/325/385, 385/510, 97/325/385, 71, 97/202/325/360/385/510, 502, 71/97/103/325, 100/325/385/502/510, 71/137/175/325/360/385/502, 71/276/325/385/510, 71/175/360/385/502, 100/175/325/385/489/502, 71/175/360/385/485/502, 95/113, 494/536, 95/99/113/182/184/264/386, 184/536, 95/142/144/182/184/386/449, 144/204/386/400/449, 113/449/536, 536, 95/386/536, 264, 99/113/386/449, 95/386, 95/99/204/386, 113/142/386/449, 182/184/264/494, 113/142/182/494, 113, 99/264/449, 95, 95/99, 99/113/449, 95/113/449/536, 95/99/113, 386/536, 99/182/184, 142/386/400/536, 35/264/449/536, 142/144/220, 99/142, 29/102/182/276/336/390/459/502/532, 41/262/336/502, 29/97/150/262/336/502/532, 29, 532, 102/390/459/502/532, 29/262/276/336/495, 29/182/262/459/532, 92/99/102/502, 29/97/102/182/502/532, 336/459/495/502/532, 150/182/336/390, 150/262/495, 29/495, 29/102/182/262/502, 29/336/459/502/532, 182/495/502/532, 102/336, 150/182/262/495, 459/495/502/532, 41/182/390/495, 29/41/182/336/495, 97/102/495, 182/262/495, 29/262/502, 29/102/502, 182/336/495, 29/150/182/262/459/495, 502/532, 386, 497, 99, 512, 69, 507, 445, 75, 487, 184, 90, 235, 434, 489, 86, 182, 446, 490, 299, 102, 230, 68, 141, 97, 316, 522, 224, 478, 480, 368, 412, 449, 265, or 494, wherein the amino acid positions are relative to the reference sequence corresponding to residues 40-536 of SEQ ID NO: 1150, or relative to the reference sequence corresponding to SEQ ID NO: 1150.

In some embodiments, the amino acid sequence of the engineered glucocerebrosidase comprises at least one substitution set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

In some embodiments, the amino acid sequence of the engineered glucocerebrosidase comprises at least a substitution or substitution set of an engineered glucocerebrosidase set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising a substitution or substitution set of an engineered glucocerebrosidase set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

In some embodiments, the amino acid sequence of the engineered glucocerebrosidase comprises residues 40-536 of an engineered glucocerebrosidase set forth in Tables 3.1, 3.2, 4.1, 4.2, 4.3, 4.4, 5.1, 6.1, 6.2, 12.1, 12.2, and 13.1, or an amino acid sequence comprising an engineered glucocerebrosidase set forth in Tables 3.1, 3.2, 4.1, 4.2, 4.3, 4.4, 5.1, 6.1, 6.2, 12.1, 12.2, and 13.1.

In some embodiments, the engineered glucocerebrosidase comprises an amino acid sequence comprising residues 40-536 of an even-numbered SEQ ID NO. of SEQ ID NOs: 4-1648, or comprises an even-numbered SEQ ID NO. of SEQ ID NOs: 4-1648, optionally wherein the amino acid sequence has 1, 2, 3, 4, 5, 6, 7, 8, 9, or up to 10 substitutions.

In some embodiments, the engineered glucocerebrosidase has glucocerebrosidase activity. In some embodiments, the engineered glucocerebrosidase has glucocerebrosidase activity and one or more improved properties compared to a reference glucocerebrosidase.

In some embodiments, the engineered glucocerebrosidase has glucocerebrosidase activity and an improved property selected from: i) increased enzymatic activity; ii) increased expression or expression efficiency; iii) increased stability at neutral or basic pH; iv) increased stability at acidic pH, v) increased stability to serum or plasma; vi) increased thermostability; vii) increased uptake and/or intracellular stability in cells such as macrophages and viii) reduced immunogenicity; or any combination of i), ii), iii), iv), v), vi), vii), and viii) as compared to a reference glucocerebrosidase having a sequence corresponding to residues 40-536 of SEQ ID NO: 2, 54, 220, 984, or 1150, or a sequence corresponding to SEQ ID NO: 2, 54, 220, 984, or 1150. In some embodiments, the improved property is in comparison to the reference glucocerebrosidase having a sequence corresponding to residues 40-536 of SEQ ID NO: 2, or to the sequence corresponding to SEQ ID NO: 2.

In a further aspect, the present disclosure provides recombinant polynucleotides encoding the engineered glucocerebrosidases. In some embodiments, the recombinant polynucleotide comprises a polynucleotide sequence encoding an engineered glucocerebrosidase of the present disclosure.

In some embodiments, the recombinant polynucleotide comprises a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 118-1608 of SEQ ID NO. 53, 219, 983, or 1149, or to a reference polynucleotide sequence corresponding to SEQ ID NO: 53, 219, 983, or 1149, wherein the recombinant polynucleotide encodes a glucocerebrosidase.

In some embodiments, the recombinant polynucleotide comprises a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 118-1608 of an odd-numbered SEQ ID NO. of SEQ ID NOs: 3-1647, or to a reference polynucleotide sequence corresponding to an odd-numbered SEQ ID NO. of SEQ ID NOs: 3-1647, wherein the recombinant polynucleotide encodes a glucocerebrosidase.

In some embodiments, the recombinant polynucleotide comprises a polynucleotide sequence codon-optimized for expression of the encoded glucocerebrosidase.

In some embodiments, the recombinant polynucleotide comprises a polynucleotide sequence comprising residues 118-1608 of an odd-numbered SEQ ID NO. of SEQ ID NOs: 3-1647, or a polynucleotide sequence comprising an odd-numbered SEQ ID NO. of SEQ ID NOs: 3-1647.

In some embodiments, the recombinant polynucleotide comprises a polynucleotide sequence comprising nucleotide residues 118-1608 of SEQ ID NO: 53, 219, 983, or 1149, or comprises SEQ ID NO: 53, 219, 983, or 1149.

In a further aspect, the recombinant polynucleotides are provided in an expression vector. In some embodiments, an expression vector comprises a recombinant polynucleotide encoding an engineered glucocerebrosidase described herein.

In some embodiments, the recombinant polynucleotide in the expression vector is operably linked to a control sequence. In some embodiments, the control sequence comprises at least a promoter, particularly a heterologous promoter. In some embodiments, the expression vector comprises a viral vector, particularly for introduction of a recombinant polynucleotide encoding an engineered glucocerebrosidase into cells or administration into a subject.

In another aspect, the present disclosure provides host cells comprising an expression vector comprising a recombinant polynucleotide encoding an engineered glucocerebrosidase disclosed herein. In some embodiments, the host cell is a bacterial cell, fungal cell, insect cell, or mammalian cell, in particular a human cell.

In another aspect, the present disclosure further provides a method of producing an engineered glucocerebrosidase, comprising culturing a host cell under suitable culture conditions such that the engineered glucocerebrosidase is expressed or produced. In some embodiments, the method further comprises a step of recovering the engineered glucocerebrosidase from the culture media and/or host cells. In some embodiments, the method further comprises purifying the engineered glucocerebrosidase.

In another aspect, the engineered glucocerebrosidase or the recombinant polynucleotide is formulated as a pharmaceutical composition. In some embodiments, the pharmaceutical composition comprises an engineered glucocerebrosidase or a recombinant polynucleotide encoding the engineered glucocerebrosidase, or an expression vector thereof, and a pharmaceutically acceptable excipient or carrier. In some embodiments, the pharmaceutical composition is suitable for parenteral injection or infusion to a human.

In some embodiments, the pharmaceutical composition is a gene therapy composition comprising a recombinant polynucleotide encoding an engineered glucocerebrosidase. In some embodiments, the gene therapy composition comprises a viral vector comprising a recombinant polynucleotide encoding an engineered glucocerebrosidase. In some embodiments, the viral vector is adenovirus, adeno-associated virus, lentivirus, retrovirus, or herpes virus vector.

In some embodiments, the gene therapy composition comprises a recombinant polynucleotide encoding an engineered glucocerebrosidase formulated with liposomes, cationic polymers, dendrimers, or conjugated to a cell-penetrating peptide.

In some embodiments, an engineered glucocerebrosidase or a recombinant polynucleotide encoding the engineered glucocerebrosidase is used for treating and/or preventing the symptoms of deficiency in glucocerebrosidase activity in a subject. In some embodiments, a method or use for treating a deficiency in glucocerebrosidase activity in a subject comprises administering to a subject in need thereof an effective amount of an engineered glucocerebrosidase, a recombinant polynucleotide encoding an engineered glucocerebrosidase, or a gene therapy composition comprising a recombinant polynucleotide encoding an engineered glucocerebrosidase described herein.

In some embodiments, use of an engineered glucocerebrosidase, or a recombinant polynucleotide encoding an engineered glucocerebrosidase or a gene therapy composition thereof is for the preparation of a medicament for treating a deficiency in glucocerebrosidase activity in a subject.

In some embodiments, the subject having a deficiency in glucocerebrosidase activity is afflicted with Gaucher disease or Parkinson's disease.

### BRIEF DESCRIPTION OF THE DRAWINGS

FIG. 1 provides graphs showing expression as determined by Western blot and the GCase Activity (4-MU-βGLU hydrolysis) in the supernatant of cultures of Expi293F (Panel A and Panel B), ExpiCHO (Panel C and Panel D) and HepG2 (Panel E and Panel F) for five GCase variants.

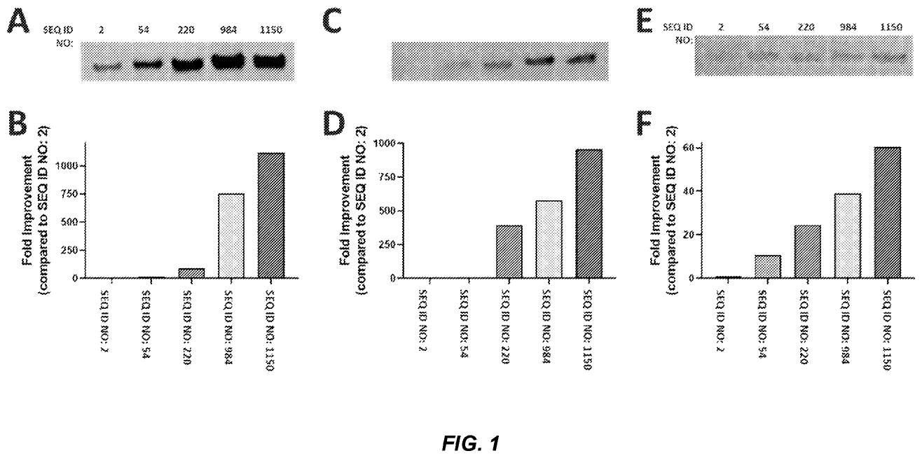

FIG. 2 provides a graph showing the remaining GCase Activity (4-MU-βGLU hydrolysis) of supernatants of eleven GCase variants following incubation with a neutral pH solution. GCase material used in experiment was derived from high throughput expression cultures of Expi293F transfected with DNA for the eleven GCase variants.

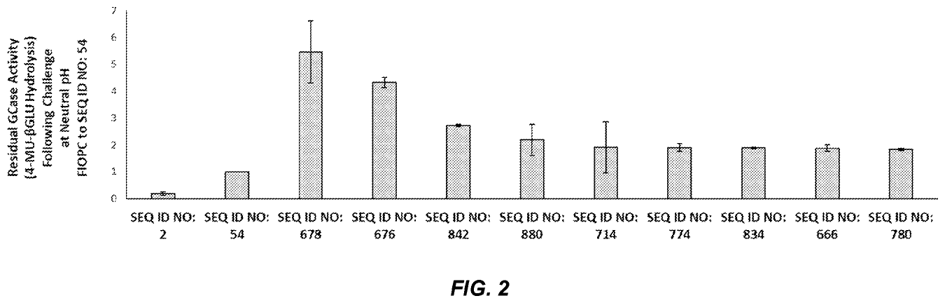

FIG. 3 provides a graph showing the remaining GCase Activity (4-MU-βGLU hydrolysis) of supernatants of eleven GCase variants following incubation at 47° C. for one hour. GCase material used in experiment was derived from high throughput expression cultures of Expi293F transfected with DNA for the eleven GCase variants.

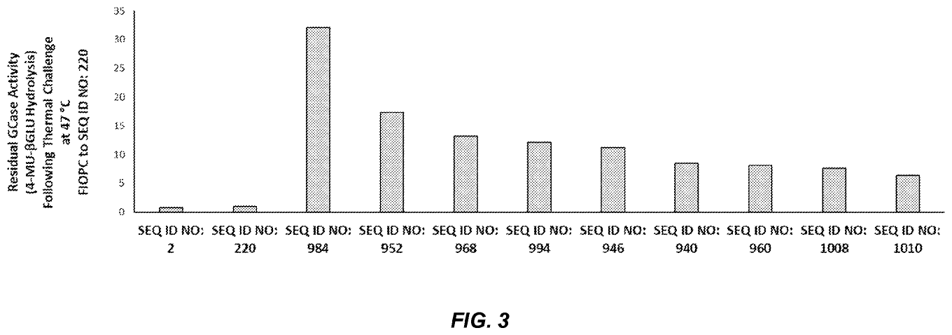

FIG. 4 provides a graph showing the remaining GCase Activity (4-MU-βGLU hydrolysis) of supernatants of eleven GCase variants following incubation with human serum at 37° C. for one hour. GCase material used in experiment was derived from high throughput expression cultures of Expi293F transfected with DNA for the eleven GCase variants.

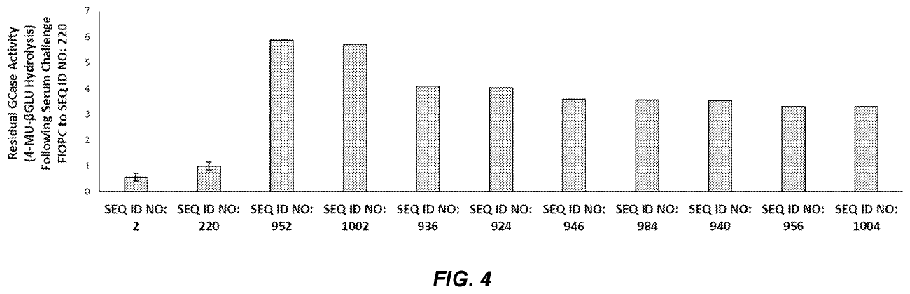

FIG. 5 provides graphs showing the cross-correction capacity of GCase variants, wherein HepG2 cells are transfected with DNA encoding for GCase SEQ ID NO: 2, 54, 220, or 984, and macrophages are co-cultured in a trans-well context. 4-MU-βGLU hydrolysis activity was assessed in each compartment (Panel A—HepG2 cell lysates; Panel B—secreted protein from HepG2 in the conditioned media; and Panel C—evidence of uptake in macrophage lysates) and normalized to the activity of GCase SEQ ID NO: 2.

FIG. 6 provides graphs showing the cross-correction capacity of GCase variants when delivered as vectorized AAV9 constructs, wherein HepG2 cells are transduced with AAV packaged DNA of SEQ ID NO: 1 or 983, and macrophages are co-cultured in a trans-well context. 4-MU-βGLU hydrolysis activity was assessed in each compartment (Panel A—HepG2 cell lysates; Panel B—secreted protein from HepG2 in the conditioned media; and Panel C—evidence of uptake in macrophage lysates). In the macrophages, GCase-induced clearance of GL 1 (Panel D), and lyso-GL1 (Panel E) was determined.

FIG. 7 provides graphs showing the stability and residual activity of two GCase variants when challenged with (Panel A) incubation in serum, (Panel B) incubation in artificial CSF, and (Panel C) when incubated for 1 hour across a thermal gradient, as described in Example 11.

FIG. 8 provides graphs showing the transduction capacity of GCase variants when delivered as vectorized AAV9 constructs, wherein HepG2 cells (Panel A—secreted protein; Panel B—lysates), Expi293F (Panel C—secreted protein; Panel D—lysates), and RAW264.7 (Panel E—secreted protein; Panel F—lysates) are transduced with DNA of SEQ ID NO: 1 or 983 in the presence of absence of etoposide; resulting GCase activity is measured using the standard 4-MU-βGLU hydrolysis activity in the conditioned media (Panel A, Panel C, Panel E) or in the lysates (Panel B, Panel D, Panel F) for each cell line.

FIG. 9 provides graphs showing the GCase activity tissues of WT or D409V/D409V mice following dosing with vehicle or AAV9 packaged SEQ ID NO: 1 or SEQ ID 983. GCase activity in the plasma (Panel A—pre-dosing; Panel B—at 14 days; and Panel C—at 28 days), in the liver (Panel D), in the spleen (Panel E), and in the brain (Panel F), as described in Example 8. Data are represented at mean±/−standard deviation. Treatment groups were analyzed by one-way ANOVA multiple comparisons and significant differences are indicated: *: P<0.05; **: P<0.01; ***: P<0.005.

FIG. 10 provides graphs showing the GL1 abundance in tissues of WT or D409V/D409V mice following dosing with vehicle or AAV9 packaged SEQ ID NO: 1 or SEQ ID NO: 983. GL1 was measured in the liver (Panel A), in the spleen (Panel B), and in the brain (Panel C), as described in Example 8. GL 1 is the sum of analyzed 16:0, 18:0, and 24:1 isoforms. Data are represented at mean±/−standard deviation. Treatment groups were analyzed by one-way ANOVA multiple comparisons and significant differences are indicated: *: P<0.05; **: P<0.01; ****: P<0.001.

### DETAILED DESCRIPTION

The present disclosure provides engineered glucocerebrosidase variants. In some embodiments, the engineered glucocerebrosidase variants have glucocerebrosidase activity and is characterized by an improved enzyme property, including but not limited to, increased enzyme activity, increased expression efficiency, increased stability at neutral or basic pH, increased stability at acid pH, increased serum stability, enhanced uptake into cells, such as macrophages, and/or reduced immunogenicity. Further provided are recombinant polynucleotides encoding the engineered glucocerebrosidases and use of the engineered glucocerebrosidases, e.g., as enzyme replacement therapy, or the recombinant polynucleotides, e.g., as gene therapy, for treating a deficiency in glucocerebrosidase, such as Gaucher disease.

### Abbreviations and Definitions

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art.

It is to be understood that the present invention is not limited to the particular methodology, protocols, and reagents described, as these may vary, depending upon the context they are used by those of skill in the art. Accordingly, the terms defined immediately below are more fully described by reference to the application as a whole.

As used herein, the singular “a,” “an,” and “the” include the plural references, unless the context clearly indicates otherwise.

In addition, the term “comprising” and its cognates are used in their inclusive sense (i.e., equivalent to the term “including” and its corresponding cognates).

It is to be further understood that where description of embodiments use the term “comprising” and its cognates, the embodiments can also be described using language “consisting essentially of” or “consisting of.”

Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

The term “about” means an acceptable error for a particular value. In some instances, “about” means within 0.05%, 0.5%, 1.0%, or 2.0%, of a given value range. In some instances, “about” means within 1, 2, 3, or 4 standard deviations of a given value. In some instances, “about” encompasses values that are within 2.5%, 3%, 3.5%, 4%, 4.5%, 5%, 5.5%, 6%, 6.5%, 7%, 7.5%, 8%, 8.5%, 9%, 9.5%, or 10% of a given value.

Furthermore, the headings provided herein are not limitations of the various aspects or embodiments of the invention which can be had by reference to the application as a whole. Accordingly, the terms defined immediately below are more fully defined by reference to the application as a whole. Nonetheless, in order to facilitate understanding of the invention, a number of terms are defined below.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Gaucher disease” or “GD” refers to a metabolic disorder associated with a deficiency in enzyme glucocerebrosidase, which results in the accumulation of certain lipids, particularly the glycolipid glucocerebroside, in cells and certain tissues and organs.

“Glucocerebrosidase,” “β-glucocerebrosidase,” “acid β-glucosidase,” and “GCase” are used interchangeably herein to refer to a glycoside hydrolases with glucosylceramidase activity that cleaves the β-glycosyl linkage of glucocerebroside to produce glucose and ceramide. In humans, GCase is encoded by the GBA1 gene. In the lysosome, the wild-type GCase is membrane-associated and interacts with the activator protein saposin C (SapC). GCase enzyme is classified in EC 3.2.1.45.

“Protein,” “polypeptide,” and “peptide” are used interchangeably to denote a polymer of at least two amino acids covalently linked by an amide bond, regardless of length or post-translational modification (e.g., glycosylation or phosphorylation).

“Amino acids” are referred to herein by either their commonly known three-letter symbols or by the one-letter symbols recommended by IUPAC-IUB Biochemical Nomenclature Commission. The abbreviations used for the genetically encoded amino acids are conventional and are as follows: alanine (Ala or A), arginine (Arg or R), asparagine (Asn or N), aspartate (Asp or D), cysteine (Cys or C), glycine (Gly or G), glutamate (Glu or E), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (Cα). For example, whereas “Ala” designates alanine without specifying the configuration about the α carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

“Fusion protein,” and “chimeric protein” and “chimera” refer to hybrid proteins created through the joining of two or more polynucleotides that originally encode separate proteins. In some embodiments, fusion proteins are created by recombinant technology (e.g., molecular biology techniques known in the art).

“Mature protein” or “mature polypeptide” refers to the final processed biological protein or polypeptide or product.

“Pro-protein,” “pro-polypeptide,” or “pro-peptide” refers to a precursor protein, polypeptide, or peptide that is processed by post-translational modification, to form a biologically active protein, polypeptide, or peptide. In some embodiments, the post translational modification is a cleavage reaction to form the protein, polypeptide, or peptide. “Pro-enzyme” refers to a precursor polypeptide that is processed by post-translational modification, in particular a cleavage reaction, to form an active enzyme.

“Pre-pro-protein,” “pre-pro-polypeptide,” or “pre-pro-peptide” refers to a precursor protein, polypeptide, or peptide that includes a signal sequence or signal peptide and which can be processed by posttranslational modification, in particular a cleavage reaction, to generate a pro-protein, pro-polypeptide, or pro-peptide. Generally, a cleavage reaction removes a signal sequence to generate a pro-protein, pro-polypeptide, or pro-peptide. “Pre-pro-enzyme” refers to a precursor protein, polypeptide, or peptide that is processed by post-translational modification, in particular a cleavage reaction that removes a signal sequence, to form a pro-enzyme.

“Full-length” in context of a protein or polypeptide refers to the protein or polypeptide which is not processed to alter the amino acid sequence of the entire protein or polypeptide. For example, a full-length protein is the entire protein encoded in the corresponding mRNA.

“Polynucleotide” is used herein to denote a polymer comprising at least two nucleotides where the nucleotides are either deoxyribonucleotides or ribonucleotides or mixtures of deoxyribonucleotides and ribonucleotides. In some embodiments, the abbreviations used for the genetically encoding nucleosides are conventional and are as follow: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated. The term “DNA” refers to deoxyribonucleic acid. The term “RNA” refers to ribonucleic acid.

“Engineered,” “recombinant,” “non-naturally occurring,” and “variant,” when used with reference to a cell, a polynucleotide or a polypeptide refers to a material or a material corresponding to the natural or native form of the material that has been modified in a manner that would not otherwise exist in nature or is identical thereto but produced or derived from synthetic materials and/or by manipulation using recombinant techniques.

“Wild-type” and “naturally-occurring” refer to the form found in nature. For example, a wild-type polypeptide or polynucleotide sequence is a sequence present in an organism that can be isolated from a source in nature and which has not been intentionally modified by human manipulation.

“Coding sequence” refers to that part of a nucleic acid (e.g., a gene) that encodes an amino acid sequence of a polypeptide or protein.

“Percent (%) sequence identity” is used herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA, 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms, which are described by Altschul et al. (See, Altschul et al., J. Mol. Biol., 1990, 215:403-410; and Altschul et al., 1977, Nucleic Acids Res., 1977, 3389-3402, respectively). Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (See, Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (See, Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For instance, the phrase “a reference sequence corresponding to SEQ ID NO: 2, having a proline at the residue corresponding to X51” (or “a reference sequence corresponding to SEQ ID NO: 2, having a proline at the residue corresponding to position 51”) refers to a reference sequence in which the corresponding residue at position X51 in SEQ ID NO: 2 (e.g., a serine), has been changed to proline.

“Comparison window” refers to a conceptual segment of contiguous nucleotide positions or amino acids residues wherein a sequence may be compared to a reference sequence. In some embodiments, the comparison window is at least 15 to 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. In some embodiments, the comparison window can be longer than 15-20 contiguous residues, and includes, optionally 30, 40, 50, 100, or longer windows.

“Corresponding to,” “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered glucocerebrosidase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Mutation” refers to the alteration of a nucleic acid sequence. In some embodiments, mutations result in changes to the encoded polypeptide sequence (i.e., as compared to the original sequence without the mutation). In some embodiments, the mutation comprises a substitution, such that a different amino acid is produced. In some alternative embodiments, the mutation comprises an addition, such that an amino acid is added (e.g., insertion) to the original polypeptide sequence. In some further embodiments, the mutation comprises a deletion, such that an amino acid is deleted from the original polypeptide sequence. Any number of mutations may be present in a given sequence. In some embodiments, the “substitution” comprises the deletion of an amino acid, and can be denoted by “−” symbol.

“Amino acid difference” and “residue difference” refer to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X51 as compared to SEQ ID NO: 2” (or a “residue difference at position 51 as compared to SEQ ID NO: 2”) refers to a difference of the amino acid residue at the polypeptide position corresponding to position 51 of SEQ ID NO: 2. Thus, if the reference polypeptide of SEQ ID NO: 2 has a serine at position 51, then a “residue difference at position X51 as compared to SEQ ID NO: 2” refers to an amino acid substitution of any residue other than serine at the position of the polypeptide corresponding to position 51 of SEQ ID NO: 2. In some instances herein, the specific amino acid residue difference at a position is indicated as “XnY” where “Xn” specified the corresponding residue and position of the reference polypeptide (as described above), and “Y” is the single letter identifier of the amino acid found in the engineered polypeptide (i.e., the different residue than in the reference polypeptide). In some instances (e.g., in the Tables in the Examples), the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some embodiments, the amino acid difference, e.g., a substitution, is denoted by the abbreviation “nB,” without the identifier for the residue in the reference sequence. In some embodiments, the phrase “an amino acid residue nB” denotes the presence of the amino residue in the engineered polypeptide, which may or may not be a substitution in context of a reference sequence.

In some instances, a polypeptide of the present disclosure can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X46M/X46R, X46M/R, or 46M/R). The present disclosure includes engineered polypeptide sequences comprising one or more amino acid differences that include either/or both conservative and non-conservative amino acid substitutions, as well as insertions and deletions of amino acids in the sequence.

“Amino acid substitution set” and “substitution set” refers to a group of amino acid substitutions within a polypeptide sequence. In some embodiments, substitution sets comprise 2, 3, 4, 5, 6, 7, 8, 9, 10, 11, 12, 13, 14, 15, or more amino acid substitutions. In some embodiments, a substitution set refers to the set of amino acid substitutions that is present in any of the engineered glucocerebrodisase polypeptides, such as those listed in any of the Tables in the Examples. In these substitution sets, the individual substitutions are separated by a semicolon (“;”; e.g., K46R;P68V or abbreviated 46R;68V) or slash (“/”; e.g., K46R/P68V or abbreviated 46R/68V).

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered enzyme. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Functional fragment” or a “biologically active fragment” used interchangeably herein refers to a polypeptide that has an amino-terminal and/or carboxy-terminal deletion(s) and/or internal deletions, but where the remaining amino acid sequence is identical to the corresponding positions in the sequence to which it is being compared (e.g., a full-length engineered glucocerebrosidase of the present disclosure) and that retains substantially all of the activity of the full-length polypeptide.

“Isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The engineered glucocerebrosidase polypeptides may be present within a cell, present in the cellular medium, or prepared in various forms, such as lysates or isolated preparations. As such, in some embodiments, the engineered glucocerebrosidase polypeptides can be an isolated polypeptide.

“Substantially pure” or “purified” polypeptide or protein refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure glucocerebrosidase composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated engineered glucocerebrosidase polypeptides are substantially pure polypeptide compositions.

“Improved enzyme property” refers to an engineered glucocerebrosidase polypeptide that exhibits an improvement in any enzyme property as compared to a reference glucocerebrosidase polypeptide and/or as a wild-type glucocerebrosidase polypeptide or another engineered glucocerebrosidase polypeptide. Improved properties include but are not limited to such properties as increased protein expression, increased thermoactivity, increased thermostability, increased pH activity, increased stability, increased enzymatic activity, increased substrate specificity or affinity, increased specific activity, increased resistance to substrate or end-product inhibition, increased chemical stability, improved solvent stability, increased tolerance to acidic, neutral, or basic pH, increased tolerance to proteolytic activity (i.e., reduced sensitivity to proteolysis), reduced aggregation, increased solubility, reduced immunogenicity, improved post-translational modification (e.g., glycosylation), altered temperature profile, increased lysosomal stability, etc.

“Increased enzymatic activity” or “enhanced catalytic activity” refers to an improved property of the engineered glucocerebrosidase polypeptides, which can be represented by an increase in specific activity (e.g., product produced/time/weight protein) or an increase in percent conversion of the substrate to the product (e.g., percent conversion of starting amount of substrate to product in a specified time period using a specified amount of glucocerebrosidase) as compared to the reference glucocerebrosidase enzyme. Exemplary methods to determine enzyme activity are provided in the Examples. Any property relating to enzyme activity may be affected, including the classical enzyme properties of Km, Vmax or kcat, changes of which can lead to increased enzymatic activity.

“Improved tolerance to acidic pH” means that an engineered glucocerebrosidase that has increased stability (higher retained activity after exposure to an acidic pH, e.g., pH 2-6.6, for a specified period of time, e.g., 1 hour, up to 24 hr) as compared to a reference glucocerebrosidase or another enzyme.

“Improved tolerance to neutral pH or basic pH” means that an engineered glucocerebrosidase that has increased stability (higher retained activity after exposure to pH 7 or higher for a specified period of time (e.g., 1 hour, up to 24 hr)) as compared to a reference glucocerebrosidase or another enzyme.

“Improved cellular uptake” means that an engineered glucocerebrosidase provided herein exhibits increased uptake (e.g., via endocytosis or transport) into cells, as compared to a reference glucocerebrosidase (including wild-type glucocerebrosidase) or another enzyme. In some embodiments, the cells are cultured GD patient cells (higher retained intracellular activity after incubation with cultured cells over a specified period of time, as compared to a reference glucocerebrosidase or another enzyme). In some additional embodiments, the engineered glucocerebrosidase provided herein exhibits greater retained intracellular activity with cultured cells over a specific period of time as compared to a reference glucocerebrosidase (including wild-type glucocerebrosidase) or another enzyme. In some additional embodiments, the time period is about 4 hr, while in some other embodiments, the time period is less than 4 hr (e.g., 1, 2, or 3 hr), and in some alternative embodiments, the time period is more than 4 hr (e.g., 5, 6, 7, 8, or more hr).

“Reduced immunogenicity” and “decreased immunogenicity” mean that an engineered glucocerebrosidase that induces a reduced immune response as compared to a wild-type or another reference glucocerebrosidase.

“Deimmunized” as used herein, refers to the manipulation of a protein sequence to create a variant that is predicted to be not as immunogenic as the wild-type or reference protein. In some embodiments, the predicted deimmunization is complete, in that the variant protein is predicted to not stimulate an immune response in patients to whom the variant protein is administered. This response can be measured by various methods including but not limited to, the presence or abundance of anti-drug antibodies, the presence or abundance of neutralizing antibodies, the presence of an anaphylactic response, peptide presentation on major histocompatibility complex-II (MHC-II) proteins, or the prevalence or intensity of cytokine release upon administration of the protein. In some embodiments, the variant protein is less immunogenic than the wild-type or reference protein. In some embodiments, deimmunization involves modifications to subsequences of proteins (e.g., epitopes) that are recognized by human leukocyte antigen (HLA) receptors. In some embodiments, these epitopes are removed by changing their amino acid sequences to produce a deimmunized variant protein in which such subsequences are no longer recognized by the HLA receptors. In some other embodiments, these epitopes retain binding affinity to HLA receptors, but are not presented. In some embodiments, the deimmunized protein shows lower levels of response in biochemical and cell-biological predictors of human immunological responses including dendritic-cell T-cell activation assays, or (HLA) peptide binding assays. In some embodiments, these epitopes are removed by changing their amino acid sequence to produce a deimmunized variant protein in which the epitopes are no longer recognized by T-cell receptors. In still other embodiments the deimmunized protein induces anergy in its corresponding T-cells, activates T regulatory cells, or results in clonal deletion of recognizing B-cells. In some embodiments, a total immunogenicity score (TIS) reflects the overall predicted immunogenicity of the variant (i.e., a higher score indicates a higher level of predicted immunogenicity).

“Physiological pH” as used herein means the pH range generally found in a subject's (e.g., human) blood.

“Basic pH” (e.g., used with reference to improved stability to basic pH conditions or increased tolerance to basic pH) means a pH>7, such as a pH range >7 to 11.

“Acidic pH” (e.g., used with reference to improved stability to acidic pH conditions or increased tolerance to acidic pH) means a pH<7, in particular in the pH range of about 2 to <7.

“Hybridization stringency” relates to hybridization conditions, such as washing conditions, in the hybridization of nucleic acids. Generally, hybridization reactions are performed under conditions of lower stringency, followed by washes of varying but higher stringency (see, e.g., Sambrook et al., Molecular Cloning: A Laboratory Manual, Cold Spring Harbor Laboratory Press, New York, 2001; Ausubel et al., Current Protocols in Molecular Biology, John Wiley & Sons, 2003). The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tm as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Another high stringency condition is hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

“Codon optimized” refers to changes in the codons of the polynucleotide encoding a protein to those preferentially used in a particular organism such that the encoded protein is more efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the engineered glucocerebrosidase polypeptide may be codon optimized for optimal production from the host organism selected for expression.

“Control sequence” refers herein to include all components, which are necessary or advantageous for the expression of a polynucleotide and/or polypeptide of the present disclosure. Each control sequence may be native or foreign to the nucleic acid sequence encoding the polypeptide. Such control sequences include, but are not limited to, a leader, polyadenylation sequence, pro-peptide sequence, promoter sequence, signal peptide sequence, initiation sequence and transcription terminator. In some embodiments, at a minimum, the control sequences include a promoter, and transcriptional and translational stop signals.

“Operably linked” or “operatively linked” is defined herein as a configuration in which a control sequence is appropriately placed (i.e., in a functional relationship) at a position relative to a polynucleotide of interest such that the control sequence directs or regulates the expression of the polynucleotide, and where appropriate, the encoded polypeptide of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Suitable reaction conditions” refers to those conditions in the enzymatic conversion reaction solution (e.g., ranges of enzyme loading, substrate loading, temperature, pH, buffers, co-solvents, etc.) under which a glucocerebrosidase polypeptide of the present application is capable of converting a substrate to the desired product. Exemplary “suitable reaction conditions” are provided in the present application and illustrated by the Examples. “Substrate” in the context of an enzymatic conversion reaction process refers to the compound or molecule acted on by the glucocerebrosidase polypeptide. “Product” in the context of an enzymatic conversion process refers to the compound or molecule resulting from the action of the glucocerebrosidase polypeptide on a substrate.

“Culturing” refers to the growing of a population of cells under any suitable conditions (e.g., using a liquid, gel or solid medium).

“Vector” refers to a polynucleotide construct for introducing a polynucleotide sequence into a cell. In some embodiments, the vector is an expression vector that is operably linked to a suitable control sequence capable of effecting the expression in a suitable host of the polynucleotide, and where relevant, the polypeptide encoded in the polynucleotide sequence. In some embodiments, an “expression vector” has a promoter sequence operably linked to the polynucleotide sequence (e.g., transgene) to drive expression in a host cell, and in some embodiments, also comprises a transcription terminator sequence.

“Gene therapy vector” refers to vehicles or carriers suitable for delivery of polynucleotide sequences to cells for therapeutic purposes. In some embodiments, the vectors encapsulate genes (e.g., transgenes) or polynucleotide sequences for delivery to cells or tissues, including but not limited to adenovirus (AV), adeno-associated virus (AAV), lentivirus (LV), and non-viral vectors, such as liposomes. It is not intended that the present disclosure be limited to any specific gene therapy vector, as any vehicle suitable for a given setting finds use herein. The gene therapy vector may be designed to deliver genes to a specific species or host or cells, or may find more general applicability.

“Expression” includes any step involved in the production of the polypeptide including, but not limited to, transcription, post-transcriptional modification, translation, and post-translational modification. In some embodiments, the term also encompasses secretion of the polypeptide from a cell.

“Produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

“Heterologous” refers to the relationship between two or more nucleic acid or protein sequences (e.g., a promoter sequence, signal peptide, terminator sequence, etc.) that are derived from different sources and are not associated in nature.

“Host cell” and “host strain” refer to suitable hosts for expression vectors comprising a recombinant polynucleotide provided herein (e.g., the polynucleotides encoding the glucocerebrosidase variants). In some embodiments, the host cells are prokaryotic or eukaryotic cells that have been transformed or transfected with vectors constructed using recombinant DNA techniques as known in the art.

“Therapeutic” refers to a compound administered to a subject who shows signs or symptoms of pathology having beneficial or desirable medical effects.

“Gene therapy” refers to the delivery of a gene, polydeoxyribonucleotide, or polynucleotide sequence(s) with a gene therapy vector or as a formulation to cells or tissues for the modification of those cells or tissues for the treatment or prevention of a disease. In some embodiments, gene therapy may be temporary, for example by introduction of the gene, polydeoxyribonucleotide, or polynucleotide sequence(s) into cells that result in transitory presence of the gene, polydeoxyribonucleotide, or polynucleotide. In some embodiments, gene therapy may include replacing a mutated gene that causes disease with a healthy copy of the gene, or inactivating, or “knocking out,” a mutated gene that is functioning improperly; or providing a functional copy of a gene sufficient to treat and/or prevent the disease or condition. In some embodiments, gene therapy is used in the treatment of disease in patients.

“mRNA therapy” refers to the delivery of an mRNA polyribonucleotide to cells or tissues for the modification of those cells or tissues for the treatment or prevention of a disease. In some embodiments, the mRNA polynucleotide for delivery to cells or tissue are formulated, for instance, but not limited to, in liposomes. In some embodiments, mRNA therapy is used in the treatment of disease in patients.

“Cell therapy” refers to the delivery of living cells that have been modified exogenously to patients to provide a missing gene for the treatment or prevention of a disease. The modified cells are then reintroduced into the body.

“Effective amount” means an amount sufficient to produce the desired result. One of general skill in the art may determine what the effective amount by using routine experimentation.

“Subject” encompasses mammals such as humans, non-human primates, livestock, companion animals (e.g., cats, dogs, rodents), and laboratory animals (e.g., rodents and lagomorphs). “Patient” means any subject that is being assessed for, treated for, or is experiencing disease.

“Composition” and “formulation” encompass products comprising at least one engineered glucocerebrosidase of the present disclosure, or a recombinant polynucleotide encoding the engineered glucocerebrosidase intended for any suitable use (e.g., pharmaceutical compositions, etc.).

“Administration” and “administering” an engineered glucocerebrosidase, or a composition thereof, mean providing the engineered glucocerebrosidase of the present disclosure, or the compositions thereof, to a subject (e.g., to a subject suffering from the effects of Gaucher disease).

“Pharmaceutical composition” refers to a composition suitable for pharmaceutical use in a mammalian subject (e.g., human) comprising a pharmaceutically effective amount of an engineered glucocerebrosidase polypeptide disclosed herein, and an acceptable carrier and/or excipient.

“Pharmaceutically acceptable” means a material that can be administered to a subject without causing any undesirable biological effects or interacting in a deleterious manner with any of the components in which it is contained and that possesses the desired biological activity.

“Carrier” when used in reference to a pharmaceutical composition means any of the standard pharmaceutical carrier, buffers, and excipients, such as stabilizers, preservatives, and adjuvants.

“Excipient” refers to any pharmaceutically acceptable additive, carrier, diluent, adjuvant, or other ingredient, other than the active pharmaceutical ingredient (API; e.g., the engineered glucocerebrosidase polypeptides of the present disclosure). Excipients are typically included for formulation and/or administration purposes.

“Therapeutically effective amount” when used in reference to symptoms of disease/condition refers to the amount and/or concentration of a compound (e.g., engineered glucocerebrosidase polypeptides) that ameliorates, attenuates, or eliminates one or more symptom of a disease/condition or prevents or delays the onset of symptom(s). A “therapeutically effective amount” when used in reference to a disease/condition refers to the amount and/or concentration of a composition (e.g., engineered glucocerebrosidase polypeptides) that ameliorates, attenuates, or eliminates the disease/condition. In some embodiments, the term is use in reference to the amount of a composition that elicits the biological (e.g., medical) response by a tissue, system, or animal subject that is sought by the researcher, physician, veterinarian, or other clinician.

“Treating” or “treatment” of a disease, disorder, or syndrome, as used herein, includes (i) preventing the disease, disorder, or syndrome from occurring in a subject, i.e., causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As such, the terms “treating,” “treat” and “treatment” encompass preventative (e.g., prophylactic), as well as palliative treatment. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

### Engineered Glucocerebrosidase Polypeptides

In one aspect, the present disclosure provides engineered glucocerebrosidases with improved properties. As discussed above, in some embodiments, the engineered glucocerebrosidases are characterized by, among others, improved activity, improved expression efficiency, increased stability at neutral and acidic pH, increased serum stability, enhanced uptake into cells, and/or reduced immunogenicity.

In some embodiments, an engineered glucocerebrosidase, or biologically active fragment thereof, comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 40-536 of SEQ ID NO: 2-1648, or to a reference sequence corresponding to SEQ ID NO: 2-1648, wherein the amino acid sequence comprises one or more substitutions relative to a reference sequence corresponding to residues 40-536 of SEQ ID NO: 2, 54, 220, 984, or 1150, or relative to a reference sequence corresponding to SEQ ID NO: 2, 54, 220, 984, or 1150.

In some embodiments, the engineered glucocerebrosidase, or biologically fragment thereof, comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 2, 54, 220, 984, or 1150, or to the reference sequence corresponding to SEQ ID NO: 2, 54, 220, 984, or 1150, wherein the amino acid sequence comprises one or more substitutions relative to the reference sequence corresponding to residues 40-536 of SEQ ID NO: 2, 54, 220, 984, or 1150, or relative to the reference sequence corresponding to SEQ ID NO: 2, 54, 220, 984, or 1150.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 2, or to the reference sequence corresponding to SEQ ID NO: 2, wherein the amino acid sequence comprises one or more substitutions relative to the reference sequence corresponding to residues 40-536 of SEQ ID NO: 2, or relative to the reference sequence corresponding to SEQ ID NO: 2.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, 220, 984, or 1150, or to the reference sequence corresponding to SEQ ID NO: 54, 220, 984, or 1150, wherein the amino acid sequence comprises one or more substitutions relative to the reference sequence corresponding to residues 40-536 of SEQ ID NO: 2, or relative to the reference sequence corresponding to SEQ ID NO: 2.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 4-1648, or to the reference sequence corresponding to an even-numbered SEQ ID NO. of SEQ ID NOs: 4-1648, wherein the amino acid sequence comprises one or more substitutions relative to the reference sequence corresponding to residues 40-536 of SEQ ID NO: 2, or relative to the reference sequence corresponding to SEQ ID NO: 2.

In some embodiments, the engineered glucocerebrosidase comprises an amino acid sequence having 95%, 96%, 97%, 98%, 99%, or more sequence identity to the reference sequence corresponding to residues 40-536 of an even-numbered SEQ ID NO. of SEQ ID NOs: 4-198 and 1556-1648, or to the reference sequence corresponding to an even-numbered SEQ ID NO. of SEQ ID NOs: 4-198 and 1556-1648, wherein the amino acid sequence comprises one or more substitutions relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution at amino acid position 23, 29, 35, 40, 41, 42, 44, 46, 50, 51, 53, 59, 63, 65, 68, 69, 70, 71, 75, 77, 78, 79, 82, 84, 86, 88, 89, 90, 91, 92, 93, 94, 95, 96, 97, 98, 99, 100, 101, 102, 103, 104, 105, 106, 107, 108, 109, 110, 111, 113, 114, 115, 116, 117, 133, 134, 137, 138, 141, 142, 144, 150, 151, 154, 169, 171, 172, 175, 180, 182, 184, 186, 189, 190, 191, 194, 195, 201, 202, 204, 206, 207, 211, 212, 214, 220, 222, 224, 230, 233, 235, 236, 239, 243, 246, 258, 260, 261, 262, 263, 264, 265, 271, 276, 298, 299, 301, 314, 315, 316, 319, 325, 329, 332, 333, 335, 336, 337, 338, 339, 341, 342, 359, 360, 361, 368, 370, 372, 373, 374, 382, 385, 386, 390, 400, 404, 408, 412, 413, 415, 434, 445, 446, 449, 459, 467, 468, 469, 471, 473, 474, 476, 477, 478, 479, 480, 481, 484, 485, 487, 489, 490, 493, 494, 495, 497, 502, 504, 505, 506, 507, 508, 509, 510, 512, 514, 515, 516, 518, 519, 520, 522, 524, 525, 529, 532, 533, 534, or 536, or combinations thereof, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution 23A, 29I, 35T, 40H/S, 41H, 42A, 44V, 46M/R, 50A, 51P, 53L, 59N, 63L, 65L/M/S/V, 68F/L/Q/T/V/W/Y, 69D, 70L, 71F/G/H/L/M/R/S/T/Y, 75A/E/H/N, 77G/H/I/N/R/T, 78L/M, 79I/R/V, 82S, 84A/G/K, 86E/H, 88L, 89C/D, 90A/M, 91I/T, 92A/E/G/H/K/Q, 93S, 94E/N, 95G/H/L/T/W, 96D/E/G/I/M/N/R/S/V, 97E/I/K/M/N/Q/R, 98P, 99E/F/G/K/L/R/S/V/W, 100N/R, 101L/Q/R/S/T/V/W, 102A/E/N/P/S, 103A/L/P/S, 104M/V, 105I/M/T, 106W, 107A/C/D/E/M/Q/S, 108I, 109D/G/L/M/R, 110A/H/Q, 111F/H/L, 113G/I/N/P/Q/R, 114Y, 115M/R/V, 116E/M/Q/R/V/Y, 117I/T, 133M, 134S, 137Q/V, 138S, 141D/E, 142A/M/N/Y, 144M, 150T, 151L, 154E, 169V, 171C/P/V, 172S, 175D, 180C, 182D/E/G/H/M/S/T, 184I/Q/V, 186G, 189R/S, 190W, 191N/R, 194H/L/Q, 195M/W, 201Q/R/T, 202Q, 204Q, 206F/I/R, 207S/D, 211I/L/N, 212A/N, 214F, 220A/G, 222I, 224M, 230F/L/Y, 233A/G/Q/R, 235T, 236M, 239I/R/T, 243K/L/M/R/V, 246K/R, 258T, 260R, 261K, 262Q, 263G, 264I, 265N/R/S/T, 271T, 276T, 298W, 299V, 301K, 314D/G/R, 315T, 316H, 319F/I/V, 325V, 329L, 332R, 333C/L/T, 335M, 336A/E/G/R, 337N, 338R, 339A, 341R/S, 342Q, 359E/P, 360E, 361L/V/W/Y, 368D, 370R, 372D, 373A/V/Y, 374T/V, 382A, 385G/H/N/Q/R, 386I/L/P, 390A, 400E/Q/T, 404Q, 408D/E, 412N/W, 413W/Y, 415C, 434G/Q, 445G/N, 446H/N, 449E/K/L/M/R/S/V, 459M, 467H/L/M/N/R, 468G, 469E, 471E/F/L/R/S/T/V, 473C/I/L, 474A/D/S, 476A, 477G/H/R/S/V, 478N, 479A/L/R, 480G, 481H/K/T, 484E, 485M/S/T, 487S, 489A/E/G/L/N/Q/T, 490G/I/N/Q/R/S/T/V, 493T, 494A/T, 495G/S, 497A/M, 502K/Q, 504G/P/T/W, 505I/L/R, 506T, 507A/I/L/M/R/T/Y, 508L/Q/R/S/T, 509F/G/I/R/V/W, 510S, 512H/L/Q, 514E, 515G/T, 516L, 518E/L/S/V, 519I, 520D/K/R/S/V, 522S/T/V, 524K/S, 525C/Q, 529M/Q, 532V, 533Y, 534K, or 536A/C/D/H/I/L/M/P/R/T/W, or combinations thereof, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution G23A, L29I, S35T, A40H/S, R41H, P42A, I44V, K46M/R, Y50A, S51P, V53L, A59N, D63L, F65L/M/S/V, P68F/L/Q/V/T/W/Y, T69D, F70L, P71F/G/H/L/M/R/S/T/Y, T75A/E/H/N, S77G/H/I/N/R/T, R78L/M, Y79I/R/V, T82S, S84A/G/K, R86E/H, M88L, E89C/D, L90A/M, S91I/T, M92A/E/G/H/K/Q, G93S, P94E/N, 195G/H/L/T/W, Q96D/E/G/I/M/N/R/S/V, A97E/I/K/M/N/Q/R, N98P, H99E/F/G/K/L/R/S/V/W, T100N/R, G101L/Q/R/S/T/V/W, T102A/E/N/P/S, G103A/L/P/S, L104M/V, L105I/M/T, L106W, T107A/C/D/E/M/Q/S, L108I, Q109D/G/L/M/R, P110A/H/Q, E111F/H/L, K113G/I/N/P/Q/R, F114Y, Q115M/R/V, K116E/M/Q/R/V/Y, V117I/T, L133M, A134S, P137Q/V, P138S, N141D/E, L142A/M/N/Y, L144M, E150T, E151L, G154E, I169V, T171C/P/V, Y172S, A175D, D180C, Q182D/E/G/H/M/S/T, H184I/Q/V, F186G, P189R/S, E190W, E191N/R, K194H/L/Q, L195M/W, H201Q/R/T, R202Q, L204Q, L206F/I/R, A207D/S, V211I/L/N, S212A/N, L214F, S220A/G, T222I, L224M, V230F/L/Y, K233A/G/Q/R, S235T, L236M, Q239I/R/T, I243K/L/M/R/V, Q246K/R, A258T, A260R, E261K, H262Q, K263G, L264I, Q265N/R/S/T, A271T, S276T, F298W, 1299V, R301K, N314D/G/R, V315T, R316H, M319F/I/V, L325V, H329L, K332R, V333C/L/T, L335M, T336A/E/G/R, D337N, P338R, E339A, A341R/S, K342Q, A359E/P, K360E, A361L/V/W/Y, R368D, F370R, N372D, T373A/V/Y, M374T/V, V382A, K385G/H/N/Q/R, F386I/L/P, S390A, M400E/Q/T, H404Q, T408D/E, Y412N/W, H413W/Y, V415C, R434G/Q, I445G/N, T446H/N, T449E/K/L/M/R/S/V, L459M, P467H/L/M/N/R, E468G, G469E, Q471E/F/L/R/S/T/V, V473C/I/L, G474A/D/S, V476A, A477G/H/R/S/V, S478N, Q479A/L/R, K480G, N481H/K/T, D484E, A485M/S/T, A487S, M489A/E/G/L/N/Q/T, H490G/I/N/Q/R/S/T/V, G493T, S494A/T, A495G/S, V497A/M, R502K/Q, S504G/P/T/W, K505I/L/R, D506T, V507A/I/L/M/R/T/Y, P508L/Q/R/S/T, L509F/G/I/R/V/W, T510S, K512H/L/Q, P514E, A515G/T, V516L, F518E/L/S/V, L519I, E520D/K/R/S/V, I522S/T/V, P524K/S, G525C/Q, H529M/Q, L532V, W533Y, R534K, or Q536A/C/D/H/I/L/M/P/R/T/W, or combinations thereof, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution at amino acid position 65, 68, 70, 99, 154, 195, 207, 214, 230, 263, 319, 337, 361, 374, 408, 471, or 489, or combinations thereof, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution 65L/M/S/V, 68F/L/Q/T/V/W/Y, 70L, 99E/F/G/K/L/R/S/V/W, 154E, 195M/W, 207S/D, 214F, 230F/L/Y, 263M, 319F/I/V, 337N, 361L/V/W/Y, 374T/V, 408D/E, 471E/F/L/R/S/T/V, or 489A/E/G/L/N/Q/T, or combinations thereof, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution 665L/V, 68L, 70L, 99R, 154E, 195W, 207S, 214F, 230L/Y, 263G, 319I, 337N, 361W, 374T, 408E, 471T, or 489L, or combinations thereof, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution at amino acid position 70, 207, 214, or 489, or combinations thereof, wherein the amino acid positions are relative to the reference sequence corresponding to residues 40-536 of SEQ ID NO: 2, or relative to the reference sequence corresponding to SEQ ID NO: 2. In some embodiments, the amino acid sequence of the engineered glucocerebrosidase comprises at least a substitution 70L, 207S/D, 214F, or 489A/E/G/L/N/Q/T, or combinations thereof, wherein the amino acid positions are relative to the reference sequence corresponding to residues 40-536 of SEQ ID NO: 2, or relative to the reference sequence corresponding to SEQ ID NO: 2. In some embodiments, the amino acid sequence of the engineered glucocerebrosidase comprises at least a substitution 70L, 207S, 214F, or 489L, or combinations thereof, wherein the amino acid positions are relative to the reference sequence corresponding to residues 40-536 of SEQ ID NO: 2, or relative to the reference sequence corresponding to SEQ ID NO: 2. In some embodiments, the amino acid sequence of the engineered glucocerebrosidase comprises at least a substitution set at amino acid positions 70/207/214/489, wherein the amino acid positions are relative to the reference sequence corresponding to residues 40-536 of SEQ ID NO: 2, or relative to the reference sequence corresponding to SEQ ID NO: 2. In some embodiments, the amino acid sequence of the engineered glucocerebrosidase comprises at least a substitution set 70L/207S/214F/489L, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution at amino acid position 65, 68, 99, 154, 230, or 319, or combinations thereof, wherein the amino acid positions are relative to the reference sequence corresponding to residues 40-536 of SEQ ID NO: 2, or relative to the reference sequence corresponding to SEQ ID NO: 2. In some embodiments, the amino acid sequence of the engineered glucocerebrosidase comprises at least a substitution 65L/M/S/V, 68F/L/Q/T/V/W/Y, 99E/F/G/K/L/R/S/V/W, 154E, 230F/L/Y, or 319F/I/V, or combinations thereof, wherein the amino acid positions are relative to the reference sequence corresponding to residues 40-536 of SEQ ID NO: 2, or relative to the reference sequence corresponding to SEQ ID NO: 2. In some embodiments, the amino acid sequence of the engineered glucocerebrosidase comprises at least a substitution 65L, 68L, 99R, 154E, 230L, or 319I, or combinations thereof, wherein the amino acid positions are relative to the reference sequence corresponding to residues 40-536 of SEQ ID NO: 2, or relative to the reference sequence corresponding to SEQ ID NO: 2. In some embodiments, the amino acid sequence of the engineered glucocerebrosidase comprises at least a substitution set at amino acid positions 65/68/99/154/230/319, wherein the amino acid positions are relative to the reference sequence corresponding to residues 40-536 of SEQ ID NO: 2, or relative to the reference sequence corresponding to SEQ ID NO: 2. In some embodiments, the amino acid sequence of the engineered glucocerebrosidase comprises at least a substitution set 65L/68L/99R/154E/230L/319I, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution at amino acid position 65, 195, 230, 263, 337, 361, 374, or 471, or combinations thereof, wherein the amino acid positions are relative to the reference sequence corresponding to residues 40-536 of SEQ ID NO: 2, or relative to the reference sequence corresponding to SEQ ID NO: 2. In some embodiments, the amino acid sequence of the engineered glucocerebrosidase comprises at least a substitution 65L/M/S/V, 195M/W, 230F/L/Y, 263G, 337N, 361L/V/W/Y, 374T/V, or 471E/F/L/R/S/T/V, or combinations thereof, wherein the amino acid positions are relative to the reference sequence corresponding to residues 40-536 of SEQ ID NO: 2, or relative to the reference sequence corresponding to SEQ ID NO: 2. In some embodiments, the amino acid sequence of the engineered glucocerebrosidase comprises at least a substitution 65V, 195W, 230Y, 263G, 337N, 361W, 374T, or 471T, or combinations thereof, wherein the amino acid positions are relative to the reference sequence corresponding to residues 40-536 of SEQ ID NO: 2, or relative to the reference sequence corresponding to SEQ ID NO: 2. In some embodiments, the amino acid sequence of the engineered glucocerebrosidase comprises at least a substitution set amino acid positions 65/195/230/263/337/361/374/471, wherein the amino acid positions are relative to the reference sequence corresponding to residues 40-536 of SEQ ID NO: 2, or relative to the reference sequence corresponding to SEQ ID NO: 2. In some embodiments, the amino acid sequence of the engineered glucocerebrosidase comprises at least a substitution set 65V/195W/230Y/263G/337N/361W/374T/471T, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution at amino acid position 408, wherein the amino acid positions are relative to the reference sequence corresponding to residues 40-536 of SEQ ID NO: 2, or relative to the reference sequence corresponding to SEQ ID NO: 2. In some embodiments, the amino acid sequence of the engineered glucocerebrosidase comprises at least a substitution 408D/E, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebroside comprises at least a substitution or substitution set at amino acid position(s) 265/372/489, 243, 23, 109, 265, 109/195/243, 23/70/207/214/265, 23/70, 23/214/243/265, 65/70/109/214/319/372/481, 23/265/319, 23/65/70/141/319/481, 23/265, 372, 214, 23/65/70/109/141/214, 319, 65/372, 109/195, 23/65, 207/265/319, 109/207/214/372, 23/319/372/489, 65/109/214/243, 23/141/195, 70/207/214/489, 65/70/265/319/372, 214/265, 265/319, 65/243, 65/70/195, 489, 23/65/141, 23/372, 70/141, 23/195, 214/319, 23/207/214/243/265/481, 65/214/243/265/319, 214/243, 243/265, 265/489, 214/243/265, 207/243/372, 195/243, 65/481, 207/214/243/319, 70/141/195/265, 23/265/372, 46/99/134/230/373/449, 68/211/230, 373/449, 46/68/336, 68/230/314/373, 68, 68/336/373, 99/314, 46, 46/230/261, 373, 230/314, 68/211, 68/336/449, 46/336, 336/449, 46/68/336/373, 46/99, 99/261, 99, 68/134, 46/68/134/230/336/373, 46/68, 68/99/211/230, 99/134, 68/99/336, 449, 68/99, 46/230/336, 68/154/449, 336/373/449, 134/336, 68/99/230, 46/68/134/154, 68/99/230/373/449, 134, 46/68/154/211/230/261/336, 68/99/154/261/336, 230/449, 46/68/314, 154, 68/449, 99/449, 68/154/230/314/336/449, 46/68/154/314/336/373, 46/230, 336, or 46/99/230/373, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebroside comprises at least a substitution or substitution set 265T/372D/489L, 243K, 23A, 109D, 265T, 109D/195M/243K, 23A/70L/207S/214F/265T, 23A/70L, 23A/214F/243K/265T, 65L/70L/109D/214F/319I/372D/481T, 23A/265T/319I, 23A/65L/70L/141D/319I/481T, 23A/265T, 372D, 214F, 23A/65L/70L/109D/141D/214F, 319I, 65L/372D, 109D/195M, 23A/65L, 207S/265T/319I, 109D/207S/214F/372D, 23A/319I/372D/489L, 65L/109D/214F/243K, 23A/141D/195M, 70L/207S/214F/489L, 65L/70L/265T/319I/372D, 214F/265T, 265T/319I, 65L/243K, 65L/70L/195M, 489L, 23A/65L/141D, 23A/372D, 70L/141D, 23A/195M, 214F/319I, 23A/207S/214F/243K/265T/481T, 65L/214F/243K/265T/319I, 214F/243K, 243K/265T, 265T/489L, 214F/243K/265T, 207S/243K/372D, 195M/243K, 65L/481T, 207S/214F/243K/319I, 70L/141D/195M/265T, 23A/265T/372D, 46R/99R/134S/230L/373Y/449V, 68L/211L/230L, 373Y/449V, 46R/68V/336A, 68V/230L/314D/373Y, 68V, 68L/336A/373Y, 99R/314D, 46R, 46R/230L/261K, 373Y, 230L/314D, 68L/211L, 68V/336A/449V, 46R/336A, 336A/449V, 46R/68V/336A/373Y, 46R/99R, 99R/261K, 99R, 68V/134S, 46R/68V/134S/230L/336A/373Y, 46R/68V, 68L/99R/211L/230L, 99R/134S, 68V/99R/336A, 449V, 68V/99R, 46R/230L/336A, 68V/154E/449V, 336A/373Y/449V, 134S/336A, 68V/99R/230L, 68L, 46R/68V/134S/154E, 68V/99R/230L/373Y/449V, 134S, 46R/68V/154E/211L/230L/261K/336A, 68V/99R/154E/261K/336A, 230L/449V, 46R/68L/314D, 154E, 68L/449V, 99R/449V, 68V/154E/230L/314D/336A/449V, 46R/68V/154E/314D/336A/373Y, 46R/230L, 336A, or 46R/99R/230L/373Y, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebroside comprises at least a substitution or substitution set Q265T/N372D/M489L, 1243K, G23A, Q109D, Q265T, Q109D/L195M/1243K, G23A/F70L/A207S/L214F/Q265T, G23A/F70L, G23A/L214F/1243K/Q265T, F65L/F70L/Q109D/L214F/M319I/N372D/N481T, G23A/Q265T/M319I, G23A/F65L/F70L/N141D/M319I/N481T, G23A/Q265T, N372D, L214F, G23A/F65L/F70L/Q109D/N141D/L214F, M319I, F65L/N372D, Q109D/L195M, G23A/F65L, A207S/Q265T/M319I, Q109D/A207S/L214F/N372D, G23A/M319I/N372D/M489L, F65L/Q109D/L214F/1243K, G23A/N141D/L195M, F70L/A207S/L214F/M489L, F65L/F70L/Q265T/M319I/N372D, L214F/Q265T, Q265T/M319I, F65L/1243K, F65L/F70L/L195M, M489L, G23A/F65L/N141D, G23A/N372D, F70L/N141D, G23A/L195M, L214F/M319I, G23A/A207S/L214F/1243K/Q265T/N481T, F65L/L214F/1243K/Q265T/M319I, L214F/1243K, I243K/Q265T, Q265T/M489L, L214F/1243K/Q265T, A207S/1243K/N372D, L195M/1243K, F65L/N481T, A207S/L214F/1243K/M319I, F70L/N141D/L195M/Q265T, G23A/Q265T/N372D, K46R/H99R/A134S/V230L/T373Y/T449V, P68L/V211L/V230L, T373Y/T449V, K46R/P68V/T336A, P68V/V230L/N314D/T373Y, P68V, P68L/T336A/T373Y, H99R/N314D, K46R, K46R/V230L/E261K, T373Y, V230L/N314D, P68L/V211L, P68V/T336A/T449V, K46R/T336A, T336A/T449V, K46R/P68V/T336A/T373Y, K46R/H99R, H99R/E261K, H99R, P68V/A134S, K46R/P68V/A134S/V230L/T336A/T373Y, K46R/P68V, P68L/H99R/V211L/V230L, H99R/A134S, P68V/H99R/T336A, T449V, P68V/H99R, K46R/V230L/T336A, P68V/G154E/T449V, T336A/T373Y/T449V, A134S/T336A, P68V/H99R/V230L, P68L, K46R/P68V/A134S/G154E, P68V/H99R/V230L/T373Y/T449V, A134S, K46R/P68V/G154E/V211L/V230L/E261K/T336A, P68V/H99R/G154E/E261K/T336A, V230L/T449V, K46R/P68L/N314D, G154E, P68L/T449V, H99R/T449V, P68V/G154E/V230L/N314D/T336A/T449V, K46R/P68V/G154E/N314D/T336A/T373Y, K46R/V230L, T336A, or K46R/H99R/V230L/T373Y, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebroside comprises at least a substitution or substitution set at amino acid position(s) 65/68/70/99/154/195/207/214/230/263/319/337/361/374/408/471/489, 65/68/70/99/154/195/207/214/230/263/319/337/361/374/408, 65/68/154/195/214/230/319/337/374/408, 65/68/70/99/154/195/207/214/230/263/374/408, 65/68/70/154/195/214/230/319/374/408/489, 65/68/70/99/154/195/207/214/230/319/337/361/374/408/471/489, 65/68/70/99/154/195/207/214/230/263/319/337/374/408, 65/68/154/195/207/214/230/263/319/337/361/374/408/471/489, 65/68/70/99/154/195/207/214/230/263/319/337/361/374/408/489, 65/68/70/99/154/195/207/214/230/374/408/471, 68/70/99/154/195/207/214/230/263/319/408, 65/68/70/207/230/374/408, 65/68/70/214/230/263/337/374/408, 214/230/263/374/408, 65/68/70/99/154/195/207/230/408, 65/68/70/99/154/207/230/319/408, 65/68/70/214/230/408, 65/68/70/195/214/230/263/361/471, 195/207/230/319/374/408/489, 65/68/70/214/230/408/489, 65/68/70/195/207/230/408, 207/230/319/374/408, 65/68/70/99/195/207/230/408, 195/214/230/263/408, 195/207/230/374/408, 65/68/70/99/154/195/214/408, 207/214/230/319/374/408, 207/214/230/374/408, 154/207/230/374/408/489, 207/230/263/408, 207/230/408/471, 207/230/374/408, 214/230/319/408, 214/230/374/408/489, 207/230/408, 65/68/70/154/207/408, 207/230/263/337/408, 195/214/230/408, 207/230/408/489, 214/230/408, 230/361/408, 207/214/408, 214/263/408, 230/374/408, 207/408, 195/214/263, 374/408, or 408, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebroside comprises at least a substitution or substitution set 65V/68L/70L/99R/154E/195W/207S/214F/230Y/263G/319I/337N/361W/374T/408E/471T/489L, 65V/68L/70L/99R/154E/195W/207S/214F/230Y/263G/319I/337N/361W/374T/408E, 65V/68L/154E/195W/214F/230Y/319I/337N/374T/408E, 65V/68L/70L/99R/154E/195W/207S/214F/230Y/263G/374T/408E, 65V/68L/70L/154E/195W/214F/230Y/319I/374T/408E/489L, 65V/68L/70L/99R/154E/195W/207S/214F/230Y/319I/337N/361W/374T/408E/471T/489L, 65V/68L/70L/99R/154E/195W/207S/214F/230Y/263G/319I/337N/374T/408E, 65V/68L/154E/195W/207S/214F/230Y/263G/319I/337N/361W/374T/408E/471T/489L, 65V/68L/70L/99R/154E/195W/207S/214F/230Y/263G/319I/337N/361W/374T/408E/489L, 65V/68L/70L/99R/154E/195W/207S/214F/230Y/374T/408E/471T, 68L/70L/99R/154E/195W/207S/214F/230Y/263G/319I/408E, 65V/68L/70L/207S/230Y/374T/408E, 65V/68L/70L/214F/230Y/263G/337N/374T/408E, 214F/230Y/263G/374T/408E, 65V/68L/70L/99R/154E/195W/207S/230Y/408E, 65V/68L/70L/99R/154E/207S/230Y/319I/408E, 65V/68L/70L/214F/230Y/408E, 65V/68L/70L/195W/214F/230Y/263G/361W/471T, 195W/207S/230Y/319I/374T/408E/489L, 65V/68L/70L/214F/230Y/408E/489L, 65V/68L/70L/195W/207S/230Y/408E, 207S/230Y/319I/374T/408E, 65V/68L/70L/99R/195W/207S/230Y/408E, 195W/214F/230Y/263G/408E, 195W/207S/230Y/374T/408E, 65V/68L/70L/99R/154E/195W/214F/408E, 207S/214F/230Y/319I/374T/408E, 207S/214F/230Y/374T/408E, 154E/207S/230Y/374T/408E/489L, 207S/230Y/263G/408E, 207S/230Y/408E/471T, 207S/230Y/374T/408E, 214F/230Y/319I/408E, 214F/230Y/374T/408E/489L, 207S/230Y/408E, 65V/68L/70L/154E/207S/408E, 207S/230Y/263G/337N/408E, 195W/214F/230Y/408E, 207S/230Y/408E/489L, 214F/230Y/408E, 230Y/361W/408E, 207S/214F/408E, 214F/263G/408E, 230Y/374T/408E, 207S/408E, 195W/214F/263G, 374T/408E, or 408E, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebroside comprises at least a substitution or substitution set F65V/P68L/F70L/H99R/G154E/L195W/A207S/L214F/V230Y/K263G/M319I/D337N/A361W/M374T/T408E/Q471T/M489L, F65V/P68L/F70L/H99R/G154E/L195W/A207S/L214F/V230Y/K263G/M319I/D337N/A361W/M374T/T408E, F65V/P68L/G154E/L195W/L214F/V230Y/M319I/D337N/M374T/T408E, F65V/P68L/F70L/H99R/G154E/L195W/A207S/L214F/V230Y/K263G/M374T/T408E, F65V/P68L/F70L/G154E/L195W/L214F/V230Y/M319I/M374T/T408E/M489L, F65V/P68L/F70L/H99R/G154E/L195W/A207S/L214F/V230Y/M319I/D337N/A361W/M374T/T408E/Q471 T/M489L, F65V/P68L/F70L/H99R/G154E/L195W/A207S/L214F/V230Y/K263G/M319I/D337N/M374T/T408E, F65V/P68L/G154E/L195W/A207S/L214F/V230Y/K263G/M319I/D337N/A361W/M374T/T408E/Q471T/M489L, F65V/P68L/F70L/H99R/G154E/L195W/A207S/L214F/V230Y/K263G/M319I/D337N/A361W/M374T/T408E/M489L, F65V/P68L/F70L/H99R/G154E/L195W/A207S/L214F/V230Y/M374T/T408E/Q471T, P68L/F70L/H99R/G154E/L195W/A207S/L214F/V230Y/K263G/M319I/T408E, F65V/P68L/F70L/A207S/V230Y/M374T/T408E, F65V/P68L/F70L/L214F/V230Y/K263G/D337N/M374T/T408E, L214F/V230Y/K263G/M374T/T408E, F65V/P68L/F70L/H99R/G154E/L195W/A207S/V230Y/T408E, F65V/P68L/F70L/H99R/G154E/A207S/V230Y/M319I/T408E, F65V/P68L/F70L/L214F/V230Y/T408E, F65V/P68L/F70L/L195W/L214F/V230Y/K263G/A361W/Q471T, L195W/A207S/V230Y/M319I/M374T/T408E/M489L, F65V/P68L/F70L/L214F/V230Y/T408E/M489L, F65V/P68L/F70L/L195W/A207S/V230Y/T408E, A207S/V230Y/M319I/M374T/T408E, F65V/P68L/F70L/H99R/L195W/A207S/V230Y/T408E, L195W/L214F/V230Y/K263G/T408E, L195W/A207S/V230Y/M374T/T408E, F65V/P68L/F70L/H99R/G154E/L195W/L214F/T408E, A207S/L214F/V230Y/M319I/M374T/T408E, A207S/L214F/V230Y/M374T/T408E, G154E/A207S/V230Y/M374T/T408E/M489L, A207S/V230Y/K263G/T408E, A207S/V230Y/T408E/Q471T, A207S/V230Y/M374T/T408E, L214F/V230Y/M319I/T408E, L214F/V230Y/M374T/T408E/M489L, A207S/V230Y/T408E, F65V/P68L/F70L/G154E/A207S/T408E, A207S/V230Y/K263G/D337N/T408E, L195W/L214F/V230Y/T408E, A207S/V230Y/T408E/M489L, L214F/V230Y/T408E, V230Y/A361W/T408E, A207S/L214F/T408E, L214F/K263G/T408E, V230Y/M374T/T408E, A207S/T408E, L195W/L214F/K263G, M374T/T408E, or T408E, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set at amino acid position(s) 68/70/207/214/336/489, 65/70/99/207/214/319/489, 65/68/70/207/214/319/489, 68/70/207/214/319/489, 68/70/207/214/489, 65/70/207/214/319/489, 70/195/207/214/489, 65/154/70/207/214/319/489, 70/207/214/489, 65/68/70/99/154/207/214/230/319/489, 68/70/207/214/230/489, 70/195/207/214/230/489, 65/70/207/214/230/489, 70/141/207/214/319/489, 70/154/207/214/336/489, 68/70/154/207/214/489, 68/70/195/207/214/230/319/336/489, 68/70/99/195/207/214/230/319/489, 68/70/195/207/214/489, 65/68/70/207/214/489, 68/70/141/207/214/230/319/489, 65/68/70/99/207/214/230/319/489, 68/70/99/141/207/214/319/489, 68/70/154/207/214/319/489, 70/141/154/207/214/489, 68/70/99/207/214/489, 70/99/207/214/230/319/489, 70/99/207/214/489, 70/207/214/230/489, 68/70/207/214/230/319/489, 70/154/207/214/319/489, 65/68/70/154/207/214/319/489, 70/141/207/214/489, 70/207/214/336/489, 70/141/207/214/230/489, 68/70/154/207/214/336/489, 65/68/70/99/207/214/319/489, 70/154/207/214/489, 70/207/214/319/489, 70/207/214/230/319/489, 70/99/141/207/214/319/489, 70/207/214/489/509, 70/108/207/214/489, 70/108/117/207/214/489/509, 70/207/214/489/519, 70/108/207/214/489/519, 70/88/207/214/489, 70/117/207/214/413/489/509, 70/207/214/489/509/519, 70/207/214/413/489, 70/108/117/207/214/315/489, 70/117/207/214/271/489, 70/117/207/214/413/489/509/529, 70/207/214/413/489/509, 70/207/214/489/469, 70/207/214/489/524, 70/207/214/489/507, 70/207/214/489/525, 70/104/207/214/489, 70/95/207/214/489, 70/207/214/359/489, 70/207/214/374/489, 70/107/207/214/489, 70/89/207/214/489, 70/207/214/260/489, 70/110/207/214/489, 70/186/207/214/489, 70/207/214/370/489, 70/191/207/214/489, 70/207/214/333/489, 70/189/207/214/489, 70/207/214/468/489, 70/207/214/473/489, 70/115/207/214/489, 53/70/207/214/489, 70/207/214/449/489, 70/207/214/489/518, 70/207/214/236/489, 70/207/214/481/489, 59/70/207/214/220/489, 70/207/214/258/489, 70/207/214/467/489, 51/70/207/214/489, 70/207/214/415/489, 70/103/207/214/489, 70/207/214/489/493, 70/78/207/214/489, 65/70/207/214/489, 70/207/214/335/489, 70/207/214/489/520, 70/101/207/214/489, 70/207/214/341/489, 70/207/214/471/489, 70/207/211/214/489, 70/206/207/214/489, 70/93/207/214/489, 70/207/214/489/490, 70/71/207/214/489, 70/79/207/214/489, 70/180/207/214/489, 70/172/207/214/489, 70/142/207/214/489, 70/207/214/489/529, 70/77/207/214/489, 70/207/212/214/489, 70/207/214/474/489, 70/106/207/214/489, 70/207/214/386/489/507, 70/117/207/214/489, 70/207/214/373/489, 70/207/214/337/489, 70/107/207/214/246/489, 70/207/214/489/516, 70/207/214/233/489, 70/91/207/214/489, 70/194/207/214/489, 70/84/207/214/489, 70/207/214/361/489, 70/171/207/214/489, 70/207/214/489/536, 50/70/207/214/489, 70/116/207/214/489, 70/207/214/239/489, 44/70/207/214/489, 40/70/207/214/489, 70/113/207/214/489, 70/207/214/243/489, 70/169/207/214/489, 70/207/214/489/504, 70/207/214/489/505, 70/109/207/214/489, 70/207/214/479/489, 70/207/214/477/489, 70/97/207/214/489, 70/182/207/214/489, 70/111/207/214/489, 70/207/214/489/508, 70/207/214/314/489, 70/102/207/214/489, 70/207/214/338/489, 70/96/207/214/489, 70/138/207/214/489, 42/70/207/214/489, 70/207/214/263/489, 70/207/214/222/489, 70/207/214/489/522, 70/207/214/265/489, 70/207/214/489/514, 70/207/214/489/515, 70/207/214/246/489, 70/207/214, 46/70/207/214/489, 70/133/207/214/489, 70/207/214/332/489, 70/207/214/489/506, 70/207/214/489/512, 70/137/207/214/489, 70/190/207/214/489, 70/105/207/214/489, 70/207/214/301/489, 70/207/214/329/489, 70/207/214/339/489, or 70/151/207/214/489, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set 68L/70L/207S/214F/336A/489L, 65L/70L/99R/207S/214F/319I/489L, 65L/68L/70L/207S/214F/319I/489L, 68V/70L/207S/214F/319I/489L, 68V/70L/207S/214F/489L, 65L/70L/207S/214F/319I/489L, 70L/195M/207S/214F/489L, 65L/154E/70L/207S/214F/319I/489L, 70L/207S/214F/489L, 70L/207S/214F/489L, 65L/68L/70L/99R/154E/207S/214F/230L/319I/489L, 68L/70L/207S/214F/230L/489L, 65L/68V/70L/207S/214F/319I/489L, 70L/195M/207S/214F/230L/489L, 65L/70L/207S/214F/230L/489L, 70L/141D/207S/214F/319I/489L, 70L/154E/207S/214F/336A/489L, 68L/70L/207S/214F/319I/489L, 68V/70L/154E/207S/214F/489L, 68V/70L/195M/207S/214F/230L/319I/336A/489L, 68V/70L/99R/195M/207S/214F/230L/319I/489L, 68V/70L/195M/207S/214F/489L, 65L/68V/70L/207S/214F/489L, 68L/70L/141D/207S/214F/230L/319I/489L, 65L/68V/70L/99R/207S/214F/230L/319I/489L, 68L/70L/99R/141D/207S/214F/319I/489L, 68V/70L/154E/207S/214F/319I/489L, 70L/141D/154E/207S/214F/489L, 68V/70L/99R/207S/214F/489L, 70L/99R/207S/214F/230L/319I/489L, 70L/99R/207S/214F/489L, 70L/207S/214F/230L/489L, 68V/70L/207S/214F/230L/319I/489L, 68L/70L/207S/214F/489L, 70L/154E/207S/214F/319I/489L, 65L/68L/70L/154E/207S/214F/319I/489L, 70L/141D/207S/214F/489L, 68V/70L/207S/214F/230L/489L, 70L/207S/214F/336A/489L, 70L/141D/207S/214F/230L/489L, 68V/70L/154E/207S/214F/336A/489L, 65L/68L/70L/207S/214F/489L, 65L/68V/70L/99R/207S/214F/319I/489L, 70L/154E/207S/214F/489L, 70L/207S/214F/319I/489L, 70L/207S/214F/230L/319I/489L, 68L/70L/154E/207S/214F/489L, 70L/99R/141D/207S/214F/319I/489L, 68L/70L/154E/207S/214F/319I/489L, 70L/207S/214F/489L/509F, 70L/108I/207S/214F/489L, 70L/108I/117I/207S/214F/489L/509F, 70L/207S/214F/489L/519I, 70L/108I/207S/214F/489L/519I, 70L/88L/207S/214F/489L, 70L/117I/207S/214F/413Y/489L/509F, 70L/207S/214F/489L/509F/519I, 70L/207S/214F/413Y/489L, 70L/108I/117I/207S/214F/315T/489L, 70L/117I/207S/214F/271T/489L, 70L/117I/207S/214F/413Y/489L/509F/529Q, 70L/207S/214F/413Y/489L/509F, 70L/207S/214F/489L/469E, 70L/207S/214F/489L/524K, 70L/207S/214F/489L/507M, 70L/207S/214F/489L/509I, 70L/207S/214F/489L/525Q, 70L/207S/214F/489A, 70L/104M/207S/214F/489L, 70L/207S/214F/489L/507A, 70L/95G/207S/214F/489L, 70L/207S/214F/359E/489L, 70L/207S/214F/374V/489L, 70L/207S/214F/489L/509W, 70L/107D/207S/214F/489L, 70L/89C/207S/214F/489L, 70L/207S/214F/260R/489L, 70L/110Q/207S/214F/489L, 70L/186G/207S/214F/489L, 70L/207S/214F/370R/489L, 70L/207S/214F/489L/509R, 70L/191R/207S/214F/489L, 70L/207S/214F/333L/489L, 70L/189R/207S/214F/489L, 70L/207S/214F/468G/489L, 70L/207S/214F/473I/489L, 70L/115M/207S/214F/489L, 70L/191N/207S/214F/489L, 53L/70L/207S/214F/489L, 70L/207S/214F/489L/524S, 70L/207S/214F/449L/489L, 70L/207S/214F/489L/518E, 70L/207S/214F/236M/489L, 70L/207S/214F/481H/489L, 70L/115R/207S/214F/489L, 59N/70L/207S/214F/220G/489L, 70L/207S/214F/489G, 70L/207S/214F/258T/489L, 70L/207S/214F/467N/489L, 51P/70L/207S/214F/489L, 70L/207S/214F/415C/489L, 70L/103S/207S/214F/489L, 70L/207S/214F/489L/493T, 70L/207S/214F/489T, 70L/207S/214F/489L/518V, 70L/78L/207S/214F/489L, 70L/195W/207S/214F/489L, 65M/70L/207S/214F/489L, 70L/207S/214F/413W/489L, 70L/207S/214F/335M/489L, 70L/207S/214F/489L/520K, 70L/107E/207S/214F/489L, 70L/101Q/207S/214F/489L, 70L/207S/214F/449K/489L, 70L/207S/214F/481K/489L, 70L/104V/207S/214F/489L, 70L/207S/214F/341R/489L, 70L/78M/207S/214F/489L, 70L/207S/214F/489L/507L, 70L/207S/214F/449M/489L, 70L/207S/214F/4711/489L, 70L/207S/211L/214F/489L, 70L/207S/214F/489L/520D, 70L/103P/207S/214F/489L, 70L/206F/207S/214F/489L, 70L/93S/207S/214F/489L, 70L/207S/214F/489L/490S, 70L/207S/214F/473C/489L, 70L/207S/214F/489L/507R, 70L/207S/211I/214F/489L, 70L/71S/207S/214F/489L, 70L/79R/207S/214F/489L, 70L/180C/207S/214F/489L, 70L/207S/214F/489L/509G, 70L/206R/207S/214F/489L, 70L/207S/211N/214F/489L, 70L/172S/207S/214F/489L, 70L/207S/214F/489L/490R, 70L/207D/214F/489L, 70L/115V/207S/214F/489L, 63L/70L/207S/214F/489L, 70L/142A/207S/214F/489L, 70L/207S/214F/489L/529M, 70L/71R/207S/214F/489L, 70L/207S/214F/473L/489L, 70L/95T/207S/214F/489L, 70L/107A/207S/214F/489L, 70L/207S/214F/489L/520V, 70L/101L/207S/214F/489L, 70L/207S/214F/489L/520S, 70L/71H/207S/214F/489L, 70L/89D/207S/214F/489L, 70L/77I/207S/214F/489L, 70L/207S/214F/489L/507Y, 70L/107C/207S/214F/489L, 70L/207S/214F/489L/518S, 70L/207S/212A/214F/489L, 70L/71F/207S/214F/489L, 70L/207S/214F/489L/525C, 70L/207S/214F/449R/489L, 70L/107M/207S/214F/489L, 70L/207S/214F/467H/489L, 70L/207S/214F/474D/489L, 70L/101S/207S/214F/489L, 70L/106W/207S/214F/489L, 70L/207S/214F/386L/489L/507M, 70L/207S/214F/471S/489L, 70L/107Q/207S/214F/489L, 68Q/70L/207S/214F/489L, 70L/79I/207S/214F/489L, 70L/207S/214F/489L/518L, 70L/207S/214F/474A/489L, 70L/107S/207S/214F/489L, 70L/117T/207S/214F/489L, 70L/207S/214F/467M/489L, 70L/207S/214F/471R/489L, 70L/207S/214F/341S/489L, 70L/207S/214F/489L/490G, 70L/207S/214F/471F/489L, 70L/207S/214F/489L/490T, 70L/207S/214F/333T/489L, 70L/101R/207S/214F/489L, 70L/207S/214F/373V/489L, 70L/101V/207S/214F/489L, 70L/101W/207S/214F/489L, 65S/70L/207S/214F/489L, 70L/207S/214F/489L/490V, 70L/207S/214F/337N/489L, 70L/207S/214F/374T/489L, 68Y/70L/207S/214F/489L, 70L/95L/207S/214F/489L, 70L/71G/207S/214F/489L, 70L/207S/214F/471V/489L, 70L/107M/207S/214F/246R/489L, 70L/110A/207S/214F/489L, 70L/142Y/207S/214F/489L, 65V/70L/207S/214F/489L, 70L/71M/207S/214F/489L, 70L/77G/207S/214F/489L, 70L/77R/207S/214F/489L, 70L/207S/214F/230Y/489L, 70L/207S/214F/489L/490Q, 70L/101T/207S/214F/489L, 70L/103A/207S/214F/489L, 70L/207S/214F/489L/507T, 70L/207S/214F/471T/489L, 70L/207S/214F/489L/509V, 70L/207S/214F/489L/520R, 70L/207S/214F/319F/489L, 70L/142N/207S/214F/489L, 70L/79V/207S/214F/489L, 70L/207S/214F/467L/489L, 70L/77N/207S/214F/489L, 70L/71L/207S/214F/489L, 68W/70L/207S/214F/489L, 70L/189S/207S/214F/489L, 70L/207S/214F/359P/489L, 70L/71Y/207S/214F/489L, 70L/77T/207S/214F/489L, 70L/142M/207S/214F/489L, 70L/207S/214F/373A/489L, 70L/207S/214F/319V/489L, 70L/207S/212N/214F/489L, 70L/110H/207S/214F/489L, 70L/207S/214F/333C/489L, 70L/207S/214F/489L/490I, 68F/70L/207S/214F/489L, 70L/207S/214F/474S/489L, 70L/103L/207S/214F/489L, 70L/77H/207S/214F/489L, 70L/207S/214F/471E/489L, 70L/207S/214F/489L/516L, 70L/207S/214F/467R/489L, 70L/207S/214F/233R/489L, 70L/91T/207S/214F/489L, 70L/194L/207S/214F/489L, 70L/84G/207S/214F/489L, 70L/207S/214F/361V/489L, 70L/207S/214F/233A/489L, 70L/171V/207S/214F/489L, 70L/207S/214F/489L/536L, 50A/70L/207S/214F/489L, 70L/116R/207S/214F/489L, 70L/207S/214F/239T/489L, 44V/70L/207S/214F/489L, 40S/70L/207S/214F/489L, 70L/113N/207S/214F/489L, 70L/91I/207S/214F/489L, 70L/99F/207S/214F/489L, 70L/207S/214F/243L/489L, 70L/169V/207S/214F/489L, 70L/207S/214F/489L/504P, 70L/207S/214F/489L/505R, 70L/109R/207S/214F/489L, 70L/207S/214F/489L/536T, 70L/207S/214F/479A/489L, 70L/207S/214F/479L/489L, 70L/207S/214F/477H/489L, 70L/84K/207S/214F/489L, 70L/97N/207S/214F/489L, 70L/97K/207S/214F/489L, 70L/182M/207S/214F/489L, 70L/111H/207S/214F/489L, 70L/207S/214F/361Y/489L, 70L/207S/214F/489L/508R, 70L/207S/214F/314R/489L, 70L/207S/214F/336G/489L, 70L/102E/207S/214F/489L, 70L/207S/214F/338R/489L, 70L/207S/214F/489L/505L, 70L/99L/207S/214F/489L, 70L/96S/207S/214F/489L, 70L/138S/207S/214F/489L, 70L/207S/214F/489L/508T, 70L/96I/207S/214F/489L, 70L/207S/214F/489L/508Q, 70L/207S/214F/243M/489L, 70L/111L/207S/214F/489L, 70L/207S/214F/489L/508S, 70L/207S/214F/479R/489L, 42A/70L/207S/214F/489L, 70L/207S/214F/239I/489L, 70L/97I/207S/214F/489L, 70L/116E/207S/214F/489L, 70L/113I/207S/214F/489L, 70L/207S/214F/477V/489L, 70L/207S/214F/489L/536I, 70L/207S/214F/263G/489L, 70L/99S/207S/214F/489L, 70L/207S/214F/222I/489L, 70L/207S/214F/489L/536D, 70L/207S/214F/477G/489L, 70L/207S/214F/489L/536W, 70L/207S/214F/489L/536C, 70L/207S/214F/336R/489L, 70L/207S/214F/243R/489L, 70L/116V/207S/214F/489L, 70L/207S/214F/489L/536R, 70L/207S/214F/489L/522V, 70L/102S/207S/214F/489L, 70L/97R/207S/214F/489L, 40H/70L/207S/214F/489L, 70L/207S/214F/243V/489L, 70L/207S/214F/265S/489L, 70L/116Q/207S/214F/489L, 70L/207S/214F/489L/514E, 70L/207S/214F/489L/508L, 70L/207S/214F/489L/515T, 70L/207S/214F/246R/489L, 70L/207S/214F/361W/489L, 70L/111F/207S/214F/489L, 70L/207S/214F, 70L/207S/214F/477R/489L, 70L/116Y/207S/214F/489L, 46R/70L/207S/214F/489L, 70L/133M/207S/214F/489L, 70L/207S/214F/332R/489L, 70L/207S/214F/239R/489L, 70L/109G/207S/214F/489L, 70L/207S/214F/489L/506T, 70L/207S/214F/489L/504G, 70L/207S/214F/233Q/489L, 70L/96R/207S/214F/489L, 70L/99G/207S/214F/489L, 70L/207S/214F/489L/504T, 70L/96G/207S/214F/489L, 70L/97M/207S/214F/489L, 70L/109L/207S/214F/489L, 70L/207S/214F/489L/515G, 70L/207S/214F/233G/489L, 70L/84A/207S/214F/489L, 70L/102N/207S/214F/489L, 70L/207S/214F/489L/504W, 70L/207S/214F/265R/489L, 70L/99V/207S/214F/489L, 70L/207S/214F/489L/512L, 70L/137V/207S/214F/489L, 70L/207S/214F/489L/536A, 70L/113R/207S/214F/489L, 70L/171P/207S/214F/489L, 70L/190W/207S/214F/489L, 70L/207S/214F/314G/489L, 46M/70L/207S/214F/489L, 70L/182H/207S/214F/489L, 70L/207S/214F/246K/489L, 70L/105T/207S/214F/489L, 70L/207S/214F/301K/489L, 70L/207S/214F/489L/522T, 70L/207S/214F/329L/489L, 70L/96V/207S/214F/489L, 70L/207S/214F/339A/489L, 70L/207S/214F/477S/489L, 70L/113G/207S/214F/489L, 70L/207S/214F/361L/489L, 70L/207S/214F/489L/505I, 70L/105M/207S/214F/489L, 70L/116M/207S/214F/489L, 70L/96M/207S/214F/489L, 70L/109M/207S/214F/489L, 70L/171C/207S/214F/489L, 70L/207S/214F/489L/536M, or 70L/151L/207S/214F/489L, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set of an engineered glucocerebrosidase set forth in Tables 4.1, 4.2, 4.3 and 4.4, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set at amino acid position(s) 65/68/70/99/154/195/207/214/230/319/361/489, 65/68/70/99/154/207/214/230/319/489/514, 65/68/70/79/99/154/195/207/214/230/263/319/361/489, 65/68/70/99/154/207/214/230/319/374/489, 65/68/70/79/99/154/195/207/214/230/319/489, 65/68/70/99/154/207/214/230/319/489, 65/68/70/99/154/207/214/230/233/263/319/337/359/361/374/489, 65/68/70/79/99/154/207/214/230/319/489/536, 65/68/70/79/99/154/207/214/230/233/239/319/359/361/471/489, 65/68/70/99/154/207/214/230/319/471/489, 65/68/70/99/154/195/207/214/230/233/319/337/374/489/514, 65/68/70/99/154/195/207/214/230/233/319/359/489/536, 65/68/70/99/154/195/207/214/230/263/276/319/359/361/374/471/489, 65/68/70/79/99/154/207/214/230/319/489, 65/68/70/99/154/195/207/214/230/319/233/337/374/489/536, 65/68/70/99/154/195/207/214/230/319/489, 65/68/70/99/154/195/207/214/230/319/337/359/361/489, 65/68/70/99/154/207/214/230/263/319/489, 65/68/70/99/154/207/214/230/233/319/489, 65/68/70/99/154/195/207/214/230/319/337/361/374/471/489, 65/68/70/99/154/207/214/230/319/374/471/489, 65/68/70/99/154/207/214/230/319/374/489/536, 65/68/70/99/154/207/214/230/239/319/489, 65/68/70/99/154/207/214/230/233/319/489/536, 65/68/70/99/154/207/214/230/319/263/374/489, 65/68/70/99/154/195/207/214/230/263/319/337/361/374/471/489, 65/68/70/79/99/154/207/214/230/319/374/489, 65/68/70/99/154/207/214/230/319/361/374/471/489/536, 65/68/70/79/99/154/207/214/230/319/359/374/489, 65/68/70/99/154/207/214/230/319/489/536, 65/68/70/99/154/207/214/230/319/359/361/374/489/514, 65/68/70/99/154/195/207/214/230/239/263/361/374/319/489/514, 65/68/70/99/154/195/207/214/230/263/319/374/489, 65/68/70/99/154/195/207/214/230/319/374/489, 65/68/70/99/154/207/214/230/233/319/361/489/536, 65/68/70/99/154/207/214/230/233/319/374/471/489, 65/68/70/99/154/207/214/230/233/319/359/361/471/489, 65/68/70/99/154/207/214/230/319/359/471/489, or 65/68/70/99/154/207/214/230/319/359/489, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set 65V/68L/70L/99R/154E/195W/207S/214F/230L/319I/361W/489L, 65L/68L/70L/99R/154E/207S/214F/230L/319I/489L/514E, 65L/68L/70L/79V/99R/154E/195W/207S/214F/230L/263G/319I/361W/489L, 65V/68L/70L/99R/154E/207S/214F/230L/319I/374T/489L, 65L/68L/70L/79V/99R/154E/195W/207S/214F/230L/319I/489L, 65V/68L/70L/99R/154E/207S/214F/230Y/319I/489L, 65V/68L/70L/99R/154E/207S/214F/230L/233A/263G/319I/337N/359P/361W/374T/489L, 65L/68L/70L/79V/99R/154E/207S/214F/230L/319I/489L/536D, 65L/68L/70L/79V/99R/154E/207S/214F/230Y/233A/239R/319I/359P/361W/471T/489L, 65V/68L/70L/99R/154E/207S/214F/230L/319I/471T/489L, 65L/68L/70L/99R/154E/207S/214F/230L/319I/374T/489L, 65V/68L/70L/99R/154E/195W/207S/214F/230Y/233A/319I/337N/374T/489L/514E, 65V/68L/70L/99R/154E/195W/207S/214F/230L/233A/319I/359P/489L/536D, 65L/68L/70L/99R/154E/207S/214F/230Y/319I/489L, 65V/68L/70L/99R/154E/195W/207S/214F/230L/263G/276T/319I/359P/361W/374T/471T/489L, 65L/68L/70L/79V/99R/154E/207S/214F/230L/319I/489L, 65L/68L/70L/99R/154E/195W/207S/214F/230Y/319I/233A/337N/374T/489L/536D, 65L/68L/70L/99R/154E/195W/207S/214F/230Y/319I/489L, 65L/68L/70L/99R/154E/195W/207S/214F/230Y/319I/337N/359P/361W/489L, 65L/68L/70L/99R/154E/207S/214F/230Y/263G/319I/489I, 65V/68L/70L/99R/154E/207S/214F/230Y/233G/319I/489L, 65L/68L/70L/99R/154E/207S/214F/230L/263G/319I/489L, 65L/68L/70L/99R/154E/195W/207S/214F/230Y/319I/337N/361W/374T/471T/489L, 65V/68L/70L/99R/154E/195W/207S/214F/230L/319I/489L, 65L/68L/70L/99R/154E/207S/214F/230L/319I/374T/471T/489L, 65L/68L/70L/99R/154E/207S/214F/230L/319I/374T/489L/536D, 65L/68L/70L/99R/154E/207S/214F/230L/239R/319I/489L, 65V/68L/70L/99R/154E/207S/214F/230L/319I/489L, 65L/68L/70L/99R/154E/207S/214F/230Y/233G/319I/489L/536L, 65L/68L/70L/99R/154E/207S/214F/230Y/319I/263G/374T/489L, 65V/68L/70L/99R/154E/195W/207S/214F/230Y/263G/319I/337N/361W/374T/471T/489L, 65L/68L/70L/79V/99R/154E/207S/214F/230L/319I/374T/489L, 65V/68L/70L/99R/154E/207S/214F/230L/319I/361W/374T/471T/489L/536D, 65L/68L/70L/99R/154E/195W/207S/214F/230L/319I/489L, 65L/68L/70L/99R/154E/207S/214F/230L/233G/319I/489L, 65L/68L/70L/79V/99R/154E/207S/214F/230Y/319I/359P/374T/489L, 65L/68L/70L/99R/154E/207S/214F/230L/319I/489L/536D, 65L/68L/70L/99R/154E/207S/214F/230Y/319I/359P/361W/374T/489L/514E, 65V/68L/70L/79V/99R/154E/207S/214F/230Y/319I/489L, 65L/68L/70L/99R/154E/195W/207S/214F/230L/239R/263G/361W/374T/319I/489L/514E, 65L/68L/70L/99R/154E/195W/207S/214F/230L/263G/319I/374T/489L, 65L/68L/70L/99R/154E/195W/207S/214F/230L/319I/374T/489L, 65V/68L/70L/99R/154E/207S/214F/230Y/233A/319I/361W/489L/536L, 65L/68L/70L/99R/154E/207S/214F/230Y/233A/319I/374T/471T/489L, 65V/68L/70L/99R/154E/207S/214F/230L/233A/319I/359P/361W/471T/489L, 65L/68L/70L/99R/154E/207S/214F/230L/319I/489L/536L, 65V/68L/70L/99R/154E/207S/214F/230L/319I/359P/471T/489L, 65V/68L/70L/79V/99R/154E/207S/214F/230L/319I/489L, or 65V/68L/70L/99R/154E/207S/214F/230L/319I/359P/489L, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set of an engineered glucocerebrosidase set forth in Table 5.1, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set at amino acid position(s) 65/68/70/99/154/195/207/214/230/263/319/337/359/361/374/471/489, 65/68/70/79/99/154/195/207/214/230/263/319/337/359/361/374/471/489, 65/68/70/79/97/99/154/182/195/207/214/230/263/319/337/361/374/471/489, 65/68/70/97/99/154/195/207/214/230/263/319/337/359/361/374/471/489, 65/68/70/97/99/154/195/207/214/230/263/319/337/361/374/471/489, 65/68/70/79/99/154/182/195/207/214/230/263/319/337/361/374/471/489, 65/68/70/99/154/195/201/207/214/230/263/319/337/361/374/471/489, 65/68/70/99/154/195/207/214/230/263/319/337/361/374/404/471/489, 65/68/70/99/154/195/207/214/230/263/319/337/361/374/471/484/489, 65/68/70/99/154/195/207/214/230/263/319/337/361/374/446/471/489, 65/68/70/99/154/195/207/214/230/263/319/337/361/374/471/489/494/495, 65/68/70/99/154/195/207/214/230/263/319/337/361/374/471/489/495, 65/68/70/99/154/195/207/214/230/263/319/337/361/374/412/471/489, 65/68/70/99/154/195/207/214/230/263/319/337/361/374/385/471/489, 65/68/70/99/100/154/195/207/214/230/263/319/337/361/374/471/489, 65/68/70/99/154/184/195/207/214/230/263/319/337/361/374/471/489, 65/68/70/99/154/385/195/207/214/230/263/319/337/361/374/471/489, 65/68/70/82/99/154/195/207/214/230/263/319/337/361/374/471/489, 65/68/70/75/99/154/195/207/214/230/263/319/337/361/374/471/489, 65/68/70/71/99/154/195/207/214/230/263/319/337/361/374/471/489, 41/65/68/70/99/154/195/207/214/230/263/319/337/361/374/471/489, 65/68/70/99/154/195/207/214/230/263/319/337/361/374/471/489, 65/68/70/94/99/154/195/207/214/230/263/319/337/361/374/471/489, 65/68/70/92/99/154/195/207/214/230/263/319/337/361/374/471/489, 65/68/70/98/99/154/195/207/214/230/263/319/337/361/374/471/489, 65/68/70/99/114/154/195/207/214/230/263/319/337/361/374/471/489, 65/68/70/95/99/154/195/207/214/230/263/319/337/361/374/471/489, 65/68/70/99/102/154/195/207/214/230/263/319/337/361/374/471/489, 65/68/70/96/99/154/195/207/214/230/263/319/337/361/374/471/489, 65/68/70/99/105/154/195/207/214/230/263/319/337/361/374/471/489, 65/68/70/99/113/154/195/207/214/230/263/319/337/361/374/471/489, 65/68/70/99/154/195/207/214/230/263/319/325/337/361/374/471/489, 65/68/70/99/154/195/207/214/230/263/319/336/337/361/374/471/489, 65/68/70/99/154/195/207/214/230/262/263/319/337/361/374/471/489, 65/68/70/99/154/195/207/214/230/263/319/337/361/374/386/471/489, 65/68/70/99/154/195/207/214/230/263/319/337/360/361/374/471/489, 65/68/70/99/154/195/207/214/230/263/319/337/361/374/400/471/489, 65/68/70/99/154/195/207/214/230/263/319/337/361/374/342/471/489, 65/68/70/99/154/195/207/214/230/263/319/337/361/374/390/471/489, 65/68/70/99/154/195/207/214/230/263/319/337/361/264/374/471/489, 65/68/70/99/154/195/207/214/230/263/298/319/337/361/374/471/489, 65/68/70/99/154/195/207/214/230/263/319/337/361/374/382/471/489, 65/68/70/99/154/195/207/214/230/263/319/337/361/374/408/471/489, 65/68/70/99/154/195/207/214/230/263/319/337/361/374/449/471/489, 65/68/70/99/154/195/207/214/230/263/319/337/361/374/459/471/489, 65/68/70/99/154/195/207/214/230/263/319/337/361/374/434/471/489, 65/68/70/99/154/195/207/214/230/263/319/337/361/374/471/489/502, 29/65/68/70/99/154/195/207/214/230/263/319/337/361/374/471/489/534, 65/68/70/99/154/195/207/214/230/263/319/337/361/374/471/489, 65/68/70/99/154/195/207/214/230/263/319/337/361/374/471/489/497, 65/68/70/99/154/195/207/214/230/263/319/337/361/374/471/489/533, 65/68/70/99/154/195/207/214/230/263/319/337/361/374/471/489/536, 65/68/70/99/154/195/207/214/230/263/319/337/361/374/471/485/489, 65/68/70/99/154/195/207/214/230/263/319/337/361/374/471/489/510, 65/68/70/99/154/195/207/214/230/263/319/337/361/374/471/489/532, 65/68/70/99/154/195/207/214/230/263/319/337/361/374/471/489/534, 65/68/70/99/154/195/207/214/230/263/319/337/361/374/471/489/494, 65/68/70/99/154/182/195/207/214/230/263/319/337/361/374/471/489, 65/68/70/99/154/195/204/207/214/230/263/319/337/361/374/471/489, 65/68/70/99/154/194/195/207/214/230/263/319/337/361/374/471/489, 65/68/70/99/154/195/202/207/214/230/263/319/337/361/374/471/489, 65/68/70/99/154/195/207/214/230/263/319/337/361/374/471/489, 65/68/70/99/144/154/195/207/214/230/263/319/337/361/374/471/489, 65/68/70/99/154/175/195/207/214/230/263/319/337/361/374/471/489, 65/68/70/99/154/195/207/214/220/230/263/319/337/361/374/471/489, 65/68/70/99/150/154/195/207/214/230/263/319/337/361/374/471/489, or 65/68/70/99/154/195/206/207/214/230/263/319/337/361/374/471/489, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set 65V/68L/70L/99R/154E/195W/207S/214F/230Y/263G/319I/337N/359P/361W/374T/471T/489L, 65V/68L/70L/79V/99R/154E/195W/207S/214F/230Y/263G/319I/337N/359P/361W/374T/471T/489L, 65V/68L/70L/79V/97R/99R/154E/182M/195W/207S/214F/230Y/263G/319I/337N/361W/374T/471T/489L, 65V/68L/70L/97R/99R/154E/195W/207S/214F/230Y/263G/319I/337N/359P/361W/374T/471T/489L, 65V/68L/70L/97R/99R/154E/195W/207S/214F/230Y/263G/319I/337N/361W/374T/471T/489L, 65V/68L/70L/79V/99R/154E/182M/195W/207S/214F/230Y/263G/319I/337N/361W/374T/471T/489L, 65V/68L/70L/99R/154E/195W/201T/207S/214F/230Y/263G/319I/337N/361W/374T/471T/489L, 65V/68L/70L/99R/154E/195W/207S/214F/230Y/263G/319I/337N/361W/374T/404Q/471T/489L, 65V/68L/70L/99R/154E/195W/207S/214F/230Y/263G/319I/337N/361W/374T/471T/484E/489L, 65V/68L/70L/99R/154E/195W/201R/207S/214F/230Y/263G/319I/337N/361W/374T/471T/489L, 65V/68L/70L/99R/154E/195W/207S/214F/230Y/263G/319I/337N/361W/374T/446N/471T/489L, 65V/68L/70L/99R/154E/195W/207S/214F/230Y/263G/319I/337N/361W/374T/471T/489L/494T/495G, 65V/68L/70L/99R/154E/195W/207S/214F/230Y/263G/319I/337N/361W/374T/471T/489L/495G, 65V/68L/70L/99R/154E/195W/207S/214F/230Y/263G/319I/337N/361W/374T/412W/471T/489L, 65V/68L/70L/99R/154E/195W/207S/214F/230Y/263G/319I/337N/361W/374T/385N/471T/489L, 65V/68L/70L/99R/154E/195W/207S/214F/230Y/263G/319I/337N/361W/374T/385G/471T/489L, 65V/68L/70L/99R/100N/154E/195W/207S/214F/230Y/263G/319I/337N/361W/374T/471T/489L, 65V/68L/70L/99R/154E/184V/195W/207S/214F/230Y/263G/319I/337N/361W/374T/471T/489L, 65V/68L/70L/99R/154E/385H/195W/207S/214F/230Y/263G/319I/337N/361W/374T/471T/489L, 65V/68L/70L/82S/99R/154E/195W/207S/214F/230Y/263G/319I/337N/361W/374T/471T/489L, 65V/68L/70L/75A/99R/154E/195W/207S/214F/230Y/263G/319I/337N/361W/374T/471T/489L, 65V/68L/70L/71T/99R/154E/195W/207S/214F/230Y/263G/319I/337N/361W/374T/471T/489L, 41H/65V/68L/70L/99R/154E/195W/207S/214F/230Y/263G/319I/337N/361W/374T/471T/489L, 65V/68L/70L/A97K/99R/154E/195W/207S/214F/230Y/263G/319I/337N/361W/374T/471T/489I, 65V/68L/70L/99E/154E/195W/207S/214F/230Y/263G/319I/337N/361W/374T/471T/489L, 65V/68L/70L/97Q/99R/154E/195W/207S/214F/230Y/263G/319I/337N/361W/374T/471T/489L, 65V/68L/70L/P94N/99R/154E/195W/207S/214F/230Y/263G/319I/337N/361W/374T/471T/489L, 65V/68L/70L/99R/100R/154E/195W/207S/214F/230Y/263G/319I/337N/361W/374T/471T/489L, 65V/68L/70L/92A/99R/154E/195W/207S/214F/230Y/263G/319I/337N/361W/374T/471T/489L, 65V/68L/70L/98P/99R/154E/195W/207S/214F/230Y/263G/319I/337N/361W/374T/471T/489L, 65V/68L/70L/99W/154E/195W/207S/214F/230Y/263G/319I/337N/361W/374T/471T/489L, 65V/68L/70L/92G/99R/154E/195W/207S/214F/230Y/263G/319I/337N/361W/374T/471T/489L, 65V/68L/70L/99R/114Y/154E/195W/207S/214F/230Y/263G/319I/337N/361W/374T/471T/489L, 65V/68L/70L/195H/99R/154E/195W/207S/214F/230Y/263G/319I/337N/361W/374T/471T/489L, 65V/68L/70L/99R/T102A/154E/195W/207S/214F/230Y/263G/319I/337N/361W/374T/471T/489L, 65V/68L/70L/M92K/99R/154E/195W/207S/214F/230Y/263G/319I/337N/361W/374T/471T/489L, 65V/68L/70L/96D/99R/154E/195W/207S/214F/230Y/263G/319I/337N/361W/374T/471T/489L, 65V/68L/70L/94E/99R/154E/195W/207S/214F/230Y/263G/319I/337N/361W/374T/471T/489L, 65V/68L/70L/96E/99R/154E/195W/207S/214F/230Y/263G/319I/337N/361W/374T/471T/489L, 65V/68L/70L/195W/99R/154E/195W/207S/214F/230Y/263G/319I/337N/361W/374T/471T/489L, 65V/68L/70L/99R/105I/154E/195W/207S/214F/230Y/263G/319I/337N/361W/374T/471T/489L, 65V/68L/70L/99R/113Q/154E/195W/207S/214F/230Y/263G/319I/337N/361W/374T/471T/489L, 65V/68L/70L/92H/99R/154E/195W/207S/214F/230Y/263G/319I/337N/361W/374T/471T/489L, 65V/68L/70L/92Q/99R/154E/195W/207S/214F/230Y/263G/319I/337N/361W/374T/471T/489L, 65V/68L/70L/92E/99R/154E/195W/207S/214F/230Y/263G/319I/337N/361W/374T/471T/489L, 65V/68L/70L/99R/154E/195W/207S/214F/230Y/263G/319I/L325V/337N/361W/374T/471T/489L, 65V/68L/70L/99R/154E/195W/207S/214F/230Y/263G/319I/336E/337N/361W/374T/471T/489L, 65V/68L/70L/99R/154E/195W/207S/214F/230Y/263G/319I/337N/361W/374T/385Q/471T/489L, 65V/68L/70L/99R/154E/195W/207S/214F/230Y/262Q/263G/319I/337N/361W/374T/471T/489L, 65V/68L/70L/99R/154E/195W/207S/214F/230Y/263G/319I/337N/361W/374T/386I/471T/489L, 65V/68L/70L/99R/154E/195W/207S/214F/230Y/263G/319I/337N/360E/361W/374T/471T/489L, 65V/68L/70L/99R/154E/195W/207S/214F/230Y/263G/319I/337N/361W/374T/400T/471T/489L, 65V/68L/70L/99R/154E/195W/207S/214F/230Y/263G/319I/337N/361W/374T/342Q/471T/489L, 65V/68L/70L/99R/154E/195W/207S/214F/230Y/263G/319I/337N/361W/374T/400E/471T/489L, 65V/68L/70L/99R/154E/195W/207S/214F/230Y/263G/319I/337N/361W/374T/400Q/471T/489L, 65V/68L/70L/99R/154E/195W/207S/214F/230Y/263G/319I/337N/361W/374T/390A/471T/489L, 65V/68L/70L/99R/154E/195W/207S/214F/230Y/263G/319I/337N/361W/264I/374T/471T/489L, 65V/68L/70L/99R/154E/195W/207S/214F/230Y/263G/298W/319I/337N/361W/374T/471T/489L, 65V/68L/70L/99R/154E/195W/207S/214F/230Y/263G/319I/337N/361W/374T/382A/471T/489L, 65V/68L/70L/99R/154E/195W/207S/214F/230Y/263G/319I/337N/361W/374T/408D/471T/489L, 65V/68L/70L/99R/154E/195W/207S/214F/230Y/263G/319I/337N/361W/374T/449E/471T/489L, 65V/68L/70L/99R/154E/195W/207S/214F/230Y/263G/319I/337N/361W/374T/459M/471T/489L, 65V/68L/70L/99R/154E/195W/207S/214F/230Y/263G/319I/337N/361W/374T/434G/471T/489I, 65V/68L/70L/99R/154E/195W/207S/214F/230Y/263G/319I/337N/361W/374T/471T/489L, 65V/68L/70L/99R/154E/195W/207S/214F/230Y/263G/319I/337N/361W/374T/408E/471T/489L, 65V/68L/70L/99R/154E/195W/207S/214F/230Y/263G/319I/337N/361W/374T/471T/489L/502K, 29I/65V/68L/70L/99R/154E/195W/207S/214F/230Y/263G/319I/337N/361W/374T/471T/489L/534K, 65V/68L/70L/99R/154E/195W/207S/214F/230Y/263G/319I/337N/361W/374T/471T/489L/502Q, 65V/68L/70L/99R/154E/195W/207S/214F/230Y/263G/319I/337N/361W/374T/471T/489L, 65V/68L/70L/99R/154E/195W/207S/214F/230Y/263G/319I/337N/361W/374T/471T/489L/497A, 65V/68L/70L/99R/154E/195W/207S/214F/230Y/263G/319I/337N/361W/374T/471T/489L/533Y, 65V/68L/70L/99R/154E/195W/207S/214F/230Y/263G/319I/337N/361W/374T/471T/489L, 65V/68L/70L/99R/154E/195W/207S/214F/230Y/263G/319I/337N/361W/374T/471T/489L/536P, 65V/68L/70L/99R/154E/195W/207S/214F/230Y/263G/319I/337N/361W/374T/471T/489L/536H, 65V/68L/70L/99R/154E/195W/207S/214F/230Y/263G/319I/337N/361W/374T/471T/485M/489L, 65V/68L/70L/99R/154E/195W/207S/214F/230Y/263G/319I/337N/361W/374T/471T/489L/510S, 65V/68L/70L/99R/154E/195W/207S/214F/230Y/263G/319I/337N/361W/374T/471T/485T/489L, 65V/68L/70L/99R/154E/195W/207S/214F/230Y/263G/319I/337N/361W/374T/471T/489L/532V, 65V/68L/70L/99R/154E/195W/207S/214F/230Y/263G/319I/337N/361W/374T/471T/489E, 65V/68L/70L/99R/154E/195W/207S/214F/230Y/263G/319I/337N/361W/374T/471T/489L/534K, 65V/68L/70L/99R/154E/195W/207S/214F/230Y/263G/319I/337N/361W/374T/471T/489L/494T, 65V/68L/70L/99R/154E/195W/207S/214F/230Y/263G/319I/337N/361W/374T/471T/489L, 65V/68L/70L/99R/154E/182G/195W/207S/214F/230Y/263G/319I/337N/361W/374T/471T/489L, 65V/68L/70L/99R/154E/195W/204Q/207S/214F/230Y/263G/319I/337N/361W/374T/471T/489L, 65V/68L/70L/99R/154E/194H/195W/207S/214F/230Y/263G/319I/337N/361W/374T/471T/489L, 65V/68L/70L/99R/154E/184Q/195W/207S/214F/230Y/263G/319I/337N/361W/374T/471T/489L, 65V/68L/70L/99R/154E/195W/202Q/207S/214F/230Y/263G/319I/337N/361W/374T/471T/489L, 65V/68L/70L/99R/154E/182S/195W/207S/214F/230Y/263G/319I/337N/361W/374T/471T/489L, 65V/68L/70L/99R/154E/195W/207S/214F/230Y/263G/319I/337N/361W/374T/471T/489L, 65V/68L/70L/99R/144M/154E/195W/207S/214F/230Y/263G/319I/337N/361W/374T/471T/489L, 65V/68L/70L/99R/154E/195W/H201Q/207S/214F/230Y/263G/319I/337N/361W/374T/471T/489L, 65V/68L/70L/99R/154E/182D/195W/207S/214F/230Y/263G/319I/337N/361W/374T/471T/489L, 65V/68L/70L/99R/154E/175D/195W/207S/214F/230Y/263G/319I/337N/361W/374T/471T/489L, 65V/68L/70L/99R/154E/182T/195W/207S/214F/230Y/263G/319I/337N/361W/374T/471T/489L, 65V/68L/70L/99R/154E/194Q/195W/207S/214F/230Y/263G/319I/337N/361W/374T/471T/489L, 65V/68L/70L/99R/154E/195W/207S/214F/220A/230Y/263G/319I/337N/361W/374T/471T/489L, 65V/68L/70L/99R/150T/154E/195W/207S/214F/230Y/263G/319I/337N/361W/374T/471T/489L, or 65V/68L/70L/99R/154E/195W/206I/207S/214F/230Y/263G/319I/337N/361W/374T/471T/489L, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set of an engineered glucocerebrosidase set forth in Table 6.1 and 6.2, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set at amino acid position(s) 65/68/70/96/99/154/194/195/207/214/230/263/319/337/361/374/382/408/471/484/489, 65/68/70/99/154/195/207/214/230/263/319/337/359/361/374/382/408/412/471/489/494/495, 65/68/70/92/96/99/154/175/182/195/207/214/230/263/319/337/361/374/382/408/471/489, 65/68/70/96/99/154/195/207/214/230/263/319/337/359/361/374/382/408/471/484/489/494/495, 65/68/70/92/99/154/175/194/195/207/214/230/263/319/337/359/361/374/382/408/471/489, 65/68/70/99/154/195/207/214/230/263/319/337/359/361/374/382/400/408/412/471/489, 65/68/70/92/96/99/154/182/195/207/214/230/263/319/337/361/374/382/400/408/471/484/489, 65/68/70/92/96/99/154/195/207/214/230/263/319/337/361/374/382/408/471/489/494/495, 65/68/70/92/96/99/154/175/195/207/214/230/263/319/337/361/374/382/408/471/489/494/495, 65/68/70/96/99/154/182/195/207/214/230/263/319/337/359/361/374/382/408/471/484/489, 65/68/70/92/96/99/154/195/207/214/230/263/319/337/361/374/382/408/471/489, 65/68/70/92/99/154/195/207/214/230/263/319/337/361/374/382/408/471/489/494/495/534, 65/68/70/92/96/99/154/195/207/214/230/263/319/337/361/374/382/400/408/471/484/489, 65/68/70/92/96/99/154/175/195/207/214/230/263/319/337/361/374/382/408/471/489, 65/68/70/92/99/154/175/195/207/214/230/263/319/337/361/374/382/400/408/471/484/489, 65/68/70/92/96/99/154/175/182/195/207/214/230/263/319/337/361/374/382/408/412/471/484/489, 65/68/70/92/96/99/154/195/201/207/214/230/263/319/337/361/374/382/408/471/484/489, 65/68/70/92/99/154/182/194/195/207/214/230/263/319/337/361/374/382/408/471/489, 65/68/70/92/96/99/154/175/194/195/207/214/230/263/319/337/359/361/374/382/400/408/471/489, 65/68/70/92/99/154/195/207/214/230/263/319/337/359/361/374/382/408/471/489, 65/68/70/96/99/154/195/207/214/230/263/319/337/361/374/382/408/471/489, 65/68/70/92/99/154/175/195/207/214/230/263/319/337/361/374/382/408/471/484/489/494/495, 65/68/70/96/99/154/195/201/207/214/230/263/319/337/359/361/374/382/408/471/489, 65/68/70/92/99/154/195/207/214/230/263/319/337/361/374/408/471/489, 65/68/70/99/154/194/195/207/214/230/263/319/337/361/374/408/471/489, 65/68/70/99/154/195/201/207/214/230/263/319/337/361/374/408/471/489, 65/68/70/99/154/195/207/214/230/263/319/337/359/361/374/408/471/489, 65/68/70/96/99/154/195/207/214/230/263/319/337/361/374/408/471/489, 65/68/70/92/96/99/154/195/207/214/230/263/319/337/361/374/382/400/408/471/484/489/495, 65/68/70/99/154/195/207/214/230/263/319/337/361/374/382/408/471/489/495, 65/68/70/99/154/195/207/214/230/263/319/337/361/374/382/408/471/489, 65/68/70/92/99/154/195/207/214/230/263/319/337/361/374/382/408/471/489, 65/68/70/71/99/154/175/195/207/214/230/263/319/325/337/361/374/385/408/471/489, 65/68/70/99/154/195/207/214/230/263/319/325/337/361/374/385/408/471/489, 65/68/70/71/99/154/195/207/214/230/263/319/325/337/361/374/408/471/489/502, 65/68/70/71/99/154/175/195/207/214/230/263/319/325/337/361/374/385/408/471/489/502, 65/68/70/99/154/195/202/207/214/230/263/319/325/337/361/374/385/408/471/489, 65/68/70/97/99/154/175/195/207/214/230/263/319/337/361/374/385/408/471/489/510, 65/68/70/99/154/195/207/214/230/263/319/325/337/361/374/385/408/471/489/510, 65/68/70/99/100/154/175/195/207/214/230/263/319/325337/361/374/408/471/489, 65/68/70/71/99/154/175/195/207/214/230/263/319/325/337/360/361/374/385/408/471/489, 65/68/70/97/99/154/175/195/207/214/230/263/319/337/361/374/408/471/489/510, 65/68/70/71/97/99/100/154/175/195/207/214/230/263/319/325/337/361/374/385/408/471/489, 65/68/70/71/99/154/175/195/207/214/230/263/319/337/361/374/408/471/489/510, 65/68/70/71/97/99/154/195/207/214/230/263/319/325/337/361/374/408/471/489/502, 65/68/70/71/99/100/154/195/207/214/230/263/319/325/337/361/374/385/408/471/489, 65/68/70/99/154/195/202/207/214/230/263/319/337/361/374/385/408/471/489/489, 65/68/70/99/154/195/207/214/230/263/319/325/337/361/374/385/408/471/489/502, 65/68/70/71/99/100/154/195/207/214/230/263/319/325/337/361/374/385/408/471/489/510, 65/68/70/71/99/100/154/175/195/207/214/230/263/319/325/337/361/374/385/408/471/489, 65/68/70/99/154/195/207/214/230/263/319/337/361/374/385/408/471/489/510, 65/68/70/97/99/154/195/207/214/230/263/319/325/337/361/374/385/408/471/489, 65/68/70/71/99/154/195/207/214/230/263/319/337/361/374/408/471/489, 65/68/70/97/99/154/195/202/207/214/230/263/319/325/337/360/361/374/385/408/471/489/510, 65/68/70/99/154/195/207/214/230/263/319/337/361/374/408/471/489/502, 65/68/70/71/97/99/103/154/195/207/214/230/263/319/325/337/361/374/408/471/489, 65/68/70/99/100/154/195/207/214/230/263/319/325/337/361/374/385/408/471/489/502/510, 65/68/70/71/99/137/154/175/195/207/214/230/263/319/325/337/360/361/374/385/408/471/489/502, 65/68/70/71/99/154/195/207/214/230/263/276/319/325/337/361/374/385/408/471/489/510, 65/68/70/71/99/154/175/195/207/214/230/263/319/337/360/361/374/385/408/471/489/502, 65/68/70/99/100/154/175/195/207/214/230/263/319/325/337/361/374/385/408/471/489/502, 65/68/70/71/99/154/175/195/207/214/230/263/319/337/360/361/374/385/408/471/485/489/502, 65/68/70/95/99/113/154/195/207/214/230/263/319/337/361/374/408/471/489, 65/68/70/99/154/195/207/214/230/263/319/337/361/374/408/471/489/494/536, 65/68/70/95/99/113/154/182/184/195/207/214/230/263/264/319/337/361/374/386/408/471/489, 65/68/70/99/154/184/195/207/214/230/263/319/337/361/374/408/471/489/536, 65/68/70/95/99/142/144/154/182/184/195/207/214/230/263/319/337/361/374/386/408/449/471/489, 65/68/70/99/144/154/195/204/207/214/230/263/319/337/361/374/386/400/408/449/471/489, 65/68/70/99/113/154/195/207/214/230/263/319/337/361/374/408/449/471/489/536, 65/68/70/99/154/195/207/214/230/263/319/337/361/374/408/471/489/536, 65/68/70/95/99/154/195/207/214/230/263/319/337/361/374/386/408/471/489/536, 65/68/70/99/154/195/207/214/230/263/264/319/337/361/374/408/471/489, 65/68/70/99/113/154/195/207/214/230/263/319/337/361/374/386/408/449/471/489, 65/68/70/95/99/154/195/207/214/230/263/319/337/361/374/386/408/471/489, 65/68/70/95/99/154/195/204/207/214/230/263/319/337/361/374/386/408/471/489, 65/68/70/99/113/142/154/195/207/214/230/263/319/337/361/374/386/408/449/471/489, 65/68/70/99/154/182/184/195/207/214/230/263/264/319/337/361/374/408/471/489/494, 65/68/70/99/113/142/154/182/195/207/214/230/263/319/337/361/374/408/471/489/494, 65/68/70/99/113/154/195/207/214/230/263/319/337/361/374/408/449/471/489, 65/68/70/99/154/195/207/214/230/263/264/319/337/361/374/408/449471/489, 65/68/70/95/99/154/195/207/214/230/263/319/337/361/374/408/471/489, 65/68/70/95/99/113/154/195/207/214/230/263/319/337/361/374/408/449/471/489/536, 65/68/70/99/154/195/207/214/230/263/319/337/361/374/386/408/471/489/536, 65/68/70/99/154/182/184/195/207/214/230/263/319/337/361/374/408/471/489, 65/68/70/99/142/154/195/207/214/230/263/319/337/361/374/386/400/408/471/489/536, 35/65/68/70/99/154/195/207/214/230/263/264/319/337/361/374/408/471/449/489/536, 65/68/70/99/142/144/154/195/207/214/220/230/263/319/337/361/374/408/471/489, 65/68/70/99/142/154/195/207/214/230/263/319/337/361/374/408/471/489, 29/65/68/70/99/102/154/182/195/207/214/230/263/276/319/336/337/361/374/390/408/459/471/489/502/532, 41/65/68/70/99/154/195/207/214/230/262/263/319/336/337/361/374/408/471/489/502, 29/65/68/70/97/99/150/154/195/207/214/230/262/263/319/336/337/361/374/408/471/489/502/53229/65/68/70/99/154/195/207/214/230/263/319/337/361/374/408/471/489, 65/68/70/99/154/195/207/214/230/263/319/337/361/374/408/471/489/532, 65/68/70/99/102/154/195/207/214/230/263/319/337/361/374/390/408/459/471/489/502/532, 29/65/68/70/99/154/195/207/214/230/262/263/276/319/336/337/361/374/408/471/489/495, 29/65/68/70/99/154/182/195/207/214/230/262/263/319/337/361/374/408/459/471/489/532, 65/68/70/92/99/102/154/195/207/214/230/263/319/337/361/374/408/471/489/502, 29/65/68/70/97/99/102/154/182/195/207/214/230/263/319/337/361/374/408/471/489/502/532, 65/68/70/99/154/195/207/214/230/263/319/336/337/361/374/408/459/471/489/495/502/532, 65/68/70/99/150/154/182/195/207/214/230/263/319/336/337/361/374/390/408/471/489, 65/68/70/99/150/154/195/207/214/230/262/263/319/337/361/374/408/471/489/495, 29/65/68/70/99/154/195/207/214/230/263/319/337/361/374/408/471/489/495, 29/65/68/70/99/102/154/182/195/207/214/230/262/263/319/337/361/374/408/471/489/502, 29/65/68/70/99/154/195/207/214/230/263/319/336/337/361/374/408/459/471/489/502/532, 65/68/70/99/154/182/195/207/214/230/263/319/337/361/374/408/471/489/495/502/532, 65/68/70/99/102/154/195/207/214/230/263/319/336/337/361/374/408/471/489, 65/68/70/99/150/154/182/195/207/214/230/262/263/319/337/361/374/408/471/489/495, 65/68/70/99/154/195/207/214/230/263/319/337/361/374/408/459/471/489/495/502/532, 41/65/68/70/99/154/182/195/207/214/230/263/319/337/361/374/390/408/471/489/495, 29/41/65/68/70/99/154/182/195/207/214/230/263/319/336/337/361/374/408/471/489/495, 65/68/70/97/99/102/154/195/207/214/230/263/319/337/361/374/408/471/489/495, 65/68/70/99/154/182/195/207/214/230/262/263/319/337/361/374/408/471/489/495, 29/65/68/70/99/154/195/207/214/230/262/263/319/337/361/374/408/471/489/502, 29/65/68/70/99/102/154/195/207/214/230/263/319/337/361/374/408/471/489/502, 65/68/70/99/154/182/195/207/214/230/263/319/336/337/361/374/408/471/489/495, 29/65/68/70/99/150/154/182/195/207/214/230/262/263/319/337/361/374/408/459/471/489/495, 65/68/70/99/154/195/207/214/230/263/319/337/361/374/408/471/489/502/532, 65/68/70/99/154/195/207/214/230/263/319/337/361/374/386/408/471/489, 65/68/70/99/154/195/207/214/230/263/319/337/361/374/408/471/489/497, 65/68/70/99/154/195/207/214/230/263/319/337/361/374/408/471/489, 65/68/70/99/154/195/207/214/230/263/319/337/361/374/408/471/489/512, 65/68/69/70/99/154/195/207/214/230/263/319/337/361/374/408/471/489, 65/68/70/99/154/195/207/214/230/263/319/337/361/374/408/471/489/507, 65/68/70/99/154/195/207/214/230/263/319/337/361/374/408/445/471/489, 65/68/70/75/99/154/195/207/214/230/263/319/337/361/374/408/471/489, 65/68/70/99/154/195/207/214/230/263/319/337/361/374/408/471/487/489, 65/68/70/99/154/184/195/207/214/230/263/319/337/361/374/408/471/489, 65/68/70/90/99/154/195/207/214/230/263/319/337/361/374/408/471/489, 65/68/70/99/154/195/207/214/230/235/263/319/337/361/374/408/471/489, 65/68/70/99/154/195/207/214/230/263/319/337/361/374/408/434/471/489, 65/68/70/86/99/154/195/207/214/230/263/319/337/361/374/408/471/489, 65/68/70/99/154/182/195/207/214/230/263/319/337/361/374/408/471/489, 65/68/70/99/154/195/207/214/230/263/319/337/361/374/408/446/471/489, 65/68/70/99/154/195/207/214/230/263/319/337/361/374/408/471/489/490, 65/68/70/99/154/195/207/214/230/263/299/319/337/361/374/408/471/489, 65/68/70/99/102/154/195/207/214/230/263/319/337/361/374/408/471/489, 65/68/70/99/141/154/195/207/214/230/263/319/337/361/374/408/471/489, 65/68/70/97/99/154/195/207/214/230/263/319/337/361/374/408/471/489, 65/68/70/99/154/195/207/214/230/263/319/337/361/374/408/471/489/522, 65/68/70/99/154/195/207/214/224/230/263/319/337/361/374/408/471/489, 65/68/70/99/154/195/207/214/230/263/319/337/361/374/408/471/478/489, 65/68/70/99/154/195/207/214/230/263/319/337/361/374/408/471/480/489, 65/68/70/99/154/195/207/214/230/263/319/337/361/374/408/412/471/489, 65/68/70/99/154/195/207/214/230/263/265/319/337/361/374/408/471/489, 65/68/70/99/154/195/207/214/230/263/319/337/361/374/408/471/489/494, 65/68/70/99/154/195/207/214/230/263/316/319/337/361/374/408/471/489, 65/68/70/99/154/195/207/214/230/263/319/337/361/368/374/408/471/489, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set 65V/68L/70L/96E/99R/154E/194Q/195W/207S/214F/230Y/263G/319I/337N/361W/374T/382A/408E/471T/484E/489L, 65V/68L/70L/99R/154E/195W/207S/214F/230Y/263G/319I/337N/359P/361W/374T/382A/408E/412W/471T/489L/494T/495G, 65V/68L/70L/92Q/96E/99R/154E/175D/182S/195W/207S/214F/230Y/263G/319I/337N/361W/374T/382A/408E/471T/489L, 65V/68L/70L/96E/99R/154E/195W/207S/214F/230Y/263G/319I/337N/359P/361W/374T/382A/408E/471T/484E/489L/494T/495G, 65V/68L/70L/92E/99R/154E/175D/194Q/195W/207S/214F/230Y/263G/319I/337N/359P/361W/374T/382A/408E/471T/489L, 65V/68L/70L/99R/154E/195W/207S/214F/230Y/263G/319I/337N/359P/361W/374T/382A/400E/408E/412W/471T/489L, 65V/68L/70L/92Q/96E/99R/154E/182S/195W/207S/214F/230Y/263G/319I/337N/361W/374T/382A/400E/408E/471T/484E/489L, 65V/68L/70L/92Q/96E/99R/154E/195W/207S/214F/230Y/263G/319I/337N/361W/374T/382A/408E/471T/489L/494T/495G, 65V/68L/70L/92Q/96E/99R/154E/175D/195W/207S/214F/230Y/263G/319I/337N/361W/374T/382A/408E/471T/489L/494T/495G, 65V/68L/70L/96E/99R/154E/182S/195W/207S/214F/230Y/263G/319I/337N/359P/361W/374T/382A/408E/471T/484E/489L, 65V/68L/70L/92E/96E/99R/154E/195W/207S/214F/230Y/263G/319I/337N/361W/374T/382A/408E/471T/489L, 65V/68L/70L/92Q/99R/154E/195W/207S/214F/230Y/263G/319I/337N/361W/374T/382A/408E/471T/489L/494T/495G/534K, 65V/68L/70L/92E/96E/99R/154E/195W/207S/214F/230Y/263G/319I/337N/361W/374T/382A/400Q/408E/471T/484E/489L, 65V/68L/70L/92Q/96E/99R/154E/175D/195W/207S/214F/230Y/263G/319I/337N/361W/374T/382A/408E/471T/489L, 65V/68L/70L/92Q/99R/154E/175D/195W/207S/214F/230Y/263G/319I/337N/361W/374T/382A/400Q/408E/471T/484E/489L, 65V/68L/70L/92E/96E/99R/154E/175D/195W/207S/214F/230Y/263G/319I/337N/361W/374T/382A/408E/471T/489L/494T/495G, 65V/68L/70L/92Q/96E/99R/154E/175D/182S/195W/207S/214F/230Y/263G/319I/337N/361W/374T/382A/408E/412W/471T/484E/489L, 65V/68L/70L/92Q/96E/99R/154E/195W/201Q/207S/214F/230Y/263G/319I/337N/361W/374T/382A/408E/471T/484E/489L, 65V/68L/70L/92E/99R/154E/182S/194Q/195W/207S/214F/230Y/263G/319I/337N/361W/374T/382A/408E/471T/489L, 65V/68L/70L/92Q/96E/99R/154E/175D/194Q/195W/207S/214F/230Y/263G/319I/337N/359P/361W/374T/382A/400E/408E/471T/489L, 65V/68L/70L/92Q/99R/154E/195W/207S/214F/230Y/263G/319I/337N/359P/361W/374T/382A/408E/471T/489L, 65V/68L/70L/96E/99R/154E/195W/207S/214F/230Y/263G/319I/337N/361W/374T/382A/408E/471T/489L, 65V/68L/70L/92Q/99R/154E/175D/195W/207S/214F/230Y/263G/319I/337N/361W/374T/382A/408E/471T/484E/489L/494T/495G, 65V/68L/70L/96E/99R/154E/195W/201Q/207S/214F/230Y/263G/319I/337N/359P/361W/374T/382A/408E/471T/489L, 65V/68L/70L/92E/99R/154E/195W/207S/214F/230Y/263G/319I/337N/361W/374T/408E/471T/489L, 65V/68L/70L/92Q/99R/154E/195W/207S/214F/230Y/263G/319I/337N/361W/374T/408E/471T/489L, 65V/68L/70L/99R/154E/194Q/195W/207S/214F/230Y/263G/319I/337N/361W/374T/408E/471T/489L, 65V/68L/70L/99R/154E/195W/201Q/207S/214F/230Y/263G/319I/337N/361W/374T/408E/471T/489L, 65V/68L/70L/99R/154E/195W/207S/214F/230Y/263G/319I/337N/359P/361W/374T/408E/471T/489L, 65V/68L/70L/96E/99R/154E/195W/207S/214F/230Y/263G/319I/337N/361W/374T/408E/471T/489L, 65V/68L/70L/92E/96E/99R/154E/195W/207S/214F/230Y/263G/319I/337N/361W/374T/382A/400E/408E/471T/484E/489L/495G, 65V/68L/70L/99R/154E/195W/207S/214F/230Y/263G/319I/337N/361W/374T/382A/408E/471T/489L/495G, 65V/68L/70L/99R/154E/195W/207S/214F/230Y/263G/319I/337N/361W/374T/382A/408E/471T/489L, 65V/68L/70L/92E/99R/154E/195W/207S/214F/230Y/263G/319I/337N/361W/374T/382A/408E/471T/489I, 65V/68L/70L/71T/99R/154E/175D/195W/207S/214F/230Y/263G/319I/325V/337N/361W/374T/385H/408E/471T/489L, 65V/68L/70L/99R/154E/195W/207S/214F/230Y/263G/319I/325V/337N/361W/374T/385H/408E/471T/489L, 65V/68L/70L/71T/99R/154E/195W/207S/214F/230Y/263G/319I/325V/337N/361W/374T/408E/471T/489L/502Q, 65V/68L/70L/71T/99R/154E/175D/195W/207S/214F/230Y/263G/319I/325V/337N/361W/374T/385H/408E/471T/489L/502Q, 65V/68L/70L/99R/154E/195W/202Q/207S/214F/230Y/263G/319I/325V/337N/361W/374T/385N/408E/471T/489L, 65V/68L/70L/97Q/99R/154E/175D/195W/207S/214F/230Y/263G/319I/337N/361W/374T/385H/408E/471T/489L/510S, 65V/68L/70L/99R/154E/195W/207S/214F/230Y/263G/319I/325V/337N/361W/374T/385Q/408E/471T/489L/510S, 65V/68L/70L/99R/100R/154E/175D/195W/207S/214F/230Y/263G/319I/325V/337N/361W/374T/408E/471T/489L, 65V/68L/70L/71T/99R/154E/175D/195W/207S/214F/230Y/263G/319I/325V/337N/360E/361W/374T/385N/408E/471T/489L, 65V/68L/70L/97Q/99R/154E/175D/195W/207S/214F/230Y/263G/319I/337N/361W/374T/408F/471T/489L/510S, 65V/68L/70L/71T/97Q/99R/100R/154E/175D/195W/207S/214F/230Y/263G/319I/325V/337N/361W/374T/385H/408E/471T/489L, 65V/68L/70L/71T/99R/154E/175D/195W/207S/214F/230Y/263G/319I/337N/361W/374T/408E/471T/489L/510S, 65V/68L/70L/71T/97Q/99R/154E/195W/207S/214F/230Y/263G/319I/325V/337N/361W/374T/408E/471T/489L/502Q, 65V/68L/70L/71T/99R/100R/154E/195W/207S/214F/230Y/263G/319I/325V/337N/361W/374T/385Q/408E/471T/489L, 65V/68L/70L/99R/154E/195W/202Q/207S/214F/230Y/263G/319I/337N/361W/374T/385N/408E/471T/489L/489E, 65V/68L/70L/99R/154E/195W/207S/214F/230Y/263G/319I/325V/337N/361W/374T/385N/408E/471T/489L/502Q, 65V/68L/70L/99R/154E/195W/207S/214F/230Y/263G/319I/325V/337N/361W/374T/385H/408E/471T/489L/502Q, 65V/68L/70L/71T/99R/100R/154E/195W/207S/214F/230Y/263G/319I/325V/337N/361W/374T/385H/408E/471T/489L/510S, 65V/68L/70L/71T/99R/100R/154E/175D/195W/207S/214F/230Y/263G/319I/325V/337N/361W/374T/385N/408E/471T/489L, 65V/68L/70L/71T/99R/100R/154E/175D/195W/207S/214F/230Y/263G/319I/325V/337N/361W/374T/385H/408E/471T/489L, 65V/68L/70L/99R/154E/195W/207S/214F/230Y/263G/319I/337N/361W/374T/385N/408E/471T/489L/510S, 65V/68L/70L/97Q/99R/154E/195W/207S/214F/230Y/263G/319I/325V/337N/361W/374T/385N/408E/471T/489L, 65V/68L/70L/71T/99R/154E/195W/207S/214F/230Y/263G/319I/337N/361W/374T/408E/471T/489L, 65V/68L/70L/97Q/99R/154E/195W/202Q/207S/214F/230Y/263G/319I/325V/337N/360E/361W/374T/385G/408E/471T/489L/510S, 65V/68L/70L/99R/154E/195W/207S/214F/230Y/263G/319I/337N/361W/374T/408E/471T/489L/502Q, 65V/68L/70L/71T/97Q/99R/103S/154E/195W/207S/214F/230Y/263G/319I/325V/337N/361W/374T/408E/471T/489L, 65V/68L/70L/99R/100R/154E/195W/207S/214F/230Y/263G/319I/325V/337N/361W/374T/385H/408E/471T/489L/502Q/510S, 65V/68L/70L/71T/99R/137Q/154E/175D/195W/207S/214F/230Y/263G/319I/325V/337N/360E/361W/374T/385H/408E/471T/489L/502Q, 65V/68L/70L/71T/99R/154E/195W/207S/214F/230Y/263G/276T/319I/325V/337N/361W/374T/385R/408E/471T/489L/510S, 65V/68L/70L/71T/99R/154E/175D/195W/207S/214F/230Y/263G/319I/337N/360E/361W/374T/385N/408E/471T/489L/502Q, 65V/68L/70L/99R/100R/154E/175D/195W/207S/214F/230Y/263G/319I/325V/337N/361W/374T/385N/408E/471T/489E/502Q, 65V/68L/70L/71T/99R/154E/175D/195W/207S/214F/230Y/263G/319I/337N/360E/361W/374T/385H/408E/471T/485S/489L/502Q, 65V/68L/70L/95W/99R/113Q/154E/195W/207S/214F/230Y/263G/319I/337N/361W/374T/408E/471T/489L, 65V/68L/70L/99R/154E/195W/207S/214F/230Y/263G/319I/337N/361W/374T/408E/471T/489L/494T/536P, 65V/68L/70L/95W/99W/113Q/154E/182D/184V/195W/207S/214F/230Y/263G/264I/319I/337N/361W/374T/386I/408E/471T/489L, 65V/68L/70L/99R/154E/184Q/195W/207S/214F/230Y/263G/319I/337N/361W/374T/408E/471T/489L/536P, 65V/68L/70L/95W/99R/142N/144M/154E/182G/184Q/195W/207S/214F/230Y/263G/319I/337N/361W/374T/386I/408E/449E/471T/489L, 65V/68L/70L/99R/144M/154E/195W/204Q/207S/214F/230Y/263G/319I/337N/361W/374T/386I/400T/408E/449E/471T/489L, 65V/68L/70L/99R/113Q/154E/195W/207S/214F/230Y/263G/319I/337N/361W/374T/408E/449E/471T/489L/536P, 65V/68L/70L/99R/154E/195W/207S/214F/230Y/263G/319I/337N/361W/374T/408E/471T/489L/536P, 65V/68L/70L/95W/99R/154E/195W/207S/214F/230Y/263G/319I/337N/361W/374T/386I/408E/471T/489L/536P, 65V/68L/70L/99R/154E/195W/207S/214F/230Y/263G/264I/319I/337N/361W/374T/408E/471T/489L, 65V/68L/70L/99W/113Q/154E/195W/207S/214F/230Y/263G/319I/337N/361W/374T/386I/408E/449E/471T/489L, 65V/68L/70L/95W/99R/154E/195W/207S/214F/230Y/263G/319I/337N/361W/374T/386I/408E/471T/489L, 65V/68L/70L/95W/99W/154E/195W/204Q/207S/214F/230Y/263G/319I/337N/361W/374T/386I/408E/471T/489L, 65V/68L/70L/99R/113Q/142N/154E/195W/207S/214F/230Y/263G/319I/337N/361W/374T/386I/408E/449E/471T/489L, 65V/68L/70L/99R/154E/182G/184Q/195W/207S/214F/230Y/263G/264I/319I/337N/361W/374T/408E/471T/489L/494T, 65V/68L/70L/99R/113Q/142N/154E/182D/195W/207S/214F/230Y/263G/319I/337N/361W/374T/408E/471T/489L/494T, 65V/68L/70L/99R/113Q/154E/195W/207S/214F/230Y/263G/319I/337N/361W/374T/408E/471T/489L, 65V/68L/70L/99W/154E/195W/207S/214F/230Y/263G/264I/319I/337N/361W/374T/408E/449E/471T/489L, 65V/68L/70L/95W/99R/154E/195W/207S/214F/230Y/263G/319I/337N/361W/374T/408E/471T/489L, 65V/68L/70L/95W/99W/154E/195W/207S/214F/230Y/263G/319I/337N/361W/374T/408E/471T/489L, 65V/68L/70L/99W/113Q/154E/195W/207S/214F/230Y/263G/319I/337N/361W/374T/408E/449E/471T/489L, 65V/68L/70L/95W/99R/113Q/154E/195W/207S/214F/230Y/263G/319I/337N/361W/374T/408E/449E/471T/489L/536P, 65V/68L/70L/95W/99W/113Q/154E/195W/207S/214F/230Y/263G/319I/337N/361W/374T/408E/471T/489L, 65V/68L/70L/99R/154E/195W/207S/214F/230Y/263G/319I/337N/361W/374T/386I/408E/471T/489L/536P, 65V/68L/70L/99W/154E/182G/184Q/195W/207S/214F/230Y/263G/319I/337N/361W/374T/408E/471T/489 L, 65V/68L/70L/99R/142N/154E/195W/207S/214F/230Y/263G/319I/337N/361W/374T/386I/400T/408E/471T/489L/536P, 35T/65V/68L/70L/99R/154E/195W/207S/214F/230Y/263G/264I/319I/337N/361W/374T/408E/471T/449E/489L/536P, 65V/68L/70L/99R/142N/144M/154E/195W/207S/214F/220A/230Y/263G/319I/337N/361W/374T/408E/471 T/489L, 65V/68L/70L/99W/142N/154E/195W/207S/214F/230Y/263G/319I/337N/361W/374T/408E/471T/489L, 29I/65V/68L/70L/99R/102A/154E/182M/195W/207S/214F/230Y/263G/276T/319I/336E/337N/361W/374T/390A/408E/459M/471T/489L/502K/532V, 41H/65V/68L/70L/99R/154E/195W/207S/214F/230Y/262Q/263G/319I/336E/337N/361W/374T/408E/471T/489L/502K, 29I/65V/68L/70L/97R/99R/150T/154E/195W/207S/214F/230Y/262Q/263G/319I/336E/337N/361W/374T/408E/471T/489L/502K/532V, 29I/65V/68L/70L/99R/154E/195W/207S/214F/230Y/263G/319I/337N/361W/374T/408E/471T/489L, 65V/68L/70L/99R/154E/195W/207S/214F/230Y/263G/319I/337N/361W/374T/408E/471T/489L/532V, 65V/68L/70L/99R/102A/154E/195W/207S/214F/230Y/263G/319I/337N/361W/374T/390A/408E/459M/471 T/489L/502K/532V, 29I/65V/68L/70L/99R/154E/195W/207S/214F/230Y/262Q/263G/276T/319I/336E/337N/361W/374T/408E/471T/489L/495G, 29I/65V/68L/70L/99R/154E/182T/195W/207S/214F/230Y/262Q/263G/319I/337N/361W/374T/408E/459M/471T/489L/532V, 65V/68L/70L/92K/99G/102A/154E/195W/207S/214F/230Y/263G/319I/337N/361W/374T/408E/471T/489L/502K, 29I/65V/68L/70L/97R/99R/102A/154E/182M/195W/207S/214F/230Y/263G/319I/337N/361W/374T/408E/471T/489L/502K/532V, 65V/68L/70L/99R/154E/195W/207S/214F/230Y/263G/319I/336E/337N/361W/374T/408E/459M/471T/489L/495S/502K/532V, 65V/68L/70L/99R/150T/154E/182T/195W/207S/214F/230Y/263G/319I/336E/337N/361W/374T/390A/408E/471T/489L, 65V/68L/70L/99R/150T/154E/195W/207S/214F/230Y/262Q/263G/319I/337N/361W/M374T/408E/471T/489 L/495G, 29I/65V/68L/70L/99R/154E/195W/207S/214F/230Y/263G/319I/337N/361W/374T/408E/471T/489L/495S, 29I/65V/68L/70L/99R/102A/154E/182T/195W/207S/214F/230Y/262Q/263G/319I/337N/361W/374T/408E/471T/489L/502K 29I/65V/68L/70L/99R/154E/195W/207S/214F/230Y/263G/319I/336E/337N/361W/374T/408E/459M/471T/489L/502K/532V, 65V/68L/70L/99R/154E/182M/195W/207S/214F/230Y/263G/319I/337N/361W/374T/408E/471T/489L/495G/502K/532V, 65V/68L/70L/99R/102A/154E/195W/207S/214F/230Y/263G/319I/336E/337N/361W/374T/408E/471T/489L, 65V/68L/70L/99R/150T/154E/182M/195W/207S/214F/230Y/262Q/263G/319I/337N/361W/374T/408E/471T/489L/495S, 65V/68L/70L/99R/154E/195W/207S/214F/230Y/263G/319I/337N/361W/374T/408E/459M/471T/489L/495S/502K/532V, 41H/65V/68L/70L/99R/154E/182M/195W/207S/214F/230Y/263G/319I/337N/361W/374T/390A/408E/471T/489L/495G, 29I/41H/65V/68L/70L/99R/154E/182T/195W/207S/214F/230Y/263G/319I/336E/337N/361W/374T/408E/471T/489L/495S, 65V/68L/70L/97R/99R/102A/154E/195W/207S/214F/230Y/263G/319I/337N/361W/374T/408E/471T/489L/495S, 65V/68L/70L/99R/154E/182T/195W/207S/214F/230Y/262Q/263G/319I/337N/361W/374T/408E/471T/489L/495S 29I/65V/68L/70L/99R/154E/195W/207S/214F/230Y/262Q/263G/319I/337N/361W/374T/408E/471T/489L/502K, 29I/65V/68L/70L/99R/102A/154E/195W/207S/214F/230Y/263G/319I/337N/361W/374T/408E/471T/489L/502K, 65V/68L/70L/99R/154E/182M/195W/207S/214F/230Y/263G/319I/336E/337N/361W/374T/408E/471T/489L/495S, 65V/68L/70L/99R/154E/195W/207S/214F/230Y/263G/319I/337N/361W/374T/408E/471T/489L/502K, 29I/65V/68L/70L/99R/150T/154E/182T/195W/207S/214F/230Y/262Q/263G/319I/337N/361W/374T/408E/459M/471T/489L/495G, 65V/68L/70L/99R/154E/195W/207S/214F/230Y/263G/319I/337N/361W/374T/408E/471T/489L/502K/532V, 65V/68L/70L/99R/154E/195W/207S/214F/230Y/263G/319I/337N/361W/374T/386P/408E/471T/489L, 65V/68L/70L/99R/154E/195W/207S/214F/230Y/263G/319I/337N/361W/374T/408E/471T/489L/497M, 65V/68L/70L/99K/154E/195W/207S/214F/230Y/263G/319I/337N/361W/374T/408E/471T/489L, 65V/68L/70L/99R/154E/195W/207S/214F/230Y/263G/319I/337N/361W/374T/408E/471T/489L/512H, 65V/68L/69D/70L/99R/154E/195W/207S/214F/230Y/263G/319I/337N/361W/374T/408E/471T/489L, 65V/68L/70L/99R/154E/195W/207S/214F/230Y/263G/319I/337N/361W/374T/408E/471T/489L/507I, 65V/68L/70L/99R/154E/195W/207S/214F/230Y/263G/319I/337N/361W/374T/408E/445N/471T/489L, 65V/68L/70L/75N/99R/154E/195W/207S/214F/230Y/263G/319I/337N/361W/374T/408E/471T/489L, 65V/68L/70L/75H/99R/154E/195W/207S/214F/230Y/263G/319I/337N/361W/374T/408E/471T/489L, 65V/68L/70L/99R/154E/195W/207S/214F/230Y/263G/319I/337N/361W/374T/408E/471T/489L/512Q, 65V/68L/70L/99R/154E/195W/207S/214F/230Y/263G/319I/337N/361W/374T/408E/471T/487S/489L, 65V/68L/70L/99R/113P/154E/195W/207S/214F/230Y/263G/319I/337N/361W/374T/408E/471T/489L, 65V/68L/70L/99R/154E/184I/195W/207S/214F/230Y/263G/319I/337N/361W/374T/408E/471T/489L, 65V/68L/70L/90M/99R/154E/195W/207S/214F/230Y/263G/319I/337N/361W/374T/408E/471T/489L, 65V/68L/70L/99R/154E/195W/207S/214F/230Y/235T/263G/319I/337N/361W/374T/408E/471T/489L, 65V/68L/70L/99R/154E/195W/207S/214F/230Y/263G/319I/337N/361W/374T/408E/434Q/471T/489L, 65V/68L/70L/99R/154E/195W/207S/214F/230Y/263G/319I/337N/361W/374T/408E/445G/471T/489L, 65V/68L/70L/99R/154E/195W/207S/214F/230Y/263G/319I/337N/361W/374T/408E/471T/489N, 65V/68L/70L/90A/99R/154E/195W/207S/214F/230Y/263G/319I/337N/361W/374T/408E/471T/489L, 65V/68L/70L/99R/154E/195W/207S/214F/230Y/263G/319I/337N/361W/374T/386L/408E/471T/489L, 65V/68L/70L/75E/99R/154E/195W/207S/214F/230Y/263G/319I/337N/361W/374T/408E/471T/489L, 65V/68L/70L/86E/99R/154E/195W/207S/214F/230Y/263G/319I/337N/361W/374T/408E/471T/489L, 65V/68L/70L/99R/154E/195W/207S/214F/230Y/263G/319I/337N/361W/374T/408E/471T/489Q, 65V/68L/70L/99R/154E/182E/195W/207S/214F/230Y/263G/319I/337N/361W/374T/408E/471T/489L, 65V/68L/70L/99R/154E/195W/207S/214F/230Y/263G/319I/337N/361W/374T/408E/446H/471T/489L, 65V/68L/70L/99R/154E/195W/207S/214F/230Y/263G/319I/337N/361W/374T/408E/471T/489L/490N, 65V/68L/70L/99R/154E/195W/207S/214F/230Y/263G/319I/337N/361W/374T/408E/471T/489L, 65V/68L/70L/99R/154E/195W/207S/214F/230Y/263G/299V/319I/337N/361W/374T/408E/471T/489L, 65V/68L/70L/99R/102P/154E/195W/207S/214F/230Y/263G/319I/337N/361W/374T/408E/471T/489L, 65V/68L/70L/99R/154E/195W/207S/214F/230F/263G/319I/337N/361W/374T/408E/471T/489L, 65V/68T/70L/99R/154E/195W/207S/214F/230Y/263G/319I/337N/361W/374T/408E/471T/489L, 65V/68L/70L/99R/141E/154E/195W/207S/214F/230Y/263G/319I/337N/361W/374T/408E/471T/489L, 65V/68L/70L/99R/154E/195W/207S/214F/230Y/263G/319I/337N/361W/374T/408E/471T/489L, 65V/68L/70L/97E/99R/154E/195W/207S/214F/230Y/263G/319I/337N/361W/374T/408E/471T/489L, 65V/68L/70L/99R/154E/195W/207S/214F/230Y/263G/319I/337N/361W/374T/408E/471T/489L/522S, 65V/68L/70L/99R/154E/195W/207S/214F/224M/230Y/263G/319I/337N/361W/374T/408E/471T/489L, 65V/68L/70L/99R/154E/195W/207S/214F/230Y/263G/319I/337N/361W/374T/408E/471T/478N/489L, 65V/68L/70L/99R/154E/195W/207S/214F/230Y/263G/319I/337N/361W/374T/408E/471T/489I, 65V/68L/70L/99R/154E/195W/207S/214F/230Y/263G/319I/337N/361W/374T/408E/471T/480G/489L, 65V/68L/70L/86H/99R/154E/195W/207S/214F/230Y/263G/319I/337N/361W/374T/408E/471T/489L, 65V/68L/70L/99R/154E/195W/207S/214F/230Y/263G/319I/337N/361W/374T/408E/412N/471T/489L, 65V/68L/70L/99R/154E/195W/207S/214F/230Y/263G/319I/337N/361W/374T/408E/449S/471T/489L, 65V/68L/70L/99R/154E/195W/207S/214F/230Y/263G/265N/319I/337N/361W/374T/408E/471T/489L, 65V/68L/70L/99R/154E/195W/207S/214F/230Y/263G/319I/337N/361W/374T/408E/471T/489L/494A, 65V/68L/70L/96N/99R/154E/195W/207S/214F/230Y/263G/319I/337N/361W/374T/408E/471T/489L, 65V/68L/70L/99R/154E/195W/207S/214F/230Y/263G/316H/319I/337N/361W/374T/408E/471T/489L, 65V/68L/70L/99R/154E/195W/207S/214F/230Y/263G/319I/337N/361W/368D/374T/408E/471T/489L, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set of an engineered glucocerebrosidase set forth in Table 12.1 and 12.2, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution at an amino acid position set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least one substitution set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set at amino acid position(s) set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, or relative to the reference sequence corresponding to SEQ ID NO: 2.

In some embodiments, the amino acid sequence of the engineered glucocerebrosidase comprises at least a substitution or substitution set of an engineered glucocerebrosidase variant set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, or relative to the reference sequence corresponding to SEQ ID NO: 2.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, 220, 984, or 1150, or to the reference sequence corresponding to SEQ ID NO: 54, 220, 984, or 1150.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 4-1648, or to the reference sequence corresponding to an even-numbered SEQ ID NO. of SEQ ID NOs: 4-1648.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, 220, 984, or 1150, or to the reference sequence corresponding to SEQ ID NO: 54, 220, 984, or 1150, wherein the amino acid sequence comprises one or more substitutions relative to the reference sequence corresponding to residues 40-536 of SEQ ID NO: 54, 220, 984, or 1150, or relative to the reference sequence corresponding to SEQ ID NO: 54, 220, 984, or 1150.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 4-1648, or to the reference sequence corresponding to an even-numbered SEQ ID NO. of SEQ ID NOs: 4-1648, wherein the amino acid sequence comprises one or more substitutions relative to the reference sequence corresponding to residues 40-536 of SEQ ID NO: 54, 220, 984, or 1150, or relative to the reference sequence corresponding to SEQ ID NO: 54, 220, 984, or 1150.

In some embodiments, the amino acid sequence of the engineered glucocerebrosidase comprises at least a substitution at amino acid position 23, 29, 35, 40, 41, 42, 44, 46, 50, 51, 53, 59, 63, 65, 68, 69, 70, 71, 75, 77, 78, 79, 82, 84, 86, 88, 89, 90, 91, 92, 93, 94, 95, 96, 97, 98, 99, 100, 101, 102, 103, 104, 105, 106, 107, 108, 109, 110, 111, 113, 114, 115, 116, 117, 133, 134, 137, 138, 141, 142, 144, 150, 151, 154, 169, 171, 172, 175, 180, 182, 184, 186, 189, 190, 191, 194, 195, 201, 202, 204, 206, 207, 211, 212, 214, 220, 222, 224, 230, 233, 235, 236, 239, 243, 246, 258, 260, 261, 262, 263, 264, 265, 271, 276, 298, 299, 301, 314, 315, 316, 319, 325, 329, 332, 333, 335, 336, 337, 338, 339, 341, 342, 359, 360, 361, 368, 370, 372, 373, 374, 382, 385, 386, 390, 400, 404, 408, 412, 413, 415, 434, 445, 446, 449, 459, 467, 468, 469, 471, 473, 474, 476, 477, 478, 479, 480, 481, 484, 485, 487, 489, 490, 493, 494, 495, 497, 502, 504, 505, 506, 507, 508, 509, 510, 512, 514, 515, 516, 518, 519, 520, 522, 524, 525, 529, 532, 533, 534, or 536, or combinations thereof, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

In some embodiments, the amino acid sequence of the engineered glucocerebrosidase comprises at least a substitution or amino acid residue 23A, 29I, 35T, 40H/S, 41H, 42A, 44V, 46M/R, 50A, 51P, 53L, 59N, 63L, 65L/M/S/V, 68F/L/Q/T/V/W/Y, 69D, 70L, 71F/G/H/L/M/R/S/T/Y, 75A/E/H/N, 77G/H/I/N/R/T, 78L/M, 79I/R/V, 82S, 84A/G/K, 86E/H, 88L, 89C/D, 90A/M, 91I/T, 92A/2E/G/H/K/Q, 93S, 94E/N, 95G/H/L/T/W, 96D/E/G/I/M/N/R/S/V, 97E/I/K/M/N/Q/R, 98P, 99E/F/G/K/L/R/S/V/W, 100N/R, 101L/Q/R/S/T/V/W, 102A/E/N/P/S, 103A/L/P/S, 104M/V, 105I/M/T, 106W, 107A/C/D/E/M/Q/S, 108I, 109D/G/L/M/R, 110A/H/Q, 111F/H/L, 113G/I/N/P/Q/R, 114Y, 115M/R/V, 116E/M/Q/R/V/Y, 117I/T, 133M, 134S, 137Q/V, 138S, 141D/E, 142A/M/N/Y, 144M, 150T, 151L, 154E, 169V, 171C/P/V, 172S, 175D, 180C, 182D/E/G/H/M/S/T, 184I/Q/V, 186G, 189R/S, 190W, 191N/R, 194H/L/Q, 195M/W, 201Q/R/T, 202Q, 204Q, 206F/I/R, 207S/D, 211I/L/N, 212A/N, 214F, 220A/G, 222I, 224M, 230F/L/Y, 233A/G/Q/R, 235T, 236M, 239I/R/T, 243K/L/M/R/V, 246K/R, 258T, 260R, 261K, 262Q, 263G, 264I, 265N/R/S/T, 271T, 276T, 298W, 299V, 301K, 314D/G/R, 315T, 316H, 319F/I/V, 325V, 329L, 332R, 333C/L/T, 335M, 336A/E/G/R, 337N, 338R, 339A, 341R/S, 342Q, 359E/P, 360E, 361L/V/W/Y, 368D, 370R, 372D, 373A/V/Y, 374T/V, 382A, 385G/H/N/Q/R, 386I/L/P, 390A, 400E/Q/T, 404Q, 408D/E, 412N/W, 413W/Y, 415C, 434G/Q, 445G/N, 446H/N, 449E/K/L/M/R/S/V, 459M, 467H/L/M/N/R, 468G, 469E, 471E/F/L/R/S/T/V, 473C/I/L, 474A/D/S, 476A, 477G/H/R/S/V, 478N, 479A/L/R, 480G, 481H/K/T, 484E, 485M/S/T, 487S, 489A/E/G/L/M/N/Q/T, 490G/I/N/Q/R/S/T/V, 493T, 494A/T, 495G/S, 497A/M, 502K/Q, 504G/P/T/W, 505I/L/R, 506T, 507A/I/L/M/R/T/Y, 508L/Q/R/S/T, 509F/G/I/R/V/W, 510S, 512H/L/Q, 514E, 515G/T, 516L, 518E/L/S/V, 519I, 520D/K/R/S/V, 522S/T/V, 524K/S, 525C/Q, 529M/Q, 532V, 533Y, or 534K, or combinations thereof, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

In some embodiments, the amino acid sequence of the engineered glucocerebrosidase comprises at least a substitution at amino acid position 65, 68, 70, 99, 154, 195, 207, 214, 230, 263, 319, 337, 361, 374, 408, 471, or 489, or combinations thereof, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

In some embodiments, the amino acid sequence of the engineered glucocerebrosidase comprises at least a substitution or amino acid residue 65L/M/S/V, 68F/L/Q/T/V/W/Y, 70L, 99E/F/G/K/L/R/S/V/W, 154E, 195M/W, 207S/D, 214F, 230F/L/Y, 263M, 319F/I/V, 337N, 361L/V/W/Y, 374T/V, 408D/E, 471E/F/L/R/S/T/V, or 489A/E/G/L/N/Q/T, or combinations thereof, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

In some embodiments, the amino acid sequence of the engineered glucocerebrosidase comprises at least a substitution or amino acid residue 665L/V, 68L, 70L, 99R, 154E, 195W, 207S, 214F, 230L/Y, 263G, 319I, 337N, 361W, 374T, 408E, 471T, or 489L, or combinations thereof, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, or to the reference sequence corresponding to SEQ ID NO: 54, wherein the amino acid sequence comprises one or more substitutions relative to the reference sequence corresponding to residues 40-536 of SEQ ID NO: 54, or relative to the reference sequence corresponding to SEQ ID NO: 54.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 200-922, or to the reference sequence corresponding to SEQ ID NO: 200-922, wherein the amino acid sequence comprises one or more substitutions relative to the reference sequence corresponding to residues 40-536 of SEQ ID NO: 54, or relative to the reference sequence corresponding to SEQ ID NO: 54.

In some embodiments, the amino acid sequence of the engineered glucocerebrosidase comprises at least a substitution or substitution set at amino acid position(s) 68/336, 65/99/319, 65/68/319, 68/319, 68, 65/319, 195, 65/154/319, 65/68/230, 65, 65/68/99/154/230/319, 68/230, 195/230, 65/230, 141/319, 154/336, 68/154, 68/195/230/319/336, 68/99/195/230/319, 68/195, 65/68, 68/141/230/319, 65/68/99/230/319, 68/99/141/319, 68/154/319, 141/154, 68/99, 99/230/319, 99, 230, 68/230/319, 154/319, 65/68/154/319, 141, 336, 141/230, 68/154/336, 65/68/99/319, 154, 319, 230/319, 68/154, 99/141/319, 509, 108, 108/117/509, 519, 108/519, 88, 117/413/509, 509/519, 413, 108/117/315, 117/271, 117/413/509/529, 413/509, 469, 524, 507, 525, 489, 104, 95, 359, 374,107, 89, 260, 110, 186, 370,191, 333, 189, 468, 473, 115, 53, 449, 518, 236, 481, 59/220, 258, 467, 51, 415, 103, 493, 78, 335, 520, 101, 341, 471, 211, 206, 93, 490, 71, 79, 180, 172, 207, 63, 142, 529, 77, 212, 474, 106, 386/507, 117, 373, 337, 107/246, 516, 233, 91, 194, 84, 361, 171, 536, 50, 116, 239, 44, 40, 113, 243, 169, 504, 505, 109, 479, 477, 97, 182, 111,508, 314, 102, 338, 96, 138, 42, 263, 222, 522, 265, 514, 515, 246, 46, 133, 332, 506, 512, 137, 190,105, 301, 329, 339, or 151, wherein the amino acid positions are relative to the reference sequence corresponding to residues 40-536 of SEQ ID NO: 54, or relative to the reference sequence corresponding to SEQ ID NO: 54.

In some embodiments, the amino acid sequence of the engineered glucocerebrosidase comprises at least a substitution or substitution set 68L/336A, 65L/99R/319I, 65L/68L/319I, 68V/319I, 68V, 65L/319I, 195M, 65L/154E/319I, 65L/68L/230L, 65L, 65L/68L/99R/154E/230L/319I, 68L/230L, 65L/68V/319I, 195M/230L, 65L/230L, 141D/319I, 154E/336A, 68L/319I, 68V/154E, 68V/195M/230L/319I/336A, 68V/99R/195M/230L/319I, 68V/195M, 65L/68V, 68L/141D/230L/319I, 65L/68V/99R/230L/319I, 68L/99R/141D/319I, 68V/154E/319I, 141D/154E, 68V/99R, 99R/230L/319I, 99R, 230L, 68V/230L/319I, 68L, 154E/319I, 65L/68L/154E/319I, 141D, 68V/230L, 336A, 141D/230L, 68V/154E/336A, 65L/68L, 65L/68V/99R/319I, 154E, 319I, 230L/319I, 68L/154E, 99R/141D/319I, 68L/154E/319I, 509F, 108I, 108I/117/509F, 519I, 108I/519I, 88L, 117I/413Y/509F, 509F/519I, 413Y, 108I/117/315T, 117I/271T, 117I/413Y/509F/529Q, 413Y/509F, 469E, 524K, 507M, 509I, 525Q, 489A, 104M, 507A, 95G, 359E, 374V, 509W, 107D, 89C, 260R, 110Q, 186G, 370R, 509R, 191R, 333L, 189R, 468G, 473I, 115M, 191N, 53L, 524S, 449L, 518E, 236M, 481H, 115R, 59N/220G, 489G, 258T, 467N, 51P, 415C, 103S, 493T, 489T, 518V, 78L, 195W, F65M, 413W, 335M, 520K, 107E, 101Q, 449K, 481K, 104V, 341R, 78M, 507L, 449M, 471L, 211L, 520D, 103P, 206F, 93S, 490S, 473C, 507R, 21H, 71S, 79R, 180C, 509G, 206R, 211N, 172S, 490R, 207D, 115V, 63L, 142A, 529M, 71R, 473L, 95T, 107A, 520V, 101L, 520S, 71H, 89D, 77I, 507Y, 107C, 518S, 212A, 71F, 525C, 449R, 107M, 467H, 474D, 101S, 106W, 386L/507M, 471S, 107Q, 68Q, 79I, 518L, 474A, 107S, 117T, 467M, 471R, 341S, 490G, 471F, 490T, 333T, 101R, 373V, 101V, 101W, 65S, 490V, 337N, 374T, 68Y, 95L, 71G, 471V, 107M/246R, 110A, 142Y, 65V, 71M, 77G, 77R, 230Y, 490Q, 101T, 103A, 507T, 471T, 509V, 520R, 319F, 142N, 79V, 467L, 77N, 71L, 68W, 189S, 359P, 71Y, 77T, 142M, 373A, 319V, 212N, 110H, 333C, 490I, 68F, 474S, 103L, 77H, 471E, 516L, 467R, 233R, 91T, 194L, 84G, 361V, 233A, 171V, 536L, 50A, 116R, 239T, 44V, 40S, 113N, 91I, 99F, 243L, 169V, 504P, 505R, 109R, 536T, 479A, 479L, 477H, 84K, 97N, 97K, 182M, 111H, 361Y, 508R, 314R, 336G, 102E, 338R, 505L, 99L, 96S, 138S, 508T, 96I, 508Q, 243M, 111L, 508S, 479R, 42A, 239I, 97I, 116E, 113I, 477V, 536I, 263G, 99S, 222I, 536D, 477G, 536W, 536C, 336R, 243R, 116V, 536R, 522V, 102S, 97R, 40H, 243V, 265S, 116Q, 514E, 508L, 515T, 246R, 361W, 111F, 489M, 477R, 116Y, 46R, 133M, 332R, 239R, 109G, 506T, 504G, 233Q, 96R, 99G, 504T, 96G, 97M, 109L, 515G, 233G, 84A, 102N, 504W, 265R, 99V, 512L, 137V, 536A, 113R, 171P, 190W, 314G, 46M, 182H, 246K, 105T, 301K, 522T, 329L, 96V, 339A, 477S, 113G, 361L, 505I, 105M, 116M, 96M, 109M, 171C, 536M, or 151L, wherein the amino acid positions are relative to the reference sequence corresponding to residues 40-536 of SEQ ID NO: 54, or relative to the reference sequence corresponding to SEQ ID NO: 54.

In some embodiments, the amino acid sequence of the engineered glucocerebrosidase comprises at least a substitution or substitution set P68L/T336A, F65L/H99R/M319I, F65L/P68L/M319I, P68V/M319I, P68V, F65L/M319I, L195M, F65L/G154E/M319I, F65L/P68L/V230L, F65L, F65L/P68L/H99R/G154E/V230L/M319I, P68L/V230L, F65L/P68V/M319I, L195M/V230L, F65L/V230L, N141D/M319I, G154E/T336A, P68L/M319I, P68V/G154E, P68V/L195M/V230L/M319I/T336A, P68V/H99R/L195M/V230L/M319I, P68V/L195M, F65L/P68V, P68L/N141D/V230L/M319I, F65L/P68V/H99R/V230L/M319I, P68L/H99R/N141D/M319I, P68V/G154E/M319I, N141D/G154E, P68V/H99R, H99R/V230L/M319I, H99R, V230L, P68V/V230L/M319I, P68L, G154E/M319I, F65L/P68L/G154E/M319I, N141D, P68V/V230L, T336A, N141D/V230L, P68V/G154E/T336A, F65L/P68L, F65L/P68V/H99R/M319I, G154E, M319I, V230L/M319I, P68L/G154E, H99R/N141D/M319I, P68L/G154E/M319I, L509F, L108I, L108I/V117I/L509F, L519I, L108I/L519I, M88L, V117I/H413Y/L509F, L509F/L519I, H413Y, L108I/V117I/V315T, V117I/A271T, V117I/H413Y/L509F/H529Q, H413Y/L509F, G469E, P524K, V507M, L509I, G525Q, L489A, L104M, V507A, 195G, A359E, M374V, L509W, T107D, E89C, A260R, P110Q, F186G, F370R, L509R, E191R, V333L, P189R, E468G, V473I, Q115M, E191N, V53L, P524S, T449L, F518E, L236M, N481H, Q115R, A59N/S220G, L489G, A258T, P467N, S51P, V415C, G103S, G493T, L489T, F518V, R78L, L195W, F65M, H413W, L335M, E520K, T107E, G101Q, T449K, N481K, L104V, A341R, R78M, V507L, T449M, Q471L, V211L, E520D, G103P, L206F, G93S, H490S, V473C, V507R, V211I, P71S, Y79R, D180C, L509G, L206R, V211N, Y172S, H490R, S207D, Q115V, D63L, L142A, H529M, P71R, V473L, 195T, T107A, E520V, G101L, E520S, P71H, E89D, S77I, V507Y, T107C, F518S, S212A, P71F, G525C, T449R, T107M, P467H, G474D, G101S, L, 106W, F386L/V507M, Q471S, T107Q, P68Q, Y79I, F518L, G474A, T107S, V117T, P467M, Q471R, A341S, H490G, Q471F, H490T, V333T, G101R, T373V, G101V, G101W, F65S, H490V, D337N, M374T, P68Y, 195L, P71G, Q471V, T107M/Q246R, P110A, L142Y, F65V, P71M, S77G, S77R, V230Y, H490Q, G101T, G103A, V507T, Q471T, L509V, E520R, M319F, L142N, Y79V, P467L, S77N, P71L, P68W, P189S, A359P, P71Y, S77T, L142M, T373A, M319V, S212N, P110H, V333C, H490I, P68F, G474S, G103L, S77H, Q471E, V516L, P467R, K233R, S91T, K194L, S84G, A361V, K233A, T171V, Q536L, Y50A, K116R, Q239T, I44V, A40S, K113N, S91I, H99F, 1243L, 1169V, S504P, K505R, Q109R, Q536T, Q479A, Q479L, A477H, S84K, A97N, A97K, Q182M, E111H, A361Y, P508R, N314R, T336G, T102E, P338R, K505L, H99L, Q96S, P138S, P508T, Q96I, P508Q, I243M, E111L, P508S, Q479R, P42A, Q239I, A97I, K116E, K113I, A477V, Q536I, K263G, H99S, T222I, Q536D, A477G, Q536W, Q536C, T336R, 1243R, K116V, Q536R, 1522V, T102S, A97R, A40H, 1243V, Q265S, K116Q, P514E, P508L, A515T, Q246R, A361W, E111F, L489M, A477R, K116Y, K46R, L133M, K332R, Q239R, Q109G, D506T, S504G, K233Q, Q96R, I199G, S504T, Q96G, A97M, Q109L, A515G, K233G, S84A, T102N, S504W, Q265R, H99V, K512L, P137V, Q536A, K113R, T171P, E190W, N314G, K46M, Q182H, Q246K, L105T, R301K, 1522T, H329L, Q96V, E339A, A477S, K113G, A361L, K505I, L105M, K116M, Q96M, Q109M, T171C, Q536M, or E151L, wherein the amino acid positions are relative to the reference sequence corresponding to residues 40-536 of SEQ ID NO: 54, or relative to the reference sequence corresponding to SEQ ID NO: 54.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 220, or to the reference sequence corresponding to SEQ ID NO: 220, wherein the amino acid sequence comprises one or more substitutions relative to the reference sequence corresponding to residues 40-536 of SEQ ID NO: 220, or relative to the reference sequence corresponding to SEQ ID NO: 220.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 924-1020, or to the reference sequence corresponding to SEQ ID NO: 924-1020, wherein the amino acid sequence comprises one or more substitutions relative to the reference sequence corresponding to residues 40-536 of SEQ ID NO: 220, or relative to the reference sequence corresponding to SEQ ID NO: 220.

In some embodiments, the amino acid sequence of the engineered glucocerebrosidase comprises at least a substitution or substitution set at amino acid position(s) 65/195/361, 514, 79/195/263/361, 65/374, 79/195, 65/230, 65/233/263/337/359/361/374, 79/536, 79/230/233/239/359/361/471, 65/471, 374, 65/195/230/233/337/374/514, 65/195/233/359/536, 230, 65/195/263/276/359/361/374/471, 79, 195/230/233/337/374/536, 195/230, 195/230/337/359/361, 230/263, 65/230/233, 263, 195/230/337/361/374/471, 65/195, 374/471, 374/536, 239, 65, 230/233/536, 230/263/374, 65/195/230/263/337/361/374/471, 79/374, 65/361/374/471/536, 195, 233, 79/230/359/374, 536, 230/359/361/374/514, 65/79/230, 195/239/263/361/374/514, 195/263/374, 195/374, 65/230/233/361/536, 230/233/374/471, 65/233/359/361/471, 65/359/471, 65/79, or 65/359, wherein the amino acid positions are relative to the reference sequence corresponding to residues 40-536 of SEQ ID NO: 220, or relative to the reference sequence corresponding to SEQ ID NO: 220.

In some embodiments, the amino acid sequence of the engineered glucocerebrosidase comprises at least a substitution or substitution set 65V/195W/361W, 514E, 79V/195W/263G/361W, 65V/374T, 79V/195W, 65V/230Y, 65V/233A/263G/337N/359P/361W/374T, 79V/536D, 79V/230Y/233A/239R/359P/361W/471T, 65V/471T, 374T, 65V/195W/230Y/233A/337N/374T/514E, 65V/195W/233A/359P/536D, 230Y, 65V/195W/263G/276T/359P/361W/374T/471T, 79V, 195W/230Y/233A/337N/374T/536D, 195W/230Y, 195W/230Y/337N/359P/361W, 230Y/263G, 65V/230Y/233G, 263G, 195W/230Y/337N/361W/374T/471T, 65V/195W, 374T/471T, 374T/536D, 239R, 65V, 230Y/233G/536L, 230Y/263G/374T, 65V/195W/230Y/263G/337N/361W/374T/471T, 79V/374T, 65V/361W/374T/471T/536D, 195W, 233G, 79V/230Y/359P/374T, 536D, 230Y/359P/361W/374T/514E, 65V/79V/230Y, 195W/239R/263G/361W/374T/514E, 195W/263G/374T, 195W/374T, 65V/230Y/233A/361W/536L, 230Y/233A/374T/471T, 65V/233A/359P/361W/471T, 536L, 65V/359P/471T, 65V/79V, or 65V/359P, wherein the amino acid positions are relative to the reference sequence corresponding to residues 40-536 of SEQ ID NO: 220, or relative to the reference sequence corresponding to SEQ ID NO: 220.

In some embodiments, the amino acid sequence of the engineered glucocerebrosidase comprises at least a substitution or substitution set L65V/L195W/A361W, P514E, Y79V/L195W/K263G/A361W, L65V/M374T, Y79V/L195W, L65V/L230Y, L65V/K233A/K263G/D337N/A359P/A361W/M374T, Y79V/Q536D, Y79V/L230Y/K233A/Q239R/A359P/A361W/Q471T, L65V/Q471T, M374T, L65V/L195W/L230Y/K233A/D337N/M374T/P514E, L65V/L195W/K233A/A359P/Q536D, L230Y, L65V/L195W/K263G/S276T/A359P/A361W/M374T/Q471T, Y79V, L195W/L230Y/K233A/D337N/M374T/Q536D, L195W/L230Y, L195W/L230Y/D337N/A359P/A361W, L230Y/K263G, L65V/L230Y/K233G, K263G, L195W/L230Y/D337N/A361W/M374T/Q471T, L65V/L195W, M374T/Q471T, M374T/Q536D, Q239R, L65V, L230Y/K233G/Q536L, L230Y/K263G/M374T, L65V/L195W/L230Y/K263G/D337N/A361W/M374T/Q471T, Y79V/M374T, L65V/A361W/M374T/Q471T/Q536D, L195W, K233G, Y79V/L230Y/A359P/M374T, Q536D, L230Y/A359P/A361W/M374T/P514E, L65V/Y79V/L230Y, L195W/Q239R/K263G/A361W/M374T/P514E, L195W/K263G/M374T, L195W/M374T, L65V/L230Y/K233A/A361W/Q536L, L230Y/K233A/M374T/Q471T, L65V/K233A/A359P/A361W/Q471T, Q536L, L65V/A359P/Q471T, L65V/Y79V, L65V/A359P, wherein the amino acid positions are relative to the reference sequence corresponding to residues 40-536 of SEQ ID NO: 220, or relative to the reference sequence corresponding to SEQ ID NO: 220.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 984, or to the reference sequence corresponding to SEQ ID NO: 984, wherein the amino acid sequence comprises one or more substitutions relative to the reference sequence corresponding to residues 40-536 of SEQ ID NO: 984, or relative to the reference sequence corresponding to SEQ ID NO: 984.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 1022-1216, or to the reference sequence corresponding to SEQ ID NO: 1022-1216, wherein the amino acid sequence comprises one or more substitutions relative to the reference sequence corresponding to residues 40-536 of SEQ ID NO: 984, or relative to the reference sequence corresponding to SEQ ID NO: 984.

In some embodiments, the amino acid sequence of the engineered glucocerebrosidase comprises at least a substitution or substitution set at amino acid position(s) 359, 79/359, 79/97/182, 97/359, 97, 79/182, 201, 404, 484, 446, 494/495, 495, 412, 385, 100, 184, 82, 75, 71, 41, 99, 94, 92, 98, 114, 95, 102, 96, 105, 113, 325, 336, 262, 386, 360, 400, 342, 390, 264, 298, 382, 408, 449, 459, 434, 476,502, 29/534, 497, 485, 533, 536, 510, 532, 489, 534, 494, 182, 137, 204, 194, 202, 144, 142,175, 220, 150, or 206, wherein the amino acid positions are relative to the reference sequence corresponding to residues 40-536 of SEQ ID NO: 984, or relative to the reference sequence corresponding to SEQ ID NO: 984.

In some embodiments, the amino acid sequence of the engineered glucocerebrosidase comprises at least a substitution or substitution set 359P, 79V/359P, 79V/97R/182M, 97R/359P, 97R, 79V/182M, 201T, 404Q, 484E, 201R, 446N, 494T/495G, 495G, 412W, 385N, 385G, 100N, 184V, 385H, 82S, 75A, 71T, 41H, 97K, 99E, 97Q, 94N, 100R, 92A, 98P, 99W, 92G, 114Y, 95H, 102A, 92K, 96D, 94E, 96E, 95W, 105I, 113Q, 92H, 92Q, 92E, 325V, 336E, 385Q, 262Q, 386I, 360E, 400T, 342Q, 400E, 400Q, 390A, 264I, 298W, 382A, 408D, 449E, 459M, 434G, 476A, 408E, 502K, 29I/534K, 502Q, 497A, 485S, 533Y, 495S, 536P, 536H, 485M, 510S, 485T, 532V, 489E, 534K, 494T, 182G, 137Q, L204Q, 194H, 184Q, 202Q, 182S, 144M, 142N, 201Q, 182D, 175D, 182T, 194Q, 220A, 150T, or 206I, wherein the amino acid positions are relative to the reference sequence corresponding to residues 40-536 of SEQ ID NO: 984, or relative to the reference sequence corresponding to SEQ ID NO: 984.

In some embodiments, the amino acid sequence of the engineered glucocerebrosidase comprises at least a substitution or substitution set A359P, Y79V/A359P, Y79V/A97R/Q182M, A97R/A359P, A97R, Y79V/Q182M, H201T, H404Q, D484E, H201R, T446N, S494T/A495G, A495G, Y412W, K385N, K385G, T100N, H184V, K385H, T82S, T75A, P71T, R41H, A97K, R99E, A97Q, P94N, T100R, M92A, N98P, R99W, M92G, F114Y, 195H, T102A, M92K, Q96D, P94E, Q96E, 195W, L105I, K113Q, M92H, M92Q, M92E, L325V, T336E, K385Q, H262Q, F386I, K360E, M400T, K342Q, M400E, M400Q, S390A, L264I, F298W, V382A, T408D, T449E, L459M, R434G, V476A, T408E, R502K, L29I/R534K, R502Q, V497A, A485S, W533Y, A495S, Q536P, Q536H, A485M, T510S, A485T, L532V, L489E, R534K, S494T, Q182G, P137Q, L204Q, K194H, H184Q, R202Q, Q182S, L144M, L142N, H201Q, Q182D, A175D, Q182T, K194Q, S220A, E150T, or L206I, wherein the amino acid positions are relative to the reference sequence corresponding to residues 40-536 of SEQ ID NO: 984, or relative to the reference sequence corresponding to SEQ ID NO: 984.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 1150, or to the reference sequence corresponding to SEQ ID NO: 1150, wherein the amino acid sequence comprises one or more substitutions relative to the reference sequence corresponding to residues 40-536 of SEQ ID NO: 1150, or relative to the reference sequence corresponding to SEQ ID NO: 1150.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 1218-1554, or to the reference sequence corresponding to SEQ ID NO: 1218-1554, wherein the amino acid sequence comprises one or more substitutions relative to the reference sequence corresponding to residues 40-536 of SEQ ID NO: 1150, or relative to the reference sequence corresponding to SEQ ID NO: 1150.

In some embodiments, the amino acid sequence of the engineered glucocerebrosidase comprises at least a substitution or substitution set at amino acid position(s) 96/194/382/484, 359/382/412/494/495, 92/96/175/182/382, 96/359/382/484/494/495, 92/175/194/359/382, 359/382/400/412, 92/96/182/382/400/484, 92/96/382/494/495, 92/96/175/382/494/495, 96/182/359/382/484, 92/96/382, 92/382/494/495/534, 92/96/382/400/484, 92/96/175/382, 92/175/382/400/484, 92/96/175/182/382/412/484, 92/96/201/382/484, 92/182/194/382, 92/96/175/194/359/382/400, 92/359/382, 96/382, 92/175/382/484/494/495, 96/201/359/382, 92, 194, 201, 359, 96, 92/96/382/400/484/495, 382/495, 382, 92/382, 71/175/325/385, 325/385, 71/325/502, 71/175/325/385/502, 202/325/385, 97/175/385/510, 325/385/510, 100/175/325, 71/175/325/360/385, 97/175/510, 71/97/100/175/325/385, 71/175/510, 71/97/325/502, 71/100/325/385, 202/385/489, 325/385/502, 71/100/325/385/510, 71/100/175/325/385, 385/510, 97/325/385, 71, 97/202/325/360/385/510, 502, 71/97/103/325, 100/325/385/502/510, 71/137/175/325/360/385/502, 71/276/325/385/510, 71/175/360/385/502, 100/175/325/385/489/502, 71/175/360/385/485/502, 95/113, 494/536, 95/99/113/182/184/264/386, 184/536, 95/142/144/182/184/386/449, 144/204/386/400/449, 113/449/536, 536, 95/386/536, 264, 99/113/386/449, 95/386, 95/99/204/386, 113/142/386/449, 182/184/264/494, 113/142/182/494, 113, 99/264/449, 95, 95/99, 99/113/449, 95/113/449/536, 95/99/113, 386/536, 99/182/184, 142/386/400/536, 35/264/449/536, 142/144/220, 99/142, 29/102/182/276/336/390/459/502/532, 41/262/336/502, 29/97/150/262/336/502/532, 29, 532, 102/390/459/502/532, 29/262/276/336/495, 29/182/262/459/532, 92/99/102/502, 29/97/102/182/502/532, 336/459/495/502/532, 150/182/336/390, 150/262/495, 29/495, 29/102/182/262/502, 29/336/459/502/532, 182/495/502/532, 102/336, 150/182/262/495, 459/495/502/532, 41/182/390/495, 29/41/182/336/495, 97/102/495, 182/262/495, 29/262/502, 29/102/502, 182/336/495, 29/150/182/262/459/495, 502/532, 386, 497, 99, 512, 69, 507, 445, 75, 487, 184, 90, 235, 434, 489, 86, 182, 446, 490, 299, 102, 230, 68, 141, 97, 316, 522, 224, 478, 480, 368, 412, 449, 265, or 494, wherein the amino acid positions are relative to the reference sequence corresponding to residues 40-536 of SEQ ID NO: 1150, or relative to the reference sequence corresponding to SEQ ID NO: 1150.

In some embodiments, the amino acid sequence of the engineered glucocerebrosidase comprises at least a substitution or substitution set 96E/194Q/382A/484E, 359P/382A/412W/494T/495G, 92Q/96E/175D/182S/382A, 96E/359P/382A/484E/494T/495G, 92E/175D/194Q/359P/382A, 359P/382A/400E/412W, 92Q/96E/182S/382A/400E/484E, 92Q/96E/382A/494T/495G, 92Q/96E/175D/382A/494T/495G, 96E/182S/359P/382A/484E, 92E/96E/382A, 92Q/382A/494T/495G/534K, 92E/96E/382A/400Q/484E, 92Q/96E/175D/382A, 92Q/175D/382A/400Q/484E, 92E/96E/175D/382A/494T/495G, 92Q/96E/175D/182S/382A/412W/484E, 92Q/96E/201Q/382A/484E, 92E/182S/194Q/382A, 92Q/96E/175D/194Q/359P/382A/400E, 92Q/359P/382A, 96E/382A, 92Q/175D/382A/484E/494T/495G, 96E/201Q/359P/382A, 92E, 92Q, 194Q, 201Q, 359P, 96E, 92E/96E/382A/400E/484E/495G, 382A/495G, 382A, 92E/382A, 71T/175D/325V/385H, 325V/385H, 71T/325V/502Q, 71T/175D/325V/385H/502Q, 202Q/325V/385N, 97Q/175D/385H/510S, 325V/385Q/510S, 100R/175D/325V, 71T/175D/325V/360E/385N, 97Q/175D/510S, 71T/97Q/100R/175D/325V/385H, 71T/175D/510S, 71T/97Q/325V/502Q, 71T/100R/325V/385Q, 202Q/385N/489E, 325V/385N/502Q, 325V/385H/502Q, 71T/100R/325V/385H/510S, 71T/100R/175D/325V/385N, 71T/100R/175D/325V/385H, 385N/510S, 97Q/325V/385N, 71T, 97Q/202Q/325V/360E/385G/510S, 502Q, 71T/97Q/103S/325V, 100R/325V/385H/502Q/510S, 71T/137Q/175D/325V/360E/385H/502Q, 71T/276T/325V/385R/510S, 71T/175D/360E/385N/502Q, 100R/175D/325V/385N/489E/502Q, 71T/175D/360E/385H/485S/502Q, 95W/113Q, 494T/536P, 95W/99W/113Q/182D/184V/264I/386I, 184Q/536P, 95W/142N/144M/182G/184Q/386I/449E, 144M/204Q/386I/400T/449E, 113Q/449E/536P, 536P, 95W/386I/536P, 264I, 99W/113Q/386I/449E, 95W/386I, 95W/99W/204Q/386I, 113Q/142N/386I/449E, 182G/184Q/264I/494T, 113Q/142N/182D/494T, 113Q, 99W/264I/449E, 95W, 95W/99W, 99W/113Q/449E, 95W/113Q/449E/536P, 95W/99W/113Q, 386I/536P, 99W/182G/184Q, 142N/386I/400T/536P, 35T/264I/449E/536P, 142N/144M/220A, 99W/142N, 29I/102A/182M/276T/336E/390A/459M/502K/532V, 41H/262Q/336E/502K, 29I/97R/150T/262Q/336E/502K/532V, 29I, 532V, 102A/390A/459M/502K/532V, 29I/262Q/276T/336E/495G, 29I/182T/262Q/459M/532V, 92K/99G/102A/502K, 29I/97R/102A/182M/502K/532V, 336E/459M/495S/502K/532V, 150T/182T/336E/390A, 150T/262Q/495G, 29I/495S, 29I/102A/182T/262Q/502K, 29I/336E/459M/502K/532V, 182M/495G/502K/532V, 102A/336E, 150T/182M/262Q/495S, 459M/495S/502K/532V, 41H/182M/390A/495G, 29I/41H/182T/336E/495S, 97R/102A/495S, 182T/262Q/495S, 29I/262Q/502K, 29I/102A/502K, 182M/336E/495S, 502K, 29I/150T/182T/262Q/459M/495G, 502K/532V, 386P, 497M, 99K, 512H, 69D, 507I, 445N, 75N, 75H, 512Q, 487S, 113P, 184I, 90M, 235T, 434Q, 445G, 489N, 90A, 386L, 75E, 86E, 489Q, 182E, 446H, 490N, 299V, 96N, 102P, 230F, 68T, 141E, 97E, 316H, 522S, 224M, 478N, 480G, 368D, 86H, 412N, 449S, 265N, or 494A, wherein the amino acid positions are relative to the reference sequence corresponding to residues 40-536 of SEQ ID NO: 1150, or relative to the reference sequence corresponding to SEQ ID NO: 1150.

In some embodiments, the amino acid sequence of the engineered glucocerebrosidase comprises at least a substitution or substitution set Q96E/K194Q/V382A/D484E, A359P/V382A/Y412W/S494T/A495G, M92Q/Q96E/A175D/Q182S/V382A, Q96E/A359P/V382A/D484E/S494T/A495G, M92E/A175D/K194Q/A359P/V382A, A359P/V382A/M400E/Y412W, M92Q/Q96E/Q182S/V382A/M400E/D484E, M92Q/Q96E/V382A/S494T/A495G, M92Q/Q96E/A175D/V382A/S494T/A495G, Q96E/Q182S/A359P/V382A/D484E, M92E/Q96E/V382A, M92Q/V382A/S494T/A495G/R534K, M92E/Q96E/V382A/M400Q/D484E, M92Q/Q96E/A175D/V382A, M92Q/A175D/V382A/M400Q/D484E, M92E/Q96E/A175D/V382A/S494T/A495G, M92Q/Q96E/A175D/Q182S/V382A/Y412W/D484E, M92Q/Q96E/H201Q/V382A/D484E, M92E/Q182S/K194Q/V382A, M92Q/Q96E/A175D/K194Q/A359P/V382A/M400E, M92Q/A359P/V382A, Q96E/V382A, M92Q/A175D/V382A/D484E/S494T/A495G, Q96E/H201Q/A359P/V382A, M92E, M92Q, K194Q, H201Q, A359P, Q96E, M92E/Q96E/V382A/M400E/D484E/A495G, V382A/A495G, V382A, M92E/V382A, P71T/A175D/L325V/K385H, L325V/K385H, P71T/L325V/R502Q, P71T/A175D/L325V/K385H/R502Q, R202Q/L325V/K385N, A97Q/A175D/K385H/T510S, L325V/K385Q/T510S, T100R/A175D/L325V, P71T/A175D/L325V/K360E/K385N, A97Q/A175D/T510S, P71T/A97Q/T100R/A175D/L325V/K385H, P71T/A175D/T510S, P71T/A97Q/L325V/R502Q, P71T/T100R/L325V/K385Q, R202Q/K385N/L489E, L325V/K385N/R502Q, L325V/K385H/R502Q, P71T/T100R/L325V/K385H/T510S, P71T/T100R/A175D/L325V/K385N, P71T/T100R/A175D/L325V/K385H, K385N/T510S, A97Q/L325V/K385N, P71T, A97Q/R202Q/L325V/K360E/K385G/T510S, R502Q, P71T/A97Q/G103S/L325V, T100R/L325V/K385H/R502Q/T510S, P71T/P137Q/A175D/L325V/K360E/K385H/R502Q, P71T/S276T/L325V/K385R/T510S, P71T/A175D/K360E/K385N/R502Q, T100R/A175D/L325V/K385N/L489E/R502Q, P71T/A175D/K360E/K385H/A485S/R502Q, 195W/K113Q, S494T/Q536P, 195W/R99W/K113Q/Q182D/H184V/L264I/F386I, H184Q/Q536P, 195W/L142N/L144M/Q182G/H184Q/F386I/T449E, L144M/L204Q/F386I/M400T/T449E, K113Q/T449E/Q536P, Q536P, 195W/F386I/Q536P, L264I, R99W/K113Q/F386I/T449E, 195W/F386I, 195W/R99W/L204Q/F386I, K113Q/L142N/F386I/T449E, Q182G/H184Q/L264I/S494T, K113Q/L142N/Q182D/S494T, K113Q, R99W/L264I/T449E, 195W, 195W/R99W, R99W/K113Q/T449E, 195W/K113Q/T449E/Q536P, 195W/R99W/K113Q, F386I/Q536P, R99W/Q182G/H184Q, L142N/F386I/M400T/Q536P, S35T/L264I/T449E/Q536P, L142N/L144M/S220A, R99W/L142N, L29I/T102A/Q182M/S276T/T336E/S390A/L459M/R502K/L532V, R41H/H262Q/T336E/R502K, L29I/A97R/E150T/H262Q/T336E/R502K/L532V, L29I, L532V, T102A/S390A/L459M/R502K/L532V, L29I/H262Q/S276T/T336E/A495G, L29I/Q182T/H262Q/L459M/L532V, M92K/R99G/T102A/R502K, L29I/A97R/T102A/Q182M/R502K/L532V, T336E/L459M/A495S/R502K/L532V, E150T/Q182T/T336E/S390A, E150T/H262Q/A495G, L29I/A495S, L29I/T102A/Q182T/H262Q/R502K, L29I/T336E/L459M/R502K/L532V, Q182M/A495G/R502K/L532V, T102A/T336E, E150T/Q182M/H262Q/A495S, L459M/A495S/R502K/L532V, R41H/Q182M/S390A/A495G, L29I/R41H/Q182T/T336E/A495S, A97R/T102A/A495S, Q182T/H262Q/A495S, L29I/H262Q/R502K, L29I/T102A/R502K, Q182M/T336E/A495S, R502K, L29I/E150T/Q182T/H262Q/L459M/A495G, R502K/L532V, F386P, V497M, R99K, K512H, T69D, V507I, I445N, T75N, T75H, K512Q, A487S, K113P, H184I, L90M, S235T, R434Q, I445G, L489N, L90A, F386L, T75E, R86E, L489Q, Q182E, T446H, H490N, I299V, Q96N, T102P, Y230F, L68T, N141E, A97E, R316H, I522S, L224M, S478N, K480G, R368D, R86H, Y412N, T449S, Q265N, or S494A, wherein the amino acid positions are relative to the reference sequence corresponding to residues 40-536 of SEQ ID NO: 1150, or relative to the reference sequence corresponding to SEQ ID NO: 1150.

In some embodiments, the amino acid sequence of the engineered glucocerebrosidase comprises at least a substitution at an amino acid position set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, 54, 220, 984, or 1150, or relative to the reference sequence corresponding to SEQ ID NO: 54, 220, or 984, or 1150.

In some embodiments, the amino acid sequence of the engineered glucocerebrosidase comprises at least one substitution set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, 54, 220, 984, or 1150, or relative to the reference sequence corresponding to SEQ ID NO: 54, 220, or 984, or 1150.

In some embodiments, the amino acid sequence of the engineered glucocerebrosidase comprises at least a substitution or substitution set at amino acid position(s) set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, 54, 220, 984, or 1150, or relative to the reference sequence corresponding to SEQ ID NO: 2, 54, 220, or 984, or 1150.

In some embodiments, the amino acid sequence of the engineered glucocerebrosidase comprises at least a substitution or substitution set of an engineered glucocerebrosidase set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, 54, 220, 984, or 1150, or relative to the reference sequence corresponding to SEQ ID NO: 2, 54, 220, or 984, or 1150.

In some embodiments, the engineered glucocerebrosidase comprises an amino acid sequence having 95%, 96%, 97%, 98%, 99%, or more sequence identity to a reference sequence comprising a substitution or substitution set of an engineered glucocerebrosidase set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, 54, 220, 984, or 1150, or relative to the reference sequence corresponding to SEQ ID NO: 2, 54, 220, or 984, or 1150.

In some embodiments, the amino acid sequence of the engineered glucocerebrosidase comprises residues 40-536 of an engineered glucocerebrosidase set forth in Tables 3.1, 3.2, 4.1, 4.2, 4.3, 4.4, 5.1, 6.1, 6.2, 12.1, 12.2, and 13.1, and 6.2, or an amino acid sequence comprising an engineered glucocerebrosidase set forth in Tables 3.1, 3.2, 4.1, 4.2, 4.3, 4.4, 5.1, 6.1, 6.2, 12.1, 12.2, and 13.1.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 40-536 of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, 1410, 1412, 1414, 1416, 1418, 1420, 1422, 1424, 1426, 1428, 1430, 1432, 1434, 1436, 1438, 1440, 1442, 1444, 1446, 1448, 1450, 1452, 1454, 1456, 1458, 1460, 1462, 1464, 1466, 1468, 1470, 1472, 1474, 1476, 1478, 1480, 1482, 1484, 1486, 1488, 1490, 1492, 1494, 1496, 1498, 1500, 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 1586, 1588, 1590, 1592, 1594, 1596, 1598, 1600, 1602, 1604, 1606, 1608, 1610, 1612, 1614, 1616, 1618, 1620, 1622, 1624, 1626, 1628, 1630, 1632, 1634, 1636, 1638, 1640, 1642, 1644, 1646, or 1648.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, 1410, 1412, 1414, 1416, 1418, 1420, 1422, 1424, 1426, 1428, 1430, 1432, 1434, 1436, 1438, 1440, 1442, 1444, 1446, 1448, 1450, 1452, 1454, 1456, 1458, 1460, 1462, 1464, 1466, 1468, 1470, 1472, 1474, 1476, 1478, 1480, 1482, 1484, 1486, 1488, 1490, 1492, 1494, 1496, 1498, 1500, 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 1586, 1588, 1590, 1592, 1594, 1596, 1598, 1600, 1602, 1604, 1606, 1608, 1610, 1612, 1614, 1616, 1618, 1620, 1622, 1624, 1626, 1628, 1630, 1632, 1634, 1636, 1638, 1640, 1642, 1644, 1646, or 1648.

In some embodiments, the engineered glucocerebrosidase comprises an amino acid sequence comprising residues 40-536 of an even-numbered SEQ ID NO. of SEQ ID NOs: 4-1648, or comprising an even-numbered SEQ ID NO. of SEQ ID NOs: 4-1648. In some embodiments, the amino acid sequence of the engineered glucocerebrosidase optionally includes 1, 2, 3, 4, 5, 6, 7, 8, 9, or up to 10 substitutions, insertions, and/or deletions. In some embodiments, the amino acid sequence of the engineered glucocerebrosidase optionally includes 1, 2, 3, 4, 5, 6, 7, 8, 9, or up to 10 substitutions. In some embodiments, the amino acid sequence of the engineered glucocerebrosidase optionally includes 1, 2, 3, 4, or 5 substitutions, insertions, and/or deletions. In some embodiments, the amino acid sequence of the engineered glucocerebrosidase optionally includes 1, 2, 3, 4, or 5 substitutions. In some embodiments, the amino acid substitutions comprises non-conservative and/or conservative substitutions. In some embodiments, the substitutions comprise conservative substitutions.

In some embodiments, the engineered glucocerebrosidase comprises an amino acid sequence comprising residues 40-536 of SEQ ID NO. 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, 1410, 1412, 1414, 1416, 1418, 1420, 1422, 1424, 1426, 1428, 1430, 1432, 1434, 1436, 1438, 1440, 1442, 1444, 1446, 1448, 1450, 1452, 1454, 1456, 1458, 1460, 1462, 1464, 1466, 1468, 1470, 1472, 1474, 1476, 1478, 1480, 1482, 1484, 1486, 1488, 1490, 1492, 1494, 1496, 1498, 1500, 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 1586, 1588, 1590, 1592, 1594, 1596, 1598, 1600, 1602, 1604, 1606, 1608, 1610, 1612, 1614, 1616, 1618, 1620, 1622, 1624, 1626, 1628, 1630, 1632, 1634, 1636, 1638, 1640, 1642, 1644, 1646, or 1648. In some embodiments, the amino acid sequence of the engineered glucocerebrosidase optionally includes 1, 2, 3, 4, 5, 6, 7, 8, 9, or up to 10 substitutions, insertions, and/or deletions. In some embodiments, the amino acid sequence of the engineered glucocerebrosidase optionally includes 1, 2, 3, 4, 5, 6, 7, 8, 9, or up to 10 substitutions. In some embodiments, the amino acid sequence of the engineered glucocerebrosidase optionally includes 1, 2, 3, 4, or 5 substitutions, insertions, and/or deletions. In some embodiments, the amino acid sequence of the engineered glucocerebrosidase optionally includes 1, 2, 3, 4, or 5 substitutions. In some embodiments, the amino acid substitutions comprises non-conservative and/or conservative substitutions. In some embodiments, the substitutions comprise conservative substitutions.

In some embodiments, the engineered glucocerebrosidase comprises an amino acid sequence comprising SEQ ID NO. 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, 1410, 1412, 1414, 1416, 1418, 1420, 1422, 1424, 1426, 1428, 1430, 1432, 1434, 1436, 1438, 1440, 1442, 1444, 1446, 1448, 1450, 1452, 1454, 1456, 1458, 1460, 1462, 1464, 1466, 1468, 1470, 1472, 1474, 1476, 1478, 1480, 1482, 1484, 1486, 1488, 1490, 1492, 1494, 1496, 1498, 1500, 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 1586, 1588, 1590, 1592, 1594, 1596, 1598, 1600, 1602, 1604, 1606, 1608, 1610, 1612, 1614, 1616, 1618, 1620, 1622, 1624, 1626, 1628, 1630, 1632, 1634, 1636, 1638, 1640, 1642, 1644, 1646, or 1648. In some embodiments, the amino acid sequence of the engineered glucocerebrosidase optionally includes 1, 2, 3, 4, 5, 6, 7, 8, 9, or up to 10 substitutions, insertions, and/or deletions. In some embodiments, the amino acid sequence of the engineered glucocerebrosidase optionally includes 1, 2, 3, 4, 5, 6, 7, 8, 9, or up to 10 substitutions. In some embodiments, the amino acid sequence of the engineered glucocerebrosidase optionally includes 1, 2, 3, 4, or 5 substitutions, insertions, and/or deletions. In some embodiments, the amino acid sequence of the engineered glucocerebrosidase optionally includes 1, 2, 3, 4, or 5 substitutions. In some embodiments, the amino acid substitutions comprises non-conservative and/or conservative substitutions. In some embodiments, the substitutions comprise conservative substitutions.

In some embodiments, the engineered glucocerebrosidase comprises an amino acid sequence comprising residues 40-536 of SEQ ID NO: 54, 220, 984, or 1150, or comprising SEQ ID NO: 54, 220, 984, or 1150. In some embodiments, the amino acid sequence of the engineered glucocerebrosidase optionally includes 1, 2, 3, 4, 5, 6, 7, 8, 9, or up to 10 substitutions, insertions, and/or deletions. In some embodiments, the amino acid sequence of the engineered glucocerebrosidase optionally includes 1, 2, 3, 4, 5, 6, 7, 8, 9, or up to 10 substitutions. In some embodiments, the amino acid sequence of the engineered glucocerebrosidase optionally includes 1, 2, 3, 4, or 5 substitutions, insertions, and/or deletions. In some embodiments, the amino acid sequence of the engineered glucocerebrosidase optionally includes 1, 2, 3, 4, or 5 substitutions. In some embodiments, the amino acid substitutions comprises non-conservative and/or conservative substitutions. In some embodiments, the substitutions comprise conservative substitutions.

In some embodiments, the engineered glucocerebrosidase described herein has glycoside hydrolase activity. In some embodiments, the engineered glucocerebrosidase has glucosylceramidase activity capable of cleaving the β-glycosyl linkage of glucocerebroside. In some embodiments, the engineered glucocerebrosidase has glucocerebrosidase activity and has one or more improved properties compared to a reference glucocerebrosidase. In some embodiments, exemplary improved properties are provided in the Examples.

In some embodiments, the engineered glucocerebrosidase has increased enzymatic activity compared to a reference glucocerebrosidase having a sequence corresponding to residues 40-536 of SEQ ID NO: 2, 54, 220, 984, or 1150, or a sequence corresponding to SEQ ID NO: 2, 54, 220, 984, or 1150. In some embodiments, the engineered glucocerebrosidase has at least 1.1-fold, 1.2 fold, 1.5 fold, 2-fold, 2.5-fold, 3-fold, 4-fold, 5-fold, 10-fold, 15-fold, 20-fold or more activity as compared to the reference glucocerebrosidase. In some embodiments, the increased enzymatic activity is based on the assay described in the Examples.

In some embodiments, the engineered glucocerebrosidase has increased expression or expression efficiency compared to a reference glucocerebrosidase having a sequence corresponding to residues 40-536 of SEQ ID NO: 2, 54, 220, 984, or 1150, or a sequence corresponding to SEQ ID NO: 2, 54, 220, 984, or 1150. In some embodiments, the increased expression or expression efficiency is in the liver or liver cells, or macrophages.

In some embodiments, the engineered glucocerebrosidase has increased stability at neutral or basic pH compared to the reference glucocerebrosidase having a sequence corresponding to residues 40-536 of SEQ ID NO: 2, 54, 220, 984, or 1150, or a sequence corresponding to SEQ ID NO: 2, 54, 220, 984, or 1150.

In some embodiments, the engineered glucocerebrosidase has increased stability at acidic pH compared to the reference glucocerebrosidase having a sequence corresponding to residues 40-536 of SEQ ID NO: 2, 54, 220, 984, or 1150, or a sequence corresponding to SEQ ID NO: 2, 54, 220, 984, or 1150.

In some embodiments, the engineered glucocerebrosidase has increased stability to serum and/or plasma exposure compared to the reference glucocerebrosidase having a sequence corresponding to residues 40-536 of SEQ ID NO: 2, 54, 220, 984, or 1150, or a sequence corresponding to SEQ ID NO: 2, 54, 220, 984, or 1150.

In some embodiments, the engineered glucocerebrosidase has increased thermostability compared to the reference glucocerebrosidase having a sequence corresponding to residues 40-536 of SEQ ID NO: 2, 54, 220, 984, or 1150, or a sequence corresponding to SEQ ID NO: 2, 54, 220, 984, or 1150. In some embodiments, the increased stability is at 44° C.-47° C., or 30° C.-55° C., for a defined time period, e.g., 1 hr.

In some embodiments, the engineered glucocerebrosidase is characterized by increased expression and/or secretion from cells, such as macrophages, as compared to a reference glucocerebrosidase having a sequence corresponding to residues 40-536 of SEQ ID NO: 2, 54, 220, 984, or 1150, or a sequence corresponding to SEQ ID NO: 2, 54, 220, 984, or 1150.

In some embodiments, the engineered glucocerebrosidase is characterized by increased uptake and/or intracellular stability in cells such as macrophages, as compared to a reference glucocerebrosidase having a sequence corresponding to residues 40-536 of SEQ ID NO: 2, 54, 220, 984, or 1150, or a sequence corresponding to SEQ ID NO: 2, 54, 220, 984, or 1150.

In some embodiments, the engineered glucocerebrosidase has reduced immunogenicity compared to a reference glucocerebrosidase having a sequence corresponding to residues 40-536 of SEQ ID NO: 2, 54, 220, or 984, or a sequence corresponding to SEQ ID NO: 2, 54, 220, or 984.

In some embodiments of the foregoing, the improved property is in comparison to a reference glucocerebrosidase having the sequence corresponding to residues 40-536 of SEQ ID NO: 2, or the sequence corresponding to SEQ ID NO: 2.

In some embodiments, the glucocerebrosidase of the present disclosure exhibits at least one improved property selected from: i) increased enzymatic activity; ii) increased expression or expression efficiency; iii) increased stability at neutral or basic pH; iv) increased stability at acidic pH, v) increased stability to serum or plasma; vi) increased thermostability; vii) increased uptake and/or intracellular stability in cells such as macrophages and viii) reduced immunogenicity; or any combination of i), ii), iii), iv), v), vi), vii and viii), as compared to a reference glucocerebrosidase having a sequence corresponding to residues 40-536 of SEQ ID NO: 2, 54, 220, 984, or 1150, or a sequence corresponding to SEQ ID NO: 2, 54, 220, 984, or 1150. In some embodiments, the reference glucocerebrosidase has the sequence corresponding to residues 40-536 of SEQ ID NO: 2, or the sequence corresponding to SEQ ID NO: 2.

In some embodiments, the engineered glucocerebrosidase is a “deimmunized” glucocerebrosidase (e.g., as presented in the Examples in Table 14.1). In some embodiments, the engineered glucocerebrosidase is characterized by a reduction of Total Immunogenicity Score (TIS) and/or Immunogenic Hit Count (IHC) as compared to a reference glucocerebrosidase, wherein the reference glucocerebrosidase has a sequence corresponding to SEQ ID NO: 2, 54, 220, or 984, such as provided in Table 14.1 of the Examples.

In some embodiments, a “deimmunized” engineered glucocerebrosidase comprises
an amino acid sequence comprising residues 40-536 of SEQ ID NO: 4, 12, 22, 30, 32, 38, 44, 46, 50, 54, 56, 58, 60, 64, 72, 76, 80, 82, 84, 86, 88, 90, 92, 96, 98, 200, 208, 212, 216, 218, 222, 226, 228, 232, 236, 242, 244, 254, 256, 260, 262, 266, 272, 274, 276, 278, 280, 282, 286, 292, 298, 300, 302, 304, 306, 308, 312, 314, 316, 318, 320, 322, 324, 326, 330, 334, 336, 338, 340, 342, 346, 348, 350, 352, 354, 356, 358, 360, 362, 364, 366, 368, 370, 372, 374, 376, 382, 384, 386, 388, 390, 392, 394, 396, 398, 400, 402, 404, 406, 408, 410, 412, 414, 416, 418, 422, 424, 426, 428, 430, 432, 434, 436, 438, 444, 446, 448, 450, 452, 454, 456, 458, 460, 462, 464, 466, 468, 470, 472, 476, 478, 480, 482, 484, 490, 492, 496, 500, 502, 508, 510, 512, 514, 516, 518, 524, 526, 528, 532, 534, 536, 538, 540, 542, 544, 546, 552, 554, 558, 560, 562, 564, 566, 568, 570, 572, 574, 576, 578, 580, 582, 584, 588, 590, 592, 594, 596, 600, 602, 604, 606, 608, 610, 612, 618, 620, 626, 628, 630, 632, 634, 636, 638, 640, 644, 646, 648, 652, 654, 656, 660, 662, 666, 668, 670, 672, 674, 676, 678, 680, 682, 684, 686, 688, 690, 692, 694, 696, 698, 700, 706, 708, 710, 714, 716, 718, 720, 722, 724, 726, 728, 730, 732, 734, 736, 740, 742, 746, 748, 750, 752, 756, 760, 762, 766, 768, 770, 772, 774, 776, 778, 780, 782, 784, 786, 788, 790, 794, 796, 798, 800, 802, 804, 806, 808, 810, 814, 816, 818, 822, 828, 830, 834, 836, 842, 844, 846, 850, 852, 854, 856, 858, 860, 862, 864, 866, 868, 870, 874, 876, 878, 880, 882, 884, 886, 888, 890, 892, 894, 896, 898, 900, 902, 904, 906, 910, 914, 916, 918, 920, 924, 928, 932, 936, 940, 946, 948, 952, 956, 958, 960, 964, 968, 970, 980, 984, 988, 990, 994, 998, 1002, 1004, 1006, 1008, 1012, 1016, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, or 1216, or
an amino acid sequence comprising SEQ ID NO: 4, 12, 22, 30, 32, 38, 44, 46, 50, 54, 56, 58, 60, 64, 72, 76, 80, 82, 84, 86, 88, 90, 92, 96, 98, 200, 208, 212, 216, 218, 222, 226, 228, 232, 236, 242, 244, 254, 256, 260, 262, 266, 272, 274, 276, 278, 280, 282, 286, 292, 298, 300, 302, 304, 306, 308, 312, 314, 316, 318, 320, 322, 324, 326, 330, 334, 336, 338, 340, 342, 346, 348, 350, 352, 354, 356, 358, 360, 362, 364, 366, 368, 370, 372, 374, 376, 382, 384, 386, 388, 390, 392, 394, 396, 398, 400, 402, 404, 406, 408, 410, 412, 414, 416, 418, 422, 424, 426, 428, 430, 432, 434, 436, 438, 444, 446, 448, 450, 452, 454, 456, 458, 460, 462, 464, 466, 468, 470, 472, 476, 478, 480, 482, 484, 490, 492, 496, 500, 502, 508, 510, 512, 514, 516, 518, 524, 526, 528, 532, 534, 536, 538, 540, 542, 544, 546, 552, 554, 558, 560, 562, 564, 566, 568, 570, 572, 574, 576, 578, 580, 582, 584, 588, 590, 592, 594, 596, 600, 602, 604, 606, 608, 610, 612, 618, 620, 626, 628, 630, 632, 634, 636, 638, 640, 644, 646, 648, 652, 654, 656, 660, 662, 666, 668, 670, 672, 674, 676, 678, 680, 682, 684, 686, 688, 690, 692, 694, 696, 698, 700, 706, 708, 710, 714, 716, 718, 720, 722, 724, 726, 728, 730, 732, 734, 736, 740, 742, 746, 748, 750, 752, 756, 760, 762, 766, 768, 770, 772, 774, 776, 778, 780, 782, 784, 786, 788, 790, 794, 796, 798, 800, 802, 804, 806, 808, 810, 814, 816, 818, 822, 828, 830, 834, 836, 842, 844, 846, 850, 852, 854, 856, 858, 860, 862, 864, 866, 868, 870, 874, 876, 878, 880, 882, 884, 886, 888, 890, 892, 894, 896, 898, 900, 902, 904, 906, 910, 914, 916, 918, 920, 924, 928, 932, 936, 940, 946, 948, 952, 956, 958, 960, 964, 968, 970, 980, 984, 988, 990, 994, 998, 1002, 1004, 1006, 1008, 1012, 1016, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, or 1216.

In some embodiments, the engineered glucocerebrosidase comprises a full length engineered glucocerebrosidase of any of the engineered glucocerebrosidase described herein. In some embodiments, the glucocerebrosidase comprises a “pre-pro-protein” or “pre-pro-polypeptide” of any of the engineered glucocerebrosidase described herein. In some embodiments, the signal sequence of the full length glucocerebrosidase or “pre-pro-protein” or “pre-pro-polypeptide” thereof can comprise any compatible signal sequence or signal peptide, including synthetic, mouse (e.g., IgG), or human signal sequence or signal peptide. In some embodiments, the signal sequence in the exemplary glucocerebrosidases herein is replaced with a compatible signal sequence or signal peptide, e.g., synthetic or mouse. In some embodiments, the signal sequence comprises the signal sequence in the naturally occurring full length human glucocerebrosidase.

In some embodiments, the engineered glucocerebrosidase is processed or cleaved to a pro-polypeptide or pro-protein form, for example, lacking the signal sequence. In some embodiments, the engineered glucocerebrosidase is processed or cleaved to a mature form of the engineered glucocerebrosidase, such as described herein. In some embodiments, the mature form of the engineered glucocerebrosidase comprises the active form of the engineered glucocerebrosidase.

Thus, for each and every engineered glucocerebrosidase described herein, the present disclosure provides a pre-pro-polypeptide, pro-polypeptide, or mature, biologically active form of the engineered glucocerebrosidase, including each engineered glucocerebrosidases disclosed in Tables 3.1, 3.2, 4.1, 4.2, 4.3, 4.4, 5.1, 6.1, 6.2, 12.1, 12.2, and 13.1. In some embodiments, the pro-polypeptide of the engineered glucocerebrosidase comprises an amino acid sequence from amino acid residue 21 or an amino acid sequence from amino acid residue 40 to the carboxy end of the engineered glucocerebrosidase polypeptide, e.g., amino acid residue 536.

In some embodiments, the engineered glucocerebrosidase is expressed as a fusion protein. In some embodiments, the engineered glucocerebrosidase described herein can be fused to a variety of polypeptide sequences, such as, by way of example and not limitation, polypeptide tags that can be used for detection and/or purification. In some embodiments, the fusion protein of the engineered glucocerebrosidase comprises a glycine-histidine or histidine-tag (His-tag). In some embodiments, the fusion protein of the engineered glucocerebrosidase comprises an epitope tag, such as c-myc, FLAG, V5, or hemagglutinin (HA). In some embodiments, the fusion protein of the engineered glucocerebrosidase comprises a GST, SUMO, Strep, MBP, or GFP tag. In some embodiments, the fusion is to the amino (N—) terminus of engineered glucocerebrosidase polypeptide. In some embodiments, the fusion is to the carboxy (C—) terminus of the engineered glucocerebrosidase polypeptide.

In some embodiments, for any of the engineered glucocerebrosidase disclosed herein, the engineered glucocerebrosidase is purified or is a purified preparation or composition. In some embodiments, the engineered glucocerebrosidase is provided in solution, as a lyophilizate, or immobilized on a solid substrate, porous substrate, membrane, filter, or particles.

In some embodiments, the present disclosure further provides a functional or biologically active fragment of an engineered glucocerebrosidase described herein. In some embodiments, functional or biologically active fragments have at least 80%, 81%, 82%, 83%, 84%, 85%, 86%, 87%, 88%, 89%, 90%, 91%, 92%, 93%, 94%, 95%, 96%, 97%, 98%, or 99% of the activity of the glucocerebrosidase from which it was derived (i.e., the parent engineered glucocerebrosidase). In some embodiments, a functional or biologically active fragment comprises at least 80%, 81%, 82%, 83%, 84%, 85%, 86%, 87%, 88%, 89%, 90%, 91%, 92%, 93%, 94%, 95%, 96%, 97%, 98%, or 99% of the parent sequence of the glucocerebrosidase. In some embodiments the functional fragment is truncated by less than 5, less than 10, less than 15, less than 10, less than 25, less than 30, less than 35, less than 40, less than 45, and less than 50 amino acids.

In some embodiments, the functional or biologically active fragment of the engineered glucocerebrosidase described herein includes at least a mutation or mutation set in the amino acid sequence of the engineered glucocerebrosidase described herein. Accordingly, in some embodiments, the functional or biologically active fragments of the engineered glucocerebrosidase displays the enhanced or improved property associated with the mutation or mutation set in the parent engineered glucocerebrosidase.

In some further embodiments, as discussed herein, a functional fragment or a biologically active fragment encompasses biologically processed polypeptides of an engineered glucocerebrosidase, such as functional or biologically active fragments produced upon expression in a mammalian cell or in a patient, or when an engineered glucocerebrosidase in administered to a patient, particularly a human patient. In some embodiments, the functional or biologically active fragment is derived from an engineered glucocerebrosidase presented in Tables 3.1, 3.2, 4.1, 4.2, 4.3, 4.4, 5.1, 6.1, 6.2, 12.1, 12.2, and 13.1, and in the Sequence Listing.

### Polynucleotides Encoding Engineered Polypeptides, Expression Vectors and Host Cells

In another aspect, the present disclosure provides polynucleotides encoding the engineered glucocerebrosidase described herein. In some embodiments, the polynucleotides are operatively linked to one or more heterologous or homologous regulatory sequences that control gene expression to create a recombinant polynucleotide capable of expressing the engineered glucocerebrosidase. Expression constructs containing a heterologous polynucleotide encoding the engineered glucocerebrosidase can be introduced into appropriate host cells to express the corresponding engineered glucocerebrosidase polypeptide.

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode the engineered glucocerebrosidase. Thus, having knowledge of a particular amino acid sequence, those skilled in the art could make any number of different nucleic acids by simply modifying the sequence of one or more codons in a way which does not change the amino acid sequence of the protein. In this regard, the present invention specifically contemplates each and every possible variation of polynucleotides that could be made encoding the engineered glucocerebrosidase described herein by selecting combinations based on the possible codon choices, and all such variations are to be considered specifically disclosed for any polypeptide described herein, including the engineered glucocerebrosidase variants provided in Tables 3.1, 3.2, 4.1, 4.2, 4.3, 4.4, 5.1, 6.1, 6.2, 12.1, 12.2, and 13.1, and the Sequence Listing.

As discussed above, it is to be understood that the present disclosure provides recombinant polynucleotides encoding each and every engineered glucocerebrosidase described herein, or a biologically or functionally active fragment, or a pre-pro-polypeptide, pro-polypeptide, or mature polypeptide thereof.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 40-536 of an even-numbered SEQ ID NO. of SEQ ID NOs: 2-1648, or to a reference sequence corresponding to an even-numbered SEQ ID NO. of SEQ ID NOs: 2-1648, wherein the amino acid sequence comprises one or more substitutions relative to the reference sequence corresponding to residues 40-536 of SEQ ID NO: 2, 54, 220, or 1150, or relative to the reference sequence corresponding to SEQ ID NO: 2, 54, 220, or 1150.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 40-536 of SEQ ID NO: 2, 54, 220, 984, or 1150, or to a reference sequence corresponding to SEQ ID NO: 2, 54, 220, or 1150, wherein the amino acid sequence comprises one or more substitutions relative to the reference sequence corresponding to residues 40-536 of SEQ ID NO: 2, 54, 220, or 1150, or relative to the reference sequence corresponding to SEQ ID NO: 2, 54, 220, or 1150.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 2, or to the reference sequence corresponding to SEQ ID NO: 2, wherein the amino acid sequence comprises one or more substitutions relative to the reference sequence corresponding to residues 40-536 of SEQ ID NO: 2, or relative to the reference sequence corresponding to SEQ ID NO: 2.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, 220, 984, or 1150, or to the reference sequence corresponding to SEQ ID NO: 54, 220, 984, or 1150, wherein the amino acid sequence comprises one or more substitutions relative to the reference sequence corresponding to residues 40-536 of SEQ ID NO: 2, or relative to the reference sequence corresponding to SEQ ID NO: 2.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 4-1648, or to the reference sequence corresponding to an even-numbered SEQ ID NO. of SEQ ID NOs: 4-1648, wherein the amino acid sequence comprises one or more substitutions relative to the reference sequence corresponding to residues 40-536 of SEQ ID NO: 2, or relative to the reference sequence corresponding to SEQ ID NO: 2.

In some embodiments, the recombinant polynucleotide comprises a polynucleotide sequence encoding an engineered glucocerebrosidase comprising an amino acid sequence comprising at least a substitution at amino acid position 23, 29, 35, 40, 41, 42, 44, 46, 50, 51, 53, 59, 63, 65, 68, 69, 70, 71, 75, 77, 78, 79, 82, 84, 86, 88, 89, 90, 91, 92, 93, 94, 95, 96, 97, 98, 99, 100, 101, 102, 103, 104, 105, 106, 107, 108, 109, 110, 111, 113, 114, 115, 116, 117, 133, 134, 137, 138, 141, 142, 144, 150, 151, 154, 169, 171, 172, 175, 180, 182, 184, 186, 189, 190, 191, 194, 195, 201, 202, 204, 206, 207, 211, 212, 214, 220, 222, 224, 230, 233, 235, 236, 239, 243, 246, 258, 260, 261, 262, 263, 264, 265, 271, 276, 298, 299, 301, 314, 315, 316, 319, 325, 329, 332, 333, 335, 336, 337, 338, 339, 341, 342, 359, 360, 361, 368, 370, 372, 373, 374, 382, 385, 386, 390, 400, 404, 408, 412, 413, 415, 434, 445, 446, 449, 459, 467, 468, 469, 471, 473, 474, 476, 477, 478, 479, 480, 481, 484, 485, 487, 489, 490, 493, 494, 495, 497, 502, 504, 505, 506, 507, 508, 509, 510, 512, 514, 515, 516, 518, 519, 520, 522, 524, 525, 529, 532, 533, 534, or 536, or combinations thereof, wherein the amino acid positions are relative to the reference sequence corresponding to residues 40-536 of SEQ ID NO: 2, or relative to the reference sequence corresponding to SEQ ID NO: 2.

In some embodiments, the recombinant polynucleotide comprises a polynucleotide sequence encoding an engineered glucocerebrosidase comprising an amino acid sequence comprising at least a substitution at amino acid position 65, 68, 70, 99, 154, 195, 207, 214, 230, 263, 319, 337, 361, 374, 408, 471, or 489, or combinations thereof, wherein the amino acid positions are relative to the reference sequence corresponding to residues 40-536 of SEQ ID NO: 2, or relative to the reference sequence corresponding to SEQ ID NO: 2.

In some embodiments, the recombinant polynucleotide comprises a polynucleotide sequence encoding an engineered glucocerebrosidase comprising an amino acid sequence comprising at least a substitution at amino acid position 70, 207, 214, or 489, or combinations thereof, wherein the amino acid positions are relative to the reference sequence corresponding to residues 40-536 of SEQ ID NO: 2, or relative to the reference sequence corresponding to SEQ ID NO: 2.

In some embodiments, the recombinant polynucleotide comprises a polynucleotide sequence encoding an engineered glucocerebrosidase comprising an amino acid sequence comprising at least a substitution at amino acid position 65, 68, 99, 154, 230, or 319, or combinations thereof, wherein the amino acid positions are relative to the reference sequence corresponding to residues 40-536 of SEQ ID NO: 2, or relative to the reference sequence corresponding to SEQ ID NO: 2.

In some embodiments, the recombinant polynucleotide comprises a polynucleotide sequence encoding an engineered glucocerebrosidase comprising an amino acid sequence comprising at least a substitution at amino acid position 65, 195, 230, 263, 337, 361, 374, or 471, or combinations thereof, wherein the amino acid positions are relative to the reference sequence corresponding to residues 40-536 of SEQ ID NO: 2, or relative to the reference sequence corresponding to SEQ ID NO: 2.

In some embodiments, the recombinant polynucleotide comprises a polynucleotide sequence encoding an engineered glucocerebrosidase comprising an amino acid sequence comprising at least a substitution set at amino acid position(s) 265/372/489, 243, 23, 109, 265, 109/195/243, 23/70/207/214/265, 23/70, 23/214/243/265, 65/70/109/214/319/372/481, 23/265/319, 23/65/70/141/319/481, 23/265, 372, 214, 23/65/70/109/141/214, 319, 65/372, 109/195, 23/65, 207/265/319, 109/207/214/372, 23/319/372/489, 65/109/214/243, 23/141/195, 70/207/214/489, 65/70/265/319/372, 214/265, 265/319, 65/243, 65/70/195, 489, 23/65/141, 23/372, 70/141, 23/195, 214/319, 23/207/214/243/265/481, 65/214/243/265/319, 214/243, 243/265, 265/489, 214/243/265, 207/243/372, 195/243, 65/481, 207/214/243/319, 70/141/195/265, 23/265/372, 46/99/134/230/373/449, 68/211/230, 373/449, 46/68/336, 68/230/314/373, 68, 68/336/373, 99/314, 46, 46/230/261, 373, 230/314, 68/211, 68/336/449, 46/336, 336/449, 46/68/336/373, 46/99, 99/261, 99, 68/134, 46/68/134/230/336/373, 46/68, 68/99/211/230, 99/134, 68/99/336, 449, 68/99, 46/230/336, 68/154/449, 336/373/449, 134/336, 68/99/230, 46/68/134/154, 68/99/230/373/449, 134, 46/68/154/211/230/261/336, 68/99/154/261/336, 230/449, 46/68/314, 154, 68/449, 99/449, 68/154/230/314/336/449, 46/68/154/314/336/373, 46/230, 336, or 46/99/230/373, wherein the amino acid positions are relative to the reference sequence corresponding to residues 40-536 of SEQ ID NO: 2, or relative to the reference sequence corresponding to SEQ ID NO: 2.

In some embodiments, the recombinant polynucleotide comprises a polynucleotide sequence encoding an engineered glucocerebrosidase comprising an amino acid sequence comprising at least a substitution set at amino acid position(s) 65/68/70/99/154/195/207/214/230/263/319/337/361/374/408/471/489, 65/68/70/99/154/195/207/214/230/263/319/337/361/374/408, 65/68/154/195/214/230/319/337/374/408, 65/68/70/99/154/195/207/214/230/263/374/408, 65/68/70/154/195/214/230/319/374/408/489, 65/68/70/99/154/195/207/214/230/319/337/361/374/408/471/489, 65/68/70/99/154/195/207/214/230/263/319/337/374/408, 65/68/154/195/207/214/230/263/319/337/361/374/408/471/489, 65/68/70/99/154/195/207/214/230/263/319/337/361/374/408/489, 65/68/70/99/154/195/207/214/230/374/408/471, 68/70/99/154/195/207/214/230/263/319/408, 65/68/70/207/230/374/408, 65/68/70/214/230/263/337/374/408, 214/230/263/374/408, 65/68/70/99/154/195/207/230/408, 65/68/70/99/154/207/230/319/408, 65/68/70/214/230/408, 65/68/70/195/214/230/263/361/471, 195/207/230/319/374/408/489, 65/68/70/214/230/408/489, 65/68/70/195/207/230/408, 207/230/319/374/408, 65/68/70/99/195/207/230/408, 195/214/230/263/408, 195/207/230/374/408, 65/68/70/99/154/195/214/408, 207/214/230/319/374/408, 207/214/230/374/408, 154/207/230/374/408/489, 207/230/263/408, 207/230/408/471, 207/230/374/408, 214/230/319/408, 214/230/374/408/489, 207/230/408, 65/68/70/154/207/408, 207/230/263/337/408, 195/214/230/408, 207/230/408/489, 214/230/408, 230/361/408, 207/214/408, 214/263/408, 230/374/408, 207/408, 195/214/263, 374/408, or 408, wherein the amino acid positions are relative to the reference sequence corresponding to residues 40-536 of SEQ ID NO: 2, or relative to the reference sequence corresponding to SEQ ID NO: 2.

In some embodiments, the recombinant polynucleotide comprises a polynucleotide sequence encoding an engineered glucocerebrosidase comprising an amino acid sequence comprising at least a substitution at an amino acid position set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, or relative to the reference sequence corresponding to SEQ ID NO: 2.

In some embodiments, the recombinant polynucleotide comprises a polynucleotide sequence encoding an engineered glucocerebrosidase comprising at least one substitution set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, or relative to the reference sequence corresponding to SEQ ID NO: 2.

In some embodiments, the recombinant polynucleotide comprises a polynucleotide sequence encoding an engineered glucocerebrosidase comprising an amino acid sequence comprising at least a substitution or substitution set at amino acid position(s) set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, or relative to the reference sequence corresponding to SEQ ID NO: 2.

In some embodiments, the recombinant polynucleotide comprises a polynucleotide sequence encoding an engineered glucocerebrosidase comprising an amino acid sequence comprising at least a substitution or substitution set of an engineered glucocerebrosidase variant set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, or relative to the reference sequence corresponding to SEQ ID NO: 2.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, 220, 984, or 1150, or to the reference sequence corresponding to SEQ ID NO: 54, 220, 984, or 1150.

In some embodiments, the recombinant polynucleotide comprises a polynucleotide sequence encoding the engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 4-1648, or to the reference sequence corresponding to an even-numbered SEQ ID NO. of SEQ ID NOs: 4-1648.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, 220, 984, or 1150, or to the reference sequence corresponding to SEQ ID NO: 54, 220, 984, or 1150, wherein the amino acid sequence comprises one or more substitutions relative to the reference sequence corresponding to residues 40-536 of SEQ ID NO: 54, 220, 984, or 1150, or relative to the reference sequence corresponding to SEQ ID NO: 54, 220, 984, or 1150.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 4-1648, or to the reference sequence corresponding to an even-numbered SEQ ID NO. of SEQ ID NOs: 4-1648, wherein the amino acid sequence comprises one or more substitutions relative to the reference sequence corresponding to residues 40-536 of SEQ ID NO: 54, 220, 984, or 1150, or relative to the reference sequence corresponding to SEQ ID NO: 54, 220, 984, or 1150.

In some embodiments, the recombinant polynucleotide comprises a polynucleotide sequence encoding the engineered glucocerebrosidase comprising an amino acid sequence comprising at least a substitution at amino acid position 23, 29, 35, 40, 41, 42, 44, 46, 50, 51, 53, 59, 63, 65, 68, 69, 70, 71, 75, 77, 78, 79, 82, 84, 86, 88, 89, 90, 91, 92, 93, 94, 95, 96, 97, 98, 99, 100, 101, 102, 103, 104, 105, 106, 107, 108, 109, 110, 111, 113, 114, 115, 116, 117, 133, 134, 137, 138, 141, 142, 144, 150, 151, 154, 169, 171, 172, 175, 180, 182, 184, 186, 189, 190, 191, 194, 195, 201, 202, 204, 206, 207, 211, 212, 214, 220, 222, 224, 230, 233, 235, 236, 239, 243, 246, 258, 260, 261, 262, 263, 264, 265, 271, 276, 298, 299, 301, 314, 315, 316, 319, 325, 329, 332, 333, 335, 336, 337, 338, 339, 341, 342, 359, 360, 361, 368, 370, 372, 373, 374, 382, 385, 386, 390, 400, 404, 408, 412, 413, 415, 434, 445, 446, 449, 459, 467, 468, 469, 471, 473, 474, 476, 477, 478, 479, 480, 481, 484, 485, 487, 489, 490, 493, 494, 495, 497, 502, 504, 505, 506, 507, 508, 509, 510, 512, 514, 515, 516, 518, 519, 520, 522, 524, 525, 529, 532, 533, 534, or 536, or combinations thereof, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, or to the reference sequence corresponding to SEQ ID NO: 54, wherein the amino acid sequence comprises one or more substitutions relative to the reference sequence corresponding to residues 40-536 of SEQ ID NO: 54, or relative to the reference sequence corresponding to SEQ ID NO: 54.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 200-922, or to the reference sequence corresponding to SEQ ID NO: 200-922, wherein the amino acid sequence comprises one or more substitutions relative to the reference sequence corresponding to residues 40-536 of SEQ ID NO: 54, or relative to the reference sequence corresponding to SEQ ID NO: 54.

In some embodiments, the recombinant polynucleotide comprises a polynucleotide sequence encoding the engineered glucocerebrosidase comprising an amino acid sequence comprising at least a substitution or substitution set at amino acid position(s) 68/336, 65/99/319, 65/68/319, 68/319, 68, 65/319, 195, 65/154/319, 65/68/230, 65, 65/68/99/154/230/319, 68/230, 195/230, 65/230, 141/319, 154/336, 68/154, 68/195/230/319/336, 68/99/195/230/319, 68/195, 65/68, 68/141/230/319, 65/68/99/230/319, 68/99/141/319, 68/154/319, 141/154, 68/99, 99/230/319, 99, 230, 68/230/319, 154/319, 65/68/154/319, 141, 336, 141/230, 68/154/336, 65/68/99/319, 154, 319, 230/319, 68/154, 99/141/319, 509, 108, 108/117/509, 519, 108/519, 88, 117/413/509, 509/519, 413, 108/117/315, 117/271, 117/413/509/529, 413/509, 469, 524, 507, 525, 489, 104, 95, 359, 374,107, 89, 260, 110, 186, 370,191, 333, 189, 468, 473, 115, 53, 449, 518, 236, 481, 59/220, 258, 467, 51, 415, 103, 493, 78, 335, 520, 101, 341, 471, 211, 206, 93, 490, 71, 79, 180, 172, 207, 63, 142, 529, 77, 212, 474, 106, 386/507, 117, 373, 337, 107/246, 516, 233, 91, 194, 84, 361, 171, 536, 50, 116, 239, 44, 40, 113, 243, 169, 504, 505, 109, 479, 477, 97, 182, 111,508, 314, 102, 338, 96, 138, 42, 263, 222, 522, 265, 514, 515, 246, 46, 133, 332, 506, 512, 137, 190,105, 301, 329, 339, or 151, wherein the amino acid positions are relative to the reference sequence corresponding to residues 40-536 of SEQ ID NO: 54, or relative to the reference sequence corresponding to SEQ ID NO: 54.

In some embodiments, the recombinant polynucleotide comprises a polynucleotide sequence encoding the engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 220, or to the reference sequence corresponding to SEQ ID NO: 220, wherein the amino acid sequence comprises one or more substitutions relative to the reference sequence corresponding to residues 40-536 of SEQ ID NO: 220, or relative to the reference sequence corresponding to SEQ ID NO: 220.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 924-1020, or to the reference sequence corresponding to SEQ ID NO: 924-1020, wherein the amino acid sequence comprises one or more substitutions relative to the reference sequence corresponding to residues 40-536 of SEQ ID NO: 220, or relative to the reference sequence corresponding to SEQ ID NO: 220.

In some embodiments, the recombinant polynucleotide comprises a polynucleotide sequence encoding an engineered glucocerebrosidase comprising an amino acid sequence comprising at least a substitution or substitution set at amino acid position(s) 65/195/361, 514, 79/195/263/361, 65/374, 79/195, 65/230, 65/233/263/337/359/361/374, 79/536, 79/230/233/239/359/361/471, 65/471, 374, 65/195/230/233/337/374/514, 65/195/233/359/536, 230, 65/195/263/276/359/361/374/471, 79, 195/230/233/337/374/536, 195/230, 195/230/337/359/361, 230/263, 65/230/233, 263, 195/230/337/361/374/471, 65/195, 374/471, 374/536, 239, 65, 230/233/536, 230/263/374, 65/195/230/263/337/361/374/471, 79/374, 65/361/374/471/536, 195, 233, 79/230/359/374, 536, 230/359/361/374/514, 65/79/230, 195/239/263/361/374/514, 195/263/374, 195/374, 65/230/233/361/536, 230/233/374/471, 65/233/359/361/471, 65/359/471, 65/79, or 65/359, wherein the amino acid positions are relative to the reference sequence corresponding to residues 40-536 of SEQ ID NO: 220, or relative to the reference sequence corresponding to SEQ ID NO: 220.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 984, or to the reference sequence corresponding to SEQ ID NO: 984, wherein the amino acid sequence comprises one or more substitutions relative to the reference sequence corresponding to residues 40-536 of SEQ ID NO: 984, or relative to the reference sequence corresponding to SEQ ID NO: 984.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 1022-1216, or to the reference sequence corresponding to SEQ ID NO: 1022-1216, wherein the amino acid sequence comprises one or more substitutions relative to the reference sequence corresponding to residues 40-536 of SEQ ID NO: 984, or relative to the reference sequence corresponding to SEQ ID NO: 984.

In some embodiments, the recombinant polynucleotide comprises a polynucleotide sequence encoding an engineered glucocerebrosidase comprising an amino acid sequence comprising at least a substitution or substitution set at amino acid position(s) 359, 79/359, 79/97/182, 97/359, 97, 79/182, 201, 404, 484, 446, 494/495, 495, 412, 385, 100, 184, 82, 75, 71, 41, 99, 94, 92, 98, 114, 95, 102, 96, 105, 113, 325, 336, 262, 386, 360, 400, 342, 390, 264, 298, 382, 408, 449, 459, 434, 476,502, 29/534, 497, 485, 533, 536, 510, 532, 489, 534, 494, 182, 137, 204, 194, 202, 144, 142, 175, 220, 150, or 206, wherein the amino acid positions are relative to the reference sequence corresponding to residues 40-536 of SEQ ID NO: 984, or relative to the reference sequence corresponding to SEQ ID NO: 984.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 1150, or to the reference sequence corresponding to SEQ ID NO: 1150, wherein the amino acid sequence comprises one or more substitutions relative to the reference sequence corresponding to residues 40-536 of SEQ ID NO: 1150, or relative to the reference sequence corresponding to SEQ ID NO: 1150.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 1218-1554, or to the reference sequence corresponding to SEQ ID NO: 1218-1554, wherein the amino acid sequence comprises one or more substitutions relative to the reference sequence corresponding to residues 40-536 of SEQ ID NO: 1150, or relative to the reference sequence corresponding to SEQ ID NO: 1150.

In some embodiments, the recombinant polynucleotide comprises a polynucleotide sequence encoding an engineered glucocerebrosidase comprising an amino acid sequence comprising at least a substitution or substitution set at amino acid position(s) 96/194/382/484, 359/382/412/494/495, 92/96/175/182/382, 96/359/382/484/494/495, 92/175/194/359/382, 359/382/400/412, 92/96/182/382/400/484, 92/96/382/494/495, 92/96/175/382/494/495, 96/182/359/382/484, 92/96/382, 92/382/494/495/534, 92/96/382/400/484, 92/96/175/382, 92/175/382/400/484, 92/96/175/182/382/412/484, 92/96/201/382/484, 92/182/194/382, 92/96/175/194/359/382/400, 92/359/382, 96/382, 92/175/382/484/494/495, 96/201/359/382, 92, 194, 201, 359, 96, 92/96/382/400/484/495, 382/495, 382, 92/382, 71/175/325/385, 325/385, 71/325/502, 71/175/325/385/502, 202/325/385, 97/175/385/510, 325/385/510, 100/175/325, 71/175/325/360/385, 97/175/510, 71/97/100/175/325/385, 71/175/510, 71/97/325/502, 71/100/325/385, 202/385/489, 325/385/502, 71/100/325/385/510, 71/100/175/325/385, 385/510, 97/325/385, 71, 97/202/325/360/385/510, 502, 71/97/103/325, 100/325/385/502/510, 71/137/175/325/360/385/502, 71/276/325/385/510, 71/175/360/385/502, 100/175/325/385/489/502, 71/175/360/385/485/502, 95/113, 494/536, 95/99/113/182/184/264/386, 184/536, 95/142/144/182/184/386/449, 144/204/386/400/449, 113/449/536, 536, 95/386/536, 264, 99/113/386/449, 95/386, 95/99/204/386, 113/142/386/449, 182/184/264/494, 113/142/182/494, 113, 99/264/449, 95, 95/99, 99/113/449, 95/113/449/536, 95/99/113, 386/536, 99/182/184, 142/386/400/536, 35/264/449/536, 142/144/220, 99/142, 29/102/182/276/336/390/459/502/532, 41/262/336/502, 29/97/150/262/336/502/532, 29, 532, 102/390/459/502/532, 29/262/276/336/495, 29/182/262/459/532, 92/99/102/502, 29/97/102/182/502/532, 336/459/495/502/532, 150/182/336/390, 150/262/495, 29/495, 29/102/182/262/502, 29/336/459/502/532, 182/495/502/532, 102/336, 150/182/262/495, 459/495/502/532, 41/182/390/495, 29/41/182/336/495, 97/102/495, 182/262/495, 29/262/502, 29/102/502, 182/336/495, 29/150/182/262/459/495, 502/532, 386, 497, 99, 512, 69, 507, 445, 75, 487, 184, 90, 235, 434, 489, 86, 182, 446, 490, 299, 102, 230, 68, 141, 97, 316, 522, 224, 478, 480, 368, 412, 449, 265, or 494, wherein the amino acid positions are relative to the reference sequence corresponding to residues 40-536 of SEQ ID NO: 1150, or relative to the reference sequence corresponding to SEQ ID NO: 1150.

In some embodiments, the recombinant polynucleotide comprises a polynucleotide sequence encoding an engineered glucocerebrosidase comprising an amino acid sequence comprising at least a substitution at an amino acid position set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, 54, 220, 984, or 1150, or relative to the reference sequence corresponding to SEQ ID NO: 2, 54, 220, or 984, or 1150.

In some embodiments, the recombinant polynucleotide comprises a polynucleotide sequence encoding the engineered glucocerebrosidase comprising an amino acid sequence comprising at least one substitution set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, 54, 220, 984, or 1150, or relative to the reference sequence corresponding to SEQ ID NO: 2, 54, 220, or 984, or 1150.

In some embodiments, the recombinant polynucleotide comprises a polynucleotide sequence encoding an engineered glucocerebrosidase comprising an amino acid sequence comprising at least a substitution or substitution set at amino acid position(s) set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, 54, 220, 984, or 1150, or relative to the reference sequence corresponding to SEQ ID NO: 2, 54, 220, or 984, or 1150.

In some embodiments, the recombinant polynucleotide comprises a polynucleotide sequence encoding an engineered glucocerebrosidase comprising an amino acid sequence comprising at least a substitution or substitution set of an engineered glucocerebrosidase set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, 54, 220, 984, or 1150, or relative to the reference sequence corresponding to SEQ ID NO: 2, 54, 220, or 984, or 1150.

In some embodiments, the recombinant polynucleotide comprises a polynucleotide sequence encoding an engineered glucocerebrosidase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising a substitution or substitution set of an engineered glucocerebrosidase set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

In some embodiments, the recombinant polynucleotide comprises a polynucleotide sequence encoding an engineered glucocerebrosidase comprising an amino acid sequence comprising residues 40-536 of an engineered glucocerebrosidase set forth in Tables 3.1, 3.2, 4.1, 4.2, 4.3, 4.4, 5.1, 6.1, and 6.2, or an amino acid sequence comprising an engineered glucocerebrosidase set forth in Tables 3.1, 3.2, 4.1, 4.2, 4.3, 4.4, 5.1, 6.1, and 6.2.

In some embodiments, the recombinant polynucleotide comprises a polynucleotide sequence encoding the engineered glucocerebrosidase comprising an amino acid sequence comprising residues 40-536 of SEQ ID NO: 54, 220, 984, or 1150, or comprising SEQ ID NO: 54, 220, 984, or 1150.

In some embodiments, the recombinant polynucleotide comprises a polynucleotide sequence encoding the engineered glucocerebrosidase comprising an amino acid sequence comprising residues 40-536 of an even-numbered SEQ ID NO. of SEQ ID NOs: 4-1648, or comprising an even-numbered SEQ ID NO. of SEQ ID NOs: 4-1648.

In some embodiments, the recombinant polynucleotide comprises a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 118-1608 of an odd-numbered SEQ ID NO. of SEQ ID NOs: 3-1647, or to a reference sequence corresponding to an odd-numbered SEQ ID NO. of SEQ ID NOs: 3-1647, wherein the recombinant polynucleotide encodes a glucocerebrosidase.

In some embodiments, the recombinant polynucleotide comprises a polynucleotide sequence having 95%, 96%, 97%, 98%, 99%, or more sequence identity to a reference polynucleotide sequence corresponding to nucleotide residues 118-1608 of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 872, 873, 875, 877, 879, 881, 883, 885, 887, 889, 891, 893, 895, 897, 899, 901, 903, 905, 907, 909, 911, 913, 915, 917, 919, 921, 219, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1471, 1473, 1475, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, 1617, 1619, 1621, 1623, 1625, 1627, 1629, 1631, 1633, 1635, 1637, 1639, 1641, 1643, 1645, or 1647, wherein the recombinant polynucleotide encodes an glucocerebrosidase.

In some embodiments, the engineered glucocerebrosidase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 872, 873, 875, 877, 879, 881, 883, 885, 887, 889, 891, 893, 895, 897, 899, 901, 903, 905, 907, 909, 911, 913, 915, 917, 919, 921, 219, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1471, 1473, 1475, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, 1617, 1619, 1621, 1623, 1625, 1627, 1629, 1631, 1633, 1635, 1637, 1639, 1641, 1643, 1645, or 1647, wherein the recombinant polynucleotide encodes an glucocerebrosidase.

In some embodiments, the recombinant polynucleotide comprises a polynucleotide sequence codon-optimized for expression of the encoded glucocerebrosidase. In some embodiments, the polynucleotide sequence is codon-optimized for expression in mammalian cells. In some embodiments, the polynucleotide sequence is codon optimized for expression in a bacterial cell, fungal cell, insect cell, or mammalian cell, particularly a human cell.

In some embodiments, the recombinant polynucleotide comprises a polynucleotide sequence comprising residues 118-1608 of an odd-numbered SEQ ID NO. of SEQ ID NOs: 3-1647, or a polynucleotide sequence comprising an odd-numbered SEQ ID NO. of SEQ ID NOs: 3-1647.

In some embodiments, the recombinant polynucleotide comprises a polynucleotide sequence comprising nucleotide residues 118-1608 of SEQ ID NO. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 872, 873, 875, 877, 879, 881, 883, 885, 887, 889, 891, 893, 895, 897, 899, 901, 903, 905, 907, 909, 911, 913, 915, 917, 919, 921, 219, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1471, 1473, 1475, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, 1617, 1619, 1621, 1623, 1625, 1627, 1629, 1631, 1633, 1635, 1637, 1639, 1641, 1643, 1645, or 1647.

In some embodiments, the recombinant polynucleotide comprises a polynucleotide sequence comprising SEQ ID NO. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 872, 873, 875, 877, 879, 881, 883, 885, 887, 889, 891, 893, 895, 897, 899, 901, 903, 905, 907, 909, 911, 913, 915, 917, 919, 921, 219, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1471, 1473, 1475, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, 1617, 1619, 1621, 1623, 1625, 1627, 1629, 1631, 1633, 1635, 1637, 1639, 1641, 1643, 1645, or 1647.

In some embodiments, the recombinant polynucleotide comprises a polynucleotide sequence comprising nucleotide residues 118-1608 of SEQ ID NO: 53, 219, 983, or 1149, or a polynucleotide sequence comprising SEQ ID NO: 53, 219, 983, or 1149.

In some embodiments, the recombinant polynucleotide comprises a polynucleotide sequence encoding a “deimmunized” engineered glucocerebrosidase comprising
an amino acid sequence comprising residues 40-536 of SEQ ID NO: 4, 12, 22, 30, 32, 38, 44, 46, 50, 54, 56, 58, 60, 64, 72, 76, 80, 82, 84, 86, 88, 90, 92, 96, 98, 200, 208, 212, 216, 218, 222, 226, 228, 232, 236, 242, 244, 254, 256, 260, 262, 266, 272, 274, 276, 278, 280, 282, 286, 292, 298, 300, 302, 304, 306, 308, 312, 314, 316, 318, 320, 322, 324, 326, 330, 334, 336, 338, 340, 342, 346, 348, 350, 352, 354, 356, 358, 360, 362, 364, 366, 368, 370, 372, 374, 376, 382, 384, 386, 388, 390, 392, 394, 396, 398, 400, 402, 404, 406, 408, 410, 412, 414, 416, 418, 422, 424, 426, 428, 430, 432, 434, 436, 438, 444, 446, 448, 450, 452, 454, 456, 458, 460, 462, 464, 466, 468, 470, 472, 476, 478, 480, 482, 484, 490, 492, 496, 500, 502, 508, 510, 512, 514, 516, 518, 524, 526, 528, 532, 534, 536, 538, 540, 542, 544, 546, 552, 554, 558, 560, 562, 564, 566, 568, 570, 572, 574, 576, 578, 580, 582, 584, 588, 590, 592, 594, 596, 600, 602, 604, 606, 608, 610, 612, 618, 620, 626, 628, 630, 632, 634, 636, 638, 640, 644, 646, 648, 652, 654, 656, 660, 662, 666, 668, 670, 672, 674, 676, 678, 680, 682, 684, 686, 688, 690, 692, 694, 696, 698, 700, 706, 708, 710, 714, 716, 718, 720, 722, 724, 726, 728, 730, 732, 734, 736, 740, 742, 746, 748, 750, 752, 756, 760, 762, 766, 768, 770, 772, 774, 776, 778, 780, 782, 784, 786, 788, 790, 794, 796, 798, 800, 802, 804, 806, 808, 810, 814, 816, 818, 822, 828, 830, 834, 836, 842, 844, 846, 850, 852, 854, 856, 858, 860, 862, 864, 866, 868, 870, 874, 876, 878, 880, 882, 884, 886, 888, 890, 892, 894, 896, 898, 900, 902, 904, 906, 910, 914, 916, 918, 920, 924, 928, 932, 936, 940, 946, 948, 952, 956, 958, 960, 964, 968, 970, 980, 984, 988, 990, 994, 998, 1002, 1004, 1006, 1008, 1012, 1016, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, or 1216, or
an amino acid sequence comprising SEQ ID NO: 4, 12, 22, 30, 32, 38, 44, 46, 50, 54, 56, 58, 60, 64, 72, 76, 80, 82, 84, 86, 88, 90, 92, 96, 98, 200, 208, 212, 216, 218, 222, 226, 228, 232, 236, 242, 244, 254, 256, 260, 262, 266, 272, 274, 276, 278, 280, 282, 286, 292, 298, 300, 302, 304, 306, 308, 312, 314, 316, 318, 320, 322, 324, 326, 330, 334, 336, 338, 340, 342, 346, 348, 350, 352, 354, 356, 358, 360, 362, 364, 366, 368, 370, 372, 374, 376, 382, 384, 386, 388, 390, 392, 394, 396, 398, 400, 402, 404, 406, 408, 410, 412, 414, 416, 418, 422, 424, 426, 428, 430, 432, 434, 436, 438, 444, 446, 448, 450, 452, 454, 456, 458, 460, 462, 464, 466, 468, 470, 472, 476, 478, 480, 482, 484, 490, 492, 496, 500, 502, 508, 510, 512, 514, 516, 518, 524, 526, 528, 532, 534, 536, 538, 540, 542, 544, 546, 552, 554, 558, 560, 562, 564, 566, 568, 570, 572, 574, 576, 578, 580, 582, 584, 588, 590, 592, 594, 596, 600, 602, 604, 606, 608, 610, 612, 618, 620, 626, 628, 630, 632, 634, 636, 638, 640, 644, 646, 648, 652, 654, 656, 660, 662, 666, 668, 670, 672, 674, 676, 678, 680, 682, 684, 686, 688, 690, 692, 694, 696, 698, 700, 706, 708, 710, 714, 716, 718, 720, 722, 724, 726, 728, 730, 732, 734, 736, 740, 742, 746, 748, 750, 752, 756, 760, 762, 766, 768, 770, 772, 774, 776, 778, 780, 782, 784, 786, 788, 790, 794, 796, 798, 800, 802, 804, 806, 808, 810, 814, 816, 818, 822, 828, 830, 834, 836, 842, 844, 846, 850, 852, 854, 856, 858, 860, 862, 864, 866, 868, 870, 874, 876, 878, 880, 882, 884, 886, 888, 890, 892, 894, 896, 898, 900, 902, 904, 906, 910, 914, 916, 918, 920, 924, 928, 932, 936, 940, 946, 948, 952, 956, 958, 960, 964, 968, 970, 980, 984, 988, 990, 994, 998, 1002, 1004, 1006, 1008, 1012, 1016, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, or 1216.

In some embodiments, the polynucleotide sequence of the recombinant polynucleotide includes a stop codon, as known in the art, for termination of translation of the engineered polypeptide. In some embodiments, the polynucleotide sequence of the recombinant polynucleotide includes at least one stop codon following the codon for the carboxy terminal amino acid of the engineered polypeptide. In some embodiments, the polynucleotide sequence of the recombinant polynucleotide includes two stop codons in tandem following the codon for the carboxy terminal amino acid of the engineered polypeptide. In some embodiments, the stop codons are selected from standard stop codons, e.g., TAG, TAA and TGA in the DNA sequence, and UAG, UAA and UGA in the corresponding RNA sequence. In some embodiments, the stop codon is a stop codon preferably used in mammalian genes, e.g., TGA/UGA (see, e.g., Seoighe et al., J Molecular Evolution, 2020, 88:549-561).

In some embodiments, the recombinant polynucleotide hybridizes under highly stringent conditions to a reference polynucleotide sequence described herein encoding an engineered glucocerebrosidase e.g., a recombinant polynucleotide provided in Tables 3.1, 3.2, 4.1, 4.2, 4.3, 4.4, 5.1, 6.1, 6.2, 12.1, 12.2, and 13.1, or a reverse complement thereof. In some embodiments, the reference polynucleotide sequence corresponds to nucleotide residues 118-1608 of SEQ ID NO: 53, 219, 983, or 1149, or the sequence corresponding to SEQ ID NO: 53, 219, 983, or 1149, or a reverse complement thereof, or a polynucleotide sequence encoding any of the other engineered glucocerebrosidase provided herein. In some embodiments, the recombinant polynucleotide encodes a glucocerebrosidase and hybridizes under highly stringent conditions to a reference polynucleotide sequence corresponding to nucleotide residues 118-1608 of an odd-numbered SEQ ID NO. of SEQ ID NOs: 3-1647, or to a reference polynucleotide sequence corresponding to an odd-numbered SEQ ID NO. of SEQ ID NOs: 3-1647, or a reverse complement thereof.

In some embodiments, the recombinant polynucleotide hybridizes under highly stringent conditions to a reverse complement of a reference polynucleotide sequence encoding an engineered glucocerebrosidase, wherein the engineered glucocerebrosidase comprises an amino acid sequence having one or more amino acid differences as compared to SEQ ID NO: 2, 54, 220, 984, or 1150 at residue positions selected from any positions as set forth in Tables 3.1, 3.2, 4.1, 4.2, 4.3, 4.4, 5.1, 6.1, 6.2, 12.1, 12.2, and 13.1. In some embodiments, the recombinant polynucleotide that hybridizes under highly stringent conditions to a reverse complement of a reference polynucleotide encoding an engineered glucocerebrosidase polypeptide described herein encodes a glucocerebrosidase polypeptide having one or more amino acid differences present in an engineered glucocerebrosidase having an amino acid sequence corresponding to residues 40-536 of an even numbered SEQ ID NO. of SEQ ID NOs: 4-1648, or an amino acid sequence comprising an even numbered SEQ ID NO. of SEQ ID NOs: 4-1648, wherein the amino acid differences are relative to SEQ ID NO: 2, 54, 220, or 984, or 1150.

In some embodiments, the polynucleotide that hybridizes under highly stringent conditions comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 118-1608 of SEQ ID NO: 53, 219, 983, or 1149, or to a reference polynucleotide sequence corresponding to SEQ ID NO: 53, 219, 983, or 1149, wherein the recombinant polynucleotide encodes a glucocerebrosidase. In some additional embodiments, the polynucleotide that hybridizes under highly stringent conditions comprises a polynucleotide sequence having at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 118-1608 of an odd-numbered SEQ ID NO. of SEQ ID NOs: 3-1647, or to a reference polynucleotide sequence corresponding to an odd-numbered SEQ ID NO. of SEQ ID NOs: 3-1647, wherein the recombinant polynucleotide encodes a glucocerebrosidase.

In some embodiments, a recombinant polynucleotide encoding any of the engineered glucocerebrosidase provided herein is manipulated in a variety of ways to provide for expression of the engineered polypeptide. In some embodiments, the polynucleotides encoding the polypeptides are provided as expression vectors where one or more control sequences is present to regulate the expression of the polynucleotides and/or polypeptides. The techniques for modifying polynucleotides and nucleic acid sequences utilizing recombinant DNA methods are well known in the art. In some embodiments, the control sequences include among others, promoter sequences, Kozak sequence, leader sequences, polyadenylation sequences, pro-peptide sequences, signal peptide sequences, DNA based regulatory elements for gene therapy retention and transcription terminators.

In some embodiments, suitable promoters are selected based on the host cell selection. For bacterial host cells, suitable promoters for directing transcription of the nucleic acid constructs of the present disclosure, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (see, e.g., Villa-Kamaroff et al., Proc. Natl Acad. Sci. USA, 1978, 75:3727-3731), as well as the tac promoter (see, e.g., DeBoer et al., Proc. Natl Acad. Sci. USA, 1983, 80:21-25). Exemplary promoters for filamentous fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (see, e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (see, e.g., Romanos et al., Yeast, 1992, 8:423-488). Exemplary promoters for use in insect cells include, but are not limited to, polyhedrin, p10, ELT, OpIE2, and hr5/ie1 promoters. Exemplary promoters for use in mammalian cells include, but are not limited to, those from cytomegalovirus (CMV), chicken β-actin promoter fused with the CMV enhancer, Simian vacuolating virus 40 (SV40), from Homo sapiens phosphoglycerate kinase, beta actin, elongation factor-la or glyceraldehyde-3-phosphate dehydrogenase, and from Gallus β-actin.

In some embodiments, the control sequence is a suitable transcription terminator sequence (i.e., a sequence recognized by a host cell to terminate transcription). In some embodiments, the terminator sequence is operably linked to the 3′ terminus of the nucleic acid sequence encoding the glucocerebrosidase polypeptide. Any suitable terminator which is functional in the host cell of choice finds use in the present invention. For bacterial expression, the transcription terminators can be a Rho-dependent terminators that rely on a Rho transcription factor, or a Rho-independent, or intrinsic terminators, which do not require a transcription factor. Exemplary bacterial transcription terminators are described in Peters et al., J Mol Biol., 2011, 412(5): 793-813. Exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (see, e.g., Romanos et al., Yeast, 1992, 8(6): 423-88). Exemplary terminators for insect cells and mammalian cells include, but are not limited to, those from cytomegalovirus (CMV), Simian virus 40 (SV40), from Homo sapiens growth hormone hGH, from bovine growth hormone BGH, and from human or rabbit beta globulin.

In some embodiments, the control sequence is a suitable leader sequence, 5′-cap modification, 5′ UTR, etc. In some embodiments, these regulatory sequence elements mediate binding to molecules involved in mRNA trafficking and translation, inhibit 5′-exonucleolytic degradation and confer resistance to de-capping. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells include, but are not limited to those obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). Suitable leaders for mammalian host cells include but are not limited to the 5′-UTR element present in orthopoxvirus mRNA.

In some embodiments, the control sequence comprises a 3′ untranslated nucleic acid region and polyadenylation tail nucleic acid sequence, sequences operably linked to the 3′ terminus of the protein coding nucleic acid sequence, which mediate binding to proteins involved in mRNA trafficking and translation and mRNA half-life. Any polyadenylation sequence and 3′ UTR which is functional in the host cell of choice may be used in the present invention. Exemplary polyadenylation sequences for filamentous fungal host cells include, but are not limited to those from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are also known in the art (See e.g., Guo and Sherman, Mol. Cell. Biol., 1995, 15:5983-5990). Useful polyadenylation and 3′ UTR sequences for insect and mammalian host cells include, but are not limited to, OpIE2 polyA sequence, D. melanogaster metallothionein (Mt) poly A signal sequence, D. melanogaster alcohol dehydrogenase (adh), SV40 poly A signal sequence, and the 3′-UTRs of α- and β-globin mRNAs harboring sequence elements that increase the stability and translation of mRNA.

In some embodiments, the control sequence is also a signal peptide (i.e., a coding region that codes for an amino acid sequence linked to the amino terminus of a polypeptide and directs the encoded polypeptide into the cell's secretory pathway). In some embodiments, the 5′ end of the coding sequence of the nucleic acid sequence inherently contains a signal peptide coding region naturally linked in translation reading frame with the segment of the coding region that encodes the secreted polypeptide. Alternatively, in some embodiments, the 5′ end of the coding sequence contains a signal peptide coding region that is foreign to the coding sequence. Any suitable signal peptide coding region which directs the expressed polypeptide into the secretory pathway of a host cell of choice finds use for expression of the engineered polypeptide(s). Effective signal peptide coding regions for bacterial host cells are the signal peptide coding regions include, but are not limited to those obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (see, e.g., Simonen and Palva, Microbiol. Rev., 1993, 57:109-137). In some embodiments, effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase. Useful signal peptides for insect and mammalian host cells include but are not limited to, those from the genes for immunoglobulin gamma (IgG) and the signal peptide in a human secreted protein, such as human beta-galactosidase polypeptide. In some embodiments, the signal peptide is the naturally occurring signal peptide in human glucocerebrosidase.

In some embodiments, regulatory sequences are also utilized. These sequences facilitate the regulation of the expression of the polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter.

In another aspect, the present disclosure also provides an expression vector comprising a recombinant polynucleotide encoding an engineered glucocerebrosidase, and one or more expression regulating regions such as a promoter and a terminator, a replication origin, etc., depending on the type of hosts into which they are to be introduced. In some embodiments, the various nucleic acid and control sequences described above are joined together to produce a recombinant expression vector which includes one or more convenient restriction sites to allow for insertion or substitution of the nucleic acid sequence encoding the engineered glucocerebrosidase at such sites. Alternatively, the polynucleotide sequence(s) of the present disclosure are expressed by inserting the polynucleotide sequence or a nucleic acid construct comprising the polynucleotide sequence into an appropriate vector for expression. In creating the expression vector, the coding sequence is located in the vector so that the coding sequence is operably linked with the appropriate control sequences for expression.

In some embodiments, the expression vector may be any vector (e.g., a plasmid or virus, including but not limited to adenovirus (AV), adeno-associated virus (AAV), lentivirus (LV), or retrovirus), that can be conveniently subjected to recombinant DNA procedures and can result in the expression of the engineered glucocerebrosidase polynucleotide sequence. The choice of the vector will typically depend on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

In some embodiments, the recombinant polynucleotides may be provided on a non-replicating expression vector or plasmid. In some embodiments, the non-replicating expression vector or plasmid can be based on viral vectors defective in replication (see, e.g., Travieso et al., npj Vaccines, 2022, Vol. 7, Article 75).

In some embodiments, the expression vector preferably contains one or more selectable markers, which permit easy selection of transformed cells. A “selectable marker” is a gene the product of which provides for biocide or viral resistance, resistance to heavy metals, prototrophy to auxotrophs, and the like. Suitable markers for yeast host cells include, but are not limited to ADE2, HIS3, LEU2, LYS2, MET3, TRP1, and URA3. Selectable markers for use in a filamentous fungal host cell include, but are not limited to, amdS (acetamidase), argB (ornithine carbamoyltransferases), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof.

In another aspect, the present disclosure provides a host cell comprising a polynucleotide encoding at least one engineered glucocerebrosidase described herein, the polynucleotide being operatively linked to one or more control sequences for expression of the engineered glucocerebrosidases(s) in the host cell. Host cells for use in expressing the polypeptides encoded by the expression vectors of the present invention are well known in the art and include but are not limited to, bacterial cells, fungal cells, such as yeast cells (e.g., Saccharomyces cerevisiae and Pichia pastoris, e.g., ATCC Accession No. 201178); insect cells (e.g., Drosophila S2 and Spodoptera Sf9 cells), plant cells, animal cells (e.g., CHO, CHO-K1, COS, and BHK), and human cells (e.g., HEK293T, human fibroblast, THP-1, Jurkat and Bowes melanoma cell lines). In some embodiments, the host cell is a human cell obtained or derived from a subject with Gaucher disease.

Accordingly, in another aspect, the present disclosure provides a method for producing the engineered glucocerebrosidases, where the method comprises culturing a host cell capable of expressing a polynucleotide encoding the engineered glucocerebrosidase under conditions suitable for expression of the polypeptide. In some embodiments, the method further comprises isolating the engineered glucocerebrosidase, such as from the culture media and/or cells. In some embodiments, the method further comprises purifying the expressed engineered glucocerebrosidase.

Appropriate culture media and growth conditions for the above-described host cells are well known in the art. Polynucleotides for expression of the engineered glucocerebrosidases may be introduced into cells by various methods known in the art. Techniques include, among others, electroporation, biolistic particle bombardment, liposome mediated transfection, calcium chloride transfection, and protoplast fusion.

In some embodiments, the glucocerebrosidase polypeptide expressed in a host cell is recovered from the cells and/or the culture medium using any one or more of the known techniques for protein purification, including, among others, lysozyme or detergent treatment, sonication, filtration, salting-out, ultra-centrifugation, and chromatography, such as described herein.

Chromatographic techniques for isolation/purification of the glucocerebrosidase polypeptides include, among others, reverse phase chromatography, high-performance liquid chromatography, ion-exchange chromatography, hydrophobic-interaction chromatography, size-exclusion chromatography, gel electrophoresis, and affinity chromatography. Conditions for purifying the glucocerebrosidase depends, in part, on factors such as net charge, hydrophobicity, hydrophilicity, molecular weight, molecular shape, etc., and will be apparent to those having skill in the art. In some embodiments, affinity techniques may be used to isolate the glucocerebrosidase. For affinity chromatography purification, an antibody that specifically binds glucocerebrosidase polypeptide may be used. In some embodiments, an affinity tag, e.g., His-tag, can be introduced into the glucocerebrosidase polypeptide for purposes of isolation/purification.

In some embodiments, recombinant polypeptides (e.g., glucocerebrosidase variants) can be produced using any suitable methods known the art. For example, there is a wide variety of different mutagenesis techniques known to those skilled in the art. Methods are available to make specific substitutions at defined amino acids (site-directed), specific or random mutations in a localized region of the gene (region-specific), or random mutagenesis over the entire gene (e.g., saturation mutagenesis). Numerous suitable methods are known to those in the art to generate protein variants, including but not limited to site-directed mutagenesis of single-stranded DNA or double-stranded DNA using PCR, cassette mutagenesis, gene synthesis, error-prone PCR, shuffling, and chemical saturation mutagenesis, or any other suitable method known in the art. Non-limiting examples of methods used for DNA and protein engineering are provided in the following patents: U.S. Pat. Nos. 6,117,679; 6,420,175; 6,376,246; 6,586,182; 7,747,391; 7,747,393; 7,783,428; and 8,383,346. After the variants are produced, they can be screened for any desired property (e.g., high or increased activity, increased thermal activity, increased stability, increased serum tolerance, increased uptake by mammalian cell, increased pH stability, etc.). Exemplary methods are provided in the Examples.

In some embodiments, the engineered glucocerebrosidases with the properties disclosed herein can be obtained by subjecting the polynucleotide encoding the naturally occurring or engineered glucocerebrosidase to mutagenesis and/or directed evolution methods known in the art, and as described herein. An exemplary directed evolution technique is mutagenesis and/or DNA shuffling (See e.g., Stemmer, Proc. Natl. Acad. Sci. USA, 1994, 91:10747-10751; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (see e.g., Zhao et al., Nat. Biotechnol., 1998, 16:258-261), mutagenic PCR (see e.g., Caldwell et al., PCR Methods Appl., 1994, 3: S136-S140), and cassette mutagenesis (see e.g., Black et al., Proc. Natl. Acad. Sci. USA, 1996, 93:3525-3529).

For example, mutagenesis and directed evolution methods can be applied to polynucleotides to generate variant libraries that can be expressed, screened, and assayed. Mutagenesis and directed evolution methods are known in the art (see, e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,489,146, 6,506,602, 6,506,603, 6,519,065, 6,521,453, 6,528,311, 6,537,746, 6,573,098, 6,576,467, 6,579,678, 6,586,182, 6,602,986, 6,613,514, 6,653,072, 6,716,631, 6,946,296, 6,961,664, 6,995,017, 7,024,312, 7,058,515, 7,105,297, 7,148,054, 7,288,375, 7,421,347, 7,430,477, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,873,499, 7,904,249, 7,957,912, 8,383,346, 8,504,498, 8,849,575, 8,876,066, 8,768,871, 9,593,326, and all related non-US counterparts; Ling et al., Anal. Biochem., 1997, 254 (2): 157-78; Dale et al., Meth. Mol. Biol., 1996, 57:369-74; Smith, Ann. Rev. Genet., 1985, 19:423-462; Botstein et al., Science, 1985, 229:1193-1201; Carter, Biochem. J., 1986, 237:1-7; Kramer et al., 1984, Cell, 38:879-887; Wells et al., Gene, 1985, 34:315-323; Minshull et al., Curr. Op. Chem. Biol., 1999, 3:284-290; Christians et al., Nat. Biotechnol., 1999, 17:259-264; Crameri et al., Nature, 1998, 391:288-291; Crameri, et al., Nat. Biotechnol., 1997, 15:436-438; Zhang et al., Proc. Nat. Acad. Sci. USA, 1997, 94:4504-4509; Crameri et al., Nat. Biotechnol., 1996, 14:315-319; Stemmer, Nature, 1994, 370:389-391; Stemmer, Proc. Nat. Acad. Sci. USA, 1994, 91:10747-10751; U.S. patent publication Nos. 2008/0220990, US 2009/0312196, US2014/0005057, US2014/0214391, US2014/0221216; US2015/0050658, US2015/0133307, US2015/0134315 and all related non-US counterparts; WO 95/22625, WO 97/0078, WO 97/35966, WO 98/27230, WO 00/42651, WO 01/75767, and WO 2009/152336; all of which are incorporated herein by reference).

In some embodiments, the polypeptide variants obtained following mutagenesis treatment are screened by subjecting the protein variants to a defined temperature, pH, or other assay conditions (e.g., exposure to serum), and measuring the amount of protein activity remaining after the treatments or other assay conditions. The polynucleotide encoding the engineered glucocerebrosidase is sequenced to identify the nucleotide sequence changes (if any), and used to express the protein in a different or the same host cell. Measuring protein activity from the expression libraries can be performed using any suitable method known in the art, such as provided in the Examples.

In some embodiments, for engineered polypeptides of known sequence, the polynucleotides encoding the polypeptide can be prepared by standard solid-phase methods, according to known synthetic methods. In some embodiments, polynucleotide fragments can be individually synthesized, then joined (e.g., by enzymatic or chemical litigation methods, or polymerase mediated methods) to form any desired continuous sequence (see, e.g., Hughes et al., Cold Spring Harb Perspect Biol. 2017 January; 9(1):a023812). For example, polynucleotides and oligonucleotides disclosed herein can be prepared by chemical synthesis using the classical phosphoramidite method (See e.g., Beaucage et al., Tetra. Lett., 1981, 22:1859-69; and Matthes et al., EMBO J., 1984, 3:801-05), as it is typically practiced in automated synthetic methods. The synthesized oligonucleotides can be annealed, ligated, and cloned in appropriate vectors.

In some embodiments, a method for preparing the engineered glucocerebrosidase can comprise: (a) synthesizing a polynucleotide encoding a polypeptide comprising an amino acid sequence selected from the amino acid sequence of any variant described herein, including variants provided in Tables 3.1, 3.2, 4.1, 4.2, 4.3, 4.4, 5.1, 6.1, 6.2, 12.1, 12.2, and 13.1, or the amino acid sequence corresponding to residues 40-536 of an even-numbered SEQ ID NO. of SEQ ID NOs: 4-1648, or the amino acid sequence corresponding to an even-numbered SEQ ID NO. of SEQ ID NOs: 4-1648, and (b) expressing the engineered glucocerebrosidase encoded by the polynucleotide. In some embodiments of the method, the amino acid sequence encoded by the polynucleotide can optionally have one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions can be conservative or non-conservative substitutions.

The expressed engineered glucocerebrosidase can be assessed for any desired improved property (e.g., activity/potency, selectivity, stability, serum/plasma stability, acid pH tolerance, basic pH tolerance, protease sensitivity, uptake, intracellular stability, etc.), using any suitable assay known in the art, including but not limited to the assays and conditions described herein.

In some embodiments, once the engineered glucocerebrosidases are recovered or isolated from the recombinant host cells or cell culture medium, they are further purified by any suitable method(s) known in the art. In some additional embodiments, the purified engineered glucocerebrosidase polypeptides are combined with other ingredients and compounds to provide compositions and formulations comprising the engineered glucocerebrosidase as appropriate for different applications and uses (e.g., pharmaceutical compositions).

In some additional embodiments, the purified engineered glucocerebrosidase, or the formulated engineered glucocerebrosidase is lyophilized. In some embodiments, the engineered glucocerebrosidases are directly produced within a body (i.e., cells within a body, such as a human or another animal) and are not purified. However, in some alternative embodiments, the engineered glucocerebrosidases are produced within a body (i.e., cells within a body, such as a human or another animal) and are collected from the body using methods known in the art.

### Pharmaceutical Compositions

In a further aspect, the present disclosure provides pharmaceutical compositions comprising an engineered glucocerebrosidase or a recombinant polynucleotide encoding the engineered glucocerebrosidase described herein. The pharmaceutical composition may vary depending on the therapeutic form or agent used, e.g., polypeptide or polynucleotide.

In some embodiments, the pharmaceutically composition is formulated for administration by any suitable route, such as oral, topical, intradermal, transdermal, intravenous, intraperitoneal, intrathecal intracerebral, subcutaneous, intramuscular, intranasal, and/or intraocular (e.g., intravitreal/retinal) administration. In some embodiments, the engineered glucocerebrosidases are provided in a form suitable for injection or infusion (i.e., in an injectable formulation).

In some embodiments, a pharmaceutical composition is prepared as a solid, semi-solid, or liquid, depending on the mode of administration. In some embodiments, the pharmaceutical composition is any suitable form, including but not limited to, pills, tablets, gel tabs, capsules, dragees, powders, soft gels, sol-gels, gels, emulsions, implants, sprays, aerosols, solutions, suspensions (including but not limited to oil-based suspensions, oil-in water emulsions, etc.), slurries, syrups, controlled release formulations. In some embodiments, the engineered glucocerebrosidase is a lyophilizate or powder, where the enzyme is reconstituted by addition of an appropriate solution for administration.

In some embodiments, the pharmaceutical composition comprises an engineered glucocerebrosidase or a recombinant polynucleotide encoding the engineered glucocerebrosidase, and a pharmaceutically acceptable carrier or excipient. In some embodiments, the pharmaceutical composition includes pharmaceutically acceptable components such as diluents, buffers, excipients, salts, emulsifiers, preservatives, stabilizers, fillers, and other ingredients. In some embodiments, the excipients and/or carriers may include, but not limited to, sugars (e.g., lactose, sucrose, mannitol, and/or sorbitol), starches, cellulose (e.g., methyl cellulose, hydroxypropylmethyl cellulose, sodium carboxy-methylcellulose), gums (e.g., arabic, tragacanth, guar, etc.), and/or proteins (e.g., gelatin, collagen, etc.). In some embodiments, the excipient and/or carrier, includes diluents such as water, saline, glycerol and ethanol. In some embodiments, the excipients or carriers comprise pharmaceutically acceptable salts, for example, mineral acid salts such as hydrochlorides, hydrobromides, phosphates, sulfates, and the like; and the salts of organic acids such as acetates, propionates, malonates, and benzoates. A description of pharmaceutically acceptable excipients and carriers is available in Remington's Pharmaceutical Sciences (Mack Pub. Co., N.J. 1991) and Remington: The Science and Practice of Pharmacy, A. Adejare ed., 23 Ed., Academic Press 2020).

In some embodiments, the engineered glucocerebrosidases are provided in biocompatible matrices such as sol-gels, including silica-based (e.g., oxysilane) sol-gels. In some embodiments, the engineered glucocerebrosidases are encapsulated. In some alternative embodiments, the engineered glucocerebrosidases are encapsulated microparticles or nanoparticles (e.g., nanotubes, nanotubules, nanocapsules, or microcapsules, microspheres, liposomes, etc.).

In some embodiments, the engineered glucocerebrosidases are modified by glycosylation, chemical crosslinking reagents, pegylation (i.e., modified with polyethylene glycol (PEG) or activated PEG, etc.) or other compounds (see e.g., Ikeda, Amino Acids, 2005, 29:283-287; U.S. Pat. Nos. 7,531,341, 7,534,595, and 7,560,263; US Pat. Publication Nos. 2013/0039898, 2012/0177722, etc.).

In some embodiments, the pharmaceutical composition comprises a recombinant polynucleotide encoding an engineered glucocerebrosidase described herein, such as for use in gene therapy. In some embodiments, the recombinant polynucleotide is RNA or DNA.

In some embodiments, the pharmaceutical composition comprises a polyribonucleotide (e.g., mRNA) in a delivery vehicle, for example liposomes or nanoparticles, cationic polymers, dendrimers, etc. In some embodiments, the mRNA is delivered to cells formulated in RNA virus delivery systems, such as alphavirus, rabies virus, vesicular stomatitis virus, Sendai virus, and retrovirus (see, e.g., Nishimura et al., J Biol Chem., 2007, 282:27383-27391; Segel et al., Science, 2021, 373, 882-889).

In some embodiments, the pharmaceutical composition is a gene therapy composition comprising a viral vector, wherein the viral vector comprises a recombinant polynucleotide encoding an engineered glucocerebrosidase. In some embodiments, viral delivery vectors, include but are not limited to, adenovirus (AV), adeno-associated virus (AAV), lentivirus (LV), retrovirus, or herpes simplex virus (HSV) based vectors. In some embodiments, the gene therapy composition comprises non-viral delivery compositions, where the recombinant polynucleotide, e.g., in an expression vector, is formulated with liposomes, cationic polymers, dendrimers, or conjugated to cell-penetrating peptides.

In some embodiments, the viral delivery vector is a recombinant AAV vector, for example, based on AAV1, AAV2, AAV3, AAV4, AAV5, AAV6, AAV7, AAV8, AAV9, AAV10, AAV11, AAV12, AAV13, AAVrh10, or AAVhu68. In some embodiments, the AAV vector is tropic for the CNS. In some preferred embodiments, the AAV vector is an AAV9 viral vector. In some embodiments, the AAV viral vector further comprises an inverted terminal repeat flanking the transgene comprising the recombinant polynucleotide encoding an engineered galactosidase described herein. In some embodiments, the inverted terminal repeat can be any viral inverted terminal repeat or synthetic sequence that forms a hairpin structure and functions as an inverted terminal repeat (i.e., mediates the desired functions, such as replication, virus packaging, integration and/or provirus rescue). The ITR can be an AAV ITR or a non-AAV ITR sequence, such as those of other parvoviruses (e.g., canine parvovirus (CPV), mouse parvovirus (MVM), human parvovirus B-19) or any other suitable virus sequence. For example, the SV40 hairpin that serves as the origin of SV40 replication can be used as an ITR. In some embodiments, the ITR can be partially or completely synthetic, such as the “double-D sequence” as described in U.S. Pat. No. 5,478,745. In some embodiments, the inverted terminal repeat is an AAV terminal repeat, such as from any AAV, including but not limited to serotypes 1, 2, 3, 3B, 4, 5, 6, 7, 8, 9, 10, 11, 12 or 13, or any other known AAV. In some embodiments, the inverted terminal repeat is from AAV9.

In some embodiments, the transgene comprising a recombinant polynucleotide encoding the engineered glucocerebrosidase is operably linked to a suitable promoter, as described herein. In some embodiments, exemplary promoters for expression in mammalian cells include, among others, cytomegalovirus (CMV), chicken β-actin promoter fused with the CMV enhancer (CAG), Simian Virus 40 (SV40), or promoters for phosphoglycerate kinase, beta actin, elongation factor-la or glyceraldehyde-3-phosphate dehydrogenase. In some preferred embodiments, the promoter for the viral vector comprises the CAG synthetic promoter.

In some embodiments, the gene therapy vector comprises an AAV9 vector, wherein the AAV9 vector comprises a CAG promoter operably linked to a transgene encoding an engineered beta-galactosidase described herein, wherein the viral vector further comprises AAV9 inverted terminal repeats flanking the CAG promoter-transgene.

In some embodiments, the gene therapy composition is based on CRISPR/CAS9 or related gene editing systems to insert a recombinant polynucleotide encoding an engineered glucocerebrosidase in the genome of a cell. In some embodiments, gene therapy composition comprises an expression vector encoding CRISPR, an expression vector for an appropriate guide RNA (gRNA), and a recombinant polynucleotide encoding an engineered glucocerebrosidase, wherein the recombinant polynucleotide is capable of homologous recombination or non-homologous end joining (NHEJ) with the cellular genome. In some embodiments, polynucleotide sequences are added to the ends of the recombinant polynucleotide encoding the engineered glucocerebrosidase to facilitate recombination or NHEJ using the CRISPR/Cas9 gene editing system.

In some additional embodiments, the engineered glucocerebrosidases are provided for delivery to cells or tissues via cell therapy, where the polynucleotide encoding the engineered glucocerebrosidases is introduced into exogenous cell and that cell (or cells) are introduced into a recipient or subject (e.g., a patient exhibiting deficiency in glucocerebrosidase). In some embodiments, the cell is a stem cell, particularly a human stem cell. In some embodiments, the stem cell is a hematopoietic stem cell, wherein a recombinant polynucleotide encoding an engineered glucocerebrosidase is introduced into a hematopoietic stem cell and the gene-modified hematopoietic stem cell is administered to a subject. In some embodiments, the stem cell is a neural stem cell, wherein a recombinant polynucleotide encoding an engineered glucocerebrosidase is introduced into a neural stem cell, and the gene-modified neural stem cell is administered to a subject.

### Uses and Methods

In another aspect, the engineered glucocerebrosidase or the recombinant polynucleotide encoding an engineered glucocerebrosidase, or pharmaceutical compositions thereof, is used to treat or prevent the symptoms of a deficiency in glucocerebrosidase in a subject.

In some embodiments, the treatment is based on enzyme replacement therapy (ERT). In some embodiments, a method of treating and/or preventing the symptoms of a deficiency in glucocerebrosidase activity in a subject comprises administering to a subject in need thereof an effective amount of an engineered glucocerebrosidase of the present disclosure to elevate levels of glucocerebrosidase activity sufficient to ameliorate the deficiency of glucocerebrosidase in the subject. In some embodiments, the treatment ameliorates the symptoms of a deficiency of glucocerebrosidase in the subject. In some embodiments, the engineered glucocerebrosidase comprises an amino acid sequence comprising residues 40-536 of an engineered glucocerebrosidase described herein. In some embodiments, the engineered glucocerebrosidase is administered at a dose of about 2.6-130 U/kg body weight or about 2.5 to 60 U/kg body weight, wherein the units are based on a standard substrate, e.g., 4-methylumbelliferyl-β-D-glucosylpyranoside.

In some embodiments, a subject with a deficiency in glucocerebrosidase activity is treated using a gene therapy composition comprising a recombinant polynucleotide encoding an engineered glucocerebrosidase. In some embodiments, a method of treating a subject having a deficiency in glucocerebrosidase activity comprises administering to a subject in need thereof an effective amount of a gene therapy composition comprising a recombinant polynucleotide encoding an engineered glucocerebrosidase. In some embodiments, the gene therapy composition comprises a viral vector comprising a recombinant polynucleotide encoding the engineered glucocerebrosidase. In some embodiments, the viral vector is an adenovirus adeno-associated virus, lentivirus, retrovirus, or herpes simplex virus vector. In some embodiments, the gene therapy is based on delivery using an AAV vector, including, among others, AAV1, AAV2, AAV3, AAV4, AAV5, AAV6, AAV7, AAV8, AAV9, AAV10, AAV11, AAV12, AAV13, AAVrh10, or AAVhu68.

In some embodiments, the gene therapy composition is a non-viral vector based gene therapy composition, such as liposomes or nanoparticles. In some embodiments, a method of treating a subject having a deficiency in glucocerebrosidase activity comprises administering to a subject in need thereof an effective amount of gene therapy composition comprising a liposome or nanoparticle, wherein the liposome or nanoparticle comprises an expression vector comprising a recombinant polynucleotide encoding an engineered glucocerebrosidase. In some embodiments, the expression vector is capable of expressing the encoded engineered glucocerebrosidase in mammalian cells.

In some embodiments, the present disclosure further provides use of an engineered glucocerebrosidase or a recombinant polynucleotide encoding the engineered glucocerebrosidase for the treatment of a deficiency of glucocerebrosidase in a subject.

In some embodiments, the present disclosure also provides use of an engineered glucocerebrosidase or a recombinant polynucleotide encoding the engineered glucocerebrosidase for the preparation of a medicament for treating a deficiency in glucocerebrosidase activity in a subject.

In some embodiments, the subject for treatment with an engineered glucocerebrosidase or a recombinant polynucleotide encoding the engineered glucocerebrosidase is afflicted with Gaucher disease. In some embodiments, the subject with Gaucher disease is diagnosed with type 1 GD.

In some embodiments, the subject for treatment with an engineered glucocerebrosidase or a recombinant polynucleotide encoding the engineered glucocerebrosidase is afflicted with Parkinson's disease.

### EXAMPLES

The following Examples, including experiments and results achieved, are provided for illustrative purposes only and are not to be construed as limiting the present invention.

In the experimental disclosure below, the following abbreviations apply where applicable: ppm (parts per million); M (molar); mM (millimolar), uM and μM (micromolar); nM (nanomolar); mol (moles); gm and g (gram); mg (milligrams); ug and μg (micrograms); L and l (liter); ml and mL (milliliter); ul, uL, μl, and μL (microliter); cm (centimeters); mm (millimeters); um and μm (micrometers); sec (seconds); min(s) (minute(s)); h(s) and hr(s) (hour(s)); U (units); MW (molecular weight); rpm (rotations per minute); ° C. (degrees Celsius); DNA (deoxyribonucleic acid); RNA (ribonucleic acid); and FIOPC (fold improvements over positive control).

### Example 1

### GBA1 Gene Acquisition and Construction of Expression Vectors

In this Example, GBA1 gene acquisition and expression vector construction are described. A synthetic gene coding for a WT human GCase (Uniprot ID P04062), including the native signal peptide sequence, was designed for optimized gene expression in Homo sapiens (SEQ ID NO: 1) using a CMV promoter and a BGH poly (A) signal. Directed evolution was used to generate specific gene variants derived from SEQ ID NO: 1 (see e.g., U.S. Pat. No. 8,383,346 and WO2010/144103).

### Example 2

### High-Throughput Growth of Suspension Mammalian Cells and GCase Assays

### High-Throughput (HTP) Growth and Expression of GCase and GCase Variants in Suspension Mammalian Cells (Expi293F™)

Expi293F™ cells (ThermoFisher Scientific) were transfected with DNA encoding wild-type GCase or GCase variants using the stable cationic polymer method with PEI Transporter 5 Transfection Reagent (Cat #26008-50) in Expi293™ Expression Medium (ThermoFisher Scientific). Expi93F™ cells were seeded into Axygen 1.1 mL deep well plates (Corning, P-DW-11-C-S), at 1×10⁶ cells/400 μL. Cells were subjected to transient transfection and returned to a shaking incubator with 8% CO₂ and 80% humidity for 2-6 days to allow for expression and secretion of GCase variants into the conditioned media. Conditioned media was harvested by centrifugation of expression plates and transfer of conditioned media into a BioRad Hardshell PCR Plate (BioRad, HSP9601) for activity, stability, or uptake into cell analysis.

### HTP Analysis of Conditioned Media

GCase variant activity, resulting from expression efficiency and stability to the neutral pH expression conditions, was determined by measuring the hydrolysis of 4-methylumbelliferyl-β-D-glucopyranoside (4-MU-βGLU). For the unchallenged assay, 5-25 μL of Expi293F™ clarified conditioned media produced as described above was mixed with 25-50 μL of 2.5-5 mM 4-MU-βGLU in Assay Buffer (50 mM citric acid, 176 mM K₂HPO₄, 0.01% Tween-20 and 10 mM sodium taurocholate, at pH 5), in a 96-well, black, opaque bottom plate. The reactions were incubated at 25-37° C. for 15-60 min with agitation at 400 rpm, and then quenched with 0.5 M sodium carbonate or glycine pH 10-10.5. Hydrolysis was analyzed using an En Vision microplate reader (Perkin Elmer) monitoring fluorescence (Ex. 355 nm, Em. 460 nm). Unchallenged activity FIOPC was calculated by dividing normalized GCase variant by the activity of the reference polypeptide with the indicated SEQ ID NO.

HTP Challenge with Neutral pH

GCase variants were challenged with neutral pH to simulate the pH conditions that the variants encounter in the blood following secretion from expression in the liver or administration to a patient as an enzyme replacement therapy. 12.5 μL of HTP clarified conditioned media from Expi293F expression plates was transferred into 37.5 μL of McIlvaine's Buffer (pH 7.2-7.4; Mellvaine, J. Biol. Chem., 1921, 49:183-186) and incubated for 1-2 hr at 37° C., 400 rpm. Residual activity post-challenge was then assessed with 50 μL of assay buffer (2.5-5 mM 4-MU-βGLU in 50 mM citric acid, 176 mM K₂HPO₄, 0.01% Tween-20 and 10 mM sodium taurocholate, at pH 5). Plates were sealed and incubated for 15-150 min at 37° C., 400 rpm and then quenched with 0.5 M sodium carbonate or glycine pH 10-10.5. Hydrolysis was analyzed using an En Vision microplate reader (Perkin Elmer) monitoring fluorescence (Ex. 355 nm, Em. 460 nm). Neutral pH stability FIOPC was calculated by dividing normalized GCase variant activity following challenge by the activity of the reference polypeptide with the indicated SEQ ID NO following challenge.

HTP Challenge with Serum

GCase variants were challenged with serum to simulate the pH conditions that the variants encounter in the blood following secretion from expression in the liver or administration to a patient as an enzyme replacement therapy. 12.5 μL of HTP clarified conditioned media from Expi293F expression plates was transferred into 37.5 μL of serum (Innovative Research ISERABHI100ML-Pooled Human AB Serum Plasma Derived Heat Inactivated) and incubated for 1-2 hr at 37° C., 400 rpm. Residual activity post-challenge was then assessed with 50 μL of assay buffer (2.5-5 mM 4-MU-βGLU in 50 mM citric acid, 176 mM K₂HPO₄, 0.01% Tween-20 and 10 mM sodium taurocholate, at pH 5). Plates were sealed and incubated for 15 to 150 min at 37° C., 400 rpm and then quenched with 0.5 M sodium carbonate or glycine pH 10-10.5. Hydrolysis was analyzed using an En Vision microplate reader (Perkin Elmer) monitoring fluorescence (Ex. 355 nm, Em. 460 nm). Serum stability FIOPC was calculated by dividing normalized GCase variant activity following challenge by the activity of the reference polypeptide with the indicated SEQ ID NO following challenge.

### HTP Challenge for Thermal Stability

GCase variants were assessed for thermal stability, a general measure of stability to the variety of conditions encountered during the life cycle of an exogenously delivered protein. 25 μL of HTP clarified conditioned media from Expi293F expression plates was incubated in BioRad hardshell plates for 1 hour at 44-47° C. Residual activity post-challenge was then assessed with 50 μL of assay buffer (2.5-5 mM 4-MU-βGLU in 50 mM citric acid, 176 mM K₂HPO₄, 0.01% Tween-20 and 10 mM sodium taurocholate, at pH 5). Plates were sealed and incubated for 15 to 60 min at 37° C., 400 rpm and then quenched with 0.5 M sodium carbonate or glycine pH 10-10.5. Hydrolysis was analyzed using an En Vision microplate reader (Perkin Elmer) monitoring fluorescence (Ex. 355 nm, Em. 460 nm). Thermal stability FIOPC was calculated by dividing normalized GCase variant activity following challenge by the activity of the reference polypeptide with the indicated SEQ ID NO following challenge.

HTP Challenge with Acidic pH

GCase variants were challenged with acidic pH to simulate the pH conditions that the variants encounter in the lysosome. 12.5 μL of HTP clarified conditioned media from Expi293F expression plates was transferred into 37.5 μL of Mellvaine's Buffer (pH 3-4.5; Mellvaine, J. Biol. Chem., 1921, 49:183-186) and incubated for 1-2 hr at 37° C., 400 rpm. Residual activity post-challenge was then assessed with 50 μL of assay buffer (2.5-5 mM 4-MU-βGLU in 50 mM citric acid, 176 mM K₂HPO₄, 0.01% Tween-20 and 10 mM sodium taurocholate, at pH 5). Plates were sealed and incubated for 15 to 150 min at 37° C., 400 rpm and then quenched with 0.5 M sodium carbonate or glycine pH 10-10.5. Hydrolysis was analyzed using an En Vision microplate reader (Perkin Elmer) monitoring fluorescence (Ex. 355 nm, Em. 460 nm). Acidic pH stability FIOPC was calculated by dividing normalized GCase variant activity following challenge by the activity of the reference polypeptide with the indicated SEQ ID NO following challenge.

### HTP Analysis of GCase Uptake and Activity in Lysates of Macrophages

Uptake of GCase into macrophages is key for the activity of existing enzyme replacement therapies and accumulation of sphingolipids within Gaucher cells drives pathology of the peripheral disease in patients. As such, GCase variants were screened for improved uptake into macrophages, and/or improved intracellular stability, as measured by evidence of increased observed GCase activity levels in macrophage lysates following treatment with HTP expression material. GCase variants from HTP Expi293F™ expression in clarified conditioned media were incubated with target cells and assayed for residual intracellular activity after 24-72 hr. For these experiments, macrophages (RAW264.7; ATCC TIB-71) were used (either containing functional endogenous GCase and observing supraphysiological levels of activity or using cells wherein the GBA1 gene had been knocked out). Macrophages were seeded into 96-well COSTAR® plates (3904, Corning) in and allowed to grow to confluency in 200 μL complete growth medium (Gibco™ Dulbecco's Modified Eagle Medium+1% Penicillin/Streptomycin, and 10% heat-inactivated fetal bovine serum). RAW264.7 macrophages were then treated with 50 μL of conditioned media produced by Expi293F™ cells transiently transfected as described above and allowed to incubate for 24-48 hr at 37° C., 5% CO₂. GCase containing conditioned media was removed, the cells were washed 2× with 150 μL 1×DPBS/well. Cells were lysed via addition of 50 μL of assay buffer (2.5-5 mM 4-MU-βGLU in 50 mM citric acid, 176 mM K₂HPO₄, 0.01% Tween-20 and 10 mM sodium taurocholate, at pH 5). The plates were sealed and shaken vigorously (>1000 rpm) at room temperature for 30-60 min. Reaction was quenched with 0.5 M glycine at pH 10. Hydrolysis was analyzed using a Envision® microplate reader monitoring fluorescence (Ex. 355 nm, Em. 460 nm). Liberation of 4-methylumbelliferone was analyzed using an En Vision microplate reader (Perkin Elmer) monitoring fluorescence. Cellular uptake FIOPC was calculated by dividing normalized GCase variant intracellular activity by the activity of the reference polypeptide with the indicated SEQ ID NO.

HTP Challenge with Artificial Cerebrospinal Fluid

GCase variants were challenged with artificial cerebrospinal fluid (aCSF) to simulate the conditions that the variants encounter in the central nervous system following expression in neurons or administration to a patient as an enzyme replacement therapy. 12.5 μL of HTP clarified conditioned media from Expi293F™ expression plates was transferred into 37.5 μL of aCSF (Tocris 3525) and incubated for 1-168 hr at 37° C., 400 rpm. Residual activity post-challenge was then assessed with 50 μL of assay buffer (1.25-5 mM 4-MU-βGLU in 50 mM citric acid, 176 mM K₂HPO₄, 0.01% Tween-20 and 10 mM sodium taurocholate, at pH 5). Plates were sealed and incubated for 15 to 150 min at 37° C., 400 rpm and then quenched with 0.5 M sodium carbonate or glycine pH 10-10.5. Hydrolysis was analyzed using an En Vision microplate reader (Perkin Elmer) monitoring fluorescence (Ex. 355 nm, Em. 460 nm). aCSF stability FIOPC was calculated by dividing normalized GCase variant activity following challenge by the activity of the reference polypeptide with the indicated SEQ ID NO following challenge.

### High-Throughput (HTP) Growth of GCase and GCase Variants in Adherent Liver-Derived Mammalian Cells

Many gene therapies utilize an AAV that transduces the liver, resulting in secretion of the replacement protein into circulation. Protein variants with increased protein expression and secretion from liver-derived cells could be advantageous. To identify variants with improved expression in liver-derived cells, HepG2 cells (ATCC HB-8065) were transfected with DNA encoding wild-type GCase or GCase variants using the stable cationic polymer method. HepG2 cells (ThermoFisher Scientific) were seeded into 96-well COSTAR® plates (3904, Corning), in Minimal Essential Media (MEM; Gibco #11095-080) supplemented with 10% fetal bovine serum (Corning #35-016-CV) at densities of 4×10⁴ cells/250 μL. Cells were subjected to transient transfection with PEI Transporter 5 Transfection Reagent (Cat #26008-50) and returned to a static incubator with 5% CO₂ and humidity for 3-4 days to allow for expression and secretion of GCase variants into the conditioned media. Activity was assessed by transfer of 10-25 μL of HTP conditioned media into 25-50 μL of assay buffer (2.5-5 mM 4-MU-βGLU, 50 mM citric acid, 176 mM K₂HPO₄, 0.01% Tween-20 and 10 mM sodium taurocholate, at pH 5). Plates were sealed and incubated for 2-4 hr at 37° C., 400 rpm, prior to quenching with 0.5 M sodium carbonate or glycine pH 10-10.5. Hydrolysis was analyzed using an En Vision microplate reader (Perkin Elmer) monitoring fluorescence (Ex. 355 nm, Em. 460 nm). Liver Expression and Activity FIOPC was calculated by dividing normalized GCase variant by the activity of the reference polypeptide with the indicated SEQ ID NO.

### Example 3

### GCase Variants of SEQ ID NO: 2

In this Example, experiments for evolution and screening of GCase enzyme variants derived from SEQ ID NO: 2 for improved GCase activity after a series of challenges are described. Libraries of variant GBA1 genes encoded based off SEQ ID NO: 1 were constructed and expressed in Expi293F cells for assessment of GCase 4-MU-βGLU activity (“Unchallenged Activity FIOPC”, described in Example 2), as well as after neutral pH challenge (“Neutral pH Stability and Activity FIOPC”, as described in Example 2), and thermal challenge (“Thermal Stability and Activity FIOPC”). Libraries of variant GBA1 genes encoded based off SEQ ID NO: 1 were also expressed in HepG2 cells for assessment of GCase 4-MU-βGLU activity (“Liver Expression and Activity FIOPC”, described in Example 2). The results of these assays are presented in Tables 3.1 and 3.2.

TABLE 3.1
Activity of GCase Variants Relative to SEQ ID NO: 2¹

| | | | Neutral | | |
|---|---|---|---|---|---|
| | | | pH | | Liver |
| | Amino Acid | Un- | Stability | Thermal | Expression |
| SEQ | Differences | challenged | and | Stability | and |
| ID NO: | (Relative to | Activity | Activity | and Activity | Activity |
| (nt/aa) | SEQ ID NO: 2) | FIOPC | FIOPC | FIOPC | FIOPC |
| 1/2 | | + | + | + | + |
| 3/4 | Q265T/N372D/M489L | ++ | + | + | + |
| 5/6 | I243K | + | + | | |
| 7/8 | G23A | ++ | ++ | + | + |
| 9/10 | Q109D | ++ | + | | + |
| 11/12 | Q265T | ++ | ++ | + | + |
| 13/14 | Q109D/L195M/I243K | ++ | ++ | + | ++ |
| 15/16 | G23A/F70L/A207S/L214F/Q265T | +++ | ++++ | +++ | +++ |
| 17/18 | G23A/F70L | ++ | ++ | ++ | ++ |
| 19/20 | G23A/L214F/I243K/Q265T | ++ | ++ | ++ | ++ |
| 21/22 | F65L/F70L/Q109D/L214F/M319I/N372D/ | +++ | ++++ | ++++ | ++++ |
| | N481T | | | | |
| 23/24 | G23A/Q265T/M319I | +++ | +++ | ++ | +++ |
| 25/26 | G23A/F65L/F70L/N141D/M319I/N481T | +++ | +++ | +++ | ++ |
| 27/28 | G23A/Q265T | ++ | ++ | ++ | + |
| 29/30 | N372D | ++ | + | + | + |
| 31/32 | L214F | + | ++ | ++ | |
| 33/34 | G23A/F65L/F70L/Q109D/N141D/L214F | +++ | ++++ | +++ | |
| 35/36 | M319I | ++ | +++ | ++ | ++ |
| 37/38 | F65L/N372D | ++ | ++ | ++ | |
| 39/40 | Q109D/L195M | | | ++ | ++ |
| 41/42 | G23A/F65L | ++ | ++ | ++ | ++ |
| 43/44 | A207S/Q265T/M319I | +++ | ++++ | +++ | +++ |
| 45/46 | Q109D/A207S/L214F/N372D | +++ | ++++ | ++ | +++ |
| 47/48 | G23A/M319I/N372D/M489L | +++ | +++ | ++ | +++ |
| 49/50 | F65L/Q109D/L214F/I243K | +++ | +++ | ++ | +++ |
| 51/52 | G23A/N141D/L195M | ++ | ++ | ++ | ++ |
| 53/54 | F70L/A207S/L214F/M489L | +++ | ++++ | +++ | + |
| 55/56 | F65L/F70L/Q265T/M319I/N372D | +++ | ++++ | +++ | +++ |
| 57/58 | L214F/Q265T | +++ | +++ | ++ | +++ |
| 59/60 | Q265T/M319I | +++ | +++ | ++ | ++ |
| 61/62 | F65L/I243K | ++ | + | ++ | + |
| 63/64 | F65L/F70L/L195M | ++ | ++ | ++ | ++ |
| 65/66 | M489L | ++ | | + | |
| 67/68 | G23A/F65L/N141D | ++ | ++ | ++ | |
| 69/70 | G23A/N372D | ++ | ++ | + | + |
| 71/72 | F70L/N141D | ++ | ++ | ++ | ++ |
| 73/74 | G23A/L195M | ++ | ++ | ++ | + |
| 75/76 | L214F/M319I | +++ | +++ | ++++ | +++ |
| 77/78 | G23A/A207S/L214F/I243K/Q265T/N481T | +++ | +++ | ++ | +++ |
| 79/80 | F65L/L214F/I243K/Q265T/M319I | ++++ | ++++ | ++++ | ++++ |
| 81/82 | L214F/I243K | ++ | ++ | ++ | ++ |
| 83/84 | I243K/Q265T | ++ | | | ++ |
| 85/86 | Q265T/M489L | ++ | ++ | +++ | + |
| 87/88 | L214F/I243K/Q265T | +++ | +++ | | +++ |
| 89/90 | A207S/I243K/N372D | +++ | ++ | | ++ |
| 91/92 | L195M/I243K | ++ | ++ | ++ | ++ |
| 93/94 | F65L/N481T | ++ | ++ | | ++ |
| 95/96 | A207S/L214F/I243K/M319I | +++ | ++++ | | ++++ |
| 97/98 | F70L/N141D/L195M/Q265T | ++ | ++ | ++ | ++ |
| 99/100 | G23A/Q265T/N372D | ++ | + | | + |

Levels of increased activity were determined relative to the reference polypeptide of SEQ ID NO: 2 and defined as follows:
“+” >0.9,
“++” >1.15,
“+++” >3,
“++++” >6

TABLE 3.2
Activity of GCase Variants Relative to SEQ ID NO: 2¹

| | | | Neutral pH | Thermal | Liver |
|---|---|---|---|---|---|
| | Amino Acid | Un- | Stability | Stability | Expression |
| SEQ ID | Differences | challenged | and | and | and |
| NO: | (Relative to | Activity | Activity | Activity | Activity |
| (nt/aa) | SEQ ID NO: 2) | FIOPC | FIOPC | FIOPC | FIOPC |
| 1/2 | | + | + | + | + |
| 101/102 | K46R/H99R/A134S/V230L/T373Y/T449V | ++ | +++ | ++ | +++ |
| 103/104 | P68L/V211L/V230L | ++ | +++ | ++ | ++ |
| 105/106 | T373Y/T449V | | | + | ++ |
| 107/108 | K46R/P68V/T336A | ++ | ++ | ++ | ++ |
| 109/110 | P68V/V230L/N314D/T373Y | ++ | +++ | ++ | +++ |
| 111/112 | P68V | ++ | ++ | ++ | ++ |
| 113/114 | P68L/T336A/T373Y | ++ | ++ | ++ | ++ |
| 115/116 | H99R/N314D | | + | + | + |
| 117/118 | K46R | | + | | |
| 119/120 | K46R/V230L/E261K | + | ++ | + | |
| 121/122 | T373Y | | | | + |
| 123/124 | V230L/N314D | + | +++ | + | + |
| 125/126 | P68L/V211L | | ++ | + | + |
| 127/128 | P68V/T336A/T449V | ++ | ++ | ++ | ++ |
| 129/130 | K46R/T336A | | ++ | + | + |
| 131/132 | T336A/T449V | ++ | ++ | + | ++ |
| 133/134 | K46R/P68V/T336A/T373Y | ++ | ++ | ++ | ++ |
| 135/136 | K46R/H99R | | + | + | + |
| 137/138 | H99R/E261K | | | + | |
| 139/140 | H99R | ++ | ++ | + | ++ |
| 141/142 | P68V/A134S | ++ | ++ | ++ | ++ |
| 143/144 | K46R/P68V/A134S/V230L/T336A/T373Y | +++ | ++++ | ++ | +++ |
| 145/146 | K46R/P68V | ++ | ++ | ++ | ++ |
| 147/148 | P68L/H99R/V211L/V230L | +++ | +++ | ++ | +++ |
| 149/150 | H99R/A134S | ++ | + | ++ | ++ |
| 151/152 | P68V/H99R/T336A | ++ | ++ | ++ | +++ |
| 153/154 | T449V | + | + | + | + |
| 155/156 | P68V/H99R | ++ | ++ | ++ | ++ |
| 157/158 | K46R/V230L/T336A | ++ | +++ | ++ | ++ |
| 159/160 | P68V/G154E/T449V | ++ | +++ | ++ | ++ |
| 161/162 | T336A/T373Y/T449V | | ++ | + | ++ |
| 163/164 | A134S/T336A | ++ | ++ | + | ++ |
| 165/166 | P68V/H99R/V230L | +++ | +++ | ++ | ++ |
| 167/168 | P68L | ++ | ++ | + | ++ |
| 169/170 | K46R/P68V/A134S/G154E | ++ | +++ | ++ | ++ |
| 171/172 | P68V/H99R/V230L/T373Y/T449V | +++ | +++ | ++ | +++ |
| 173/174 | A134S | + | ++ | + | + |
| 175/176 | K46R/P68V/G154E/V211L/V230L/E261K/ | +++ | ++++ | ++ | +++ |
| | T336A | | | | |
| 177/178 | P68V/H99R/G154E/E261K/T336A | ++ | +++ | +++ | +++ |
| 179/180 | V230L/T449V | + | +++ | + | +++ |
| 181/182 | K46R/P68L/N314D | | ++ | + | ++ |
| 183/184 | G154E | + | ++ | ++ | ++ |
| 185/186 | P68L/T449V | ++ | ++ | ++ | ++ |
| 187/188 | H99R/T449V | + | ++ | + | + |
| 189/190 | P68V/G154E/V230L/N314D/T336A/T449V | +++ | ++++ | +++ | +++ |
| 191/192 | K46R/P68V/G154E/N314D/T336A/T373Y | ++ | +++ | ++ | +++ |
| 193/194 | K46R/V230L | ++ | +++ | + | ++ |
| 195/196 | T336A | + | ++ | + | ++ |
| 197/198 | K46R/H99R/V230L/T373Y | ++ | +++ | + | ++ |

¹Levels of increased activity were determined relative to the reference polypeptide of SEQ ID NO: 2 and defined as follows: “+” >0.9, “++” >1.15, “+++” >2, “++++” >4

### Example 4

### GCase Variants of SEQ ID NO: 54

In this Example, experiments for evolution and screening of GCase enzyme variants derived from SEQ ID NO: 54 for improved GCase activity after a series of challenges are described. Libraries of variant GBA1 genes encoded based off SEQ ID NO: 53 were constructed and expressed in Expi293F cells for assessment of GCase 4-MU-βGLU activity (“Unchallenged Activity FIOPC”, described in Example 2), as well as after neutral pH challenge (“Neutral pH Stability and Activity FIOPC”, as described in Example 2), and/or serum challenge (“Serum Stability and Activity FIOPC”, as described in Example 2), and/or thermal challenge (“Thermal Stability and Activity FIOPC”, as described in Example 2), and/or acidic pH challenge (“Acidic pH Stability and Activity FIOPC”, as described in Example 2). Libraries of variant GBA1 genes encoded based off SEQ ID NO: 53 were also expressed in HepG2 cells for assessment of GCase 4-MU-βGLU activity (“Liver Expression and Activity FIOPC”, described in Example 2). The results of these assays are presented in Tables 4.1, 4.2, 4-3, and 4.4.

TABLE 4.1
Activity of GCase Variants Relative to SEQ ID NO: 54¹

| | | | Neutral pH | Thermal | Liver |
|---|---|---|---|---|---|
| | Amino Acid | Un- | Stability | Stability | Expression |
| SEQ ID | Differences | challenged | and | and | and |
| NO: | (Relative to | Activity | Activity | Activity | Activity |
| (nt/aa) | SEQ ID NO: 54) | FIOPC | FIOPC | FIOPC | FIOPC |
| 53/54 | | + | + | + | + |
| 199/200 | P68L/T336A | + | ++ | ++ | ++ |
| 201/202 | F65L/H99R/M319I | ++ | +++ | +++ | ++ |
| 203/204 | F65L/P68L/M319I | ++ | +++ | +++ | ++ |
| 205/206 | P68V/M319I | ++ | +++ | +++ | +++ |
| 207/208 | P68V | + | ++ | ++ | + |
| 209/210 | F65L/M319I | ++ | +++ | +++ | +++ |
| 211/212 | L195M | + | | + | + |
| 213/214 | F65L/G154E/M319I | ++ | +++ | ++++ | +++ |
| 215/216 | F65L/P68L/V230L | + | +++ | +++ | ++ |
| 217/218 | F65L | + | ++ | ++ | ++ |
| 219/220 | F65L/P68L/H99R/G154E/V230L/M319I | ++ | +++ | ++++ | +++ |
| 221/222 | P68L/V230L | + | +++ | ++ | ++ |
| 223/224 | F65L/P68V/M319I | ++ | +++ | +++ | +++ |
| 225/226 | L195M/V230L | + | +++ | ++ | ++ |
| 227/228 | F65L/V230L | + | +++ | +++ | +++ |
| 229/230 | N141D/M319I | + | +++ | +++ | +++ |
| 231/232 | G154E/T336A | + | + | ++ | ++ |
| 233/234 | P68L/M319I | + | +++ | +++ | +++ |
| 235/236 | P68V/G154E | + | ++ | +++ | ++ |
| 237/238 | P68V/L195M/V230L/M319I/T336A | ++ | +++ | ++++ | +++ |
| 239/240 | P68V/H99R/L195M/V230L/M319I | + | +++ | ++++ | +++ |
| 241/242 | P68V/L195M | + | | ++ | ++ |
| 243/244 | F65L/P68V | + | ++ | ++ | ++ |
| 245/246 | P68L/N141D/V230L/M319I | ++ | +++ | ++++ | +++ |
| 247/248 | F65L/P68V/H99R/V230L/M319I | ++ | +++ | ++++ | +++ |
| 249/250 | P68L/H99R/N141D/M319I | + | ++ | ++++ | +++ |
| 251/252 | P68V/G154E/M319I | ++ | +++ | ++++ | +++ |
| 253/254 | N141D/G154E | + | + | ++ | ++ |
| 255/256 | P68V/H99R | + | ++ | ++ | |
| 257/258 | H99R/V230L/M319I | + | +++ | ++++ | ++ |
| 259/260 | H99R | + | + | + | |
| 261/262 | V230L | + | +++ | ++ | ++ |
| 263/264 | P68V/V230L/M319I | ++ | +++ | ++++ | +++ |
| 265/266 | P68L | + | ++ | ++ | + |
| 267/268 | G154E/M319I | + | ++ | +++ | ++ |
| 269/270 | F65L/P68L/G154E/M319I | ++ | +++ | ++++ | +++ |
| 271/272 | N141D | + | + | ++ | |
| 273/274 | P68V/V230L | + | +++ | +++ | +++ |
| 275/276 | T336A | + | | + | |
| 277/278 | N141D/V230L | + | +++ | ++ | ++ |
| 279/280 | P68V/G154E/T336A | + | ++ | +++ | ++ |
| 281/282 | F65L/P68L | + | ++ | ++ | + |
| 283/284 | F65L/P68V/H99R/M319I | ++ | +++ | ++++ | +++ |
| 285/286 | G154E | + | + | ++ | + |
| 287/288 | M319I | + | ++ | +++ | +++ |
| 289/290 | V230L/M319I | ++ | +++ | ++++ | +++ |
| 291/292 | P68L/G154E | + | ++ | ++ | |
| 293/294 | H99R/N141D/M319I | + | ++ | +++ | +++ |
| 295/296 | P68L/G154E/M319I | ++ | +++ | +++ | ++ |

Levels of increased activity were determined relative to the reference polypeptide of SEQ ID NO: 54 and defined as follows: “+” >0.9, “++” >1.15, “+++” >2, “++++” >5

TABLE 4.2
Activity of GCase Variants Relative to SEQ ID NO: 54¹

| | | | Neutral pH | Thermal | Liver |
|---|---|---|---|---|---|
| | Amino Acid | | Stability | Stability | Expression |
| SEQ ID | Differences | Un-challenged | and | and | and |
| NO: | (Relative to | Activity | Activity | Activity | Activity |
| (nt/aa) | SEQ ID NO: 54) | FIOPC | FIOPC | FIOPC | FIOPC |
| 53/54 | | + | + | + | + |
| 297/298 | L509F | + | + | + | + |
| 299/300 | L108I | + | + | + | ++ |
| 301/302 | L108I/V117I/L509F | + | + | + | |
| 303/304 | L519I | + | + | + | + |
| 305/306 | L108I/L519I | + | | + | |
| 307/308 | M88L | + | | | + |
| 309/310 | V117I/H413Y/L509F | + | | | + |
| 311/312 | L509F/L519I | + | | + | |
| 313/314 | H413Y | + | | | + |
| 315/316 | L108I/V117I/V315T | + | | | |
| 317/318 | V117I/A271T | + | | | |
| 319/320 | V117I/H413Y/L509F/H529Q | + | | + | |
| 321/322 | H413Y/L509F | + | | | |

¹Levels of increased activity were determined relative to the reference polypeptide of SEQ ID NO: 54 and defined as follows: “+” >0.9, “++” >1.15

TABLE 4.3
Activity of GCase Variants Relative to SEQ ID NO: 54¹

| | | | Neutral | |
|---|---|---|---|---|
| | | | pH | Thermal |
| | Amino Acid | Un- | Stability | Stability |
| SEQ ID | Differences | challenged | and | and |
| NO: | (Relative to | Activity | Activity | Activity |
| (nt/aa) | SEQ ID NO: 54) | FIOPC | FIOPC | FIOPC |
| 53/54 | | + | + | + |
| 323/324 | G469E | + | + | + |
| 325/326 | P524K | | | + |
| 327/328 | V507M | + | + | + |
| 329/330 | L509I | + | ++ | + |
| 331/332 | G525Q | | | + |
| 333/334 | L489A | | | + |
| 335/336 | L104M | + | + | + |
| 337/338 | V507A | + | + | + |
| 339/340 | I95G | | | + |
| 341/342 | A359E | + | | + |
| 343/344 | M374V | + | ++ | + |
| 345/346 | L509W | | | + |
| 347/348 | T107D | + | + | + |
| 349/350 | E89C | + | + | + |
| 351/352 | A260R | + | | + |
| 353/354 | P110Q | + | ++ | + |
| 355/356 | F186G | | | + |
| 357/358 | F370R | | + | + |
| 359/360 | L509R | + | + | + |
| 361/362 | E191R | + | + | + |
| 363/364 | V333L | | | + |
| 365/366 | P189R | | | + |
| 367/368 | E468G | | | + |
| 369/370 | V473I | + | + | ++ |
| 371/372 | Q115M | | | + |
| 373/374 | E191N | | | ++ |
| 375/376 | V53L | | | ++ |
| 377/378 | P524S | + | | |
| 379/380 | T449L | | | ++ |
| 381/382 | F518E | | | ++ |
| 383/384 | L236M | | | ++ |
| 385/386 | N481H | + | | |
| 387/388 | Q115R | | | + |
| 389/390 | A59N/S220G | | | ++ |
| 391/392 | L489G | | | + |
| 393/394 | A258T | | | ++ |
| 395/396 | P467N | | | + |
| 397/398 | S51P | | | + |
| 399/400 | V415C | | | ++ |
| 401/402 | G103S | + | + | + |
| 403/404 | G493T | | + | |
| 405/406 | L489T | + | | + |
| 407/408 | F518V | + | + | + |
| 409/410 | R78L | + | + | + |
| 411/412 | L195W | + | ++ | +++ |
| 413/414 | F65M | + | ++ | + |
| 415/416 | H413W | + | | + |
| 417/418 | L335M | | | + |
| 419/420 | E520K | + | + | + |
| 421/422 | T107E | + | + | + |
| 423/424 | G101Q | + | + | + |
| 425/426 | T449K | + | + | + |
| 427/428 | N481K | | + | |
| 429/430 | L104V | + | + | + |
| 431/432 | A341R | + | | + |
| 433/434 | R78M | + | | + |
| 435/436 | V507L | | + | |
| 437/438 | T449M | | | + |
| 439/440 | Q471L | + | ++ | + |
| 441/442 | V211L | | + | + |
| 443/444 | E520D | + | | + |
| 445/446 | G103P | | + | |
| 447/448 | L206F | + | + | + |
| 449/450 | G93S | | | + |
| 451/452 | H490S | | + | + |
| 453/454 | V473C | | + | |
| 455/456 | V507R | | | + |
| 457/458 | V211I | + | ++ | + |
| 459/460 | P71S | + | + | + |
| 461/462 | Y79R | | + | |
| 463/464 | D180C | ++ | +++ | + |
| 465/466 | L509G | + | + | |
| 467/468 | L206R | | + | + |
| 469/470 | V211N | + | + | + |
| 471/472 | Y172S | | ++ | + |
| 473/474 | H490R | ++ | ++ | + |
| 475/476 | S207D | + | ++ | + |
| 477/478 | Q115V | + | ++ | + |
| 479/480 | D63L | | | + |
| 481/482 | L142A | + | ++ | + |
| 483/484 | H529M | + | | + |
| 485/486 | P71R | + | ++ | + |
| 487/488 | V473L | + | + | + |
| 489/490 | I95T | | | + |
| 491/492 | T107A | + | + | + |
| 493/494 | E520V | | | + |
| 495/496 | G101L | | + | + |
| 497/498 | E520S | + | + | + |
| 499/500 | P71H | + | ++ | + |
| 501/502 | E89D | + | + | + |
| 503/504 | S77I | ++ | +++ | ++ |
| 505/506 | V507Y | + | + | |
| 507/508 | T107C | | | + |
| 509/510 | F518S | + | + | + |
| 511/512 | S212A | + | ++ | + |
| 513/514 | P71F | + | + | |
| 515/516 | G525C | | | + |
| 517/518 | T449R | | | |
| 519/520 | T107M | | | + |
| 521/522 | P467H | + | | + |
| 523/524 | G474D | + | + | |
| 525/526 | G101S | + | + | + |
| 527/528 | L106W | + | + | + |
| 529/530 | F386L/V507M | | + | |
| 531/532 | Q471S | + | | |
| 533/534 | T107Q | + | | + |
| 535/536 | P68Q | | + | |
| 537/538 | Y79I | + | +++ | + |
| 539/540 | F518L | + | + | |
| 541/542 | G474A | + | + | |
| 543/544 | T107S | + | + | + |
| 545/546 | V117T | | | + |
| 547/548 | P467M | | + | |
| 549/550 | Q471R | + | + | + |
| 551/552 | A341S | + | + | + |
| 553/554 | H490G | + | | + |
| 555/556 | Q471F | + | + | |
| 557/558 | H490T | + | | + |
| 559/560 | V333T | + | | |
| 561/562 | G101R | + | | + |
| 563/564 | T373V | + | + | + |
| 565/566 | G101V | + | ++ | + |
| 567/568 | G101W | + | ++ | + |
| 569/570 | F65S | + | ++ | + |
| 571/572 | H490V | + | + | + |
| 573/574 | D337N | + | ++ | ++ |
| 575/576 | M374T | + | ++ | ++ |
| 577/578 | P68Y | + | + | + |
| 579/580 | I95L | + | ++ | + |
| 581/582 | P71G | + | ++ | + |
| 583/584 | Q471V | + | ++ | + |
| 585/586 | T107M/Q246R | + | | + |
| 587/588 | P110A | + | + | |
| 589/590 | L142Y | + | | |
| 591/592 | F65V | ++ | +++ | +++ |
| 593/594 | P71M | + | + | + |
| 595/596 | S77G | + | | |
| 597/598 | S77R | + | + | + |
| 599/600 | V230Y | +++ | +++ | +++ |
| 601/602 | H490Q | + | + | + |
| 603/604 | G101T | + | + | + |
| 605/606 | G103A | + | + | |
| 607/608 | V507T | | + | |
| 609/610 | Q471T | + | +++ | + |
| 611/612 | L509V | + | + | + |
| 613/614 | E520R | + | | |
| 615/616 | M319F | | + | |
| 617/618 | L142N | + | + | + |
| 619/620 | Y79V | + | ++ | ++ |
| 621/622 | P467L | + | + | + |
| 623/624 | S77N | + | + | + |
| 625/626 | P71L | + | + | |
| 627/628 | P68W | + | | + |
| 629/630 | P189S | | | + |
| 631/632 | A359P | ++ | +++ | ++ |
| 633/634 | P71Y | + | | |
| 635/636 | S77T | + | ++ | + |
| 637/638 | L142M | + | + | |
| 639/640 | T373A | | | + |
| 641/642 | M319V | + | ++ | ++ |
| 643/644 | S212N | + | + | + |
| 645/646 | P110H | + | | |
| 647/648 | V333C | | | + |
| 649/650 | H490I | | | + |
| 651/652 | P68F | + | + | + |
| 653/654 | G474S | + | + | + |
| 655/656 | G103L | | | + |
| 657/658 | S77H | + | + | + |
| 659/660 | Q471E | + | + | + |
| 661/662 | V516L | + | + | + |
| 663/664 | P467R | | | + |

¹Levels of increased activity were determined relative to the reference polypeptide of SEQ ID NO: 54 and defined as follows: “+” >=0.9, “++” >1.15, “+++” >1.5

TABLE 4.4
Activity of GCase Variants Relative to SEQ ID NO: 54¹

| | | | Neutral | | | |
|---|---|---|---|---|---|---|
| | Amino Acid | | pH | Serum | Thermal | Acidic pH |
| | Amino Acid | Un- | Stability | Stability | Stability | Stability |
| SEQ ID | Differences | challenged | and | and | and | and |
| NO: | (Relative to | Activity | Activity | Activity | Activity | Activity |
| (nt/aa) | SEQ ID NO: 54) | FIOPC¹ | FIOPC¹ | FIOPC¹ | FIOPC¹ | FIOPC¹ |
| 53/54 | | + | + | + | + | + |
| 665/666 | K233R | + | +++ | ++ | ++ | |
| 667/668 | S91T | | | | + | |
| 669/670 | K194L | + | + | + | + | |
| 671/672 | S84G | ++ | + | +++ | + | ++ |
| 673/674 | A361V | | | | + | |
| 675/676 | K233A | ++ | ++++ | ++++ | + | +++ |
| 677/678 | T171V | +++ | ++++ | ++++ | +++ | ++++ |
| 679/680 | Q536L | ++ | ++ | +++ | + | +++ |
| 681/682 | Y50A | | | | | + |
| 683/684 | K116R | + | + | ++ | ++ | + |
| 685/686 | Q239T | + | + | + | + | + |
| 687/688 | I44V | + | | | + | |
| 689/690 | A40S | | + | | ++ | |
| 691/692 | K113N | + | | + | | |
| 693/694 | S91I | | | | ++++ | ++ |
| 695/696 | H99F | + | + | + | + | |
| 697/698 | I243L | | | | + | |
| 699/700 | I169V | + | + | ++ | + | |
| 701/702 | S504P | + | ++ | ++ | + | + |
| 703/704 | K505R | ++ | ++ | +++ | + | ++ |
| 705/706 | Q109R | + | + | | + | |
| 707/708 | Q536T | + | ++ | ++ | + | + |
| 709/710 | Q479A | + | ++ | + | + | + |
| 711/712 | Q479L | + | + | + | | + |
| 713/714 | A477H | + | +++ | +++ | ++ | ++ |
| 715/716 | S84K | | | | + | + |
| 717/718 | A97N | | | | + | |
| 719/720 | A97K | + | ++ | + | + | + |
| 721/722 | Q182M | ++ | ++ | ++ | + | + |
| 723/724 | E111H | + | ++ | ++ | + | ++ |
| 725/726 | A361Y | + | ++ | +++ | +++ | +++ |
| 727/728 | P508R | ++ | ++ | ++ | + | ++ |
| 729/730 | N314R | | + | + | | |
| 731/732 | T336G | + | + | | + | ++ |
| 733/734 | T102E | | | | + | + |
| 735/736 | P338R | ++ | ++ | +++ | + | ++ |
| 737/738 | K505L | ++ | ++ | ++ | ++ | ++ |
| 739/740 | H99L | + | ++ | + | | + |
| 741/742 | Q96S | ++ | ++ | ++ | + | ++ |
| 743/744 | P138S | ++ | ++ | ++ | + | ++ |
| 745/746 | P508T | + | +++ | ++ | + | + |
| 747/748 | Q96I | + | ++ | ++ | + | + |
| 749/750 | P508Q | + | ++ | ++ | +++ | ++ |
| 751/752 | I243M | | | | + | |
| 753/754 | E111L | + | + | + | | + |
| 755/756 | P508S | + | +++ | +++ | ++++ | +++ |
| 757/758 | Q479R | + | +++ | +++ | ++++ | ++ |
| 759/760 | P42A | | | | | + |
| 761/762 | Q239I | | | | + | |
| 763/764 | A97I | + | + | ++ | + | + |
| 765/766 | K116E | + | + | + | | |
| 767/768 | K113I | | + | | | + |
| 769/770 | A477V | + | | | | |
| 771/772 | Q536I | ++ | +++ | +++ | +++ | +++ |
| 773/774 | K263G | ++ | +++ | +++ | ++ | +++ |
| 775/776 | H99S | + | ++ | ++ | + | + |
| 777/778 | T222I | ++ | +++ | +++ | + | ++ |
| 779/780 | Q536D | ++ | +++ | ++ | + | ++ |
| 781/782 | A477G | + | ++ | ++ | | |
| 783/784 | Q536W | + | + | | | |
| 785/786 | Q536C | | + | + | | |
| 787/788 | T336R | + | + | | ++ | + |
| 789/790 | I243R | | | | + | |
| 791/792 | K116V | + | ++ | ++ | + | |
| 793/794 | Q536R | + | + | + | + | + |
| 795/796 | I522V | + | + | + | + | |
| 797/798 | T102S | + | + | + | + | ++ |
| 799/800 | A97R | ++ | ++ | ++ | + | + |
| 801/802 | A40H | + | ++ | + | | |
| 803/804 | I243V | | | | + | |
| 805/806 | Q265S | + | ++ | ++ | + | |
| 807/808 | K116Q | + | + | | | |
| 809/810 | P514E | + | ++ | ++ | ++++ | ++ |
| 811/812 | P508L | ++ | ++ | +++ | | |
| 813/814 | A515T | + | + | + | + | |
| 815/816 | Q246R | + | | | + | |
| 817/818 | A361W | + | ++ | +++ | ++++ | +++ |
| 819/820 | E111F | + | + | | | |
| 821/822 | L489M | + | + | + | + | |
| 823/824 | A477R | + | ++ | ++ | + | |
| 825/826 | K116Y | | + | | + | |
| 827/828 | K46R | + | + | + | | + |
| 829/830 | L133M | | | | + | |
| 831/832 | K332R | | | | + | |
| 833/834 | Q239R | ++ | +++ | +++ | +++ | +++ |
| 835/836 | Q109G | + | ++ | ++ | | ++ |
| 837/838 | D506T | + | + | + | + | |
| 839/840 | S504G | + | | | + | + |
| 841/842 | K233Q | ++ | ++++ | +++ | | ++ |
| 843/844 | Q96R | + | ++ | ++ | + | |
| 845/846 | H99G | + | + | + | + | |
| 847/848 | S504T | | | | + | + |
| 849/850 | Q96G | + | ++ | + | + | + |
| 851/852 | A97M | | + | | | |
| 853/854 | Q109L | + | ++ | ++ | | + |
| 855/856 | A515G | + | + | + | + | |
| 857/858 | K233G | + | +++ | +++ | + | + |
| 859/860 | S84A | + | | | + | + |
| 861/862 | T102N | | + | | + | + |
| 863/864 | S504W | + | + | + | ++ | |
| 865/866 | Q265R | ++ | +++ | +++ | + | + |
| 867/868 | H99V | + | + | + | + | ++ |
| 869/870 | K512L | + | + | ++ | + | + |
| 871/872 | P137V | + | + | + | + | ++ |
| 873/874 | Q536A | + | ++ | ++ | + | ++ |
| 875/876 | K113R | | | | + | |
| 877/878 | T171P | | | | + | |
| 879/880 | E190W | + | +++ | +++ | +++ | |
| 881/882 | N314G | + | + | ++ | | |
| 883/884 | K46M | | | | + | |
| 885/886 | Q182H | + | + | + | + | |
| 887/888 | Q246K | ++ | ++ | +++ | ++++ | +++ |
| 889/890 | L105T | + | + | + | | + |
| 891/892 | R301K | + | + | + | ++ | ++ |
| 893/894 | I522T | + | + | + | + | + |
| 895/896 | H329L | | | | +++ | |
| 897/898 | Q96V | + | | | + | + |
| 899/900 | E339A | | | | + | |
| 901/902 | A477S | + | + | + | ++ | + |
| 903/904 | K113G | + | + | + | | |
| 905/906 | A361L | + | ++ | ++ | ++ | ++ |
| 907/908 | K505I | | | | ++ | |
| 909/910 | L105M | + | + | ++ | + | |
| 911/912 | K116M | + | + | + | | |
| 913/914 | Q96M | + | ++ | ++ | + | ++ |
| 915/916 | Q109M | + | +++ | ++ | + | ++ |
| 917/918 | T171C | | | | +++ | |
| 919/920 | Q536M | + | +++ | ++ | + | + |
| 921/922 | E151L | | | | + | |

¹Levels of increased activity were determined relative to the reference polypeptide of SEQ ID NO: 54 and defined as follows:
“+” >0.9,
“++” >1.15,
“+++” >1.5,
“++++” >2.5

### Example 5

### GCase Variants of SEQ ID NO: 220

In this Example, experiments for evolution and screening of GCase enzyme variants derived from SEQ ID NO: 220 for improved GCase activity after a series of challenges are described. Libraries of variant GBA1 genes encoded based off SEQ ID NO: 219 were constructed and expressed in Expi293F cells for assessment of GCase 4-MU-βGLU activity (“Unchallenged Activity FIOPC”, described in Example 2), as well as after serum challenge (“Serum Stability and Activity FIOPC”, as described in Example 2), thermal challenge (“Thermal Stability and Activity FIOPC”, as described in Example 2), acidic pH challenge (“Acidic pH Stability and Activity FIOPC”, as described in Example 2), and uptake into macrophages (“Macrophage Uptake FIOPC”, as described in Example 2). Libraries of variant GBA1 genes encoded based off SEQ ID NO: 219 were also expressed in HepG2 cells for assessment of GCase 4-MU-βGLU activity (“Liver Expression and Activity FIOPC,” described in Example 2). The results of these assays are presented in Table 5.1.

TABLE 5.1
Activity of GCase Variants Relative to SEQ ID NO: 220¹

| | Amino Acid | | Serum | Thermal | Acidic pH | | Liver |
|---|---|---|---|---|---|---|---|
| | Differences | Un- | Stability | Stability | Stability | | Expression |
| SEQ ID | (Relative to | challenged | and | and | and | Macrophage | and |
| NO: | SEQ ID NO: | Activity | Activity | Activity | Activity | Uptake | Activity |
| (nt/aa) | 220) | FIOPC¹ | FIOPC¹ | FIOPC¹ | FIOPC¹ | FIOPC¹ | FIOPC¹ |
| 219/220 | | + | + | + | + | + | + |
| 923/924 | L65V/L195W/ | ++ | +++ | +++ | + | ++ | |
| | A361W | | | | | | |
| 925/926 | P514E | | ++ | + | + | + | |
| 927/928 | Y79V/L195W/ | ++ | +++ | ++ | + | ++ | ++ |
| | K263G/A361W | | | | | | |
| 929/930 | L65V/M374T | ++ | +++ | + | + | + | ++ |
| 931/932 | Y79V/L195W | + | ++ | + | + | + | ++ |
| 933/934 | L65V/L230Y | ++ | +++ | ++ | + | + | ++ |
| 935/936 | L65V/K233A/ | +++ | +++ | +++ | ++ | ++ | + |
| | K263G/D337N/ | | | | | | |
| | A359P/A361W/ | | | | | | |
| | M374T | | | | | | |
| 937/938 | Y79V/Q536D | | ++ | + | + | + | + |
| 939/940 | Y79V/L230Y/ | ++ | +++ | ++++ | + | ++ | ++ |
| | K233A/Q239R/ | | | | | | |
| | A359P/A361W/ | | | | | | |
| | Q471T | | | | | | |
| 941/942 | L65V/Q471T | + | ++ | + | + | + | + |
| 943/944 | M374T | ++ | ++ | + | + | + | ++ |
| 945/946 | L65V/L195W/ | ++ | +++ | ++++ | + | ++ | +++ |
| | L230Y/K233A/ | | | | | | |
| | D337N/M374T/ | | | | | | |
| | P514E | | | | | | |
| 947/948 | L65V/L195W/ | ++ | +++ | +++ | + | + | ++ |
| | K233A/A359P/ | | | | | | |
| | Q536D | | | | | | |
| 949/950 | L230Y | ++ | +++ | ++ | + | ++ | + |
| 951/952 | L65V/L195W/ | +++ | ++++ | ++++ | + | +++ | ++ |
| | K263G/S276T/ | | | | | | |
| | A359P/A361W/ | | | | | | |
| | M374T/Q471T | | | | | | |
| 953/954 | Y79V | + | + | + | + | + | |
| 955/956 | L195W/L230Y/ | +++ | +++ | ++++ | + | ++ | ++ |
| | K233A/D337N/ | | | | | | |
| | M374T/Q536D | | | | | | |
| 957/958 | L195W/L230Y | ++ | ++ | +++ | + | ++ | ++ |
| 959/960 | L195W/L230Y/ | +++ | +++ | ++++ | + | ++ | ++ |
| | D337N/A359P/ | | | | | | |
| | A361W | | | | | | |
| 961/962 | L230Y/K263G | ++ | ++ | + | + | + | ++ |
| 963/964 | L65V/L230Y/ | ++ | ++ | +++ | + | + | |
| | K233G | | | | | | |
| 965/966 | K263G | + | ++ | + | + | + | |
| 967/968 | L195W/L230Y/ | +++ | +++ | ++++ | + | +++ | +++ |
| | D337N/A361W/ | | | | | | |
| | M374T/Q471T | | | | | | |
| 969/970 | L65V/L195W | ++ | ++ | ++ | + | + | ++ |
| 971/972 | M374T/Q471T | ++ | ++ | + | + | + | + |
| 973/974 | M374T/Q536D | ++ | ++ | + | + | + | ++ |
| 975/976 | Q239R | + | + | + | + | + | ++ |
| 977/978 | L65V | ++ | ++ | + | + | + | + |
| 979/980 | L230Y/K233G/ | + | ++ | +++ | + | + | + |
| | Q536L | | | | | | |
| 981/982 | L230Y/K263G/ | +++ | +++ | ++ | + | ++ | ++ |
| | M374T | | | | | | |
| 983/984 | L65V/L195W/ | +++ | +++ | ++++ | + | +++ | +++ |
| | L230Y/K263G/ | | | | | | |
| | D337N/A361W/ | | | | | | |
| | M374T/Q471T | | | | | | |
| 985/986 | Y79V/M374T | ++ | ++ | + | + | + | ++ |
| 987/988 | L65V/A361W/ | ++ | +++ | +++ | + | ++ | +++ |
| | M374T/Q471T/ | | | | | | |
| | Q536D | | | | | | |
| 989/990 | L195W | + | ++ | ++ | + | + | ++ |
| 991/992 | K233G | | ++ | ++ | + | + | |
| 993/994 | Y79V/L230Y/ | ++ | +++ | ++++ | + | ++ | +++ |
| | A359P/M374T | | | | | | |
| 995/996 | Q536D | | + | + | + | + | ++ |
| 997/998 | L230Y/A359P/ | +++ | +++ | +++ | + | ++ | +++ |
| | A361W/M374T/ | | | | | | |
| | P514E | | | | | | |
| 999/1000 | L65V/Y79V/ | + | ++ | +++ | + | + | ++ |
| | L230Y | | | | | | |
| 1001/1002 | L195W/Q239R/ | +++ | ++++ | +++ | ++ | ++ | ++ |
| | K263G/A361W/ | | | | | | |
| | M374T/P514E | | | | | | |
| 1003/1004 | L195W/K263G/ | +++ | +++ | ++ | + | + | + |
| | M374T | | | | | | |
| 1005/1006 | L195W/M374T | ++ | ++ | ++ | + | + | ++ |
| 1007/1008 | L65V/L230Y/ | +++ | +++ | ++++ | ++ | ++ | ++ |
| | K233A/A361W/ | | | | | | |
| | Q536L | | | | | | |
| 1009/1010 | L230Y/K233A/ | +++ | +++ | ++++ | + | ++ | ++ |
| | M374T/Q471T | | | | | | |
| 1011/1012 | L65V/K233A/ | +++ | +++ | +++ | ++ | + | + |
| | A359P/A361W/ | | | | | | |
| | Q471T | | | | | | |
| 1013/1014 | Q536L | | + | + | + | + | + |
| 1015/1016 | L65V/A359P/ | ++ | ++ | ++ | + | + | ++ |
| | Q471T | | | | | | |
| 1017/1018 | L65V/Y79V | + | + | + | + | + | ++ |
| 1019/1020 | L65V/A359P | ++ | ++ | + | + | + | ++ |

¹Levels of increased activity were determined relative to the reference polypeptide of SEQ ID NO: 220 and defined as follows:
“+” >0.9,
“++” >1.15,
“+++” >1.5,
“++++” >3.5

### Example 6

### GCase Variants of SEQ ID NO: 984

In this Example, experiments for evolution and screening of GCase enzyme variants derived from SEQ ID NO: 984 for improved GCase activity after a series of challenges are described. Libraries of variant GBA1 genes encoded based off SEQ ID NO: 983 were constructed and expressed in Expi293F cells for assessment of GCase 4-MU-βGLU activity (“Unchallenged Activity FIOPC”, described in Example 2), as well as after serum challenge (“Serum Stability and Activity FIOPC”, as described in Example 2), thermal challenge (“Thermal Stability and Activity FIOPC”, as described in Example 2), artificial CSF challenge (“aCSF Stability and Activity FIOPC”, as described in Example 2), and uptake into macrophages (“Macrophage Uptake FIOPC”, as described in Example 2). Libraries of variant GBA1 genes encoded based off SEQ ID NO: 983 were also expressed in HepG2 cells for assessment of GCase 4-MU-βGLU activity (“Liver Expression and Activity FIOPC,” described in Example 2). The results of these assays are presented in Table 6.1 and 6.2.

TABLE 6.1
Activity of GCase Variants Relative to SEQ ID NO: 984¹

| | | | Serum | | Artificial | | |
|---|---|---|---|---|---|---|---|
| | | | Stability | Thermal | CSF | | Liver |
| | | Unchallenged | and | Stability | Stability | | Expression |
| | | Activity | Activity | and | and | Macrophage | and |
| | Amino Acid | FIOPC | FIOPC | Activity | Activity | Uptake | Activity |
| | Differences | Relative | Relative | FIOPC | FIOPC | FIOPC | FIOPC |
| SEQ ID | (Relative to | to SEQ | to SEQ | Relative | Relative to | Relative | Relative to |
| NO: | SEQ ID NO: | ID NO: | ID NO: | to SEQ ID | SEQ ID | to SEQ ID | SEQ ID |
| (nt/aa) | 984) | 984 | 984 | NO: 984 | NO: 984 | NO: 984 | NO: 984 |
| 983/984 | | + | + | + | + | + | + |
| 1021/1022 | A359P | +++ | +++ | +++ | +++ | +++ | ++ |
| 1023/1024 | Y79V/A359P | + | ++ | ++ | ++ | ++ | ++ |
| 1025/1026 | Y79V/A97R/ | ++ | + | | ++ | ++ | +++ |
| | Q182M | | | | | | |
| 1027/1028 | A97R/A359P | + | + | ++ | + | + | ++ |
| 1029/1030 | A97R | + | + | + | + | + | ++ |
| 1031/1032 | Y79V/Q182M | | + | | | | + |

Levels of increased activity were determined relative to the reference polypeptide of SEQ ID NO: 984 and defined as follows:
“+” >0.9,
“++” >1.15,
“+++” >1.5

TABLE 6.2
Activity of GCase Variants Relative to SEQ ID NO: 984¹

| | | | | | Artificial | | |
|---|---|---|---|---|---|---|---|
| | | | Serum | Thermal | CSF | | |
| | | | Stability | Stability | Stability | | Liver |
| | Amino | Unchallenged | and | and | and | Macrophage | Expression |
| | Acid | Activity | Activity | Activity | Activity | Uptake | and Activity |
| | Differences | FIOPC | FIOPC | FIOPC | FIOPC | FIOPC | FIOPC |
| SEQ ID | (Relative | Relative | Relative | Relative to | Relative to | Relative | Relative to |
| NO: | to SEQ ID | to SEQ ID | to SEQ ID | SEQ ID | SEQ ID | to SEQ ID | SEQ ID |
| (nt/aa) | NO: 984) | NO: 984 | NO: 984 | NO: 984 | NO: 984 | NO: 984 | NO: 984 |
| 983/984 | | + | + | + | + | + | + |
| 1033/1034 | H201T | + | + | | + | + | ++ |
| 1035/1036 | H404Q | ++ | ++ | ++ | ++ | ++ | ++ |
| 1037/1038 | D484E | + | + | ++ | ++ | + | + |
| 1039/1040 | H201R | | + | + | + | + | + |
| 1041/1042 | T446N | + | | + | + | + | + |
| 1043/1044 | S494T/A495G | + | ++ | ++ | ++ | ++ | ++ |
| 1045/1046 | A495G | | + | + | | + | ++ |
| 1047/1048 | Y412W | ++ | ++ | +++ | +++ | ++ | ++ |
| 1049/1050 | K385N | ++ | ++ | ++ | ++ | ++ | + |
| 1051/1052 | K385G | | + | +++ | + | + | |
| 1053/1054 | T100N | | + | + | + | ++ | + |
| 1055/1056 | H184V | + | + | ++ | + | + | ++ |
| 1057/1058 | K385H | ++ | ++ | +++ | +++ | +++ | + |
| 1059/1060 | T82S | + | + | | + | + | ++ |
| 1061/1062 | T75A | + | + | + | + | + | + |
| 1063/1064 | P71T | | + | ++ | + | ++ | |
| 1065/1066 | R41H | + | + | ++ | + | + | + |
| 1067/1068 | A97K | + | + | + | ++ | ++ | + |
| 1069/1070 | R99E | + | + | + | + | + | + |
| 1071/1072 | A97Q | + | ++ | ++ | ++ | ++ | |
| 1073/1074 | P94N | + | + | + | + | + | + |
| 1075/1076 | T100R | | + | ++ | + | ++ | |
| 1077/1078 | M92A | + | ++ | ++ | ++ | + | |
| 1079/1080 | N98P | | + | + | + | + | |
| 1081/1082 | R99W | ++ | ++ | ++ | ++ | + | + |
| 1083/1084 | M92G | | + | + | + | + | |
| 1085/1086 | F114Y | + | + | + | + | + | + |
| 1087/1088 | I95H | + | + | + | + | + | ++ |
| 1089/1090 | T102A | + | + | + | + | | ++ |
| 1091/1092 | M92K | + | ++ | ++ | ++ | + | |
| 1093/1094 | Q96D | + | + | ++ | + | | + |
| 1095/1096 | P94E | + | ++ | ++ | + | | + |
| 1097/1098 | Q96E | ++ | ++ | ++ | ++ | + | ++ |
| 1099/1100 | I95W | + | ++ | ++ | ++ | + | + |
| 1101/1102 | L105I | + | + | ++ | + | + | |
| 1103/1104 | K113Q | ++ | ++ | ++ | ++ | ++ | + |
| 1105/1106 | M92H | ++ | + | ++ | ++ | + | ++ |
| 1107/1108 | M92Q | ++ | ++ | ++ | ++ | ++ | ++ |
| 1109/1110 | M92E | ++ | ++ | ++ | ++ | ++ | + |
| 1111/1112 | L325V | +++ | +++ | +++ | +++ | +++ | ++ |
| 1113/1114 | T336E | + | ++ | ++ | ++ | + | |
| 1115/1116 | K385Q | ++ | +++ | +++ | +++ | ++ | ++ |
| 1117/1118 | H262Q | ++ | ++ | + | ++ | ++ | + |
| 1119/1120 | F386I | + | + | ++ | + | + | + |
| 1121/1122 | K360E | + | + | ++ | + | | |
| 1123/1124 | M400T | + | ++ | ++ | ++ | ++ | |
| 1125/1126 | K342Q | | + | + | + | + | |
| 1127/1128 | M400E | ++ | ++ | ++ | ++ | + | + |
| 1129/1130 | M400Q | ++ | ++ | ++ | ++ | ++ | + |
| 1131/1132 | S390A | | | ++ | | | + |
| 1133/1134 | L264I | + | + | ++ | + | + | ++ |
| 1135/1136 | F298W | ++ | ++ | | ++ | + | |
| 1137/1138 | V382A | +++ | +++ | +++ | +++ | +++ | ++ |
| 1139/1140 | T408D | ++ | +++ | +++ | +++ | +++ | |
| 1141/1142 | T449E | + | ++ | ++ | ++ | + | + |
| 1143/1144 | L459M | + | + | ++ | + | | |
| 1145/1146 | R434G | | | ++ | | | |
| 1147/1148 | V476A | | + | + | + | + | |
| 1149/1150 | T408E | +++ | +++ | ++++ | +++ | +++ | ++ |
| 1151/1152 | R502K | + | + | ++ | + | + | |
| 1153/1154 | L29I/R534K | + | + | ++ | + | + | + |
| 1155/1156 | R502Q | ++ | ++ | ++ | ++ | + | |
| 1157/1158 | V497A | + | ++ | ++ | ++ | + | + |
| 1159/1160 | A485S | + | ++ | ++ | ++ | + | |
| 1161/1162 | W533Y | + | + | + | + | + | |
| 1163/1164 | A495S | ++ | ++ | ++ | ++ | | |
| 1165/1166 | Q536P | + | ++ | ++ | ++ | ++ | |
| 1167/1168 | Q536H | + | + | + | + | | |
| 1169/1170 | A485M | | + | + | + | + | |
| 1171/1172 | T510S | + | ++ | ++ | ++ | ++ | + |
| 1173/1174 | A485T | + | + | ++ | ++ | + | + |
| 1175/1176 | L532V | | + | ++ | + | + | + |
| 1177/1178 | L489E | ++ | ++ | ++ | ++ | + | |
| 1179/1180 | R534K | ++ | ++ | ++ | ++ | ++ | ++ |
| 1181/1182 | S494T | + | ++ | ++ | ++ | ++ | |
| 1183/1184 | Q182G | ++ | ++ | ++ | ++ | ++ | + |
| 1185/1186 | P137Q | ++ | + | ++ | + | ++ | + |
| 1187/1188 | L204Q | ++ | ++ | ++ | ++ | ++ | ++ |
| 1189/1190 | K194H | + | ++ | ++ | ++ | ++ | ++ |
| 1191/1192 | H184Q | + | ++ | +++ | ++ | + | |
| 1193/1194 | R202Q | + | ++ | ++ | ++ | + | |
| 1195/1196 | Q182S | ++ | ++ | ++ | ++ | ++ | ++ |
| 1197/1198 | L144M | + | + | ++ | + | + | ++ |
| 1199/1200 | L142N | + | + | ++ | + | + | + |
| 1201/1202 | H201Q | ++ | ++ | ++ | ++ | ++ | + |
| 1203/1204 | Q182D | + | ++ | ++ | + | + | + |
| 1205/1206 | A175D | ++ | ++ | +++ | ++ | ++ | + |
| 1207/1208 | Q182T | | + | | | | ++ |
| 1209/1210 | K194Q | ++ | ++ | ++ | ++ | ++ | ++ |
| 1211/1212 | S220A | | + | ++ | + | + | |
| 1213/1214 | E150T | + | + | + | + | + | ++ |
| 1215/1216 | L206I | | + | + | + | | |

Levels of increased activity were determined relative to the reference polypeptide of SEQ ID NO: 984 and defined as follows:
“+” >0.9,
“++” >1.15,
“+++” >2,
“++++” >5

### Example 7

### Activity of Vectorized GCase Variants Compared to WT in Cellular Transductions

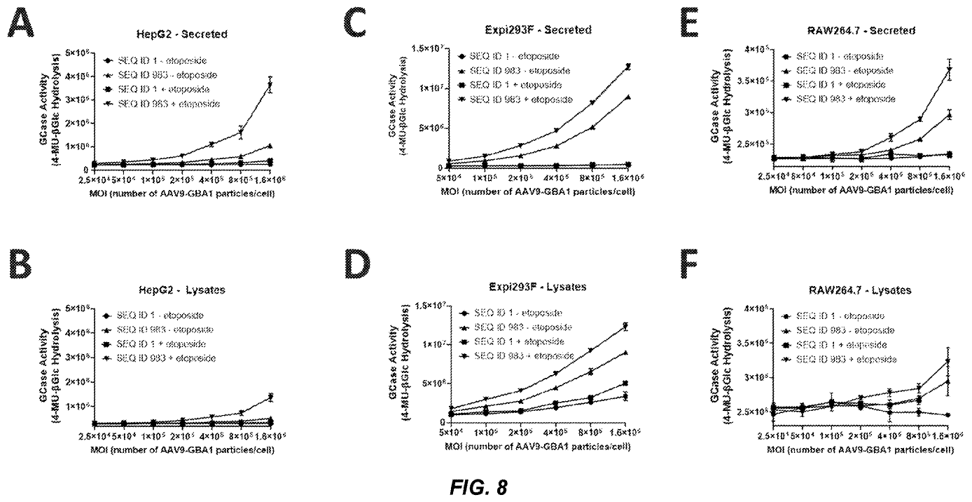

In this Example, experiments were conducted to compare the transduction efficiency of GCase variants when delivered to cells as AAV9 vectorized constructs. Cells were plated in 96-well tissue culture plates at 40,000 cells per well in cell-line appropriate complete growth media supplemented with low IgG serum (Gibco, 16250078) where appropriate; adherent cells were allowed to adhere overnight. Cells are transduced with purified AAV preparations at a Multiplicity of Infection (MOI) or several increasing MOI's based on the AAV vector titer known to result in ample transgene expression for analysis. Viral vectors based on adeno-associated virus (AAV) preferentially transduce cells in the S phase of the cell cycle. It has been shown that the topoisomerase inhibitor etoposide, increases the transduction of nondividing cells (Russell et al., Proc Natl Acad Sci USA, 1995, 92:5719-5723). Cells were treated with and without the addition of 0.4 μM etoposide to boost transduction and protein expression. At 72 hr post transduction the supernatants and cells were examined for GCase activity. FIGS. 8A-F provide graphs showing the resulting activity in the conditioned media of the secreted protein and in the lysates.

### Example 8

### Efficacy of Vectorized GCase Variants Compared to WT in Gaucher D409V/D409V Knock In Mice

In this Example, experiments were conducted using an animal model of Gaucher disease to characterize an engineered GCase variant when administered as a vectorized AAV. GCase variants SEQ ID NO: 1 and SEQ ID NO: 983 were cloned into an appropriate plasmid for vectorization into AAV9 and placed under control of a under a CAG promoter. A summary of the study design to profiles efficacy in mice is provided in Table 8.1.

TABLE 8.1
Design of AAV investigation in Gaucher D409V/D409V Knock In Mice

| Strain | Test Article | N (male) | N (female) | Route | Dose |
|---|---|---|---|---|---|
| C57BL/6 | Vehicle | 3 | 3 | IV (tail vein) | N/A |
| C57BL/6N- | Vehicle | 3 | 3 | IV (tail vein) | N/A |
| Gba<tm1.1Mjff>/J | | | | | |
| C57BL/6N- | AAV9 - SEQ ID 1 | 3 | 3 | IV (tail vein) | 2e13 vg/kg |
| Gba<tm1.1Mjff>/J | | | | | |
| C57BL/6N- | AAV9 - SEQ ID 983 | 3 | 3 | IV (tail vein) | 2e13 vg/kg |
| Gba<tm1.1Mjff>/J | | | | | |

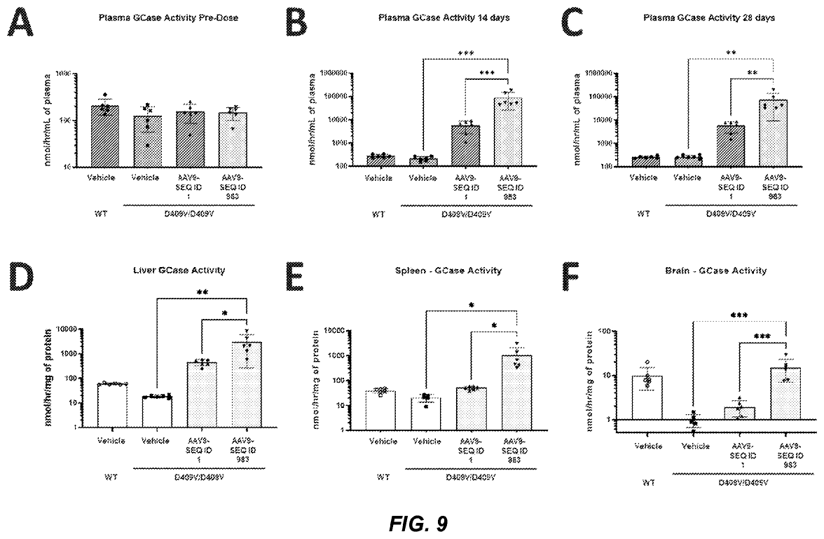

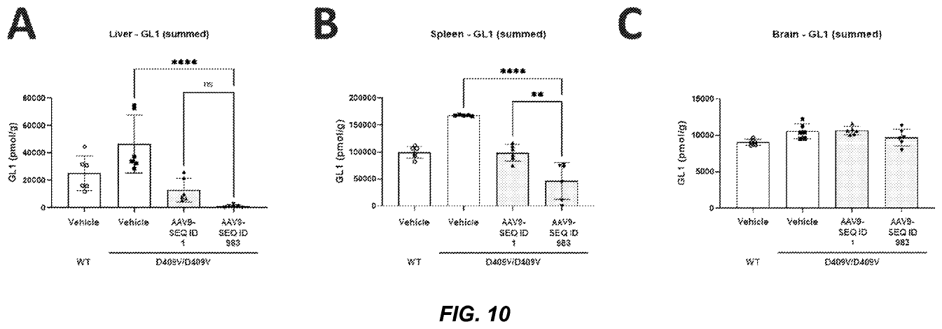

The study included 5-6 week old females and males from the C57BL/6N-Gba<tm1.1Mjff>/J knock in mouse strain (D409V/D409V KI, Jackson Laboratory, Strain 019106). Each group of animals were dosed (2e13 vg/kg; individual doses were calculated based on body weights taken on the day of dosing) by intravenous tail vein injection. Blood was drawn pre-dosing, at 2 weeks, and at 4 weeks post administration. At 4 weeks post administration the study was concluded, and the animals were euthanized. Analysis at the end of administration included GCase activity (4-MU-βGLU hydrolysis activity) in the plasma, liver, spleen, and brain, and quantitation of GL 1 in the liver, spleen, and brain. Blood was processed immediately for plasma and stored at −80° C. until assay. Plasma and tissue GCase activity was measured using the standard fluorometric 4-MU-βGLU hydrolysis activity, as described in Example 2; mice treated with vectorized SEQ ID NO: 983 displayed activity above the untreated D409V/D409V KI animals as well as those treated with vectorized SEQ ID NO: 1. FIGS. 9A-F provide graphs showing in vivo GCase enzyme activity in the plasma, liver, spleen, and brain, FIGS. 10A-C provide graphs showing GL 1 abundance in liver, spleen, and brain. Data are represented as the mean±/−standard deviation. Treatment groups were analyzed by one-way ANOVA multiple comparisons and significant differences are indicated: *: P 0.05; **: P<0.01; ***: P<0.005.

### Example 9

### In Vitro Characterization of Cross-Correction Capacity of GCase Variants in Trans-Well Cellular Models

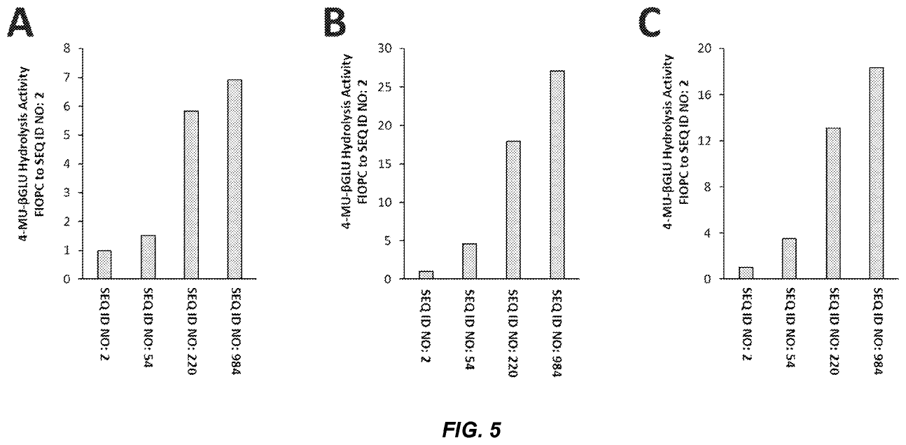

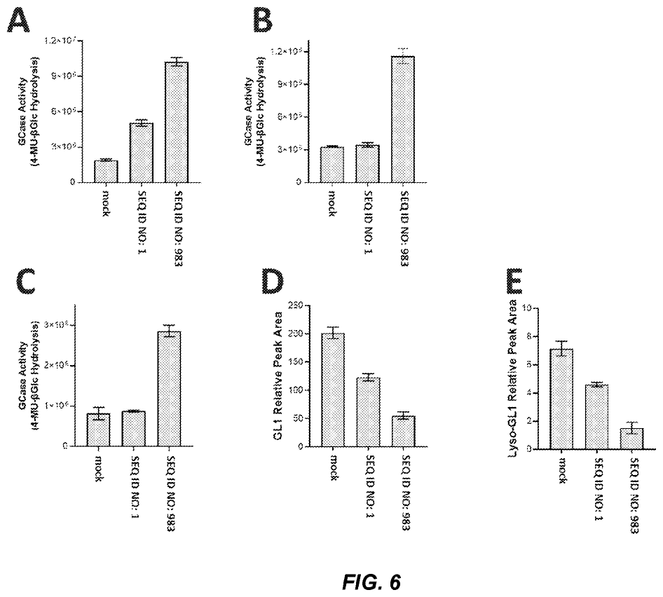

In vitro experiments were conducted to characterize the cross-correction capacity of GCase variants via either transfection with DNA or transduction with AAV vectorized constructs. HepG2 liver-derived cells were plated in the top layer of 96 well trans-well plates (Corning 3583) and allowed to adhere overnight. Cells were then transfected or transduced with DNA or vector encoding GCase variants. Separately, RAW264.7 GBA1 knockout macrophages were plated in the bottom layer of the trans-well plates and allowed to adhere. At 24 hr post transfection or transduction HepG2 cells are washed to remove residual DNA or vector and the trans-well plates are combined with HepG2 protein-producing cells in the top layer and macrophages on the lower layer. At timepoints 24-312 hr post transfection or transduction all components are assessed: HepG2 lysates for evidence of protein production, conditioned media surrounding macrophages for evidence of secreted protein, and lysates of macrophages for evidence of protein uptake (assessed via GCase 4-MU-βGLU hydrolysis activity assay) or clearance of GL1 or lyso-GL1 (assessed via LCMS). FIGS. 5A-C provide graphs showing describing the activity of GCase variants of SEQ ID NOs: 2, 54, 220, and 984 following transfection of HepG2 cells and uptake into macrophages. FIGS. 6A-E provide graphs showing describing the 4-MU-βGLU hydrolysis activity of GCase variants of SEQ ID NOs: 2 and 984 following transduction of HepG2 cells, uptake of GCase into macrophages, and clearance of GL1 and lyso-GL1.

### Example 10

### Production and Purification of GCase Variants

In this Example, production and purification of GCase variants is described.

### Production of GCase in ExpiCHO™ Cells

Milligram-scale production of GCase variants was achieved by transient transfection of ExpiCHO-S™ cells (ThermoFisher Scientific) using the lipofection method with ExpiFectamine™ CHO Reagent (ThermoFisher Scientific). ExpiCHO-S™ cells (ThermoFisher Scientific) were cultured in ExpiCHO™ Expression Medium (ThermoFisher Scientific) in shake flasks on the scale of 0.3-2 L. Cells were subjected to lipofection-mediated transfection and returned to a shaking incubator with 8% CO₂ and 70% humidity for up to 6 days to allow for expression and secretion of GCASE variants into the conditioned media. Conditioned media was harvested, clarified by centrifugation and filtration or diatomaceous earth, supplemented with protease cocktail inhibitor, and stored at −80° C. until analysis or purification.

### Production of GCase in Expi293™ Cells

Milligram-scale production of GCase variants was achieved by transient transfection of Expi293F™ cells (ThermoFisher Scientific) using the lipofection method with ExpiFectamine™ 293 Reagent (ThermoFisher Scientific). Expi293F™ cells (ThermoFisher Scientific) were cultured in Expi293™ Expression Medium (ThermoFisher Scientific) in shake flasks on the scale of 0.3-2 L. Cells were subjected to lipofection-mediated transfection and returned to a shaking incubator with 8% CO₂ and 70% humidity for up to 6 days to allow for expression and secretion of GCase variants into the conditioned media. Conditioned media was harvested, clarified by centrifugation and filtration or diatomaceous earth, supplemented with protease cocktail inhibitor, and stored at −80° C. until analysis or purification.

### Purification of GCase by Affinity Chromatography

Milligram quantities of GCase variants were produced in Expi293™ cells or ExpiCHO™ cells as described above, were purified from mammalian culture supernatant as described in the literature (Bornhorst, J. A. and Falke, J. J. Methods Enzymol., 2000, 326, 245-54). Nickel resin (Cytiva) was washed with PBS. Supernatant was loaded onto the column and bound protein was eluted with 20 mM sodium phosphate buffer, 150 mM NaCl, 500 mM imidazole at pH 7.0. Eluted protein was concentrated, and buffer exchanged into storage buffer (20 mM MES, pH 6.0). The GCase in storage buffer was sterile filtered through 0.2 μm syringe filters, and stored at −80° C. The purification process produced 2-100 mg of purified protein/L of culture supernatant.

### Normalization of Shake Flask Expressions of GCase by 4-MU-βGLU Hydrolysis Activity

In some contexts, GCase variants were evaluated without purification via normalization of 4-MU-βGLU hydrolysis activity. For high expressing GCase variants, a dilution series of crude conditioned media was designed 2×-1000× dilution into appropriate cellular expression medium and compared to the activity profile of non-diluted lower expressing GCase variants using the 4-MU-βGLU hydrolysis activity described in Example 2. GCase variant solutions were considered activity normalized if their baseline activity post-dilution was within 20% of one another.

### Example 11

### In Vitro Characterization of GCase Variants

In this Example, experiments conducted to characterize GCase variants produced as either purified proteins or activity normalized crude conditioned medias.

### Stability in Serum of GCase Variants

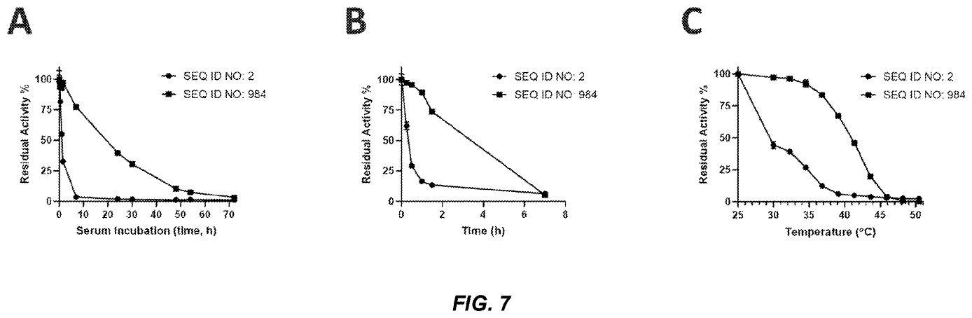

GCase variant stability to serum was determined by incubating purified variants or activity normalized crude conditioned media in 75% solutions of serum (Pooled Human AB Serum Plasma Derived Heat Inactivated, Innovative Research ISERABHI100ML) over time at 37° C. for up to 72 h. At each timepoint, a serum challenged sample was transferred into a new plate and frozen immediately at −80° C. At the conclusion of experiment residual activity of the GCase variants was determined by addition of 50 μL of 1.5-5 mM 4-MU-βGLU and incubated at 400 rpm, at 37° C. Reactions were quenched with Na₂CO₃ (0.5 M, pH 10.5) and hydrolysis was assessed by quantifying the released fluorescent methylumbelliferone, using an Envision microplate reader (cx 355/cm 460 nm). The results from this assay conducted using activity normalized conditioned media are presented in FIG. 7A.

### Stability in Artificial Cerebrospinal Fluid of GCase Variants

GCase variant stability to artificial cerebrospinal fluid (aCSF) was determined by incubating purified variants or activity normalized crude conditioned media in 75% solutions of aCSF (Tocris 3525) over time at 37° C. for up to 7 h. At each timepoint, an aCSF challenged sample was transferred into a new plate and frozen immediately at −80° C. At the conclusion of experiment residual activity of the GCase variants was determined by addition of 50 μL of 1.5-5 mM 4-MU-βGLU and incubated at 400 rpm, at 37° C. Reactions were quenched with Na₂CO₃ (0.5 M, pH 10.5) and hydrolysis was assessed by quantifying the released fluorescent methylumbelliferone, using an Envision microplate reader (ex 355/em 460 nm). The results from this assay conducted using activity normalized conditioned media are presented in FIG. 7B.

### Thermal Stability Assessment of GCase Variants

The thermal stability of GCase variants was determined by incubating purified variants or activity normalized crude conditioned media in a thermocycler for 1 hour at a thermal gradient from 30 to 55° C. and then determining residual activity compared to unchallenged GCase solutions. At the conclusion of thermal challenge, the residual activity of the GCase variants was determined by addition of 50 μL of 1.5-5 mM 4-MU-βGLU and incubated at 400 rpm, at 37° C. Reactions were quenched with Na₂CO₃ (0.5 M, pH 10.5) and hydrolysis was assessed by quantifying the released fluorescent methylumbelliferone, using an Envision microplate reader (ex 355/em 460 nm). The results from this assay conducted using activity normalized conditioned media are presented in FIG. 7C.

### Example 12

### GCase Variants of SEQ ID NO: 1150

In this Example, experiments for evolution and screening of GCase enzyme variants derived from SEQ ID NO: 1150 for improved GCase activity after a series of challenges are described. Libraries of variant GBA1 genes encoded based off SEQ ID NO: 1149 were constructed and expressed in Expi293F™ cells for assessment of GCase 4-MU-βGLU activity (“Unchallenged Activity FIOPC”, described in Example 2), as well as after serum challenge (“Serum Stability and Activity FIOPC”, as described in Example 2), thermal challenge (“Thermal Stability and Activity FIOPC”, as described in Example 2), artificial CSF challenge (“aCSF Stability and Activity FIOPC”, as described in Example 2), and/or uptake into macrophages (“Macrophage Uptake FIOPC”, as described in Example 2). The results of these assays are presented in Tables 12.1 and 12.2.

TABLE 12.1
Activity of GCase Variants Relative to SEQ ID NO: 1150

| | | | Thermal | Serum | aCSF | Mac |
|---|---|---|---|---|---|---|
| | | Baseline - | stability - | stability - | stability - | Uptake - |
| | Amino Acid | FIOPC | FIOPC | FIOPC | FIOPC | FIOPC |
| SEQ ID | Differences | Relative to | Relative to | Relative to | Relative to | Relative to |
| NO: | (Relative to | SEQ ID | SEQ ID | SEQ ID | SEQ ID | SEQ ID |
| (nt/aa) | SEQ ID NO: 1150) | NO: 1150 | NO: 1150 | NO: 1150 | NO: 1150 | NO: 1150 |
| 1217/1218 | Q96E/K194Q/V382A/ | +++ | +++ | +++ | ++ | +++ |
| | D484E | | | | | |
| 1219/1220 | A359P/V382A/Y412W/ | ++ | +++ | ++ | ++ | +++ |
| | S494T/A495G | | | | | |
| 1221/1222 | M92Q/Q96E/A175D/ | ++ | ++ | ++ | ++ | +++ |
| | Q182S/V382A | | | | | |
| 1223/1224 | Q96E/A359P/V382A/ | ++ | +++ | ++ | ++ | +++ |
| | D484E/S494T/A495G | | | | | |
| 1225/1226 | M92E/A175D/K194Q/ | ++ | ++ | + | + | +++ |
| | A359P/V382A | | | | | |
| 1227/1228 | A359P/V382A/M400E/ | +++ | +++ | +++ | ++ | +++ |
| | Y412W | | | | | |
| 1229/1230 | M92Q/Q96E/Q182S/ | ++ | +++ | ++ | ++ | +++ |
| | V382A/M400E/ | | | | | |
| | D484E | | | | | |
| 1231/1232 | M92Q/Q96E/V382A/ | +++ | +++ | ++ | ++ | +++ |
| | S494T/A495G | | | | | |
| 1233/1234 | M92Q/Q96E/A175D/ | ++ | +++ | ++ | ++ | +++ |
| | V382A/S494T/A495G | | | | | |
| 1235/1236 | Q96E/Q182S/A359P/ | +++ | +++ | ++ | ++ | +++ |
| | V382A/D484E | | | | | |
| 1237/1238 | M92E/Q96E/V382A | +++ | +++ | ++ | ++ | +++ |
| 1239/1240 | M92Q/V382A/S494T/ | +++ | +++ | ++ | ++ | +++ |
| | A495G/R534K | | | | | |
| 1241/1242 | M92E/Q96E/V382A/ | +++ | +++ | ++ | ++ | +++ |
| | M400Q/D484E | | | | | |
| 1243/1244 | M92Q/Q96E/A175D/ | +++ | +++ | +++ | ++ | +++ |
| | V382A | | | | | |
| 1245/1246 | M92Q/A175D/V382A/ | +++ | +++ | ++ | ++ | +++ |
| | M400Q/D484E | | | | | |
| 1247/1248 | M92E/Q96E/A175D/ | +++ | +++ | ++ | +++ | +++ |
| | V382A/S494T/A495G | | | | | |
| 1249/1250 | M92Q/Q96E/A175D/ | ++ | +++ | ++ | ++ | +++ |
| | Q182S/V382A/ | | | | | |
| | Y412W/D484E | | | | | |
| 1251/1252 | M92Q/Q96E/H201Q/ | +++ | +++ | ++ | +++ | +++ |
| | V382A/D484E | | | | | |
| 1253/1254 | M92E/Q182S/K194Q/ | +++ | +++ | ++ | ++ | +++ |
| | V382A | | | | | |
| 1255/1256 | M92Q/Q96E/A175D/ | +++ | +++ | +++ | +++ | +++ |
| | K194Q/A359P/V382A/ | | | | | |
| | M400E | | | | | |
| 1257/1258 | M92Q/A359P/V382A | +++ | +++ | ++ | ++ | +++ |
| 1259/1260 | Q96E/V382A | +++ | +++ | +++ | ++ | +++ |
| 1261/1262 | M92Q/A175D/V382A/ | ++ | +++ | ++ | ++ | +++ |
| | D484E/S494T/A495G | | | | | |
| 1263/1264 | Q96E/H201Q/A359P/ | ++ | ++ | +++ | ++ | ++ |
| | V382A | | | | | |
| 1265/1266 | M92E | ++ | ++ | + | + | ++ |
| 1267/1268 | M92Q | + | + | + | ++ | ++ |
| 1269/1270 | K194Q | + | ++ | | + | + |
| 1271/1272 | H201Q | ++ | ++ | + | ++ | + |
| 1273/1274 | A359P | ++ | | + | + | + |
| 1275/1276 | Q96E | + | + | | ++ | + |
| 1277/1278 | M92E/Q96E/V382A/ | ++ | +++ | ++ | ++ | +++ |
| | M400E/D484E/A495G | | | | | |
| 1279/1280 | V382A/A495G | ++ | ++ | ++ | + | +++ |
| 1281/1282 | V382A | ++ | ++ | | ++ | +++ |
| 1283/1284 | M92E/V382A | ++ | +++ | ++ | ++ | ++ |
| 1285/1286 | P71T/A175D/L325V/ | +++ | +++ | ++ | +++ | ++ |
| | K385H | | | | | |
| 1287/1288 | L325V/K385H | +++ | +++ | ++ | +++ | ++ |
| 1289/1290 | P71T/L325V/R502Q | +++ | ++ | +++ | +++ | + |
| 1291/1292 | P71T/A175D/L325V/ | ++ | +++ | + | ++ | ++ |
| | K385H/R502Q | | | | | |
| 1293/1294 | R202Q/L325V/K385N | +++ | ++ | ++ | +++ | |
| 1295/1296 | A97Q/A175D/K385H/ | ++ | ++ | | ++ | +++ |
| | T510S | | | | | |
| 1297/1298 | L325V/K385Q/T510S | ++ | ++ | ++ | ++ | ++ |
| 1299/1300 | T100R/A175D/L325V | ++ | ++ | ++ | ++ | ++ |
| 1301/1302 | P71T/A175D/L325V/ | ++ | ++ | + | ++ | + |
| | K360E/K385N | | | | | |
| 1303/1304 | A97Q/A175D/T510S | ++ | ++ | + | ++ | ++ |
| 1305/1306 | P71T/A97Q/T100R/ | ++ | ++ | | ++ | + |
| | A175D/L325V/K385H | | | | | |
| 1307/1308 | P71T/A175D/T510S | + | ++ | + | + | ++ |
| 1309/1310 | P71T/A97Q/L325V/ | ++ | ++ | ++ | ++ | |
| | R502Q | | | | | |
| 1311/1312 | P71T/T100R/L325V/ | ++ | ++ | ++ | ++ | + |
| | K385Q | | | | | |
| 1313/1314 | R202Q/K385N/L489E | ++ | ++ | + | ++ | + |
| 1315/1316 | L325V/K385N/R502Q | ++ | ++ | + | ++ | |
| 1317/1318 | L325V/K385H/R502Q | + | ++ | | ++ | + |
| 1319/1320 | P71T/T100R/L325V/ | ++ | ++ | | ++ | ++ |
| | K385H/T510S | | | | | |
| 1321/1322 | P71T/T100R/A175D/ | ++ | ++ | | ++ | |
| | L325V/K385N | | | | | |
| 1323/1324 | P71T/T100R/A175D/ | + | ++ | | ++ | ++ |
| | L325V/K385H | | | | | |
| 1325/1326 | K385N/T510S | ++ | ++ | + | + | + |
| 1327/1328 | A97Q/L325V/K385N | ++ | ++ | | ++ | |
| 1329/1330 | P71T | + | + | + | + | |
| 1331/1332 | A97Q/R202Q/L325V/ | + | ++ | ++ | + | |
| | K360E/K385G/T510S | | | | | |
| 1333/1334 | R502Q | + | + | | + | + |
| 1335/1336 | P71T/A97Q/G103S/ | + | + | | + | |
| | L325V | | | | | |
| 1337/1338 | T100R/L325V/K385H/ | | + | | + | + |
| | R502Q/T510S | | | | | |
| 1339/1340 | P71T/P137Q/A175D/ | | ++ | | + | |
| | L325V/K360E/K385H/ | | | | | |
| | R502Q | | | | | |
| 1341/1342 | P71T/S276T/L325V/ | + | ++ | + | | |
| | K385R/T510S | | | | | |
| 1343/1344 | P71T/A175D/K360E/ | | + | | | |
| | K385N/R502Q | | | | | |
| 1345/1346 | T100R/A175D/L325V/ | | ++ | | | |
| | K385N/L489E/R502Q | | | | | |
| 1347/1348 | P71T/A175D/K360E/ | | + | | | |
| | K385H/A485S/R502Q | | | | | |
| 1349/1350 | I95W/K113Q | +++ | ++ | ++ | ++ | ++ |
| 1351/1352 | S494T/Q536P | ++ | ++ | ++ | ++ | ++ |
| 1353/1354 | I95W/R99W/K113Q/ | +++ | ++ | ++ | ++ | + |
| | Q182D/H184V/L264I/ | | | | | |
| | F386I | | | | | |
| 1355/1356 | H184Q/Q536P | ++ | ++ | ++ | ++ | + |
| 1357/1358 | I95W/L142N/L144M/ | ++ | ++ | + | ++ | |
| | Q182G/H184Q/F386I/ | | | | | |
| | T449E | | | | | |
| 1359/1360 | L144M/L204Q/F386I/ | ++ | ++ | + | ++ | + |
| | M400T/T449E | | | | | |
| 1361/1362 | K113Q/T449E/Q536P | ++ | ++ | ++ | ++ | + |
| 1363/1364 | Q536P | ++ | ++ | ++ | ++ | ++ |
| 1365/1366 | I95W/F386I/Q536P | ++ | ++ | + | ++ | + |
| 1367/1368 | L264I | ++ | ++ | + | ++ | ++ |
| 1369/1370 | R99W/K113Q/F386I/ | ++ | ++ | + | ++ | + |
| | T449E | | | | | |
| 1371/1372 | I95W/F386I | ++ | ++ | + | ++ | ++ |
| 1373/1374 | I95W/R99W/L204Q/ | ++ | | + | ++ | |
| | F386I | | | | | |
| 1375/1376 | K113Q/L142N/F386I/ | ++ | ++ | + | ++ | ++ |
| | T449E | | | | | |
| 1377/1378 | Q182G/H184Q/L264I/ | ++ | ++ | + | ++ | + |
| | S494T | | | | | |
| 1379/1380 | K113Q/L142N/Q182D/ | ++ | ++ | ++ | ++ | |
| | S494T | | | | | |
| 1381/1382 | K113Q | ++ | + | ++ | ++ | + |
| 1383/1384 | R99W/L264I/T449E | ++ | ++ | + | ++ | + |
| 1385/1386 | I95W | ++ | + | + | + | ++ |
| 1387/1388 | I95W/R99W | + | + | + | + | ++ |
| 1389/1390 | R99W/K113Q/T449E | ++ | + | ++ | + | |
| 1391/1392 | I95W/K113Q/T449E/ | + | + | + | + | ++ |
| | Q536P | | | | | |
| 1393/1394 | I95W/R99W/K113Q | + | + | + | + | + |
| 1395/1396 | F386I/Q536P | ++ | ++ | | ++ | |
| 1397/1398 | R99W/Q182G/H184Q | + | + | + | | ++ |
| 1399/1400 | L142N/F386I/M400T/ | + | + | | + | ++ |
| | Q536P | | | | | |
| 1401/1402 | S35T/L264I/T449E/ | | | | + | ++ |
| | Q536P | | | | | |
| 1403/1404 | L142N/L144M/S220A | + | + | | | ++ |
| 1405/1406 | R99W/L142N | + | | | | ++ |
| 1407/1408 | L29I/T102A/Q182M/ | +++ | +++ | ++ | +++ | +++ |
| | S276T/T336E/S390A/ | | | | | |
| | L459M/R502K/ | | | | | |
| | L532V | | | | | |
| 1409/1410 | R41H/H262Q/T336E/ | +++ | ++ | ++ | ++ | ++ |
| | R502K | | | | | |
| 1411/1412 | L29I/A97R/E150T/ | ++ | ++ | ++ | ++ | ++ |
| | H262Q/T336E/R502K/ | | | | | |
| | L532V | | | | | |
| 1413/1414 | L29I | ++ | ++ | ++ | ++ | ++ |
| 1415/1416 | L532V | ++ | ++ | ++ | + | ++ |
| 1417/1418 | T102A/S390A/L459M/ | ++ | ++ | + | ++ | ++ |
| | R502K/L532V | | | | | |
| 1419/1420 | L29I/H262Q/S276T/ | ++ | ++ | ++ | ++ | ++ |
| | T336E/A495G | | | | | |
| 1421/1422 | L29I/Q182T/H262Q/ | ++ | + | ++ | ++ | ++ |
| | L459M/L532V | | | | | |
| 1423/1424 | M92K/R99G/T102A/ | ++ | ++ | + | ++ | ++ |
| | R502K | | | | | |
| 1425/1426 | L29I/A97R/T102A/ | ++ | ++ | ++ | ++ | + |
| | Q182M/R502K/L532V | | | | | |
| 1427/1428 | T336E/L459M/A495S/ | ++ | ++ | ++ | + | ++ |
| | R502K/L532V | | | | | |
| 1429/1430 | E150T/Q182T/T336E/ | ++ | ++ | ++ | + | + |
| | S390A | | | | | |
| 1431/1432 | E150T/H262Q/A495G | ++ | + | ++ | ++ | ++ |
| 1433/1434 | L29I/A495S | ++ | ++ | ++ | + | ++ |
| 1435/1436 | L29I/T102A/Q182T/ | ++ | | ++ | ++ | ++ |
| | H262Q/R502K | | | | | |
| 1437/1438 | L29I/T336E/L459M/ | ++ | ++ | ++ | + | + |
| | R502K/L532V | | | | | |
| 1439/1440 | Q182M/A495G/R502K/ | ++ | ++ | + | ++ | ++ |
| | L532V | | | | | |
| 1441/1442 | T102A/T336E | ++ | ++ | ++ | ++ | + |
| 1443/1444 | E150T/Q182M/ | ++ | + | + | + | ++ |
| | H262Q/A495S | | | | | |
| 1445/1446 | L459M/A495S/ | ++ | ++ | + | + | ++ |
| | R502K/L532V | | | | | |
| 1447/1448 | R41H/Q182M/ | ++ | ++ | + | + | ++ |
| | S390A/A495G | | | | | |
| 1449/1450 | L29I/R41H/Q182T/ | ++ | + | ++ | ++ | + |
| | T336E/A495S | | | | | |
| 1451/1452 | A97R/T102A/A495S | + | + | ++ | + | + |
| 1453/1454 | Q182T/H262Q/A495S | ++ | | ++ | + | + |
| 1455/1456 | L29I/H262Q/R502K | ++ | + | + | + | ++ |
| 1457/1458 | L29I/T102A/R502K | ++ | + | + | + | |
| 1459/1460 | Q182M/T336E/A495S | ++ | + | + | + | + |
| 1461/1462 | R502K | + | + | ++ | + | |
| 1463/1464 | L29I/E150T/Q182T/ | | | | | ++ |
| | H262Q/L459M/A495G | | | | | |
| 1465/1466 | R502K/L532V | | | | | ++ |

Levels of increased activity were determined relative to the reference polypeptide of SEQ ID NO: 1150 and defined as follows:
“+” >1,
“++” >1.2,
“+++” >1.8

TABLE 12.2
Activity of GCase Variants Relative to SEQ ID NO: 1150

| | Amino Acid | |
|---|---|---|
| SEQ ID | Differences | Mac Uptake - FIOPC |
| NO: | (Relative to | Relative to SEQ ID |
| (nt/aa) | SEQ ID NO: 1150) | NO: 1150 |
| 1467/1468 | F386P | +++ |
| 1469/1470 | V497M | +++ |
| 1471/1472 | R99K | +++ |
| 1473/1474 | K512H | +++ |
| 1475/1476 | T69D | +++ |
| 1477/1478 | V507I | +++ |
| 1479/1480 | I445N | +++ |
| 1481/1482 | T75N | ++ |
| 1483/1484 | T75H | ++ |
| 1485/1486 | K512Q | ++ |
| 1487/1488 | A487S | ++ |
| 1489/1490 | K113P | ++ |
| 1491/1492 | H184I | ++ |
| 1493/1494 | L90M | ++ |
| 1495/1496 | S235T | ++ |
| 1497/1498 | R434Q | ++ |
| 1499/1500 | I445G | ++ |
| 1501/1502 | L489N | ++ |
| 1503/1504 | L90A | ++ |
| 1505/1506 | F386L | ++ |
| 1507/1508 | T75E | ++ |
| 1509/1510 | R86E | ++ |
| 1511/1512 | L489Q | ++ |
| 1513/1514 | Q182E | ++ |
| 1515/1516 | T446H | + |
| 1517/1518 | H490N | + |
| 1519/1520 | I299V | + |
| 1521/1522 | Q96N | + |
| 1523/1524 | T102P | + |
| 1525/1526 | Y230F | + |
| 1527/1528 | L68T | + |
| 1529/1530 | N141E | + |
| 1531/1532 | A97E | + |
| 1533/1534 | R316H | + |
| 1535/1536 | I522S | + |
| 1537/1538 | L224M | + |
| 1539/1540 | S478N | + |
| 1541/1542 | K480G | + |
| 1543/1544 | R368D | + |
| 1545/1546 | R86H | + |
| 1547/1548 | Y412N | + |
| 1549/1550 | T449S | + |
| 1551/1552 | Q265N | + |
| 1553/1554 | S494A | + |

Levels of increased activity were determined relative to the reference polypeptide of SEQ ID NO: 1150 and defined as follows: “+” >1, “++” >1.2, “+++” >1.5

### Example 13

### GCase Variants of SEQ ID NO: 2

In this Example, experiments for evolution and screening of GCase enzyme variants derived from SEQ ID NO: 2 for improved GCase activity after a series of challenges are described. Libraries of variant GBA1 genes encoded based off SEQ ID NO: 1 were constructed and expressed in Expi293F™ cells for assessment of GCase 4-MU-βGLU activity (“Unchallenged Activity FIOPC”, described in Example 2), as well as after serum challenge (“Serum Stability and Activity FIOPC”, as described in Example 2), thermal challenge (“Thermal Stability and Activity FIOPC”, as described in Example 2), artificial CSF challenge (“aCSF Stability and Activity FIOPC”, as described in Example 2), and uptake into macrophages (“Macrophage Uptake FIOPC”, as described in Example 2). The results of these assays are presented in Table 13.1.

TABLE 13.1
Activity of GCase Variants Relative to SEQ ID NO: 2

| | | | Serum | | Thermal | Macrophage |
|---|---|---|---|---|---|---|
| | | Baseline - | stability - | aCSF - | stability - | uptake - |
| | | FIOPC | FIOPC | FIOPC | FIOPC | FIOPC |
| SEQ ID | Amino Acid Differences | Relative | Relative to | Relative | Relative to | Relative to |
| NO: | (Relative to | to SEQ ID | SEQ ID | to SEQ | SEQ ID | SEQ ID |
| (nt/aa) | SEQ ID NO: 2) | NO: 2 | NO: 2 | ID NO: 2 | NO: 2 | NO: 2 |
| 1149/1150 | F65V/P68L/F70L/H99R/ | ++++ | ++++ | ++++ | ++++ | ++++ |
| | G154E/L195W/A207S/ | | | | | |
| | L214F/V230Y/K263G/ | | | | | |
| | M319I/D337N/A361W/ | | | | | |
| | M374T/T408E/Q471T/M489L | | | | | |
| 1555/1556 | F65V/P68L/F70L/H99R/ | ++++ | +++- | ++++ | ++++ | ++++ |
| | G154E/L195W/A207S/ | | | | | |
| | L214F/V230Y/K263G/ | | | | | |
| | M319I/D337N/A361W/ | | | | | |
| | M374T/T408E | | | | | |
| 1557/1558 | F65V/P68L/G154E/L195W/ | ++++ | ++++ | ++++ | | ++++ |
| | L214F/V230Y/M319I/ | | | | | |
| | D337N/M374T/T408E | | | | | |
| 1559/1560 | F65V/P68L/F70L/I199R/ | ++++ | ++++ | ++++ | ++++ | ++++ |
| | G154E/L195W/A207S/ | | | | | |
| | L214F/V230Y/K263G/ | | | | | |
| | M374T/T408E | | | | | |
| 1561/1562 | F65V/P68L/F70L/G154E/ | ++++ | ++++ | ++++ | ++++ | ++++ |
| | L195W/L214F/V230Y/ | | | | | |
| | M319I/M374T/T408E/ | | | | | |
| | M489L | | | | | |
| 1563/1564 | F65V/P68L/F70L/H99R/ | ++++ | +++ | ++++ | ++++ | ++++ |
| | G154E/L195W/A207S/ | | | | | |
| | L214F/V230Y/M319I/ | | | | | |
| | D337N/A361W/M374T/ | | | | | |
| | T408E/Q471T/M489L | | | | | |
| 1565/1566 | F65V/P68L/F70L/H99R/ | ++++ | ++++ | ++++ | ++++ | ++++ |
| | G154E/L195W/A207S/ | | | | | |
| | L214F/V230Y/K263G/ | | | | | |
| | M319I/D337N/M374T/T408E | | | | | |
| 1567/1568 | F65V/P68L/G154E/L195W/ | ++++ | +++ | ++++ | ++++ | ++++ |
| | A207S/L214F/V230Y/ | | | | | |
| | K263G/M319I/D337N/ | | | | | |
| | A361W/M374T/T408E/Q471T/ | | | | | |
| | M489L | | | | | |
| 1569/1570 | F65V/P68L/F70L/H99R/ | ++++ | +++ | ++++ | ++++ | ++++ |
| | G154E/L195W/A207S/ | | | | | |
| | L214F/V230Y/K263G/ | | | | | |
| | M319I/D337N/A361W/M374T/ | | | | | |
| | T408E/M489L | | | | | |
| 1571/1572 | F65V/P68L/F70L/H99R/ | ++++ | +++ | ++++ | ++++ | ++++ |
| | G154E/L195W/A207S/ | | | | | |
| | L214F/V230Y/M374T/T408E/ | | | | | |
| | Q471T | | | | | |
| 1573/1574 | P68L/F70L/H99R/G154E/ | +++ | +++ | ++++ | ++++ | ++++ |
| | L195W/A207S/L214F/ | | | | | |
| | V230Y/K263G/M319I/T408E | | | | | |
| 1575/1576 | F65V/P68L/F70L/A207S/ | +++ | +++ | ++++ | | +++ |
| | V230Y/M374T/T408E | | | | | |
| 1577/1578 | F65V/P68L/F70L/L214F/ | +++ | +++ | ++++ | | +++ |
| | V230Y/K263G/D337N/ | | | | | |
| | M374T/T408E | | | | | |
| 1579/1580 | L214F/V230Y/K263G/ | +++ | +++ | ++++ | | +++ |
| | M374T/T408E | | | | | |
| 1581/1582 | F65V/P68L/F70L/H99R/ | +++ | +++ | ++++ | +++ | +++ |
| | G154E/L195W/A207S/ | | | | | |
| | V230Y/T408E | | | | | |
| 1583/1584 | F65V/P68L/F70L/H99R/ | +++ | +++ | ++++ | +++ | +++ |
| | G154E/A207S/V230Y/ | | | | | |
| | M319I/T408E | | | | | |
| 1585/1586 | F65V/P68L/F70L/L214F/ | +++ | +++ | ++++ | +++ | +++ |
| | V230Y/T408E | | | | | |
| 1587/1588 | F65V/P68L/F70L/L195W/ | +++ | +++ | ++++ | +++ | +++ |
| | L214F/V230Y/K263G/ | | | | | |
| | A361W/Q471T | | | | | |
| 1589/1590 | L195W/A207S/V230Y/ | +++ | +++ | ++++ | ++++ | +++ |
| | M319I/M374T/T408E/M489L | | | | | |
| 1591/1592 | F65V/P68L/F70L/L214F/ | +++ | +++ | ++++ | +++ | +++ |
| | V230Y/T408E/M489L | | | | | |
| 1593/1594 | F65V/P68L/F70L/L195W/ | +++ | +++ | +++ | +++ | +++ |
| | A207S/V230Y/T408E | | | | | |
| 1595/1596 | A207S/V230Y/M319I/ | +++ | +++ | +++ | | +++ |
| | M374T/T408E | | | | | |
| 1597/1598 | F65V/P68L/F70L/H99R/ | +++ | +++ | +++ | | +++ |
| | L195W/A207S/V230Y/ | | | | | |
| | T408E | | | | | |
| 1599/1600 | L195W/L214F/V230Y/ | +++ | +++ | +++ | +++ | +++ |
| | K263G/T408E | | | | | |
| 1601/1602 | L195W/A207S/V230Y/ | +++ | +++ | +++ | +++ | +++ |
| | M374T/T408E | | | | | |
| 1603/1604 | F65V/P68L/F70L/H99R/ | +++ | +++ | +++ | +++ | ++ |
| | G154E/L195W/L214F/ | | | | | |
| | T408E | | | | | |
| 1605/1606 | A207S/L214F/V230Y/ | +++ | +++ | +++ | ++++ | +++ |
| | M319I/M374T/T408E | | | | | |
| 1607/1608 | A207S/L214F/V230Y/ | +++ | +++ | +++ | +++ | +++ |
| | M374T/T408E | | | | | |
| 1609/1610 | G154E/A207S/V230Y/ | +++ | +++ | +++ | | ++ |
| | M374T/T408E/M489L | | | | | |
| 1611/1612 | A207S/V230Y/K263G/ | +++ | +++ | +++ | + | ++ |
| | T408E | | | | | |
| 1613/1614 | A207S/V230Y/T408E/ | +++ | ++ | +++ | + | +++ |
| | Q471T | | | | | |
| 1615/1616 | A207S/V230Y/M374T/ | +++ | ++ | +++ | + | ++ |
| | T408E | | | | | |
| 1617/1618 | L214F/V230Y/M319I/ | +++ | ++ | +++ | | +++ |
| | T408E | | | | | |
| 1619/1620 | L214F/V230Y/M374T/ | +++ | ++ | +++ | ++ | +++ |
| | T408E/M489L | | | | | |
| 1621/1622 | A207S/V230Y/T408E | +++ | ++ | +++ | | ++ |
| 1623/1624 | F65V/P68L/F70L/G154E/ | +++ | ++ | +++ | | ++ |
| | A207S/T408E | | | | | |
| 1625/1626 | A207S/V230Y/K263G/ | +++ | ++ | +++ | | ++ |
| | D337N/T408E | | | | | |
| 1627/1628 | L195W/L214F/V230Y/ | +++ | ++ | +++ | | +++ |
| | T408E | | | | | |
| 1629/1630 | A207S/V230Y/T408E/ | ++ | ++ | +++ | | ++ |
| | M489L | | | | | |
| 1631/1632 | L214F/V230Y/T408E | ++ | ++ | +++ | ++ | ++ |
| 1633/1634 | V230Y/A361W/T408E | ++ | ++ | +++ | | +++ |
| 1635/1636 | A207S/L214F/T408E | ++ | ++ | +++ | + | +++ |
| 1637/1638 | L214F/K263G/T408E | ++ | ++ | +++ | + | |
| 1639/1640 | V230Y/M374T/T408E | ++ | ++ | +++ | | + |
| 1641/1642 | A207S/T408E | ++ | + | ++ | | ++ |
| 1643/1644 | L195W/L214F/K263G | ++ | + | ++ | | |
| 1645/1646 | M374T/T408E | + | + | ++ | | + |
| 1647/1648 | T408E | + | + | + | + | ++ |

Levels of increased activity were determined relative to the reference polypeptide of SEQ ID NO: 2 and defined as follows:
“+” >1,
“++” >1.2,
“+++” >2,
“++++” >10

### Example 14

Identification of Active GCase Variants with Reduced Immunogenicity

Putative T-cell epitopes in a WT GCase of SEQ ID NO: 2 or the engineered GCase variant SEQ ID NOs: 4-1020 were identified using the Immune Epitope Database (IEDB; Immune Epitope Database and Analysis Resource website) tools, as known in the art and proprietary statistical analysis tools (see e.g., iedb.org, and Vita et al., Nucl. Acids Res., 2020, 38 (Database issue): D854-62. Epub 2009 Nov. 11). The WT GCase or the engineered GCase variant was parsed into all possible peptide 15-mer analysis frames, with each frame overlapping the last by 14 amino acids. The 15-mer analysis frames were evaluated for immunogenic potential by scoring their predicted binding to eight common class II HLA-DR alleles (DRB1*0101, DRB1*0301, DRB1*0401, DRB1*0701, DRB1*0801, DRB1*1101, DRB1*1301, and DRB1*1501) that collectively cover 77% of the world population (See e.g., iedb.org and Bui et al., 2006, BMC Bioinformatics, 7:153), using methods recommended on the IEDB website. Potential T-cell epitope clusters contained within the enzyme (i.e., sub-regions contained within GCase which have an unusually high potential for immunogenicity) were identified using statistical analysis tools, as known in the art.

GCase variants that were identified in Examples 3, 4, 5, and 6 to be active in assays described in Example 2 were analyzed for their levels of predicted immunogenicity by evaluating their binding to the eight common Class II HLA-DR alleles. The total immunogenicity score and immunogenic hit count were calculated for each variant. The total immunogenicity score (TIS) reflects the total number of predicted binding events between the eight MHC Class II alleles (described above) and peptide 15-mers across the sequence (i.e., a higher score indicates a higher level of predicted immunogenicity). The immunogenic “hit count” (IHC) indicates the number of peptide 15-mers that were predicted to bind to 4 or more of the 8 common alleles; these regions of the sequence have a particularly high potential for immunogenicity across a population (i.e., a higher score indicates a higher potential for immunogenicity). Mutations resulting in a reduced total immunogenicity score and/or an immunogenic hit count compared to the reference sequence were considered to be potential “deimmunizing mutations” and are shown in Table 14.1.

TABLE 14.1
Reduction of Total Immunogenicity Score (TIS) and Immunogenic
Hit Count (IHC) for GCase Variants Relative to SEQ ID NO: 2

| | Amino Acid | | |
|---|---|---|---|
| SEQ ID | Differences | | |
| NO: | (Relative to | TIS | IHC |
| (nt/aa) | SEQ ID NO: 2) | reduction¹ | reduction¹ |
| 3/4 | Q265T/N372D/M489L | +++ | |
| 11/12 | Q265T | ++ | |
| 21/22 | F65L/F70L/Q109D/L214F/M319I/N372D/N481T | + | |
| 29/30 | N372D | ++ | |
| 31/32 | L214F | + | + |
| 37/38 | F65L/N372D | ++ | |
| 43/44 | A207S/Q265T/M319I | + | |
| 45/46 | Q109D/A207S/L214F/N372D | ++ | + |
| 49/50 | F65L/Q109D/L214F/I243K | | + |
| 53/54 | F70L/A207S/L214F/M489L | | + |
| 55/56 | F65L/F70L/Q265T/M319I/N372D | +++ | |
| 57/58 | L214F/Q265T | +++ | + |
| 59/60 | Q265T/M319I | + | |
| 63/64 | F65L/F70L/L195M | + | |
| 71/72 | F70L/N141D | + | |
| 75/76 | L214F/M319I | + | |
| 79/80 | F65L/L214F/I243K/Q265T/M319I | ++ | |
| 81/82 | L214F/I243K | + | + |
| 83/84 | I243K/Q265T | ++ | |
| 85/86 | Q265T/M489L | + | |
| 87/88 | L214F/I243K/Q265T | +++ | + |
| 89/90 | A207S/I243K/N372D | ++ | + |
| 91/92 | L195M/I243K | + | |
| 95/96 | A207S/L214F/I243K/M319I | + | |
| 97/98 | F70L/N141D/L195M/Q265T | ++ | |
| 199/200 | P68L/F70L/A207S/L214F/T336A/M489L | | + |
| 207/208 | P68V/F70L/A207S/L214F/M489L | | + |
| 211/212 | F70L/L195M/A207S/L214F/M489L | | + |
| 215/216 | F65L/P68L/F70L/A207S/L214F/V230L/M489L | | + |
| 217/218 | F65L/F70L/A207S/L214F/M489L | | + |
| 221/222 | P68L/F70L/A207S/L214F/V230L/M489L | | + |
| 225/226 | F70L/L195M/A207S/L214F/V230L/M489L | | + |
| 227/228 | F65L/F70L/A207S/L214F/V230L/M489L | | + |
| 231/232 | F70L/G154E/A207S/L214F/T336A/M489L | | + |
| 235/236 | P68V/F70L/G154E/A207S/L214F/M489L | | + |
| 241/242 | P68V/F70L/L195M/A207S/L214F/M489L | | + |
| 243/244 | F65L/P68V/F70L/A207S/L214F/M489L | | + |
| 253/254 | F70L/N141D/G154E/A207S/L214F/M489L | | + |
| 255/256 | P68V/F70L/H99R/A207S/L214F/M489L | | + |
| 259/260 | F70L/H99R/A207S/L214F/M489L | | + |
| 261/262 | F70L/A207S/L214F/V230L/M489L | | + |
| 265/266 | P68L/F70L/A207S/L214F/M489L | | + |
| 271/272 | F70L/N141D/A207S/L214F/M489L | | + |
| 273/274 | P68V/F70L/A207S/L214F/V230L/M489L | | + |
| 275/276 | F70L/A207S/L214F/T336A/M489L | | + |
| 277/278 | F70L/N141D/A207S/L214F/V230L/M489L | | + |
| 279/280 | P68V/F70L/G154E/A207S/L214F/T336A/M489L | | + |
| 281/282 | F65L/P68L/F70L/A207S/L214F/M489L | | + |
| 285/286 | F70L/G154E/A207S/L214F/M489L | | + |
| 291/292 | P68L/F70L/G154E/A207S/L214F/M489L | | + |
| 297/298 | F70L/A207S/L214F/M489L/L509F | ++ | + |
| 299/300 | F70L/L108I/A207S/L214F/M489L | | + |
| 301/302 | F70L/L108I/V117I/A207S/L214F/M489L/L509F | | + |
| 303/304 | F70L/A207S/L214F/M489L/L519I | | + |
| 305/306 | F70L/L108I/A207S/L214F/M489L/L519I | | + |
| 307/308 | F70L/M88L/A207S/L214F/M489L | | + |
| 311/312 | F70L/A207S/L214F/M489L/L509F/L519I | ++ | + |
| 313/314 | F70L/A207S/L214F/H413Y/M489L | | + |
| 315/316 | F70L/L108I/V117I/A207S/L214F/V315T/M489L | + | + |
| 317/318 | F70L/V117I/A207S/L214F/A271T/M489L | | + |
| 319/320 | F70L/V117I/A207S/L214F/H413Y/M489L/L509F/H529Q | + | |
| 321/322 | F70L/A207S/L214F/H413Y/M489L/L509F | + | + |
| 323/324 | F70L/A207S/L214F/G469E/M489L | + | + |
| 325/326 | F70L/A207S/L214F/M489L/P524K | | + |
| 329/330 | F70L/A207S/L214F/M489L/L509I | | + |
| 333/334 | F70L/A207S/L214F/M489A | ++ | + |
| 335/336 | F70L/L104M/A207S/L214F/M489L | | + |
| 337/338 | F70L/A207S/L214F/M489L/V507A | | + |
| 339/340 | F70L/I95G/A207S/L214F/M489L | | + |
| 341/342 | F70L/A207S/L214F/A359E/M489L | + | + |
| 345/346 | F70L/A207S/L214F/M489L/L509W | ++ | + |
| 347/348 | F70L/T107D/A207S/L214F/M489L | | + |
| 349/350 | F70L/E89C/A207S/L214F/M489L | | + |
| 351/352 | F70L/A207S/L214F/A260R/M489L | ++ | +++ |
| 353/354 | F70L/P110Q/A207S/L214F/M489L | | + |
| 355/356 | F70L/F186G/A207S/L214F/M489L | | + |
| 357/358 | F70L/A207S/L214F/F370R/M489L | + | + |
| 359/360 | F70L/A207S/L214F/M489L/L509R | +++ | + |
| 361/362 | F70L/E191R/A207S/L214F/M489L | | + |
| 363/364 | F70L/A207S/L214F/V333L/M489L | | + |
| 365/366 | F70L/P189R/A207S/L214F/M489L | | + |
| 367/368 | F70L/A207S/L214F/E468G/M489L | + | + |
| 369/370 | F70L/A207S/L214F/V473I/M489L | | + |
| 371/372 | F70L/Q115M/A207S/L214F/M489L | | + |
| 373/374 | F70L/E191N/A207S/L214F/M489L | | + |
| 375/376 | V53L/F70L/A207S/L214F/M489L | + | + |
| 381/382 | F70L/A207S/L214F/M489L/F518E | +++ | + |
| 383/384 | F70L/A207S/L214F/L236M/M489L | | + |
| 385/386 | F70L/A207S/L214F/N481H/M489L | | + |
| 387/388 | F70L/Q115R/A207S/L214F/M489L | | + |
| 389/390 | A59N/F70L/A207S/L214F/S220G/M489L | + | + |
| 391/392 | F70L/A207S/L214F/M489G | ++ | + |
| 393/394 | F70L/A207S/L214F/A258T/M489L | + | + |
| 395/396 | F70L/A207S/L214F/P467N/M489L | | + |
| 397/398 | S51P/F70L/A207S/L214F/M489L | +++ | + |
| 399/400 | F70L/A207S/L214F/V415C/M489L | +++ | + |
| 401/402 | F70L/G103S/A207S/L214F/M489L | | + |
| 403/404 | F70L/A207S/L214F/M489L/G493T | + | + |
| 405/406 | F70L/A207S/L214F/M489T | ++ | + |
| 407/408 | F70L/A207S/L214F/M489L/F518V | +++ | + |
| 409/410 | F70L/R78L/A207S/L214F/M489L | + | ++ |
| 411/412 | F70L/L195W/A207S/L214F/M489L | + | ++ |
| 413/414 | F65M/F70L/A207S/L214F/M489L | | + |
| 415/416 | F70L/A207S/L214F/H413W/M489L | + | + |
| 417/418 | F70L/A207S/L214F/L335M/M489L | | + |
| 421/422 | F70L/T107E/A207S/L214F/M489L | | + |
| 423/424 | F70L/G101Q/A207S/L214F/M489L | | + |
| 425/426 | F70L/A207S/L214F/T449K/M489L | | + |
| 427/428 | F70L/A207S/L214F/N481K/M489L | | + |
| 429/430 | F70L/L104V/A207S/L214F/M489L | | + |
| 431/432 | F70L/A207S/L214F/A341R/M489L | | + |
| 433/434 | F70L/R78M/A207S/L214F/M489L | ++ | +++ |
| 435/436 | F70L/A207S/L214F/M489L/V507L | | + |
| 437/438 | F70L/A207S/L214F/T449M/M489L | | + |
| 443/444 | F70L/A207S/L214F/M489L/E520D | ++ | + |
| 445/446 | F70L/G103P/A207S/L214F/M489L | | + |
| 447/448 | F70L/L206F/A207S/L214F/M489L | | + |
| 449/450 | F70L/G93S/A207S/L214F/M489L | | + |
| 451/452 | F70L/A207S/L214F/M489L/H490S | ++ | + |
| 453/454 | F70L/A207S/L214F/V473C/M489L | ++ | + |
| 455/456 | F70L/A207S/L214F/M489L/V507R | | + |
| 457/458 | F70L/A207S/V211I/L214F/M489L | | + |
| 459/460 | F70L/P71S/A207S/L214F/M489L | | + |
| 461/462 | F70L/Y79R/A207S/L214F/M489L | +++ | +++ |
| 463/464 | F70L/D180C/A207S/L214F/M489L | | + |
| 465/466 | F70L/A207S/L214F/M489L/L509G | +++ | + |
| 467/468 | F70L/L206R/A207S/L214F/M489L | +++ | +++ |
| 469/470 | F70L/A207S/V211N/L214F/M489L | +++ | ++ |
| 471/472 | F70L/Y172S/A207S/L214F/M489L | + | + |
| 475/476 | F70L/A207D/L214F/M489L | +++ | ++ |
| 477/478 | F70L/Q115V/A207S/L214F/M489L | | + |
| 479/480 | D63L/F70L/A207S/L214F/M489L | | + |
| 481/482 | F70L/L142A/A207S/L214F/M489L | + | + |
| 483/484 | F70L/A207S/L214F/M489L/H529M | | + |
| 489/490 | F70L/I95T/A207S/L214F/M489L | | + |
| 491/492 | F70L/T107A/A207S/L214F/M489L | | + |
| 495/496 | F70L/G101L/A207S/L214F/M489L | | + |
| 499/500 | F70L/P71H/A207S/L214F/M489L | | + |
| 501/502 | F70L/E89D/A207S/L214F/M489L | | + |
| 507/508 | F70L/T107C/A207S/L214F/M489L | | + |
| 509/510 | F70L/A207S/L214F/M489L/F518S | +++ | + |
| 511/512 | F70L/A207S/S212A/L214F/M489L | | + |
| 513/514 | F70L/P71F/A207S/L214F/M489L | | + |
| 515/516 | F70L/A207S/L214F/M489L/G525C | + | + |
| 517/518 | F70L/A207S/L214F/T449R/M489L | | + |
| 523/524 | F70L/A207S/L214F/G474D/M489L | + | + |
| 525/526 | F70L/G101S/A207S/L214F/M489L | | + |
| 527/528 | F70L/L106W/A207S/L214F/M489L | | + |
| 531/532 | F70L/A207S/L214F/Q471S/M489L | | + |
| 533/534 | F70L/T107Q/A207S/L214F/M489L | | + |
| 535/536 | P68Q/F70L/A207S/L214F/M489L | | + |
| 537/538 | F70L/Y79I/A207S/L214F/M489L | | + |
| 539/540 | F70L/A207S/L214F/M489L/F518L | + | + |
| 541/542 | F70L/A207S/L214F/G474A/M489L | | + |
| 543/544 | F70L/T107S/A207S/L214F/M489L | | + |
| 545/546 | F70L/V117T/A207S/L214F/M489L | +++ | ++ |
| 551/552 | F70L/A207S/L214F/A341S/M489L | | + |
| 553/554 | F70L/A207S/L214F/M489L/H490G | + | + |
| 557/558 | F70L/A207S/L214F/M489L/H490T | + | + |
| 559/560 | F70L/A207S/L214F/V333T/M489L | | + |
| 561/562 | F70L/G101R/A207S/L214F/M489L | | + |
| 563/564 | F70L/A207S/L214F/T373V/M489L | | + |
| 565/566 | F70L/G101V/A207S/L214F/M489L | | + |
| 567/568 | F70L/G101W/A207S/L214F/M489L | | + |
| 569/570 | F65S/F70L/A207S/L214F/M489L | | + |
| 571/572 | F70L/A207S/L214F/M489L/H490V | | + |
| 573/574 | F70L/A207S/L214F/D337N/M489L | | + |
| 575/576 | F70L/A207S/L214F/M374T/M489L | + | + |
| 577/578 | P68Y/F70L/A207S/L214F/M489L | | + |
| 579/580 | F70L/I95L/A207S/L214F/M489L | | + |
| 581/582 | F70L/P71G/A207S/L214F/M489L | | + |
| 583/584 | F70L/A207S/L214F/Q471V/M489L | | + |
| 587/588 | F70L/P110A/A207S/L214F/M489L | | + |
| 589/590 | F70L/L142Y/A207S/L214F/M489L | | + |
| 591/592 | F65V/F70L/A207S/L214F/M489L | | + |
| 593/594 | F70L/P71M/A207S/L214F/M489L | | + |
| 595/596 | F70L/S77G/A207S/L214F/M489L | + | +++ |
| 599/600 | F70L/A207S/L214F/V230Y/M489L | + | + |
| 601/602 | F70L/A207S/L214F/M489L/H490Q | | + |
| 603/604 | F70L/G101T/A207S/L214F/M489L | | + |
| 605/606 | F70L/G103A/A207S/L214F/M489L | | + |
| 607/608 | F70L/A207S/L214F/M489L/V507T | | + |
| 609/610 | F70L/A207S/L214F/Q471T/M489L | | + |
| 611/612 | F70L/A207S/L214F/M489L/L509V | | + |
| 617/618 | F70L/L142N/A207S/L214F/M489L | +++ | + |
| 619/620 | F70L/Y79V/A207S/L214F/M489L | + | + |
| 625/626 | F70L/P71L/A207S/L214F/M489L | | + |
| 627/628 | P68W/F70L/A207S/L214F/M489L | | + |
| 629/630 | F70L/P189S/A207S/L214F/M489L | | + |
| 631/632 | F70L/A207S/L214F/A359P/M489L | + | ++ |
| 633/634 | F70L/P71Y/A207S/L214F/M489L | | + |
| 635/636 | F70L/S77T/A207S/L214F/M489L | | + |
| 637/638 | F70L/L142M/A207S/L214F/M489L | | + |
| 639/640 | F70L/A207S/L214F/T373A/M489L | | + |
| 643/644 | F70L/A207S/S212N/L214F/M489L | +++ | ++ |
| 645/646 | F70L/P110H/A207S/L214F/M489L | | + |
| 647/648 | F70L/A207S/L214F/V333C/M489L | ++ | ++ |
| 651/652 | P68F/F70L/A207S/L214F/M489L | | + |
| 653/654 | F70L/A207S/L214F/G474S/M489L | | + |
| 655/656 | F70L/G103L/A207S/L214F/M489L | | + |
| 659/660 | F70L/A207S/L214F/Q471E/M489L | ++ | + |
| 661/662 | F70L/A207S/L214F/M489L/V516L | | + |
| 665/666 | F70L/A207S/L214F/K233R/M489L | | + |
| 667/668 | F70L/S91T/A207S/L214F/M489L | | + |
| 669/670 | F70L/K194L/A207S/L214F/M489L | | + |
| 671/672 | F70L/S84G/A207S/L214F/M489L | ++ | +++ |
| 673/674 | F70L/A207S/L214F/A361V/M489L | | + |
| 675/676 | F70L/A207S/L214F/K233A/M489L | ++ | + |
| 677/678 | F70L/T171V/A207S/L214F/M489L | | + |
| 679/680 | F70L/A207S/L214F/M489L/Q536L | | + |
| 681/682 | Y50A/F70L/A207S/L214F/M489L | ++ | + |
| 683/684 | F70L/K116R/A207S/L214F/M489L | | + |
| 685/686 | F70L/A207S/L214F/Q239T/M489L | | + |
| 687/688 | I44V/F70L/A207S/L214F/M489L | | + |
| 689/690 | A40S/F70L/A207S/L214F/M489L | | + |
| 691/692 | F70L/K113N/A207S/L214F/M489L | | + |
| 693/694 | F70L/S91I/A207S/L214F/M489L | | + |
| 695/696 | F70L/H99F/A207S/L214F/M489L | | + |
| 697/698 | F70L/A207S/L214F/I243L/M489L | | + |
| 699/700 | F70L/I169V/A207S/L214F/M489L | + | + |
| 705/706 | F70L/Q109R/A207S/L214F/M489L | | + |
| 707/708 | F70L/A207S/L214F/M489L/Q536T | | + |
| 709/710 | F70L/A207S/L214F/Q479A/M489L | | + |
| 713/714 | F70L/A207S/L214F/A477H/M489L | | + |
| 715/716 | F70L/S84K/A207S/L214F/M489L | + | ++ |
| 717/718 | F70L/A97N/A207S/L214F/M489L | | + |
| 719/720 | F70L/A97K/A207S/L214F/M489L | | + |
| 721/722 | F70L/Q182M/A207S/L214F/M489L | | + |
| 723/724 | F70L/E111H/A207S/L214F/M489L | | + |
| 725/726 | F70L/A207S/L214F/A361Y/M489L | | + |
| 727/728 | F70L/A207S/L214F/M489L/P508R | | + |
| 729/730 | F70L/A207S/L214F/N314R/M489L | | + |
| 731/732 | F70L/A207S/L214F/T336G/M489L | + | ++ |
| 733/734 | F70L/T102E/A207S/L214F/M489L | | + |
| 735/736 | F70L/A207S/L214F/P338R/M489L | | + |
| 739/740 | F70L/H99L/A207S/L214F/M489L | | + |
| 741/742 | F70L/Q96S/A207S/L214F/M489L | | + |
| 745/746 | F70L/A207S/L214F/M489L/P508T | | + |
| 747/748 | F70L/Q96I/A207S/L214F/M489L | | + |
| 749/750 | F70L/A207S/L214F/M489L/P508Q | | + |
| 751/752 | F70L/A207S/L214F/I243M/M489L | | + |
| 755/756 | F70L/A207S/L214F/M489L/P508S | | + |
| 759/760 | P42A/F70L/A207S/L214F/M489L | | + |
| 761/762 | F70L/A207S/L214F/Q239I/M489L | | + |
| 765/766 | F70L/K116E/A207S/L214F/M489L | ++ | ++ |
| 767/768 | F70L/K113I/A207S/L214F/M489L | | + |
| 769/770 | F70L/A207S/L214F/A477V/M489L | | + |
| 771/772 | F70L/A207S/L214F/M489L/Q536I | | + |
| 773/774 | F70L/A207S/L214F/K263G/M489L | | + |
| 775/776 | F70L/H99S/A207S/L214F/M489L | | + |
| 777/778 | F70L/A207S/L214F/T222I/M489L | | + |
| 779/780 | F70L/A207S/L214F/M489L/Q536D | | + |
| 781/782 | F70L/A207S/L214F/A477G/M489L | + | + |
| 783/784 | F70L/A207S/L214F/M489L/Q536W | | + |
| 785/786 | F70L/A207S/L214F/M489L/Q536C | | + |
| 787/788 | F70L/A207S/L214F/T336R/M489L | | + |
| 789/790 | F70L/A207S/L214F/I243R/M489L | | + |
| 793/794 | F70L/A207S/L214F/M489L/Q536R | | + |
| 795/796 | F70L/A207S/L214F/M489L/I522V | ++ | + |
| 797/798 | F70L/T102S/A207S/L214F/M489L | | + |
| 799/800 | F70L/A97R/A207S/L214F/M489L | | + |
| 801/802 | A40H/F70L/A207S/L214F/M489L | | + |
| 803/804 | F70L/A207S/L214F/I243V/M489L | | + |
| 805/806 | F70L/A207S/L214F/Q265S/M489L | ++ | + |
| 807/808 | F70L/K116Q/A207S/L214F/M489L | + | + |
| 809/810 | F70L/A207S/L214F/M489L/P514E | +++ | + |
| 813/814 | F70L/A207S/L214F/M489L/A515T | +++ | + |
| 815/816 | F70L/A207S/L214F/Q246R/M489L | | + |
| 817/818 | F70L/A207S/L214F/A361W/M489L | ++ | ++ |
| 821/822 | F70L/A207S/L214F | + | + |
| 827/828 | K46R/F70L/A207S/L214F/M489L | | + |
| 829/830 | F70L/L133M/A207S/L214F/M489L | | + |
| 833/834 | F70L/A207S/L214F/Q239R/M489L | | + |
| 835/836 | F70L/Q109G/A207S/L214F/M489L | | + |
| 841/842 | F70L/A207S/L214F/K233Q/M489L | ++ | + |
| 843/844 | F70L/Q96R/A207S/L214F/M489L | | + |
| 845/846 | F70L/H99G/A207S/L214F/M489L | | + |
| 849/850 | F70L/Q96G/A207S/L214F/M489L | | + |
| 851/852 | F70L/A97M/A207S/L214F/M489L | | + |
| 853/854 | F70L/Q109L/A207S/L214F/M489L | | + |
| 855/856 | F70L/A207S/L214F/M489L/A515G | ++ | + |
| 857/858 | F70L/A207S/L214F/K233G/M489L | ++ | + |
| 859/860 | F70L/S84A/A207S/L214F/M489L | + | + |
| 861/862 | F70L/T102N/A207S/L214F/M489L | | + |
| 863/864 | F70L/A207S/L214F/M489L/S504W | + | + |
| 865/866 | F70L/A207S/L214F/Q265R/M489L | | + |
| 867/868 | F70L/H99V/A207S/L214F/M489L | | + |
| 869/870 | F70L/A207S/L214F/M489L/K512L | | + |
| 873/874 | F70L/A207S/L214F/M489L/Q536A | | + |
| 875/876 | F70L/K113R/A207S/L214F/M489L | | + |
| 877/878 | F70L/T171P/A207S/L214F/M489L | | + |
| 879/880 | F70L/E190W/A207S/L214F/M489L | | + |
| 881/882 | F70L/A207S/L214F/N314G/M489L | | + |
| 883/884 | K46M/F70L/A207S/L214F/M489L | | + |
| 885/886 | F70L/Q182H/A207S/L214F/M489L | | + |
| 887/888 | F70L/A207S/L214F/Q246K/M489L | | + |
| 889/890 | F70L/L105T/A207S/L214F/M489L | | + |
| 891/892 | F70L/A207S/L214F/R301K/M489L | | + |
| 893/894 | F70L/A207S/L214F/M489L/I522T | ++ | + |
| 895/896 | F70L/A207S/L214F/H329L/M489L | | + |
| 897/898 | F70L/Q96V/A207S/L214F/M489L | | + |
| 899/900 | F70L/A207S/L214F/E339A/M489L | | + |
| 901/902 | F70L/A207S/L214F/A477S/M489L | | + |
| 903/904 | F70L/K113G/A207S/L214F/M489L | | + |
| 905/906 | F70L/A207S/L214F/A361L/M489L | | + |
| 909/910 | F70L/L105M/A207S/L214F/M489L | | + |
| 913/914 | F70L/Q96M/A207S/L214F/M489L | | + |
| 915/916 | F70L/Q109M/A207S/L214F/M489L | | + |
| 917/918 | F70L/T171C/A207S/L214F/M489L | + | + |
| 919/920 | F70L/A207S/L214F/M489L/Q536M | | + |
| 923/924 | F65V/P68L/F70L/H99R/G154E/L195W/A207S/L214F/V230L/ | | ++ |
| | M319I/A361W/M489L | | |
| 927/928 | F65L/P68L/F70L/Y79V/H99R/G154E/L195W/A207S/L214F/ | | ++ |
| | V230L/K263G/M319I/A361W/M489L | | |
| 931/932 | F65L/P68L/F70L/Y79V/H99R/G154E/L195W/A207S/L214F/ | | ++ |
| | V230L/M319I/M489L | | |
| 935/936 | F65V/P68L/F70L/H99R/G154E/A207S/L214F/V230L/K233A/ | ++ | + |
| | K263G/M319I/D337N/A359P/A361W/M374T/M489L | | |
| 939/940 | F65L/P68L/F70L/Y79V/H99R/G154E/A207S/L214F/V230Y/ | ++ | ++ |
| | K233A/Q239R/M319I/A359P/A361W/Q471T/M489L | | |
| 945/946 | F65V/P68L/F70L/H99R/G154E/L195W/A207S/L214F/V230Y/ | +++ | + |
| | K233A/M319I/D337N/M374T/M489L/P514E | | |
| 947/948 | F65V/P68L/F70L/H99R/G154E/L195W/A207S/L214F/V230L/ | + | ++ |
| | K233A/M319I/A359P/M489L/Q536D | | |
| 951/952 | F65V/P68L/F70L/H99R/G154E/L195W/A207S/L214F/V230L/ | + | +++ |
| | K263G/S276T/M319I/A359P/A361W/M374T/Q471T/M489L | | |
| 955/956 | F65L/P68L/F70L/H99R/G154E/L195W/A207S/L214F/V230Y/ | + | + |
| | K233A/M319I/D337N/M374T/M489L/Q536D | | |
| 957/958 | F65L/P68L/F70L/H99R/G154E/L195W/A207S/L214F/V230Y/ | | + |
| | M319I/M489L | | |
| 959/960 | F65L/P68L/F70L/H99R/G154E/L195W/A207S/L214F/V230Y/ | ++ | +++ |
| | M319I/D337N/A359P/A361W/M489L | | |
| 963/964 | F65V/P68L/F70L/H99R/G154E/A207S/L214F/V230Y/K233G/ | + | |
| | M319I/M489L | | |
| 967/968 | F65L/P68L/F70L/H99R/G154E/L195W/A207S/L214F/V230Y/ | ++ | ++ |
| | M319I/D337N/A361W/M374T/Q471T/M489L | | |
| 969/970 | F65V/P68L/F70L/H99R/G154E/L195W/A207S/L214F/V230L/ | | + |
| | M319I/M489L | | |
| 979/980 | F65L/P68L/F70L/H99R/G154E/A207S/L214F/V230Y/K233G/ | + | |
| | M319I/M489L/Q536L | | |
| 983/984 | F65V/P68L/F70L/H99R/G154E/L195W/A207S/L214F/V230Y/ | ++ | ++ |
| | K263G/M319I/D337N/A361W/M374T/Q471T/M489L | | |
| 987/988 | F65V/P68L/F70L/H99R/G154E/A207S/L214F/V230L/M319I/ | | + |
| | A361W/M374T/Q471T/M489L/Q536D | | |
| 989/990 | F65L/P68L/F70L/H99R/G154E/L195W/A207S/L214F/V230L/ | | + |
| | M319I/M489L | | |
| 993/994 | F65L/P68L/F70L/Y79V/H99R/G154E/A207S/L214F/V230Y/ | + | + |
| | M319I/A359P/M374T/M489L | | |
| 997/998 | F65L/P68L/F70L/H99R/G154E/A207S/L214F/V230Y/M319I/ | +++ | + |
| | A359P/A361W/M374T/M489L/P514E | | |
| 1001/1002 | F65L/P68L/F70L/H99R/G154E/L195W/A207S/L214F/V230L/ | +++ | ++ |
| | Q239R/K263G/M319I/A361W/M374T/M489L/P514E | | |
| 1003/1004 | F65L/P68L/F70L/H99R/G154E/L195W/A207S/L214F/V230L/ | | + |
| | K263G/M319I/M374T/M489L | | |
| 1005/1006 | F65L/P68L/F70L/H99R/G154E/L195W/A207S/L214F/V230L/ | | + |
| | M319I/M374T/M489L | | |
| 1007/1008 | F65V/P68L/F70L/H99R/G154E/A207S/L214F/V230Y/K233A/ | + | + |
| | M319I/A361W/M489L/Q536L | | |
| 1011/1012 | F65V/P68L/F70L/H99R/G154E/A207S/L214F/V230L/K233A/ | ++ | ++ |
| | M319I/A359P/A361W/Q471T/M489L | | |
| 1015/1016 | F65V/P68L/F70L/H99R/G154E/A207S/L214F/V230L/M319I/ | | + |
| | A359P/Q471T/M489L | | |
| 1019/1020 | F65V/P68L/F70L/H99R/G154E/A207S/L214F/V230L/M319I/ | | + |
| | A359P/M489L | | |
| 1021/1022 | F65V/P68L/F70L/H99R/G154E/L195W/A207S/L214F/V230Y/ | +++ | +++ |
| | K263G/M319I/D337N/A359P/A361W/M374T/Q471T/M489L | | |
| 1023/1024 | F65V/P68L/F70L/Y79V/H99R/G154E/L195W/A207S/L214F/ | +++ | +++ |
| | V230Y/K263G/M319I/D337N/A359P/A361W/M374T/Q471T/M489L | | |
| 1025/1026 | F65V/P68L/F70L/Y79V/A97R/H99R/G154E/Q182M/L195W/ | +++ | +++ |
| | A207S/L214F/V230Y/K263G/M319I/D337N/A361W/M374T/Q471T/ | | |
| | M489L | | |
| 1027/1028 | F65V/P68L/F70L/A97R/H99R/G154E/L195W/A207S/L214F/ | +++ | +++ |
| | V230Y/K263G/M319I/D337N/A359P/A361W/M374T/Q471T/M489L | | |
| 1029/1030 | F65V/P68L/F70L/A97R/H99R/G154E/L195W/A207S/L214F/ | ++ | ++ |
| | V230Y/K263G/M319I/D337N/A361W/M374T/Q471T/M489L | | |
| 1031/1032 | F65V/P68L/F70L/Y79V/H99R/G154E/Q182M/L195W/A207S/ | ++ | +++ |
| | L214F/V230Y/K263G/M319I/D337N/A361W/M374T/Q471T/M489L | | |
| 1033/1034 | F65V/P68L/F70L/H99R/G154E/L195W/H201T/A207S/L214F/ | + | + |
| | V230Y/K263G/M319I/D337N/A361W/M374T/Q471T/M489L | | |
| 1035/1036 | F65V/P68L/F70L/H99R/G154E/L195W/A207S/L214F/V230Y/ | ++ | ++ |
| | K263G/M319I/D337N/A361W/M374T/H404Q/Q471T/M489L | | |
| 1037/1038 | F65V/P68L/F70L/H99R/G154E/L195W/A207S/L214F/V230Y/ | ++ | ++ |
| | K263G/M319I/D337N/A361W/M374T/Q471T/D484E/M489L | | |
| 1039/1040 | F65V/P68L/F70L/H99R/G154E/L195W/H201R/A207S/L214F/ | | + |
| | V230Y/K263G/M319I/D337N/A361W/M374T/Q471T/M489L | | |
| 1041/1042 | F65V/P68L/F70L/H99R/G154E/L195W/A207S/L214F/V230Y/ | ++ | ++ |
| | K263G/M319I/D337N/A361W/M374T/T446N/Q471T/M489L | | |
| 1043/1044 | F65V/P68L/F70L/H99R/G154E/L195W/A207S/L214F/V230Y/ | +++ | ++ |
| | K263G/M319I/D337N/A361W/M374T/Q471T/M489L/S494T/A495G | | |
| 1045/1046 | F65V/P68L/F70L/h99R/G154E/L195W/A207S/L214F/V230Y/ | +++ | ++ |
| | K263G/M319I/D337N/A361W/M374T/Q471T/M489L/A495G | | |
| 1047/1048 | F65V/P68L/F70L/H99R/G154E/L195W/A207S/L214F/V230Y/ | +++ | ++ |
| | K263G/M319I/D337N/A361W/M374T/Y412W/Q471T/M489L | | |
| 1049/1050 | F65V/P68L/F70L/H99R/G154E/L195W/A207S/L214F/V230Y/ | ++ | ++ |
| | K263G/M319I/D337N/A361W/M374T/K385N/Q471T/M489L | | |
| 1051/1052 | F65V/P68L/F70L/H99R/G154E/L195W/A207S/L214F/V230Y/ | +++ | ++ |
| | K263G/M319I/D337N/A361W/M374T/K385G/Q471T/M489L | | |
| 1053/1054 | F65V/P68L/F70L/H99R/T100N/G154E/L195W/A207S/L214F/ | ++ | ++ |
| | V230Y/K263G/M319I/D337N/A361W/M374T/Q471T/M489L | | |
| 1055/1056 | F65V/P68L/F70L/H99R/G154E/H184V/L195W/A207S/L214F/ | | ++ |
| | V230Y/K263G/M319I/D337N/A361W/M374T/Q471T/M489L | | |
| 1057/1058 | F65V/P68L/F70L/H99R/G154E/L195W/A207S/L214F/V230Y/ | ++ | ++ |
| | K263G/M319I/D337N/A361W/M374T/K385H/Q471T/M489L | | |
| 1059/1060 | F65V/P68L/F70L/T82S/H99R/G154E/L195W/A207S/L214F/ | +++ | +++ |
| | V230Y/K263G/M319I/D337N/A361W/M374T/Q471T/M489L | | |
| 1061/1062 | F65V/P68L/F70L/T75A/H99R/G154E/L195W/A207S/L214F/ | ++ | ++ |
| | V230Y/K263G/M319I/D337N/A361W/M374T/Q471T/M489L | | |
| 1063/1064 | F65V/P68L/F70L/P71T/H99R/G154E/L195W/A207S/L214F/ | ++ | ++ |
| | V230Y/K263G/M319I/D337N/A361W/M374T/Q471T/M489L | | |
| 1065/1066 | R41H/F65V/P68L/F70L/H99R/G154E/L195W/A207S/L214F/ | ++ | +++ |
| | V230Y/K263G/M319I/D337N/A361W/M374T/Q471T/M489L | | |
| 1067/1068 | F65V/P68L/F70L/A97K/H99R/G154E/L195W/A207S/L214F/ | ++ | ++ |
| | V230Y/K263G/M319I/D337N/A361W/M374T/Q471T/M489L | | |
| 1069/1070 | F65V/P68L/F70L/H99E/G154E/L195W/A207S/L214F/V230Y/ | ++ | ++ |
| | K263G/M319I/D337N/A361W/M374T/Q471T/M489L | | |
| 1071/1072 | F65V/P68L/F70L/A97Q/H99R/G154E/L195W/A207S/L214F/ | ++ | ++ |
| | V230Y/K263G/M319I/D337N/A361W/M374T/Q471T/M489L | | |
| 1073/1074 | F65V/P68L/F70L/P94N/H99R/G154E/L195W/A207S/L214F/ | ++ | ++ |
| | V230Y/K263G/M319I/D337N/A361W/M374T/Q471T/M489L | | |
| 1075/1076 | F65V/P68L/F70L/H99R/T100R/G154E/L195W/A207S/L214F/ | ++ | ++ |
| | V230Y/K263G/M319I/D337N/A361W/M374T/Q471T/M489L | | |
| 1077/1078 | F65V/P68L/F70L/M92A/H99R/G154E/L195W/A207S/L214F/ | ++ | ++ |
| | V230Y/K263G/M319I/D337N/A361W/M374T/Q471T/M489L | | |
| 1079/1080 | F65V/P68L/F70L/N98P/H99R/G154E/L195W/A207S/L214F/ | ++ | ++ |
| | V230Y/K263G/M319I/D337N/A361W/M374T/Q471T/M489L | | |
| 1081/1082 | F65V/P68L/F70L/H99W/G154E/L195W/A207S/L214F/V230Y/ | ++ | ++ |
| | K263G/M319I/D337N/A361W/M374T/Q471T/M489L | | |
| 1083/1084 | F65V/P68L/F70L/M92G/H99R/G154E/L195W/A207S/L214F/ | ++ | ++ |
| | V230Y/K263G/M319I/D337N/A361W/M374T/Q471T/M489L | | |
| 1085/1086 | F65V/P68L/F70L/H99R/F114Y/G154E/L195W/A207S/L214F/ | +++ | +++ |
| | V230Y/K263G/M319I/D337N/A361W/M374T/Q471T/M489L | | |
| 1087/1088 | F65V/P68L/F70L/I95H/H99R/G154E/L195W/A207S/L214F/ | ++ | ++ |
| | V230Y/K263G/M319I/D337N/A361W/M374T/Q471T/M489L | | |
| 1089/1090 | F65V/P68L/F70L/H99R/T102A/G154E/L195W/A207S/L214F/ | ++ | ++ |
| | V230Y/K263G/M319I/D337N/A361W/M374T/Q471T/M489L | | |
| 1091/1092 | F65V/P68L/F70L/M92K/H99R/G154E/L195W/A207S/L214F/ | ++ | ++ |
| | V230Y/K263G/M319I/D337N/A361W/M374T/Q471T/M489L | | |
| 1093/1094 | F65V/P68L/F70L/Q96D/H99R/G154E/L195W/A207S/L214F/ | ++ | ++ |
| | V230Y/K263G/M319I/D337N/A361W/M374T/Q471T/M489L | | |
| 1095/1096 | F65V/P68L/F70L/P94E/H99R/G154E/L195W/A207S/L214F/ | ++ | ++ |
| | V230Y/K263G/M319I/D337N/A361W/M374T/Q471T/M489L | | |
| 1097/1098 | F65V/P68L/F70L/Q96E/H99R/G154E/L195W/A207S/L214F/ | ++ | ++ |
| | V230Y/K263G/M319I/D337N/A361W/M374T/Q471T/M489L | | |
| 1099/1100 | F65V/P68L/F70L/I95W/H99R/G154E/L195W/A207S/L214F/ | ++ | ++ |
| | V230Y/K263G/M319I/D337N/A361W/M374T/Q471T/M489L | | |
| 1101/1102 | F65V/P68L/F70L/H99R/L105I/G154E/L195W/A207S/L214F/ | ++ | ++ |
| | V230Y/K263G/M319I/D337N/A361W/M374T/Q471T/M489L | | |
| 1103/1104 | F65V/P68L/F70L/H99R/K113Q/G154E/L195W/A207S/L214F/ | ++ | ++ |
| | V230Y/K263G/M319I/D337N/A361W/M374T/Q471T/M489L | | |
| 1105/1106 | F65V/P68L/F70L/M92H/H99R/G154E/L195W/A207S/L214F/ | ++ | ++ |
| | V230Y/K263G/M319I/D337N/A361W/M374T/Q471T/M489L | | |
| 1107/1108 | F65V/P68L/F70L/M92Q/H99R/G154E/L195W/A207S/L214F/ | ++ | ++ |
| | V230Y/K263G/M319I/D337N/A361W/M374T/Q471T/M489L | | |
| 1109/1110 | F65V/P68L/F70L/M92E/H99R/G154E/L195W/A207S/L214F/ | ++ | ++ |
| | V230Y/K263G/M319I/D337N/A361W/M374T/Q471T/M489L | | |
| 1111/1112 | F65V/P68L/F70L/H99R/G154E/L195W/A207S/L214F/V230Y/ | ++ | ++ |
| | K263G/M319I/L325V/D337N/A361W/M374T/Q471T/M489L | | |
| 1113/1114 | F65V/P68L/F70L/H99R/G154E/L195W/A207S/L214F/V230Y/ | +++ | +++ |
| | K263G/M319I/T336E/D337N/A361W/M374T/Q471T/M489L | | |
| 1115/1116 | F65V/P68L/F70L/H99R/G154E/L195W/A207S/L214F/V230Y/ | ++ | ++ |
| | K263G/M319I/D337N/A361W/M374T/K385Q/Q471T/M489L | | |
| 1117/1118 | F65V/P68L/F70L/H99R/G154E/L195W/A207S/L214F/V230Y/ | ++ | ++ |
| | H262Q/K263G/M319I/D337N/A361W/M374T/Q471T/M489L | | |
| 1119/1120 | F65V/P68L/F70L/H99R/G154E/L195W/A207S/L214F/V230Y/ | ++ | ++ |
| | K263G/M319I/D337N/A361W/M374T/F386I/Q471T/M489L | | |
| 1121/1122 | F65V/P68L/F70L/H99R/G154E/L195W/A207S/L214F/V230Y/ | +++ | +++ |
| | K263G/M319I/D337N/K360E/A361W/M374T/Q471T/M489L | | |
| 1123/1124 | F65V/P68L/F70L/H99R/G154E/L195W/A207S/L214F/V230Y/ | +++ | +++ |
| | K263G/M319I/D337N/A361W/M374T/M400T/Q471T/M489L | | |
| 1125/1126 | F65V/P68L/F70L/H99R/G154E/L195W/A207S/L214F/V230Y/ | +++ | ++ |
| | K263G/M319I/D337N/K342Q/A361W/M374T/Q471T/M489L | | |
| 1127/1128 | F65V/P68L/F70L/H99R/G154E/L195W/A207S/L214F/V230Y/ | +++ | +++ |
| | K263G/M319I/D337N/A361W/M374T/M400E/Q471T/M489L | | |
| 1129/1130 | F65V/P68L/F70L/H99R/G154E/L195W/A207S/L214F/V230Y/ | +++ | +++ |
| | K263G/M319I/D337N/A361W/M374T/M400Q/Q471T/M489L | | |
| 1131/1132 | F65V/P68L/F70L/H99R/G154E/L195W/A207S/L214F/V230Y/ | ++ | ++ |
| | K263G/M319I/D337N/A361W/M374T/S390A/Q471T/M489L | | |
| 1133/1134 | F65V/P68L/F70L/H99R/G154E/L195W/A207S/L214F/V230Y/ | ++ | ++ |
| | K263G/L264I/M319I/D337N/A361W/M374T/Q471T/M489L | | |
| 1135/1136 | F65V/P68L/F70L/H99R/G154E/L195W/A207S/L214F/V230Y/ | ++ | ++ |
| | K263G/F298W/M319I/D337N/A361W/M374T/Q471T/M489L | | |
| 1137/1138 | F65V/P68L/F70L/H99R/G154E/L195W/A207S/L214F/V230Y/ | ++ | ++ |
| | K263G/M319I/D337N/A361W/M374T/V382A/Q471T/M489L | | |
| 1139/1140 | F65V/P68L/F70L/H99R/G154E/L195W/A207S/L214F/V230Y/ | +++ | +++ |
| | K263G/M319I/D337N/A361W/M374T/T408D/Q471T/M489L | | |
| 1141/1142 | F65V/P68L/F70L/H99R/G154E/L195W/A207S/L214F/V230Y/ | ++ | ++ |
| | K263G/M319I/D337N/A361W/M374T/T449E/Q471T/M489L | | |
| 1143/1144 | F65V/P68L/F70L/H99R/G154E/L195W/A207S/L214F/V230Y/ | ++ | +++ |
| | K263G/M319I/D337N/A361W/M374T/L459M/Q471T/M489L | | |
| 1145/1146 | F65V/P68L/F70L/H99R/G154E/L195W/A207S/L214F/V230Y/ | ++ | ++ |
| | K263G/M319I/D337N/A361W/M374T/R434G/Q471T/M489L | | |
| 1147/1148 | F65V/P68L/F70L/H99R/G154E/L195W/A207S/L214F/V230Y/ | +++ | ++ |
| | K263G/M319I/D337N/A361W/M374T/Q471T/V476A/M489L | | |
| 1149/1150 | F65V/P68L/F70L/H99R/G154E/L195W/A207S/L214F/V230Y/ | +++ | +++ |
| | K263G/M319I/D337N/A361W/M374T/T408E/Q471T/M489L | | |
| 1151/1152 | F65V/P68L/F70L/H99R/G154E/L195W/A207S/L214F/V230Y/ | ++ | +++ |
| | K263G/M319I/D337N/A361W/M374T/Q471T/M489L/R502K | | |
| 1153/1154 | L29I/F65V/P68L/F70L/H99R/G154E/L195W/A207S/L214F/ | + | ++ |
| | V230Y/K263G/M319I/D337N/A361W/M374T/Q471T/M489L/R534K | | |
| 1155/1156 | F65V/P68L/F70L/H99R/G154E/L195W/A207S/L214F/V230Y/ | +++ | +++ |
| | K263G/M319I/D337N/A361W/M374T/Q471T/M489L/R502Q | | |
| 1157/1158 | F65V/P68L/F70L/H99R/G154E/L195W/A207S/L214F/V230Y/ | +++ | +++ |
| | K263G/M319I/D337N/A361W/M374T/Q471T/M489L/V497A | | |
| 1159/1160 | F65V/P68L/F70L/H99R/G154E/L195W/A207S/L214F/V230Y/ | ++ | ++ |
| | K263G/M319I/D337N/A361W/M374T/Q471T/A485S/M489L | | |
| 1161/1162 | F65V/P68L/F70L/H99R/G154E/L195W/A207S/L214F/V230Y/ | ++ | ++ |
| | K263G/M319I/D337N/A361W/M374T/Q471T/M489L/W533Y | | |
| 1163/1164 | F65V/P68L/F70L/H99R/G154E/L195W/A207S/L214F/V230Y/ | +++ | ++ |
| | K263G/M319I/D337N/A361W/M374T/Q471T/M489L/A495S | | |
| 1165/1166 | F65V/P68L/F70L/H99R/G154E/L195W/A207S/L214F/V230Y/ | ++ | ++ |
| | K263G/M319I/D337N/A361W/M374T/Q471T/M489L/Q536P | | |
| 1167/1168 | F65V/P68L/F70L/H99R/G154E/L195W/A207S/L214F/V230Y/ | ++ | ++ |
| | K263G/M319I/D337N/A361W/M374T/Q471T/M489L/Q536H | | |
| 1169/1170 | F65V/P68L/F70L/H99R/G154E/L195W/A207S/L214F/V230Y/ | ++ | ++ |
| | K263G/M319I/D337N/A361W/M374T/Q471T/A485M/M489L | | |
| 1171/1172 | F65V/P68L/F70L/H99R/G154E/L195W/A207S/L214F/V230Y/ | ++ | ++ |
| | K263G/M319I/D337N/A361W/M374T/Q471T/M489L/T510S | | |
| 1173/1174 | F65V/P68L/F70L/H99R/G154E/L195W/A207S/L214F/V230Y/ | ++ | ++ |
| | K263G/M319I/D337N/A361W/M374T/Q471T/A485T/M489L | | |
| 1175/1176 | F65V/P68L/F70L/H99R/G154E/L195W/A207S/L214F/V230Y/ | ++ | ++ |
| | K263G/M319I/D337N/A361W/M374T/Q471T/M489L/L532V | | |
| 1177/1178 | F65V/P68L/F70L/H99R/G154E/L195W/A207S/L214F/V230Y/ | +++ | ++ |
| | K263G/M319I/D337N/A361W/M374T/Q471T/M489E | | |
| 1179/1180 | F65V/P68L/F70L/H99R/G154E/L195W/A207S/L214F/V230Y/ | ++ | ++ |
| | K263G/M319I/D337N/A361W/M374T/Q471T/M489L/R534K | | |
| 1181/1182 | F65V/P68L/F70L/H99R/G154E/L195W/A207S/L214F/V230Y/ | +++ | ++ |
| | K263G/M319I/D337N/A361W/M374T/Q471T/M489L/S494T | | |
| 1183/1184 | F65V/P68L/F70L/H99R/G154E/Q182G/L195W/A207S/L214F/ | ++ | ++ |
| | V230Y/K263G/M319I/D337N/A361W/M374T/Q471T/M489L | | |
| 1185/1186 | F65V/P68L/F70L/H99R/P137Q/G154E/L195W/A207S/L214F/ | +++ | ++ |
| | V230Y/K263G/M319I/D337N/A361W/M374T/Q471T/M489L | | |
| 1187/1188 | F65V/P68L/F70L/H99R/G154E/L195W/L204Q/A207S/L214F/ | +++ | +++ |
| | V230Y/K263G/M319I/D337N/A361W/M374T/Q471T/M489L | | |
| 1189/1190 | F65V/P68L/F70L/H99R/G154E/K194H/L195W/A207S/L214F/ | ++ | ++ |
| | V230Y/K263G/M319I/D337N/A361W/M374T/Q471T/M489L | | |
| 1191/1192 | F65V/P68L/F70L/H99R/G154E/H184Q/L195W/A207S/L214F/ | ++ | ++ |
| | V230Y/K263G/M319I/D337N/A361W/M374T/Q471T/M489L | | |
| 1193/1194 | F65V/P68L/F70L/H99R/G154E/L195W/R202Q/A207S/L214F/ | +++ | +++ |
| | V230Y/K263G/M319I/D337N/A361W/M374T/Q471T/M489L | | |
| 1195/1196 | F65V/P68L/F70L/H99R/G154E/Q182S/L195W/A207S/L214F/ | ++ | ++ |
| | V230Y/K263G/M319I/D337N/A361W/M374T/Q471T/M489L | | |
| 1197/1198 | F65V/P68L/F70L/H99R/L144M/G154E/L195W/A207S/L214F/ | ++ | ++ |
| | V230Y/K263G/M319I/D337N/A361W/M374T/Q471T/M489L | | |
| 1199/1200 | F65V/P68L/F70L/H99R/L142N/G154E/L195W/A207S/L214F/ | +++ | ++ |
| | V230Y/K263G/M319I/D337N/A361W/M374T/Q471T/M489L | | |
| 1201/1202 | F65V/P68L/F70L/H99R/G154E/L195W/H201Q/A207S/L214F/ | +++ | ++ |
| | V230Y/K263G/M319I/D337N/A361W/M374T/Q471T/M489L | | |
| 1203/1204 | F65V/P68L/F70L/H99R/G154E/Q182D/L195W/A207S/L214F/ | ++ | ++ |
| | V230Y/K263G/M319I/D337N/A361W/M374T/Q471T/M489L | | |
| 1205/1206 | F65V/P68L/F70L/H99R/G154E/A175D/L195W/A207S/L214F/ | +++ | ++ |
| | V230Y/K263G/M319I/D337N/A361W/M374T/Q471T/M489L | | |
| 1207/1208 | F65V/P68L/F70L/H99R/G154E/Q182T/L195W/A207S/L214F/ | ++ | ++ |
| | V230Y/K263G/M319I/D337N/A361W/M374T/Q471T/M489L | | |
| 1209/1210 | F65V/P68L/F70L/H99R/G154E/K194Q/L195W/A207S/L214F/ | ++ | ++ |
| | V230Y/K263G/M319I/D337N/A361W/M374T/Q471T/M489L | | |
| 1211/1212 | F65V/P68L/F70L/H99R/G154E/L195W/A207S/L214F/S220A/ | ++ | ++ |
| | V230Y/K263G/M319I/D337N/A361W/M374T/Q471T/M489L | | |
| 1213/1214 | F65V/P68L/F70L/H99R/E150T/G154E/L195W/A207S/L214F/ | ++ | ++ |
| | V230Y/K263G/M319I/D337N/A361W/M374T/Q471T/M489L | | |
| 1215/1216 | F65V/P68L/F70L/H99R/G154E/L195W/L206I/A207S/L214F/ | +++ | +++ |
| | V230Y/K263G/M319I/D337N/A361W/M374T/Q471T/M489L | | |

TIS reduction was measured as a reduction in the number of counts compared to the reference polypeptide of SEQ ID NO: 2 and are defined as follows: “+” >0, “++” >5, “+++” >10. IHC reduction was measured as a reduction in the number of counts compared to the reference polypeptide of SEQ ID NO: 2 and are defined as follows: “+” >0, “++” >3, “+++” >6

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

What is claimed is:

1. An engineered glucocerebrosidase, or a biologically active fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 40-536 of SEQ ID NO: 2-1648, or to a reference sequence corresponding to SEQ ID NO: 2-1648, wherein the amino acid sequence comprises one or more substitutions relative to the reference sequence corresponding to residues 40-536 of SEQ ID NO: 2, 54, 220, 984, or 1150, or relative to the reference sequence corresponding to SEQ ID NO: 2, 54, 220, 984, or 1150.

2. The engineered glucocerebrosidase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 40-536 of SEQ ID NO: 2, 54, 220, 984, or 1150, or to a reference sequence corresponding to SEQ ID NO: 2, 54, 220, 984, or 1150, wherein the amino acid sequence comprises one or more substitutions relative to the reference sequence corresponding to residues 40-536 of SEQ ID NO: 2, 54, 220, 984, or 1150, or relative to the reference sequence corresponding to SEQ ID NO: 2, 54, 220, 984, or 1150.

3. The engineered glucocerebrosidase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 2, or to the reference sequence corresponding to SEQ ID NO: 2, wherein the amino acid sequence comprises one or more substitutions relative to the reference sequence corresponding to residues 40-536 of SEQ ID NO: 2, or relative to the reference sequence corresponding to SEQ ID NO: 2.

4. The engineered glucocerebrosidase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, 220, 984, or 1150, or to the reference sequence corresponding to SEQ ID NO: 54, 220, 984, or 1150, wherein the amino acid sequence comprises one or more substitutions relative to the reference sequence corresponding to residues 40-536 of SEQ ID NO: 2, or relative to the reference sequence corresponding to SEQ ID NO: 2.

5. The engineered glucocerebrosidase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 40-536 of an even-numbered SEQ ID NO. of SEQ ID NOs: 4-1648, or to a reference sequence corresponding to an even-numbered SEQ ID NO. of SEQ ID NOs: 4-1648, wherein the amino acid sequence comprises one or more substitutions relative to the reference sequence corresponding to residues 40-536 of SEQ ID NO: 2, or relative to the reference sequence corresponding to SEQ ID NO: 2.

6. The engineered glucocerebrosidase of any one of claims 1-5, wherein the amino acid sequence of the engineered glucocerebrosidase comprises at least a substitution at amino acid position 23, 29, 35, 40, 41, 42, 44, 46, 50, 51, 53, 59, 63, 65, 68, 69, 70, 71, 75, 77, 78, 79, 82, 84, 86, 88, 89, 90, 91, 92, 93, 94, 95, 96, 97, 98, 99, 100, 101, 102, 103, 104, 105, 106, 107, 108, 109, 110, 111, 113, 114, 115, 116, 117, 133, 134, 137, 138, 141, 142, 144, 150, 151, 154, 169, 171, 172, 175, 180, 182, 184, 186, 189, 190, 191, 194, 195, 201, 202, 204, 206, 207, 211, 212, 214, 220, 222, 224, 230, 233, 235, 236, 239, 243, 246, 258, 260, 261, 262, 263, 264, 265, 271, 276, 298, 299, 301, 314, 315, 316, 319, 325, 329, 332, 333, 335, 336, 337, 338, 339, 341, 342, 359, 360, 361, 368, 370, 372, 373, 374, 382, 385, 386, 390, 400, 404, 408, 412, 413, 415, 434, 445, 446, 449, 459, 467, 468, 469, 471, 473, 474, 476, 477, 478, 479, 480, 481, 484, 485, 487, 489, 490, 493, 494, 495, 497, 502, 504, 505, 506, 507, 508, 509, 510, 512, 514, 515, 516, 518, 519, 520, 522, 524, 525, 529, 532, 533, 534, or 536, or combinations thereof, wherein the amino acid positions are relative to the reference sequence corresponding to residues 40-536 of SEQ ID NO: 2, or relative to the reference sequence corresponding to SEQ ID NO: 2.

7. The engineered glucocerebrosidase of any one of claims 1-6, wherein the amino acid sequence of the engineered glucocerebrosidase comprises at least a substitution 23A, 29I, 35T, 40H/S, 41H, 42A, 44V, 46M/R, 50A, 51P, 53L, 59N, 63L, 65L/M/S/V, 68F/L/Q/T/V/W/Y, 69D, 70L, 71F/G/H/L/M/R/S/T/Y, 75A/E/H/N, 77G/H/I/N/R/T, 78L/M, 79I/R/V, 82S, 84A/G/K, 86E/H, 88L, 89C/D, 90A/M, 91I/T, 92A/E/G/H/K/Q, 93S, 94E/N, 95G/H/L/T/W, 96D/E/G/I/M/N/R/S/V, 97E/I/K/M/N/Q/R, 98P, 99E/F/G/K/L/R/S/V/W, 100N/R, 101L/Q/R/S/T/V/W, 102A/E/N/P/S, 103A/L/P/S, 104M/V, 105I/M/T, 106W, 107A/C/D/E/M/Q/S, 108I, 109D/G/L/M/R, 110A/H/Q, 111F/H/L, 113G/I/N/P/Q/R, 114Y, 115M/R/V, 116E/M/Q/R/V/Y, 117I/T, 133M, 134S, 137Q/V, 138S, 141D/E, 142A/M/N/Y, 144M, 150T, 151L, 154E, 169V, 171C/P/V, 172S, 175D, 180C, 182D/E/G/H/M/S/T, 184I/Q/V, 186G, 189R/S, 190W, 191N/R, 194H/L/Q, 195M/W, 201Q/R/T, 202Q, 204Q, 206F/I/R, 207S/D, 211I/L/N, 212A/N, 214F, 220A/G, 222I, 224M, 230F/L/Y, 233A/G/Q/R, 235T, 236M, 239I/R/T, 243K/L/M/R/V, 246K/R, 258T, 260R, 261K, 262Q, 263G, 264I, 265N/R/S/T, 271T, 276T, 298W, 299V, 301K, 314D/G/R, 315T, 316H, 319F/I/V, 325V, 329L, 332R, 333C/L/T, 335M, 336A/E/G/R, 337N, 338R, 339A, 341R/S, 342Q, 359E/P, 360E, 361L/V/W/Y, 368D, 370R, 372D, 373A/V/Y, 374T/V, 382A, 385G/H/N/Q/R, 386I/L/P, 390A, 400E/Q/T, 404Q, 408D/E, 412N/W, 413W/Y, 415C, 434G/Q, 445G/N, 446H/N, 449E/K/L/M/R/S/V, 459M, 467H/L/M/N/R, 468G, 469E, 471E/F/L/R/S/T/V, 473C/I/L, 474A/D/S, 476A, 477G/H/R/S/V, 478N, 479A/L/R, 480G, 481H/K/T, 484E, 485M/S/T, 487S, 489A/E/G/L/N/Q/T, 490G/I/N/Q/R/S/T/V, 493T, 494A/T, 495G/S, 497A/M, 502K/Q, 504G/P/T/W, 505I/L/R, 506T, 507A/I/L/M/R/T/Y, 508L/Q/R/S/T, 509F/G/I/R/V/W, 510S, 512H/L/Q, 514E, 515G/T, 516L, 518E/L/S/V, 519I, 520D/K/R/S/V, 522S/T/V, 524K/S, 525C/Q, 529M/Q, 532V, 533Y, 534K, or 536A/C/D/H/I/L/M/P/R/T/W, or combinations thereof, wherein the amino acid positions are relative to the reference sequence corresponding to residues 40-536 of SEQ ID NO: 2, or relative to the reference sequence corresponding to SEQ ID NO: 2.

8. The engineered glucocerebrosidase of any one of claims 1-5, wherein the amino acid sequence of the engineered glucocerebrosidase comprises at least a substitution at amino acid position 65, 68, 70, 99, 154, 195, 207, 214, 230, 263, 319, 337, 361, 374, 408, 471, or 489, or combinations thereof, wherein the amino acid positions are relative to the reference sequence corresponding to residues 40-536 of SEQ ID NO: 2, or relative to the reference sequence corresponding to SEQ ID NO: 2.

9. The engineered glucocerebrosidase of any one of claims 1-5 and 8, wherein the amino acid sequence of the engineered glucocerebrosidase comprises at least a substitution 65L/M/S/V, 68F/L/Q/T/V/W/Y, 70L,99E/F/G/K/L/R/S/V/W, 154E, 195M/W, 207S/D, 214F, 230F/L/Y, 263M, 319F/I/V, 337N, 361L/V/W/Y, 374T/V, 408D/E, 471E/F/L/R/S/T/V, or 489A/E/G/L/N/Q/T, or combinations thereof, wherein the amino acid positions are relative to the reference sequence corresponding to residues 40-536 of SEQ ID NO: 2, or relative to the reference sequence corresponding to SEQ ID NO: 2.

10. The engineered glucocerebrosidase of any one of claims 1-5 and 8, wherein the amino acid sequence of the engineered glucocerebrosidase comprises at least a substitution 665L/V, 68L, 70L, 99R, 154E, 195W, 207S, 214F, 230L/Y, 263G, 319I, 337N, 361W, 374T, 408E, 471T, or 489L, or combinations thereof, wherein the amino acid positions are relative to the reference sequence corresponding to residues 40-536 of SEQ ID NO: 2, or relative to the reference sequence corresponding to SEQ ID NO: 2.

11. The engineered glucocerebrosidase of any one of claims 1-6, wherein the amino acid sequence of the engineered glucocerebrosidase comprises at least a substitution set at amino acid position(s) 265/372/489, 243, 23, 109, 265, 109/195/243, 23/70/207/214/265, 23/70, 23/214/243/265, 65/70/109/214/319/372/481, 23/265/319, 23/65/70/141/319/481, 23/265, 372, 214, 23/65/70/109/141/214, 319, 65/372, 109/195, 23/65, 207/265/319, 109/207/214/372, 23/319/372/489, 65/109/214/243, 23/141/195, 70/207/214/489, 65/70/265/319/372, 214/265, 265/319, 65/243, 65/70/195, 489, 23/65/141, 23/372, 70/141, 23/195, 214/319, 23/207/214/243/265/481, 65/214/243/265/319, 214/243, 243/265, 265/489, 214/243/265, 207/243/372, 195/243, 65/481, 207/214/243/319, 70/141/195/265, 23/265/372, 46/99/134/230/373/449, 68/211/230, 373/449, 46/68/336, 68/230/314/373, 68, 68/336/373, 99/314, 46, 46/230/261, 373, 230/314, 68/211, 68/336/449, 46/336, 336/449, 46/68/336/373, 46/99, 99/261, 99, 68/134, 46/68/134/230/336/373, 46/68, 68/99/211/230, 99/134, 68/99/336, 449, 68/99, 46/230/336, 68/154/449, 336/373/449, 134/336, 68/99/230, 46/68/134/154, 68/99/230/373/449, 134, 46/68/154/211/230/261/336, 68/99/154/261/336, 230/449, 46/68/314, 154, 68/449, 99/449, 68/154/230/314/336/449, 46/68/154/314/336/373, 46/230, 336, or 46/99/230/373, wherein the amino acid positions are relative to the reference sequence corresponding to residues 40-536 of SEQ ID NO: 2, or relative to the reference sequence corresponding to SEQ ID NO: 2.

12. The engineered glucocerebrosidase of any one of claims 1-6 and 11, wherein the amino acid sequence of the engineered glucocerebrosidase comprises at least a substitution set 265T/372D/489L, 243K, 23A, 109D, 265T, 109D/195M/243K, 23A/70L/207S/214F/265T, 23A/70L, 23A/214F/243K/265T, 65L/70L/109D/214F/319I/372D/481T, 23A/265T/319I, 23A/65L/70L/141D/319I/481T, 23A/265T, 372D, 214F, 23A/65L/70L/109D/141D/214F, 319I, 65L/372D, 109D/195M, 23A/65L, 207S/265T/319I, 109D/207S/214F/372D, 23A/319I/372D/489L, 65L/109D/214F/243K, 23A/141D/195M, 70L/207S/214F/489L, 65L/70L/265T/319I/372D, 214F/265T, 265T/319I, 65L/243K, 65L/70L/195M, 489L, 23A/65L/141D, 23A/372D, 70L/141D, 23A/195M, 214F/319I, 23A/207S/214F/243K/265T/481T, 65L/214F/243K/265T/319I, 214F/243K, 243K/265T, 265T/489L, 214F/243K/265T, 207S/243K/372D, 195M/243K, 65L/481T, 207S/214F/243K/319I, 70L/141D/195M/265T, 23A/265T/372D, 46R/99R/134S/230L/373Y/449V, 68L/211L/230L, 373Y/449V, 46R/68V/336A, 68V/230L/314D/373Y, 68V, 68L/336A/373Y, 99R/314D, 46R, 46R/230L/261K, 373Y, 230L/314D, 68L/211L, 68V/336A/449V, 46R/336A, 336A/449V, 46R/68V/336A/373Y, 46R/99R, 99R/261K, 99R, 68V/134S, 46R/68V/134S/230L/336A/373Y, 46R/68V, 68L/99R/211L/230L, 99R/134S, 68V/99R/336A, 449V, 68V/99R, 46R/230L/336A, 68V/154E/449V, 336A/373Y/449V, 134S/336A, 68V/99R/230L, 68L, 46R/68V/134S/154E, 68V/99R/230L/373Y/449V, 134S, 46R/68V/154E/211L/230L/261K/336A, 68V/99R/154E/261K/336A, 230L/449V, 46R/68L/314D, 154E, 68L/449V, 99R/449V, 68V/154E/230L/314D/336A/449V, 46R/68V/154E/314D/336A/373Y, 46R/230L, 336A, or 46R/99R/230L/373Y, wherein the amino acid positions are relative to the reference sequence corresponding to residues 40-536 of SEQ ID NO: 2, or relative to the reference sequence corresponding to SEQ ID NO: 2.

13. The engineered glucocerebrosidase of any one of claims 1-6, wherein the amino acid sequence of the engineered glucocerebrosidase comprises at least a substitution or substitution set at amino acid position(s) 65/68/70/99/154/195/207/214/230/263/319/337/361/374/408/471/489, 65/68/70/99/154/195/207/214/230/263/319/337/361/374/408, 65/68/154/195/214/230/319/337/374/408, 65/68/70/99/154/195/207/214/230/263/374/408, 65/68/70/154/195/214/230/319/374/408/489, 65/68/70/99/154/195/207/214/230/319/337/361/374/408/471/489, 65/68/70/99/154/195/207/214/230/263/319/337/374/408, 65/68/154/195/207/214/230/263/319/337/361/374/408/471/489, 65/68/70/99/154/195/207/214/230/263/319/337/361/374/408/489, 65/68/70/99/154/195/207/214/230/374/408/471, 68/70/99/154/195/207/214/230/263/319/408, 65/68/70/207/230/374/408, 65/68/70/214/230/263/337/374/408, 214/230/263/374/408, 65/68/70/99/154/195/207/230/408, 65/68/70/99/154/207/230/319/408, 65/68/70/214/230/408, 65/68/70/195/214/230/263/361/471, 195/207/230/319/374/408/489, 65/68/70/214/230/408/489, 65/68/70/195/207/230/408, 207/230/319/374/408, 65/68/70/99/195/207/230/408, 195/214/230/263/408, 195/207/230/374/408, 65/68/70/99/154/195/214/408, 207/214/230/319/374/408, 207/214/230/374/408, 154/207/230/374/408/489, 207/230/263/408, 207/230/408/471, 207/230/374/408, 214/230/319/408, 214/230/374/408/489, 207/230/408, 65/68/70/154/207/408, 207/230/263/337/408, 195/214/230/408, 207/230/408/489, 214/230/408, 230/361/408, 207/214/408, 214/263/408, 230/374/408, 207/408, 195/214/263, 374/408, or 408, wherein the amino acid positions are relative to the reference sequence corresponding to residues 40-536 of SEQ ID NO: 2, or relative to the reference sequence corresponding to SEQ ID NO: 2.

14. The engineered glucocerebrosidase of any one of claims 1-6 and 13, wherein the amino acid sequence of the engineered glucocerebrosidase comprises at least a substitution or substitution set 65V/68L/70L/99R/154E/195W/207S/214F/230Y/263G/319I/337N/361W/374T/408E/471T/489L, 65V/68L/70L/99R/154E/195W/207S/214F/230Y/263G/319I/337N/361W/374T/408E, 65V/68L/154E/195W/214F/230Y/319I/337N/374T/408E, 65V/68L/70L/99R/154E/195W/207S/214F/230Y/263G/374T/408E, 65V/68L/70L/154E/195W/214F/230Y/319I/374T/408E/489L, 65V/68L/70L/99R/154E/195W/207S/214F/230Y/319I/337N/361W/374T/408E/471T/489L, 65V/68L/70L/99R/154E/195W/207S/214F/230Y/263G/319I/337N/374T/408E, 65V/68L/154E/195W/207S/214F/230Y/263G/319I/337N/361W/374T/408E/471T/489L, 65V/68L/70L/99R/154E/195W/207S/214F/230Y/263G/319I/337N/361W/374T/408E/489L, 65V/68L/70L/99R/154E/195W/207S/214F/230Y/374T/408E/471T, 68L/70L/99R/154E/195W/207S/214F/230Y/263G/319I/408E, 65V/68L/70L/207S/230Y/374T/408E, 65V/68L/70L/214F/230Y/263G/337N/374T/408E, 214F/230Y/263G/374T/408E, 65V/68L/70L/99R/154E/195W/207S/230Y/408E, 65V/68L/70L/99R/154E/207S/230Y/319I/408E, 65V/68L/70L/214F/230Y/408E, 65V/68L/70L/195W/214F/230Y/263G/361W/471T, 195W/207S/230Y/319I/374T/408E/489L, 65V/68L/70L/214F/230Y/408E/489L, 65V/68L/70L/195W/207S/230Y/408E, 207S/230Y/319I/374T/408E, 65V/68L/70L/99R/195W/207S/230Y/408E, 195W/214F/230Y/263G/408E, 195W/207S/230Y/374T/408E, 65V/68L/70L/99R/154E/195W/214F/408E, 207S/214F/230Y/319I/374T/408E, 207S/214F/230Y/374T/408E, 154E/207S/230Y/374T/408E/489L, 207S/230Y/263G/408E, 207S/230Y/408E/471T, 207S/230Y/374T/408E, 214F/230Y/319I/408E, 214F/230Y/374T/408E/489L, 207S/230Y/408E, 65V/68L/70L/154E/207S/408E, 207S/230Y/263G/337N/408E, 195W/214F/230Y/408E, 207S/230Y/408E/489L, 214F/230Y/408E, 230Y/361W/408E, 207S/214F/408E, 214F/263G/408E, 230Y/374T/408E, 207S/408E, 195W/214F/263G, 374T/408E, 408E, wherein the amino acid positions are relative to the reference sequence corresponding to residues 40-536 of SEQ ID NO: 2, or relative to the reference sequence corresponding to SEQ ID NO: 2.

15. The engineered glucocerebrosidase of claim 1, wherein the amino acid sequence of the engineered glucocerebrosidase comprises at least one substitution set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, or relative to the reference sequence corresponding to SEQ ID NO: 2.

16. The engineered glucocerebrosidase of claim 1, wherein the amino acid sequence of the engineered glucocerebrosidase comprises at least a substitution or substitution set of an engineered glucocerebrosidase variant set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, or relative to the reference sequence corresponding to SEQ ID NO: 2.

17. The engineered glucocerebrosidase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising a substitution or substitution set of an engineered glucocerebrosidase variant set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 2, or relative to the reference sequence corresponding to SEQ ID NO: 2.

18. The engineered glucocerebrosidase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, 220, 984, or 1150, to the reference sequence corresponding to SEQ ID NO: 54, 220, 984, or 1150.

19. The engineered glucocerebrosidase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 4-1648, or to a reference sequence corresponding to an even-numbered SEQ ID NO. of SEQ ID NOs: 4-1648.

20. The engineered glucocerebrosidase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, 220, 984, or 1150, or to the reference sequence corresponding to SEQ ID NO: 54, 220, 984, or 1150, wherein the amino acid sequence comprises one or more substitutions relative to the reference sequence corresponding to residues 40-536 of SEQ ID NO: 54, 220, 984, or 1150, or relative to the reference sequence corresponding to SEQ ID NO: 54, 220, 984, or 1150.

21. The engineered glucocerebrosidase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 4-1648, or to the reference sequence corresponding to an even-numbered SEQ ID NO. of SEQ ID NOs: 4-1648, wherein the amino acid sequence comprises one or more substitutions relative to the reference sequence corresponding to residues 40-536 of SEQ ID NO: 54, 220, 984, or 1150, or relative to the reference sequence corresponding to SEQ ID NO: 54, 220, or 984, or 1150.

22. The engineered glucocerebrosidase of claim 20 or 21, wherein the amino acid sequence of the engineered glucocerebrosidase comprises at least a substitution at amino acid position 23, 29, 35, 40, 41, 42, 44, 46, 50, 51, 53, 59, 63, 65, 68, 69, 70, 71, 75, 77, 78, 79, 82, 84, 86, 88, 89, 90, 91, 92, 93, 94, 95, 96, 97, 98, 99, 100, 101, 102, 103, 104, 105, 106, 107, 108, 109, 110, 111, 113, 114, 115, 116, 117, 133, 134, 137, 138, 141, 142, 144, 150, 151, 154, 169, 171, 172, 175, 180, 182, 184, 186, 189, 190, 191, 194, 195, 201, 202, 204, 206, 207, 211, 212, 214, 220, 222, 224, 230, 233, 235, 236, 239, 243, 246, 258, 260, 261, 262, 263, 264, 265, 271, 276, 298, 299, 301, 314, 315, 316, 319, 325, 329, 332, 333, 335, 336, 337, 338, 339, 341, 342, 359, 360, 361, 368, 370, 372, 373, 374, 382, 385, 386, 390, 400, 404, 408, 412, 413, 415, 434, 445, 446, 449, 459, 467, 468, 469, 471, 473, 474, 476, 477, 478, 479, 480, 481, 484, 485, 487, 489, 490, 493, 494, 495, 497, 502, 504, 505, 506, 507, 508, 509, 510, 512, 514, 515, 516, 518, 519, 520, 522, 524, 525, 529, 532, 533, 534, or 536, or combinations thereof, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

23. The engineered glucocerebrosidase of any one of claims 20-22, wherein the amino acid sequence of the engineered glucocerebrosidase comprises at least a substitution or amino acid residue 23A, 29I, 35T, 40H/S, 41H, 42A, 44V, 46M/R, 50A, 51P, 53L, 59N, 63L, 65L/M/S/V, 68F/L/Q/T/V/W/Y, 69D, 70L, 71F/G/H/L/M/R/S/T/Y, 75A/E/H/N, 77G/H/I/N/R/T, 78L/M, 79I/R/V, 82S, 84A/G/K, 86E/H, 88L, 89C/D, 90A/M, 91I/T, 92A/2E/G/H/K/Q, 93S, 94E/N, 95G/H/L/T/W, 96D/E/G/I/M/N/R/S/V, 97E/I/K/M/N/Q/R, 98P, 99E/F/G/K/L/R/S/V/W, 100N/R, 101L/Q/R/S/T/V/W, 102A/E/N/P/S, 103A/L/P/S, 104M/V, 105I/M/T, 106W, 107A/C/D/E/M/Q/S, 108I, 109D/G/L/M/R, 110A/H/Q, 111F/H/L, 113G/I/N/P/Q/R, 114Y, 115M/R/V, 116E/M/Q/R/V/Y, 117I/T, 133M, 134S, 137Q/V, 138S, 141D/E, 142A/M/N/Y, 144M, 150T, 151L, 154E, 169V, 171C/P/V, 172S, 175D, 180C, 182D/E/G/H/M/S/T, 184I/Q/V, 186G, 189R/S, 190W, 191N/R, 194H/L/Q, 195M/W, 201Q/R/T, 202Q, 204Q, 206F/I/R, 207S/D, 211I/L/N, 212A/N, 214F, 220A/G, 222I, 224M, 230F/L/Y, 233A/G/Q/R, 235T, 236M, 239I/R/T, 243K/L/M/R/V, 246K/R, 258T, 260R, 261K, 262Q, 263G, 264I, 265N/R/S/T, 271T, 276T, 298W, 299V, 301K, 314D/G/R, 315T, 316H, 319F/I/V, 325V, 329L, 332R, 333C/L/T, 335M, 336A/E/G/R, 337N, 338R, 339A, 341R/S, 342Q, 359E/P, 360E, 361L/V/W/Y, 368D, 370R, 372D, 373A/V/Y, 374T/V, 382A, 385G/H/N/Q/R, 386I/L/P, 390A, 400E/Q/T, 404Q, 408D/E, 412N/W, 413W/Y, 415C, 434G/Q, 445G/N, 446H/N, 449E/K/L/M/R/S/V, 459M, 467H/L/M/N/R, 468G, 469E, 471E/F/L/R/S/T/V, 473C/I/L, 474A/D/S, 476A, 477G/H/R/S/V, 478N, 479A/L/R, 480G, 481H/K/T, 484E, 485M/S/T, 487S, 489A/E/G/L/M/N/Q/T, 490G/I/N/Q/R/S/T/V, 493T, 494A/T, 495G/S, 497A/M, 502K/Q, 504G/P/T/W, 505I/L/R, 506T, 507A/I/L/M/R/T/Y, 508L/Q/R/S/T, 509F/G/I/R/V/W, 510S, 512H/L/Q, 514E, 515G/T, 516L, 518E/L/S/V, 519I, 520D/K/R/S/V, 522S/T/V, 524K/S, 525C/Q, 529M/Q, 532V, 533Y, or 534K, or combinations thereof, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

24. The engineered glucocerebrosidase of any one of claims 20-22, wherein the amino acid sequence of the engineered glucocerebrosidase comprises at least a substitution at amino acid position 65, 68, 70, 99, 154, 195, 207, 214, 230, 263, 319, 337, 361, 374, 408, 471, or 489, or combinations thereof, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

25. The engineered glucocerebrosidase of any one of claims 20-22 and 24, wherein the amino acid sequence of the engineered glucocerebrosidase comprises at least a substitution or amino acid residue 65L/M/S/V, 68F/L/Q/T/V/W/Y, 70L, 99E/F/G/K/L/R/S/V/W, 154E, 195M/W, 207S/D, 214F, 230F/L/Y, 263M, 319F/I/V, 337N, 361L/V/W/Y, 374T/V, 408D/E, 471E/F/L/R/S/T/V, or 489A/E/G/L/N/Q/T, or combinations thereof, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

26. The engineered glucocerebrosidase of any one of claims 20-22, 24, and 25, wherein the amino acid sequence of the engineered glucocerebrosidase comprises at least a substitution or amino acid residue 665L/V, 68L, 70L, 99R, 154E, 195W, 207S, 214F, 230L/Y, 263G, 319I, 337N, 361W, 374T, 408E, 471T, or 489L, or combinations thereof, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

27. The engineered glucocerebrosidase of claim 20, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 54, or to the reference sequence corresponding to SEQ ID NO: 54, wherein the amino acid sequence comprises one or more substitutions relative to the reference sequence corresponding to residues 40-536 of SEQ ID NO: 54, or relative to the reference sequence corresponding to SEQ ID NO: 54.

28. The engineered glucocerebrosidase of claim 21, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 200-922, or to the reference sequence corresponding to SEQ ID NO: 200-922, wherein the amino acid sequence comprises one or more substitutions relative to the reference sequence corresponding to residues 40-536 of SEQ ID NO: 54, or relative to the reference sequence corresponding to SEQ ID NO: 54.

29. The engineered glucocerebrosidase of claim 27 or 28, wherein the amino acid sequence of the engineered glucocerebrosidase comprises at least a substitution or substitution set at amino acid position(s) 68/336, 65/99/319, 65/68/319, 68/319, 68, 65/319, 195, 65/154/319, 65/68/230, 65, 65/68/99/154/230/319, 68/230, 195/230, 65/230, 141/319, 154/336, 68/154, 68/195/230/319/336, 68/99/195/230/319, 68/195, 65/68, 68/141/230/319, 65/68/99/230/319, 68/99/141/319, 68/154/319, 141/154, 68/99, 99/230/319, 99, 230, 68/230/319, 154/319, 65/68/154/319, 141, 336, 141/230, 68/154/336, 65/68/99/319, 154, 319, 230/319, 68/154, 99/141/319, 509, 108, 108/117/509, 519, 108/519, 88, 117/413/509, 509/519, 413, 108/117/315, 117/271, 117/413/509/529, 413/509, 469, 524, 507, 525, 489, 104, 95, 359, 374, 107, 89, 260, 110, 186, 370,191, 333, 189, 468, 473, 115, 53, 449, 518, 236, 481, 59/220, 258, 467, 51, 415, 103, 493, 78, 335, 520, 101, 341, 471, 211, 206, 93, 490, 71, 79, 180, 172, 207, 63, 142, 529, 77, 212, 474, 106, 386/507, 117, 373, 337, 107/246, 516, 233, 91, 194, 84, 361, 171, 536, 50, 116, 239, 44, 40, 113, 243, 169, 504, 505, 109, 479, 477, 97, 182, 111,508, 314, 102, 338, 96, 138, 42, 263, 222, 522, 265, 514, 515, 246, 46, 133, 332, 506, 512, 137, 190,105, 301, 329, 339, or 151, wherein the amino acid positions are relative to the reference sequence corresponding to residues 40-536 of SEQ ID NO: 54, or relative to the reference sequence corresponding to SEQ ID NO: 54.

30. The engineered glucocerebrosidase of any one of claims 27-29, wherein the amino acid sequence of the engineered glucocerebrosidase comprises at least a substitution or substitution set 68L/336A, 65L/99R/319I, 65L/68L/319I, 68V/319I, 68V, 65L/319I, 195M, 65L/154E/319I, 65L/68L/230L, 65L, 65L/68L/99R/154E/230L/319I, 68L/230L, 65L/68V/319I, 195M/230L, 65L/230L, 141D/319I, 154E/336A, 68L/319I, 68V/154E, 68V/195M/230L/319I/336A, 68V/99R/195M/230L/319I, 68V/195M, 65L/68V, 68L/141D/230L/319I, 65L/68V/99R/230L/319I, 68L/99R/141D/319I, 68V/154E/319I, 141D/154E, 68V/99R, 99R/230L/319I, 99R, 230L, 68V/230L/319I, 68L, 154E/319I, 65L/68L/154E/319I, 141D, 68V/230L, 336A, 141D/230L, 68V/154E/336A, 65L/68L, 65L/68V/99R/319I, 154E, 319I, 230L/319I, 68L/154E, 99R/141D/319I, 68L/154E/319I, 509F, 108I, 108I/117/509F, 519I, 108I/519I, 88L, 117I/413Y/509F, 509F/519I, 413Y, 108I/117/315T, 117I/271T, 117I/413Y/509F/529Q, 413 Y/509F, 469E, 524K, 507M, 509I, 525Q, 489A, 104M, 507A, 95G, 359E, 374V, 509W, 107D, 89C, 260R, 110Q, 186G, 370R, 509R, 191R, 333L, 189R, 468G, 473I, 115M, 191N, 53L, 524S, 449L, 518E, 236M, 481H, 115R, 59N/220G, 489G, 258T, 467N, 51P, 415C, 103S, 493T, 489T, 518V, 78L, 195W, F65M, 413W, 335M, 520K, 107E, 101Q, 449K, 481K, 104V, 341R, 78M, 507L, 449M, 471L, 211L, 520D, 103P, 206F, 93S, 490S, 473C, 507R, 21H, 71S, 79R, 180C, 509G, 206R, 211N, 172S, 490R, 207D, 115V, 63L, 142A, 529M, 71R, 473L, 95T, 107A, 520V, 101L, 520S, 71H, 89D, 77I, 507Y, 107C, 518S, 212A, 71F, 525C, 449R, 107M, 467H, 474D, 101S, 106W, 386L/507M, 471S, 107Q, 68Q, 79I, 518L, 474A, 107S, 117T, 467M, 471R, 341S, 490G, 471F, 490T, 333T, 101R, 373V, 101V, 101W, 65S, 490V, 337N, 374T, 68Y, 95L, 71G, 471V, 107M/246R, 110A, 142Y, 65V, 71M, 77G, 77R, 230Y, 490Q, 101T, 103A, 507T, 471T, 509V, 520R, 319F, 142N, 79V, 467L, 77N, 71L, 68W, 189S, 359P, 71Y, 77T, 142M, 373A, 319V, 212N, 110H, 333C, 490I, 68F, 474S, 103L, 77H, 471E, 516L, 467R, 233R, 91T, 194L, 84G, 361V, 233A, 171V, 536L, 50A, 116R, 239T, 44V, 40S, 113N, 91I, 99F, 243L, 169V, 504P, 505R, 109R, 536T, 479A, 479L, 477H, 84K, 97N, 97K, 182M, 111H, 361Y, 508R, 314R, 336G, 102E, 338R, 505L, 99L, 96S, 138S, 508T, 96I, 508Q, 243M, 111L, 508S, 479R, 42A, 239I, 97I, 116E, 113I, 477V, 536I, 263G, 99S, 222I, 536D, 477G, 536W, 536C, 336R, 243R, 116V, 536R, 522V, 102S, 97R, 40H, 243V, 265S, 116Q, 514E, 508L, 515T, 246R, 361W, 111F, 489M, 477R, 116Y, 46R, 133M, 332R, 239R, 109G, 506T, 504G, 233Q, 96R, 99G, 504T, 96G, 97M, 109L, 515G, 233G, 84A, 102N, 504W, 265R, 99V, 512L, 137V, 536A, 113R, 171P, 190W, 314G, 46M, 182H, 246K, 105T, 301K, 522T, 329L, 96V, 339A, 477S, 113G, 361L, 505I, 105M, 116M, 96M, 109M, 171C, 536M, or 151L, wherein the amino acid positions are relative to the reference sequence corresponding to residues 40-536 of SEQ ID NO: 54, or relative to the reference sequence corresponding to SEQ ID NO: 54.

31. The engineered glucocerebrosidase of claim 20, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 220, or to the reference sequence corresponding to SEQ ID NO: 220, wherein the amino acid sequence comprises one or more substitutions relative to the reference sequence corresponding to residues 40-536 of SEQ ID NO: 220, or relative to the reference sequence corresponding to SEQ ID NO: 220.

32. The engineered glucocerebrosidase of claim 21, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 924-1020, or to the reference sequence corresponding to SEQ ID NO: 924-1020, wherein the amino acid sequence comprises one or more substitutions relative to the reference sequence corresponding to residues 40-536 of SEQ ID NO: 220, or relative to the reference sequence corresponding to SEQ ID NO: 220.

33. The engineered glucocerebrosidase of claim 31 or 32, wherein the amino acid sequence of the engineered glucocerebrosidase comprises at least a substitution or substitution set at amino acid position(s) 65/195/361, 514, 79/195/263/361, 65/374, 79/195, 65/230, 65/233/263/337/359/361/374, 79/536, 79/230/233/239/359/361/471, 65/471, 374, 65/195/230/233/337/374/514, 65/195/233/359/536, 230, 65/195/263/276/359/361/374/471, 79, 195/230/233/337/374/536, 195/230, 195/230/337/359/361, 230/263, 65/230/233, 263, 195/230/337/361/374/471, 65/195, 374/471, 374/536, 239, 65, 230/233/536, 230/263/374, 65/195/230/263/337/361/374/471, 79/374, 65/361/374/471/536, 195, 233, 79/230/359/374, 536, 230/359/361/374/514, 65/79/230, 195/239/263/361/374/514, 195/263/374, 195/374, 65/230/233/361/536, 230/233/374/471, 65/233/359/361/471, 65/359/471, 65/79, or 65/359, wherein the amino acid positions are relative to the reference sequence corresponding to residues 40-536 of SEQ ID NO: 220, or relative to the reference sequence corresponding to SEQ ID NO: 220.

34. The engineered glucocerebrosidase of any one of claims 31-33, wherein the amino acid sequence of the engineered glucocerebrosidase comprises at least a substitution or substitution set 65V/195W/361W, 514E, 79V/195W/263G/361W, 65V/374T, 79V/195W, 65V/230Y, 65V/233A/263G/337N/359P/361W/374T, 79V/536D, 79V/230Y/233A/239R/359P/361W/471T, 65V/471T, 374T, 65V/195W/230Y/233A/337N/374T/514E, 65V/195W/233A/359P/536D, 230Y, 65V/195W/263G/276T/359P/361W/374T/471T, 79V, 195W/230Y/233A/337N/374T/536D, 195W/230Y, 195W/230Y/337N/359P/361W, 230Y/263G, 65V/230Y/233G, 263G, 195W/230Y/337N/361W/374T/471T, 65V/195W, 374T/471T, 374T/536D, 239R, 65V, 230Y/233G/536L, 230Y/263G/374T, 65V/195W/230Y/263G/337N/361W/374T/471T, 79V/374T, 65V/361W/374T/471T/536D, 195W, 233G, 79V/230Y/359P/374T, 536D, 230Y/359P/361W/374T/514E, 65V/79V/230Y, 195W/239R/263G/361W/374T/514E, 195W/263G/374T, 195W/374T, 65V/230Y/233A/361W/536L, 230Y/233A/374T/471T, 65V/233A/359P/361W/471T, 536L, 65V/359P/471T, 65V/79V, or 65V/359P, wherein the amino acid positions are relative to the reference sequence corresponding to residues 40-536 of SEQ ID NO: 220, or relative to the reference sequence corresponding to SEQ ID NO: 220.

35. The engineered glucocerebrosidase of claim 20, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 984, or to the reference sequence corresponding to SEQ ID NO: 984, wherein the amino acid sequence comprises one or more substitutions relative to the reference sequence corresponding to residues 40-536 of SEQ ID NO: 984, or relative to the reference sequence corresponding to SEQ ID NO: 984.

36. The engineered glucocerebrosidase of claim 21, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 1022-1216, or to the reference sequence corresponding to SEQ ID NO: 1022-1216, wherein the amino acid sequence comprises one or more substitutions relative to the reference sequence corresponding to residues 40-536 of SEQ ID NO: 984, or relative to the reference sequence corresponding to SEQ ID NO: 984.

37. The engineered glucocerebrosidase of claim 35 or 36, wherein the amino acid sequence of the engineered glucocerebrosidase comprises at least a substitution or substitution set at amino acid position(s) 359, 79/359, 79/97/182, 97/359, 97, 79/182, 201, 404, 484, 446, 494/495, 495, 412, 385, 100, 184, 82, 75, 71, 41, 99, 94, 92, 98, 114, 95, 102, 96, 105, 113, 325, 336, 262, 386, 360, 400, 342, 390, 264, 298, 382, 408, 449, 459, 434, 476,502, 29/534, 497, 485, 533, 536, 510, 532, 489, 534, 494, 182, 137, 204, 194, 202, 144, 142,175, 220, 150, or 206, wherein the amino acid positions are relative to the reference sequence corresponding to residues 40-536 of SEQ ID NO: 984, or relative to the reference sequence corresponding to SEQ ID NO: 984.

38. The engineered glucocerebrosidase of any one of claims 35-37, wherein the amino acid sequence of the engineered glucocerebrosidase comprises at least a substitution or substitution set 359P, 79V/359P, 79V/97R/182M, 97R/359P, 97R, 79V/182M, 201T, 404Q, 484E, 201R, 446N, 494T/495G, 495G, 412W, 385N, 385G, 100N, 184V, 385H, 82S, 75A, 71T, 41H, 97K, 99E, 97Q, 94N, 100R, 92A, 98P, 99W, 92G, 114Y, 95H, 102A, 92K, 96D, 94E, 96E, 95W, 105I, 113Q, 92H, 92Q, 92E, 325V, 336E, 385Q, 262Q, 386I, 360E, 400T, 342Q, 400E, 400Q, 390A, 264I, 298W, 382A, 408D, 449E, 459M, 434G, 476A, 408E, 502K, wherein the amino acid sequence comprises one or more substitutions relative to the reference sequence corresponding to residues 40-536 of SEQ ID NO: 984, or relative to the reference sequence corresponding to SEQ ID NO: 984.

39. The engineered glucocerebrosidase of claim 20, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of SEQ ID NO: 1150, or to the reference sequence corresponding to SEQ ID NO: 1150, wherein the amino acid sequence comprises one or more substitutions relative to the reference sequence corresponding to residues 40-536 of SEQ ID NO: 1150, or relative to the reference sequence corresponding to SEQ ID NO: 1150.

40. The engineered glucocerebrosidase of claim 21, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 40-536 of an even-numbered SEQ ID NO. of SEQ ID NOs: 1218-1554, or to the reference sequence corresponding to SEQ ID NO: 1218-1554, wherein the amino acid sequence comprises one or more substitutions relative to the reference sequence corresponding to residues 40-536 of SEQ ID NO: 1150, or relative to the reference sequence corresponding to SEQ ID NO: 1150.

41. The engineered glucocerebrosidase of claim 39 or 40, wherein the amino acid sequence comprises at least a substitution or substitution set at amino acid position(s) 96/194/382/484, 359/382/412/494/495, 92/96/175/182/382, 96/359/382/484/494/495, 92/175/194/359/382, 359/382/400/412, 92/96/182/382/400/484, 92/96/382/494/495, 92/96/175/382/494/495, 96/182/359/382/484, 92/96/382, 92/382/494/495/534, 92/96/382/400/484, 92/96/175/382, 92/175/382/400/484, 92/96/175/182/382/412/484, 92/96/201/382/484, 92/182/194/382, 92/96/175/194/359/382/400, 92/359/382, 96/382, 92/175/382/484/494/495, 96/201/359/382, 92, 194, 201, 359, 96, 92/96/382/400/484/495, 382/495, 382, 92/382, 71/175/325/385, 325/385, 71/325/502, 71/175/325/385/502, 202/325/385, 97/175/385/510, 325/385/510, 100/175/325, 71/175/325/360/385, 97/175/510, 71/97/100/175/325/385, 71/175/510, 71/97/325/502, 71/100/325/385, 202/385/489, 325/385/502, 71/100/325/385/510, 71/100/175/325/385, 385/510, 97/325/385, 71, 97/202/325/360/385/510, 502, 71/97/103/325, 100/325/385/502/510, 71/137/175/325/360/385/502, 71/276/325/385/510, 71/175/360/385/502, 100/175/325/385/489/502, 71/175/360/385/485/502, 95/113, 494/536, 95/99/113/182/184/264/386, 184/536, 95/142/144/182/184/386/449, 144/204/386/400/449, 113/449/536, 536, 95/386/536, 264, 99/113/386/449, 95/386, 95/99/204/386, 113/142/386/449, 182/184/264/494, 113/142/182/494, 113, 99/264/449, 95, 95/99, 99/113/449, 95/113/449/536, 95/99/113, 386/536, 99/182/184, 142/386/400/536, 35/264/449/536, 142/144/220, 99/142, 29/102/182/276/336/390/459/502/532, 41/262/336/502, 29/97/150/262/336/502/532, 29, 532, 102/390/459/502/532, 29/262/276/336/495, 29/182/262/459/532, 92/99/102/502, 29/97/102/182/502/532, 336/459/495/502/532, 150/182/336/390, 150/262/495, 29/495, 29/102/182/262/502, 29/336/459/502/532, 182/495/502/532, 102/336, 150/182/262/495, 459/495/502/532, 41/182/390/495, 29/41/182/336/495, 97/102/495, 182/262/495, 29/262/502, 29/102/502, 182/336/495, 29/150/182/262/459/495, 502/532, 386, 497, 99, 512, 69, 507, 445, 75, 487, 184, 90, 235, 434, 489, 86, 182, 446, 490, 299, 102, 230, 68, 141, 97, 316, 522, 224, 478, 480, 368, 412, 449, 265, or 494, wherein the amino acid positions are relative to the reference sequence corresponding to residues 40-536 of SEQ ID NO: 1150, or relative to the reference sequence corresponding to SEQ ID NO: 1150.

42. The engineered glucocerebrosidase of any one of claims 39-41, wherein the amino acid sequence comprises at least a substitution or substitution set 96E/194Q/382A/484E, 359P/382A/412W/494T/495G, 92Q/96E/175D/182S/382A, 96E/359P/382A/484E/494T/495G, 92E/175D/194Q/359P/382A, 359P/382A/400E/412W, 92Q/96E/182S/382A/400E/484E, 92Q/96E/382A/494T/495G, 92Q/96E/175D/382A/494T/495G, 96E/182S/359P/382A/484E, 92E/96E/382A, 92Q/382A/494T/495G/534K, 92E/96E/382A/400Q/484E, 92Q/96E/175D/382A, 92Q/175D/382A/400Q/484E, 92E/96E/175D/382A/494T/495G, 92Q/96E/175D/182S/382A/412W/484E, 92Q/96E/201Q/382A/484E, 92E/182S/194Q/382A, 92Q/96E/175D/194Q/359P/382A/400E, 92Q/359P/382A, 96E/382A, 92Q/175D/382A/484E/494T/495G, 96E/201Q/359P/382A, 92E, 92Q, 194Q, 201Q, 359P, 96E, 92E/96E/382A/400E/484E/495G, 382A/495G, 382A, 92E/382A, 71T/175D/325V/385H, 325V/385H, 71T/325V/502Q, 71T/175D/325V/385H/502Q, 202Q/325V/385N, 97Q/175D/385H/510S, 325V/385Q/510S, 100R/175D/325V, 71T/175D/325V/360E/385N, 97Q/175D/510S, 71T/97Q/100R/175D/325V/385H, 71T/175D/510S, 71T/97Q/325V/502Q, 71T/100R/325V/385Q, 202Q/385N/489E, 325V/385N/502Q, 325V/385H/502Q, 71T/100R/325V/385H/510S, 71T/100R/175D/325V/385N, 71T/100R/175D/325V/385H, 385N/510S, 97Q/325V/385N, 71T, 97Q/202Q/325V/360E/385G/510S, 502Q, 71T/97Q/103S/325V, 100R/325V/385H/502Q/510S, 71T/137Q/175D/325V/360E/385H/502Q, 71T/276T/325V/385R/510S, 71T/175D/360E/385N/502Q, 100R/175D/325V/385N/489E/502Q, 71T/175D/360E/385H/485S/502Q, 95W/113Q, 494T/536P, 95W/99W/113Q/182D/184V/264I/386I, 184Q/536P, 95W/142N/144M/182G/184Q/386I/449E, 144M/204Q/386I/400T/449E, 113Q/449E/536P, 536P, 95W/386I/536P, 264I, 99W/113Q/386I/449E, 95W/386I, 95W/99W/204Q/386I, 113Q/142N/386I/449E, 182G/184Q/264I/494T, 113Q/142N/182D/494T, 113Q, 99W/264I/449E, 95W, 95W/99W, 99W/113Q/449E, 95W/113Q/449E/536P, 95W/99W/113Q, 386I/536P, 99W/182G/184Q, 142N/386I/400T/536P, 35T/264I/449E/536P, 142N/144M/220A, 99W/142N, 29I/102A/182M/276T/336E/390A/459M/502K/532V, 41H/262Q/336E/502K, 29I/97R/150T/262Q/336E/502K/532V, 29I, 532V, 102A/390A/459M/502K/532V, 29I/262Q/276T/336E/495G, 29I/182T/262Q/459M/532V, 92K/99G/102A/502K, 29I/97R/102A/182M/502K/532V, 336E/459M/495S/502K/532V, 150T/182T/336E/390A, 150T/262Q/495G, 29I/495S, 29I/102A/182T/262Q/502K, 29I/336E/459M/502K/532V, 182M/495G/502K/532V, 102A/336E, 150T/182M/262Q/495S, 459M/495S/502K/532V, 41H/182M/390A/495G, 29I/41H/182T/336E/495S, 97R/102A/495S, 182T/262Q/495S, 29I/262Q/502K, 29I/102A/502K, 182M/336E/495S, 502K, 29I/150T/182T/262Q/459M/495G, 502K/532V, 386P, 497M, 99K, 512H, 69D, 507I, 445N, 75N, 75H, 512Q, 487S, 113P, 184I, 90M, 235T, 434Q, 445G, 489N, 90A, 386L, 75E, 86E, 489Q, 182E, 446H, 490N, 299V, 96N, 102P, 230F, 68T, 141E, 97E, 316H, 522S, 224M, 478N, 480G, 368D, 86H, 412N, 449S, 265N, or 494A, wherein the amino acid positions are relative to the reference sequence corresponding to residues 40-536 of SEQ ID NO: 1150, or relative to the reference sequence corresponding to SEQ ID NO: 1150.

43. The engineered glucocerebrosidase of claim 1, wherein the amino acid sequence of the engineered glucocerebrosidase comprises at least one substitution set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

44. The engineered glucocerebrosidase of claim 1, wherein the amino acid sequence of the engineered glucocerebrosidase comprises at least a substitution or substitution set of an engineered glucocerebrosidase set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

45. The engineered glucocerebrosidase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising a substitution or substitution set of an engineered glucocerebrosidase set forth in Tables 3.1, 3.2, 4.1, 4.2, 4.3, 4.4, 5.1, 6.1, 6.2, 12.1, 12.2, and 13.1, wherein the amino acid positions are relative to the reference sequence corresponding to residues 40-536 of SEQ ID NO: 54, 220, 984, or 1150, or relative to the reference sequence corresponding to SEQ ID NO: 54, 220, or 984, or 1150.

46. The engineered glucocerebrosidase of claim 1, comprising an amino acid sequence comprising residues 40-536 of an engineered glucocerebrosidase set forth in Tables 3.1, 3.2, 4.1, 4.2, 4.3, 4.4, 5.1, 6.1, and 6.2, or an amino acid sequence comprising an engineered glucocerebrosidase set forth in Tables 3.1, 3.2, 4.1, 4.2, 4.3, 4.4, 5.1, 6.1, and 6.2.

47. The engineered glucocerebrosidase of claim 1, wherein the amino acid sequence of the engineered glucocerebrosidase comprises residues 40-536 of an even-numbered SEQ ID NO. of SEQ ID NOs: 4-1648, or comprises an even-numbered SEQ ID NO. of SEQ ID NOs: 4-1648, optionally wherein the amino acid sequence has 1, 2, 3, 4, 5, 6, 7, 8, 9, or up to 10 substitutions.

48. The engineered glucocerebrosidase of claim 1, wherein the amino acid sequence of the engineered glucocerebrosidase comprises residues 40-536 of SEQ ID NO: 54, 220, 984, or 1150, or comprises SEQ ID NO: 54, 220, 984, or 1150, optionally wherein the amino acid sequence has 1, 2, 3, 4, 5, 6, 7, 8, 9, or up to 10 substitutions.

49. The engineered glucocerebrosidase of any one of claims 1-48, wherein the engineered glucocerebrosidase has glucocerebrosidase and one or more improved properties.

50. The engineered glucocerebrosidase of any one of claims 1-49, wherein the engineered glucocerebrosidase has glucocerebrosidase activity and an improved property selected from: i) increased enzymatic activity; ii) increased expression or expression efficiency; iii) increased stability at neutral or basic pH; iv) increased stability at acidic pH; v) increased stability to serum or plasma; vi) increased thermostability; vii) increased uptake and/or intracellular stability in cells such as macrophages; and viii) reduced immunogenicity, or any combination of i), ii), iii), iv), v), vi), vii) and viii) as compared to a reference glucocerebrosidase having a sequence corresponding to residues 40-536 of SEQ ID NO: 2, 54, 220, 984, or 1150, or a sequence corresponding to SEQ ID NO: 2, 54, 220, 984, or 1150.

51. The engineered glucocerebrosidase of claim 49 or 50, wherein the reference glucocerebrosidase has a sequence corresponding to residues 40-536 of SEQ ID NO: 2, or to the sequence corresponding to SEQ ID NO: 2.

52. The engineered glucocerebrosidase of claims 1-51 wherein the engineered glucocerebrosidase is a purified preparation.

53. A recombinant polynucleotide comprising a polynucleotide sequence encoding an engineered glucocerebrosidase of any one of claims 1-51.

54. The recombinant polynucleotide of claim 52, comprising a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 118-1608 of SEQ ID NO. 53, 219, 983, or 1149, or to a reference polynucleotide sequence corresponding to SEQ ID NO: 53, 219, 983, or 1149, wherein the recombinant polynucleotide encodes a glucocerebrosidase.

55. The recombinant polynucleotide of claim 52, comprising a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 118-1608 of an odd-numbered SEQ ID NO. of SEQ ID NOs: 3-1647, or to a reference polynucleotide sequence corresponding to an odd-numbered SEQ ID NO. of SEQ ID NOs: 3-1647, wherein the recombinant polynucleotide encodes a glucocerebrosidase.

56. The recombinant polynucleotide of any one of claims 53-55, wherein the polynucleotide sequence is codon-optimized for expression of the encoded glucocerebrosidase.

57. The recombinant polynucleotide of claim 53, comprising a polynucleotide sequence comprising residues 118-1608 of an odd-numbered SEQ ID NO. of SEQ ID NOs: 3-1647, or a polynucleotide sequence comprising an odd-numbered SEQ ID NO. of SEQ ID NOs: 3-1647.

58. The recombinant polynucleotide of claim 53, comprising a polynucleotide sequence comprising residues 118-1608 of SEQ ID NO: 53, 219, 983, or 1149, or comprises SEQ ID NO: 53, 219, 983, or 1149.

59. An expression vector comprising a recombinant polynucleotide of any one of claims 53-58.

60. The expression vector of claim 59, wherein the recombinant polynucleotide is operably linked to a control sequence.

61. The expression vector of claim 60, wherein the control sequence comprises at least a promoter.

62. The expression vector of any one of claims 59-61, wherein the expression vector comprises a viral vector.

63. A host cell comprising an expression vector of any one of claims 59-62.

64. The host cell of claim 63, comprising a mammalian cell.

65. The host cell of claim 64, comprising a human cell.

66. A method of producing an engineered glucocerebrosidase in a host cell, comprising culturing a host cell of any one of claims 63-65 under suitable culture conditions such that the engineered glucocerebrosidase is expressed.

67. The method of claim 66, further comprising recovering the engineered glucocerebrosidase from the culture and/or host cells.

68. The method of claim 66 or 67, further comprising purifying the engineered glucocerebrosidase.

69. A pharmaceutical composition comprising the engineered glucocerebrosidase of any one of claims 1-52, the recombinant polynucleotide of any one of claims 53-58, or the expression vector of any one of claims 59-62.

70. The pharmaceutical composition of claim 69, further comprising a pharmaceutically acceptable carrier and/or excipient.

71. The pharmaceutical composition of claim 69 or 70, wherein the composition is suitable for parenteral injection or infusion to a human.

72. A gene therapy composition comprising a recombinant polynucleotide of any one of claims 53-58.

73. The gene therapy composition of claim 72, wherein the gene therapy composition comprises a viral vector comprising the recombinant polynucleotide.

74. The gene therapy composition of claim 73, wherein the viral vector is adenovirus, adeno-associated virus, lentivirus, retrovirus, or herpes virus vector.

75. The gene therapy composition of claim 72, wherein the recombinant polynucleotide is formulated in liposomes, cationic polymers, dendrimers, or is conjugated to a cell-penetrating peptide.

76. A method for treating and/or preventing the symptoms of deficiency in glucocerebrosidase activity in a subject, comprising administering to a subject having a deficiency in glucocerebrosidase activity an effective amount of the engineered glucocerebrosidase of any one of claims 1-52, the pharmaceutical composition of any one of claims 69-71, or the gene therapy composition of any one of claims 72-75.

77. The method of claim 76, wherein the subject has Gaucher disease.

78. The method of claim 76 or 77, wherein the symptoms of deficiency in glucocerebrosidase activity are ameliorated.

79. Use of the engineered glucocerebrosidase of any one of claims 1-52, the pharmaceutical composition of any one of claims 69-71, or the gene therapy composition of any one of claims 72-75 for the treatment of a deficiency in glucocerebrosidase activity in a subject.

80. Use of the engineered glucocerebrosidase of any one of claims 1-52 or the gene therapy composition of any one of claims 72-75 in the preparation of a medicament for treating a deficiency in glucocerebrosidase activity in a subject.

81. The use of claim 79 or 80, wherein the deficiency in glucocerebrosidase activity is Gaucher disease.
